(19) United States
(12) Patent Application Publication
(10) Pub. No.: US 2022/0079924 A1
(43) Pub. Date: Mar. 17, 2022

(54) GLUCOCORTICOID RECEPTOR MODULATORS

(71) Applicant: ORIC Pharmaceuticals, Inc., South San Francisco, CA (US)

(72) Inventors: Xiaohui DU, Belmont, CA (US); John EKSTEROWICZ, Burlingame, CA (US); Valeria R. FANTIN, Burlingame, CA (US); Yosup REW, Foster City, CA (US); Daqing SUN, Foster City, CA (US); Qiuping YE, Foster City, CA (US); Haiying ZHOU, Castro Valley, CA (US); Hiroyuki KAWAI, Pacifica, CA (US); Jared MOORE, San Rafael, CA (US); Johnny PHAM, San Bruno, CA (US); Kejia WU, South San Francisco, CA (US); Liusheng ZHU, Foster City, CA (US)

(21) Appl. No.: 17/283,700

(22) PCT Filed: Oct. 9, 2019
(86) PCT No.: PCT/US2019/055464

§ 371 Date: Apr. 8, 2021

Related U.S. Application Data
(60) Provisional application No. 62/744,054, filed on Oct. 10, 2018.

(51) Int. Cl. A61K 31/427; C07D 417/14; C07D 401/14; A61K 31/4439; A61K 31/277; A61K 31/58; A61K 31/496; A61K 31/573; A61K 31/167; A61K 31/404; A61K 31/473; A61K 31/4166; A61K 33/243; A61K 31/282; A61K 31/337; A61K 31/7068; A61K 31/704; A61K 31/4745; A61K 31/519; A61K 35/17

(52) U.S. Cl. CPC A61K 31/427; A61K 35/17; C07D 401/14; A61K 31/4439; A61K 31/277; A61K 31/58; A61K 31/496; A61K 31/573; A61K 31/167; A61K 31/404; A61K 31/473; A61K 31/4166; A61K 33/243; A61K 31/282; A61K 31/337; A61K 31/7068; A61K 31/704; A61K 31/4745; A61K 31/519; C07D 417/14

(57) ABSTRACT

Described herein are glucocorticoid receptor modulators and pharmaceutical compositions comprising said compounds. The subject compounds and compositions are useful for the treatment of cancer and hypercortisolism.

### CROSS REFERENCE

This application claims the benefit of U.S. Application Ser. No. 62/744,054 filed Oct. 10, 2018, which is hereby incorporated by reference in its entirety.

### BACKGROUND

A need exists in the art for an effective treatment of cancer, neoplastic disease, and hypercortisolism.

### BRIEF SUMMARY OF THE INVENTION

Provided herein are compounds of Formula (I), (Ia), (Ib), (Ic), (II), (III), (A), (B), (C), or (D), and pharmaceutical compositions comprising said compounds. The subject compounds and compositions are useful as glucocorticoid receptor (GR) modulators. Furthermore, the subject compounds and compositions are useful for the treatment of cancer, such as prostate cancer, breast cancer, lung cancer, ovarian cancer, and hypercortisolism.

Disclosed herein is a compound having the structure of Formula (I), or a pharmaceutically acceptable salt, solvate, stereoisomer, or isotopic variant thereof:

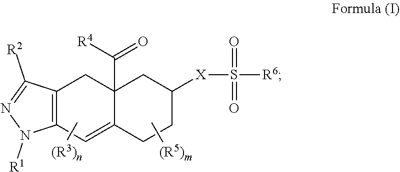

wherein:
R¹ is cycloalkyl, heterocycloalkyl, or heteroaryl; wherein the cycloalkyl, heterocycloalkyl, and heteroaryl are independently optionally substituted with one, two, or three R^{1a};
each R^{1a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{1a} on the same carbon form an oxo;
R² is hydrogen, halogen, C₁-C₆ alkyl, or C₁-C₆ haloalkyl;
each R³ is independently halogen, C₁-C₆ alkyl, or C₁-C₆ haloalkyl;
R⁴ is cycloalkyl, heterocycloalkyl, aryl, or heteroaryl; wherein the cycloalkyl, heterocycloalkyl, aryl, and heteroaryl are independently optionally substituted with one, two, or three R^{4a};
each R^{4a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{4a} on the same carbon form an oxo;
or two R^{4a} are taken together to form a cycloalkyl or a heterocycloalkyl;
each R⁵ is independently halogen, C₁-C₆ alkyl, or C₁-C₆ haloalkyl;
R⁶ is aryl, heteroaryl, cycloalkyl, or heterocycloalkyl; wherein the aryl, heteroaryl, cycloalkyl, and heterocycloalkyl are independently optionally substituted with one, two, or three R^{6a};
each R^{6a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{6a} on the same carbon form an oxo;
X is a bond, —C(R⁷)₂—, or —NR⁸—;
each R⁷ is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{7a};
each R^{7a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{7a} on the same carbon form an oxo;
R⁸ is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{8a};
each R^{8a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{8a} on the same carbon form an oxo;
each R^{a} is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{b} is independently C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{c} and R^{d} are independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three, halogen, —OH, —NH₂, or C₁-C₆ alkyl;
or R^{c} and R^{d} are taken together with the nitrogen atom to which they are attached to form a heterocycloalkyl optionally substituted with one, two, or three halogen, —OH, —NH₂, or C₁-C₆ alkyl;
m is 0-4; and
n is 0-3;
   provided that the compound is not

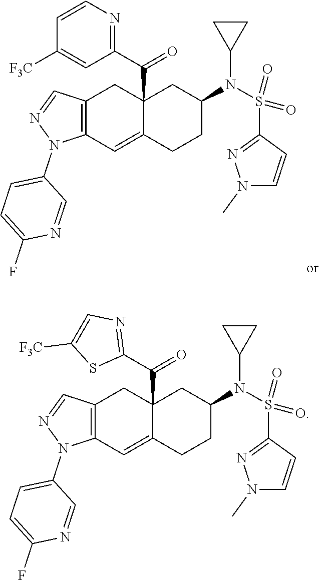 [SMILES: Cn1ccc(S(=O)(=O)N(C2CC2)[C@H]2CCC3=Cc4c(cnn4-c4ccc(F)nc4)C[C@]3(C(=O)c3cc(C(F)(F)F)ccn3)C2)n1 ; Cn1ccc(S(=O)(=O)N(C2CC2)[C@H]2CCC3=Cc4c(cnn4-c4ccc(F)nc4)C[C@]3(C(=O)c3ncc(C(F)(F)F)s3)C2)n1]

Also disclosed herein are pharmaceutical composition comprising a compound disclosed herein, or a pharmaceutically acceptable salt, solvate, stereoisomer, or isotopic variant thereof, and at least one pharmaceutically acceptable excipient.

Also disclosed herein are methods for treating or preventing cancer in a subject, the method comprising administering a therapeutically effective amount of a compound disclosed herein, or a pharmaceutically acceptable salt, solvate, stereoisomer, or isotopic variant thereof, to the subject in need thereof.

Also disclosed herein are methods of reducing incidences of cancer recurrence, the method comprising administering a therapeutically effective amount of a compound disclosed herein, or a pharmaceutically acceptable salt, solvate, stereoisomer, or isotopic variant thereof, to the subject in need thereof.

Also disclosed herein are methods for treating a therapy-resistant cancer in a subject, the method comprising administering a therapeutically effective amount of a compound disclosed herein, or a pharmaceutically acceptable salt, solvate, stereoisomer, or isotopic variant thereof, to the subject in need thereof.

Also disclosed herein are methods for treating a hypercortisolism disease or disorder in a subject, the method comprising administering a therapeutically effective amount of a compound disclosed herein, or a pharmaceutically acceptable salt, solvate, stereoisomer, or isotopic variant thereof, to the subject in need thereof.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Alkyl” refers to an optionally substituted straight-chain, or optionally substituted branched-chain saturated hydrocarbon monoradical having from one to about ten carbon atoms, or from one to six carbon atoms, wherein a sp3-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₆ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a Ch alkyl. Unless stated otherwise specifically in the specification, an alkyl group is optionally substituted for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp2-hybridized carbon of the alkenyl residue is attached to the rest of the molecule by a single bond. The group may be in either the cis or tram conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

“Hydroxyalkyl” refers to an alkyl radical, as defined above, that is substituted by one or more —OH e.g., hydroxymethyl, hydroxyethyl, hydroxypropyl, hydroxybutyl, hydroxypentyl, dihydroxymethyl, dihydroxyethyl, dihydroxypropyl, dihydroxybutyl, dihydroxypentyl, and the like.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The terms “inhibit,” “block,” “suppress,” and grammatical variants thereof are used interchangeably herein and refer to any statistically significant decrease in biological activity, including full blocking of the activity. In some embodiments, “inhibition” refers to a decrease of about 10%, about 20%, about 30%, about 40%, about 50%, about 60%, about 70%, about 80%, about 90% or about 100% in biological activity. In one aspect, the compound disclosed herein inhibit the glucocorticoid receptor activity by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or about 100%, as determined, for example, by the methods described in the Examples and/or methods known in the art.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Compounds

Described herein are compounds of Formula (I), (Ia), (Ib), (Ic), (II), (III), (A), (B), (C), or (D), or a pharmaceutically acceptable salt, solvate, stereoisomer, or isotopic variant thereof, that are GR modulators. These compounds, and compositions comprising these compounds, are useful for the treatment of cancer, neoplastic disease, and hypercortisolism diseases and disorders.

In some embodiments provided herein is a compound having the structure of Formula (I), or a pharmaceutically acceptable salt, solvate, stereoisomer, or isotopic variant thereof:

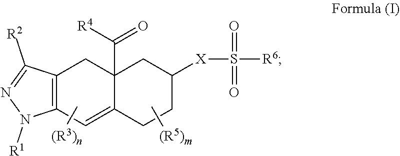

wherein:
R¹ is cycloalkyl, heterocycloalkyl, or heteroaryl; wherein the cycloalkyl, heterocycloalkyl, and heteroaryl are independently optionally substituted with one, two, or three R^{1a};
each R^{1a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{1a} on the same carbon form an oxo;
R² is hydrogen, halogen, C₁-C₆ alkyl, or C₁-C₆ haloalkyl;
each R³ is independently halogen, C₁-C₆ alkyl, or C₁-C₆ haloalkyl;
R⁴ is cycloalkyl, heterocycloalkyl, aryl, or heteroaryl; wherein the cycloalkyl, heterocycloalkyl, aryl, and heteroaryl are independently optionally substituted with one, two, or three R^{4a};
each R^{4a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{4a} on the same carbon form an oxo;
or two R^{4a} are taken together to form a cycloalkyl or a heterocycloalkyl;
each R⁵ is independently halogen, C₁-C₆ alkyl, or C₁-C₆ haloalkyl;
R⁶ is aryl, heteroaryl, cycloalkyl, or heterocycloalkyl; wherein the aryl, heteroaryl, cycloalkyl, and heterocycloalkyl are independently optionally substituted with one, two, or three R^{6a};
each R^{6a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{6a} on the same carbon form an oxo;
X is a bond, —C(R⁷)₂—, or —NR⁸—;
each R⁷ is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{7a};
each R^{7a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{7a} on the same carbon form an oxo;
R⁸ is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{8a};
each R^{8a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{8a} on the same carbon form an oxo;
each R^{a} is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{b} is independently C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{c} and R^{d} are independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three, halogen, —OH, —NH₂, or C₁-C₆ alkyl;
or R^{c} and R^{d} are taken together with the nitrogen atom to which they are attached to form a heterocycloalkyl optionally substituted with one, two, or three halogen, —OH, —NH₂, or C₁-C₆ alkyl;
m is 0-4; and
n is 0-3;
provided that the compound is not

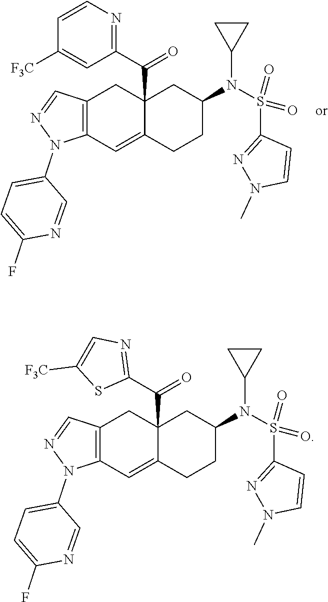 [SMILES: Cn1ccc(S(=O)(=O)N(C2CC2)[C@H]2CCC3=Cc4c(cnn4-c4ccc(F)nc4)C[C@]3(C(=O)c3cc(C(F)(F)F)ccn3)C2)n1 ; Cn1ccc(S(=O)(=O)N(C2CC2)[C@H]2CCC3=Cc4c(cnn4-c4ccc(F)nc4)C[C@]3(C(=O)c3ncc(C(F)(F)F)s3)C2)n1]

In some embodiments of a compound of Formula (I), R² is hydrogen or halogen. In some embodiments of a compound of Formula (I), R² is hydrogen or C₁-C₆ alkyl. In some embodiments of a compound of Formula (I), R² is hydrogen.

In some embodiments of a compound of Formula (I), each R³ is independently halogen or C₁-C₆ alkyl. In some embodiments of a compound of Formula (I), each R³ is independently halogen. In some embodiments of a compound of Formula (I), each R³ is independently C₁-C₆ alkyl.

In some embodiments of a compound of Formula (I), n is 0-2. In some embodiments of a compound of Formula (I), n is 0. In some embodiments of a compound of Formula (I), n is 1. In some embodiments of a compound of Formula (I), n is 2. In some embodiments of a compound of Formula (I), n is 3.

In some embodiments of a compound of Formula (I), each R⁵ is independently halogen or C₁-C₆ alkyl. In some embodiments of a compound of Formula (I), each R⁵ is independently halogen. In some embodiments of a compound of Formula (I), each R⁵ is independently C₁-C₆ alkyl.

In some embodiments of a compound of Formula (I), m is 0-2. In some embodiments of a compound of Formula (I), m is 0-3. In some embodiments of a compound of Formula (I), m is 0. In some embodiments of a compound of Formula (I), m is 1. In some embodiments of a compound of Formula (I), m is 2. In some embodiments of a compound of Formula (I), m is 3. In some embodiments of a compound of Formula (I), m is 4.

In some embodiments of a compound of Formula (I), X is a bond.

In some embodiments of a compound of Formula (I), the compound is of Formula (Ia):

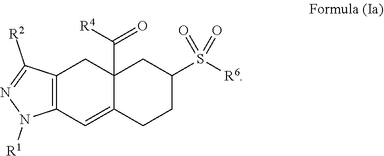

In some embodiments of a compound of Formula (I), X is —C(R⁷)₂—.

In some embodiments of a compound of Formula (I), the compound is of Formula (Ib):

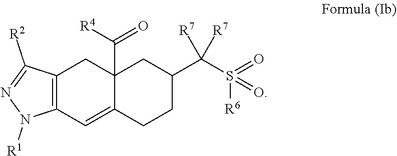

In some embodiments of a compound of Formula (I) or (Ib), each R⁷ is independently hydrogen, C₁-C₆ alkyl, C₁-C₆ haloalkyl, cycloalkyl, or heterocycloalkyl, wherein the alkyl, cycloalkyl, and heterocycloalkyl are independently optionally substituted with one, two, or three R^{7a}. In some embodiments of a compound of Formula (I) or (Ib), each R⁷ is independently hydrogen, C₁-C₆ alkyl, C₁-C₆ haloalkyl, or cycloalkyl, wherein the alkyl, and cycloalkyl are independently optionally substituted with one, two, or three R^{7a}. In some embodiments of a compound of Formula (I) or (Ib), each R⁷ is independently hydrogen, C₁-C₆ alkyl, C₁-C₆ haloalkyl, wherein the alkyls are independently optionally substituted with one, two, or three R^{7a}. In some embodiments of a compound of Formula (I) or (Ib), each R⁷ is independently hydrogen, C₁-C₆ alkyl, or C₁-C₆ haloalkyl. In some embodiments of a compound of Formula (I) or (Ib), each R⁷ is independently hydrogen or C₁-C₆ alkyl. In some embodiments of a compound of Formula (I) or (Ib), each R⁷ is independently C₁-C₆ alkyl. In some embodiments of a compound of Formula (I) or (Ib), each R⁷ is hydrogen.

In some embodiments of a compound of Formula (I) or (Ib), each R⁷ is optionally substituted with one R^{7a}. In some embodiments of a compound of Formula (I) or (Ib), each R⁷ is optionally substituted with one or two R^{7a}. In some embodiments of a compound of Formula (I) or (Ib), each R⁷ is optionally substituted with two R^{7a}. In some embodiments of a compound of Formula (I) or (Ib), each R⁷ is optionally substituted with two or three R^{7a}.

In some embodiments of a compound of Formula (I) or (Ib), each R^{7a} is independently halogen, —CN, —OR^{a}, —NR^{c}R^{d}, C₁-C₆ alkyl, C₁-C₆ haloalkyl, cycloalkyl, or heterocycloalkyl, wherein the alkyl, cycloalkyl, heterocycloalkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I) or (Ib), each R^{7a} is independently halogen, C₁-C₆ alkyl, or C₁-C₆ haloalkyl, wherein the alkyls are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I) or (Ib), each R^{7a} is independently halogen, C₁-C₆ alkyl, or C₁-C₆ haloalkyl. In some embodiments of a compound of Formula (I) or (Ib), each R^{7a} is independently halogen.

In some embodiments of a compound of Formula (I), X is —NR⁸—.

In some embodiments of a compound of Formula (I), the compound is of Formula (Ic):

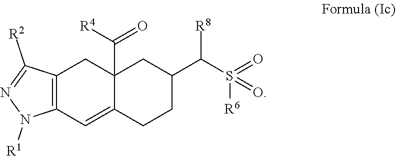

In some embodiments of a compound of Formula (I) or (Ic), R⁸ is hydrogen, C₁-C₆ alkyl, C₁-C₆ haloalkyl, C₁-C₆ hydroxyalkyl, C₁-C₆ aminoalkyl, heterocycloalkyl, aryl, or heteroaryl; wherein the alkyl, heterocycloalkyl, aryl, and heteroaryl are independently optionally substituted with one, two, or three R^{8a}. In some embodiments of a compound of Formula (I) or (Ic), R⁸ is hydrogen, C₁-C₆ alkyl, C₁-C₆ haloalkyl, cycloalkyl, or heterocycloalkyl, wherein the alkyl, cycloalkyl, and heterocycloalkyl are independently optionally substituted with one, two, or three R^{8a}. In some embodiments of a compound of Formula (I) or (Ic), R⁸ is hydrogen, C₁-C₆ alkyl, C₁-C₆ haloalkyl, or cycloalkyl, wherein the alkyl and cycloalkyl are independently optionally substituted with one, two, or three R^{8a}. In some embodiments of a compound of Formula (I) or (Ic), R⁸ is C₁-C₆ alkyl, C₁-C₆ haloalkyl, or cycloalkyl, wherein the alkyl and cycloalkyl are independently optionally substituted with one, two, or three R^{8a}. In some embodiments of a compound of Formula (I) or (Ic), R⁸ is C₁-C₆ alkyl or C₁-C₆ haloalkyl, wherein the alkyls are independently optionally substituted with one, two, or three R^{8a}. In some embodiments of a compound of Formula (I) or (Ic), R⁸ is C₁-C₆ alkyl optionally substituted with one, two, or three R^{8a}. In some embodiments of a compound of Formula (I) or (Ic), R⁸ is C₁-C₆ haloalkyl. In some embodiments of a compound of Formula (I) or (Ic), R⁸ is cycloalkyl. In some embodiments of a compound of Formula (I) or (Ic), R⁸ is C₁-C₆ alkyl.

In some embodiments of a compound of Formula (I) or (Ic), each R⁸ is optionally substituted with one R^{8a}. In some embodiments of a compound of Formula (I) or (Ic), each R⁸ is optionally substituted with one or two R^{8a}. In some embodiments of a compound of Formula (I) or (Ic), each R⁸ is optionally substituted with two R^{8a}. In some embodiments of a compound of Formula (I) or (Ic), each R⁸ is optionally substituted with two or three R^{8a}.

In some embodiments of a compound of Formula (I) or (Ic), each R^{8a} is independently halogen, —CN, —OR^{a}, —NR^{c}R^{d}, C₁-C₆ alkyl, C₁-C₆ haloalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I) or (Ic), each R^{8a} is independently halogen, —CN, —OR^{a}, —NR^{c}R^{d}, C₁-C₆ alkyl, C₁-C₆ haloalkyl, aryl, or heteroaryl; wherein the alkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I) or (Ic), each R^{8a} is independently halogen, —CN, —OR^{a}, —NR^{c}R^{d}, C₁-C₆ alkyl, C₁-C₆ haloalkyl, cycloalkyl, or heterocycloalkyl; wherein the alkyl, cycloalkyl, and heterocycloalkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I) or (Ic), each R^{8a} is independently halogen, —CN, —OR^{a}, —NR^{c}R^{d}, C₁-C₆ alkyl, C₁-C₆ haloalkyl, cycloalkyl, heterocycloalkyl, aryl, or heteroaryl. In some embodiments of a compound of Formula (I) or (Ic), each R^{8a} is independently halogen, —CN, —OR^{a}, —NR^{c}R^{d}, C₁-C₆ alkyl, C₁-C₆ haloalkyl, cycloalkyl, heterocycloalkyl, aryl, or heteroaryl. In some embodiments of a compound of Formula (I) or (Ic), each R^{8a} is independently halogen, —CN, —OR^{a}, —NR^{c}R^{d}, C₁-C₆ alkyl, or C₁-C₆ haloalkyl. In some embodiments of a compound of Formula (I) or (Ic), each R^{8a} is independently halogen, C₁-C₆ alkyl, or C₁-C₆ haloalkyl. In some embodiments of a compound of Formula (I) or (Ic), each R^{8a} is independently halogen.

In some embodiments of a compound of Formula (I), R⁶ is aryl or heteroaryl; wherein the aryl and heteroaryl are independently optionally substituted with one, two, or three R^{6a}.

In some embodiments of a compound of Formula (I), R⁶ is aryl optionally substituted with one, two, or three R^{6a}. In some embodiments of a compound of Formula (I), R⁶ is phenyl optionally substituted with one, two, or three R^{6a}.

In some embodiments of a compound of Formula (I), (Ia), (Ib), or (Ic), R⁶ is heteroaryl optionally substituted with one, two, or three R^{6a}. In some embodiments of a compound of Formula (I), (Ia), (Ib), or (Ic), R⁶ is a 5-membered heteroaryl optionally substituted with one, two, or three R^{6a}. In some embodiments of a compound of Formula (I), (Ia), (Ib), or (Ic), R⁶ is pyrrolyl, imidazolyl, pyrazolyl, or triazolyl optionally substituted with one, two, or three R^{6a}. In some embodiments of a compound of Formula (I), (Ia), (Ib), or (Ic), R⁶ is pyrrolyl, imidazolyl, or triazolyl optionally substituted with one, two, or three R^{6a}. In some embodiments of a compound of Formula (I), (Ia), (Ib), or (Ic), R⁶ is imidazolyl optionally substituted with one, two, or three R^{6a}. In some embodiments of a compound of Formula (I), (Ia), (Ib), or (Ic), R⁶ is triazolyl optionally substituted with one, two, or three R^{6a}. In some embodiments of a compound of Formula (I), (Ia), (Ib), or (Ic), R⁶ is pyrazolyl optionally substituted with one, two, or three R^{6a}. In some embodiments of a compound of Formula (I), (Ia), (Ib), or (Ic), R⁶ is not pyrazolyl. In some embodiments of a compound of Formula (I), (Ia), (Ib), or (Ic), R⁶ is a 6-membered heteroaryl optionally substituted with one, two, or three R^{6a}. In some embodiments of a compound of Formula (I), (Ia), (Ib), or (Ic), R⁶ is pyridyl optionally substituted with one, two, or three R^{6a}.

In some embodiments of a compound of Formula (I), (Ia), (Ib), or (Ic), each R⁶ is optionally substituted with one R^{6a}. In some embodiments of a compound of Formula (I), (Ia), (Ib), or (Ic), each R⁶ is optionally substituted with one or two R^{6a}. In some embodiments of a compound of Formula (I), (Ia), (Ib), or (Lc), each R⁶ is optionally substituted with two R^{6a}. In some embodiments of a compound of Formula (I), (Ia), (Ib), or (Ic), each R⁶ is optionally substituted with two or three R^{6a}.

In some embodiments of a compound of Formula (I), (Ia), (Ib), or (Ic), each R^{6a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I), (Ia), (Ib), or (Ic), each R^{6a} is independently halogen, C₁-C₆ alkyl, C₁-C₆ haloalkyl; wherein the alkyls are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I), (Ia), (Ib), or (Ic), each R^{6a} is independently halogen, C₁-C₆ alkyl, or C₁-C₆ haloalkyl. In some embodiments of a compound of Formula (I), (Ia), (Ib), or (Ic), each R^{6a} is independently C₁-C₆ alkyl. In some embodiments of a compound of Formula (I), (Ia), (Ib), or (Ic), each R^{6a} is independently halogen.

In some embodiments of a compound of Formula (I), (Ia), (Ib), or (Ic), R⁴ is heteroaryl optionally substituted with one, two, or three R^{4a}. In some embodiments of a compound of Formula (I), (Ia), (Ib), or (Ic), R⁴ is a 5-membered heteroaryl optionally substituted with one, two, or three R^{4a}. In some embodiments of a compound of Formula (I), (Ia), (Ib), or (Ic), R⁴ is thiazolyl optionally substituted with one, two, or three R^{4a}. In some embodiments of a compound of Formula (I), (Ia), (Ib), or (Ic), R⁴ is a 6-membered heteroaryl optionally substituted with one, two, or three R^{4a}. In some embodiments of a compound of Formula (I), (Ia), (Ib), or (Ic), R⁴ is pyridyl optionally substituted with one, two, or three R^{4a}. In some embodiments of a compound of Formula (I), (Ia), (Ib), or (Ic), R⁴ is thiazolyl or pyridyl; each optionally substituted with one, two, or three R^{4a}.

In some embodiments of a compound of Formula (I), (Ia), (Ib), or (Ic), each R⁴ is optionally substituted with one R^{4a}. In some embodiments of a compound of Formula (I), (Ia), (Ib), or (Ic), each R⁴ is optionally substituted with one or two R^{4a}. In some embodiments of a compound of Formula (I), (Ia), (Ib), or (Lc), each R⁴ is optionally substituted with two R^{4a}. In some embodiments of a compound of Formula (I), (Ia), (Ib), or (Ic), each R⁴ is optionally substituted with two or three R^{4a}.

In some embodiments of a compound of Formula (I), (Ia), (Ib), or (Ic), each R^{4a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I), (Ia), (Ib), or (Ic), each R^{4a} is independently halogen, C₁-C₆ alkyl, or C₁-C₆ haloalkyl. In some embodiments of a compound of Formula (I), (Ia), (Ib), or (Ic), each R^{4a} is independently halogen or C₁-C₆ alkyl. In some embodiments of a compound of Formula (I), (Ia), (Ib), or (Ic), each R^{4a} is independently halogen or C₁-C₆ haloalkyl. In some embodiments of a compound of Formula (I), (Ia), (Ib), or (Ic), each R^{4a} is independently halogen.

In some embodiments of a compound of Formula (I), (Ia), (Ib), or (Ic), two R^{4a} are taken together to form a heterocycloalkyl.

In some embodiments of a compound of Formula (I), (Ia), (Ib), or (Ic), R¹ is heterocycloalkyl or heteroaryl; wherein the heterocycloalkyl and heteroaryl are independently optionally substituted with one, two, or three R^{1a}.

In some embodiments of a compound of Formula (I), (Ia), (Ib), or (Ic), R¹ is heteroaryl optionally substituted with one, two, or three R^{1a}. In some embodiments of a compound of Formula (I), (Ia), (Ib), or (Ic), R¹ is a 6-membered heteroaryl optionally substituted with one, two, or three R^{1a}. In some embodiments of a compound of Formula (I), (Ia), (Ib), or (Ic), R¹ is pyridyl optionally substituted with one, two, or three R^{1a}. In some embodiments of a compound of Formula (I), (Ia), (Ib), or (Ic), R¹ is a 5-membered heteroaryl optionally substituted with one, two, or three R^{1a}. In some embodiments of a compound of Formula (I), (Ia), (Ib), or (Ic), R¹ is heterocycloalkyl optionally substituted with one, two, or three R^{1a}.

In some embodiments of a compound of Formula (I), (Ia), (Ib), or (Ic), each R¹ is optionally substituted with one R^{1a}. In some embodiments of a compound of Formula (I), (Ia), (Ib), or (Ic), each R¹ is optionally substituted with one or two R^{1a}. In some embodiments of a compound of Formula (I), (Ia), (Ib), or (Lc), each R¹ is optionally substituted with two R^{1a}. In some embodiments of a compound of Formula (I), (Ia), (Ib), or (Ic), each R¹ is optionally substituted with two or three R^{1a}.

In some embodiments of a compound of Formula (I), (Ia), (Ib), or (Ic), each R^{1a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl. In some embodiments of a compound of Formula (I), (Ia), (Ib), or (Ic), each R^{1a} is independently halogen, C₁-C₆ alkyl, or C₁-C₆ haloalkyl. In some embodiments of a compound of Formula (I), (Ia), (Ib), or (Ic), each R^{1a} is independently halogen or C₁-C₆ alkyl. In some embodiments of a compound of Formula (I), (Ia), (Ib), or (Ic), each R^{1a} is independently halogen.

In some embodiments also provided herein is a compound having the structure of Formula (II), or a pharmaceutically acceptable salt, solvate, stereoisomer, or isotopic variant thereof:

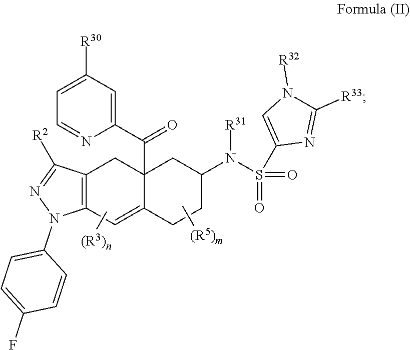

wherein:
R² is hydrogen, halogen, C₁-C₆ alkyl, or C₁-C₆ haloalkyl;
each R³ is independently halogen, C₁-C₆ alkyl, or C₁-C₆ haloalkyl;
each R⁵ is independently halogen, C₁-C₆ alkyl, or C₁-C₆ haloalkyl;
R³⁰ is F, Br, I, Me, CHF₂, or CH₂F;
R³¹ is C₃-C₄ cycloalkyl;
R³² is hydrogen, C₁-C₆ alkyl, or C₁-C₆ haloalkyl;
R³³ is hydrogen, C₁-C₆ alkyl, or C₁-C₆ haloalkyl;
m is 0-4; and
n is 0-3.

In some embodiments of a compound of Formula (II), R² is hydrogen or halogen. In some embodiments of a compound of Formula (II), R² is hydrogen or C₁-C₆ alkyl. In some embodiments of a compound of Formula (II), R² is hydrogen.

In some embodiments of a compound of Formula (II), each R³ is independently halogen or C₁-C₆ alkyl. In some embodiments of a compound of Formula (II), each R³ is independently halogen. In some embodiments of a compound of Formula (II), each R³ is independently C₁-C₆ alkyl.

In some embodiments of a compound of Formula (II), n is 0-2. In some embodiments of a compound of Formula (II), n is 0. In some embodiments of a compound of Formula (II), n is 1. In some embodiments of a compound of Formula (II), n is 2. In some embodiments of a compound of Formula (II), n is 3.

In some embodiments of a compound of Formula (II), each R⁵ is independently halogen or C₁-C₆ alkyl. In some embodiments of a compound of Formula (II), each R⁵ is independently halogen. In some embodiments of a compound of Formula (II), each R⁵ is independently C₁-C₆ alkyl.

In some embodiments of a compound of Formula (II), m is 0-2. In some embodiments of a compound of Formula (II), m is 0-3. In some embodiments of a compound of Formula (II), m is 0. In some embodiments of a compound of Formula (II), m is 1. In some embodiments of a compound of Formula (II), m is 2. In some embodiments of a compound of Formula (II), m is 3. In some embodiments of a compound of Formula (II), m is 4.

In some embodiments of a compound of Formula (II), R³⁰ is F, Br, or I. In some embodiments of a compound of Formula (II), R³⁰ is F.

In some embodiments of a compound of Formula (II), R³¹ is cyclopropyl. In some embodiments of a compound of Formula (II), R³¹ is cyclobutyl.

In some embodiments of a compound of Formula (II), R³² is hydrogen or C₁-C₆ alkyl. In some embodiments of a compound of Formula (II), R³² is hydrogen. In some embodiments of a compound of Formula (II), R³² is C₁-C₆ alkyl.

In some embodiments of a compound of Formula (II), R³³ is hydrogen or C₁-C₆ alkyl. In some embodiments of a compound of Formula (II), R³³ is hydrogen. In some embodiments of a compound of Formula (II), R³³ is C₁-C₆ alkyl.

In some embodiments also provided herein is a compound having the structure of Formula (III), or a pharmaceutically acceptable salt, solvate, stereoisomer, or isotopic variant thereof:

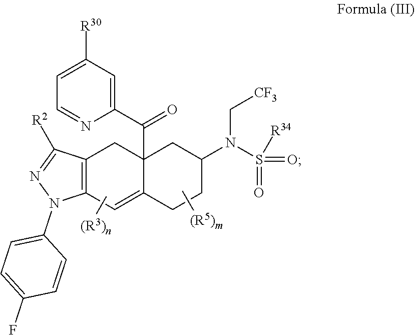

wherein:
R² is hydrogen, halogen, C₁-C₆ alkyl, or C₁-C₆ haloalkyl;
each R³ is independently halogen, C₁-C₆ alkyl, or C₁-C₆ haloalkyl;
each R⁵ is independently halogen, C₁-C₆ alkyl, or C₁-C₆ haloalkyl;
R³⁰ is F, Br, I, Me, CHF₂, or CH₂F;
R³⁴ is triazole optionally substituted with one or two R^{34a};
each R^{34a} is independently halogen, —CN, —OH, —NH₂, C₁-C₆ alkyl, C₁-C₆ haloalkyl, C₁-C₆ hydroxyalkyl, cycloalkyl, heterocycloalkyl, aryl, or heteroaryl;
m is 0-4; and
n is 0-3.

In some embodiments of a compound of Formula (III), R² is hydrogen or halogen. In some embodiments of a compound of Formula (III), R² is hydrogen or C₁-C₆ alkyl. In some embodiments of a compound of Formula (III), R² is hydrogen.

In some embodiments of a compound of Formula (III), each R³ is independently halogen or C₁-C₆ alkyl. In some embodiments of a compound of Formula (III), each R³ is independently halogen. In some embodiments of a compound of Formula (III), each R³ is independently C₁-C₆ alkyl.

In some embodiments of a compound of Formula (III), n is 0-2. In some embodiments of a compound of Formula (III), n is 0. In some embodiments of a compound of Formula (III), n is 1. In some embodiments of a compound of Formula (III), n is 2. In some embodiments of a compound of Formula (III), n is 3.

In some embodiments of a compound of Formula (III), each R⁵ is independently halogen or C₁-C₆ alkyl. In some embodiments of a compound of Formula (III), each R⁵ is independently halogen. In some embodiments of a compound of Formula (III), each R⁵ is independently C₁-C₆ alkyl.

In some embodiments of a compound of Formula (III), m is 0-2. In some embodiments of a compound of Formula (III), m is 0-3. In some embodiments of a compound of Formula (III), m is 0. In some embodiments of a compound of Formula (III), m is 1. In some embodiments of a compound of Formula (III), m is 2. In some embodiments of a compound of Formula (III), m is 3. In some embodiments of a compound of Formula (III), m is 4.

In some embodiments of a compound of Formula (III), R³⁰ is F, Br, or I. In some embodiments of a compound of Formula (III), R³⁰ is F.

In some embodiments of a compound of Formula (III), each R^{34a} is independently C₁-C₆ alkyl.

In some embodiments is a compound, or a pharmaceutically acceptable salt, solvate, stereoisomer, or isotopic variant thereof, having a structure selected from:

| Ex. | Structure |
|---|---|
| 1 | 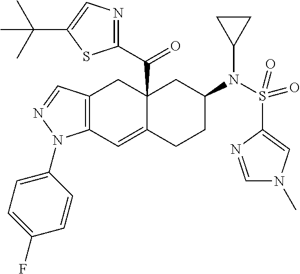 [SMILES: Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)c1] |
| 2 | 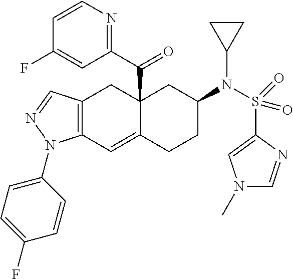 [SMILES: Cn1cnc(S(=O)(=O)N(C2CC2)[C@H]2CCC3=Cc4c(cnn4-c4ccc(F)cc4)C[C@]3(C(=O)c3cc(F)ccn3)C2)c1] |
| 3 | 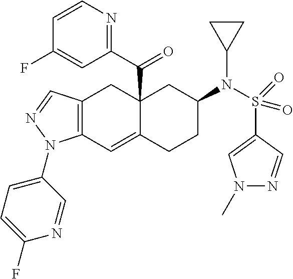 [SMILES: Cn1cc(S(=O)(=O)N(C2CC2)[C@H]2CCC3=Cc4c(cnn4-c4ccc(F)nc4)C[C@]3(C(=O)c3cc(F)ccn3)C2)cn1] |
| 4 | 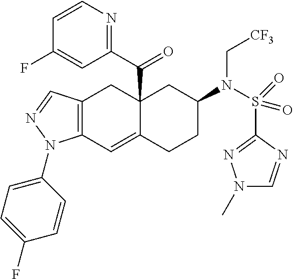 [SMILES: Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)n1] |
| 5 | 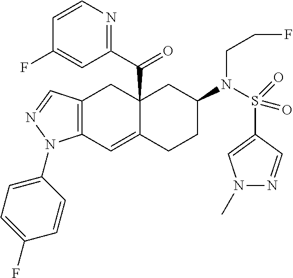 [SMILES: Cn1cc(S(=O)(=O)N(CCF)[C@H]2CCC3=Cc4c(cnn4-c4ccc(F)cc4)C[C@]3(C(=O)c3cc(F)ccn3)C2)cn1] |
| 6 | 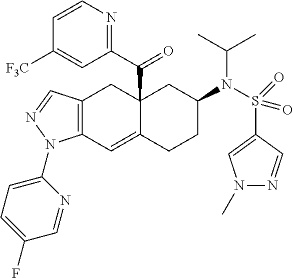 [SMILES: CC(C)N([C@H]1CCC2=Cc3c(cnn3-c3ccc(F)cn3)C[C@]2(C(=O)c2cc(C(F)(F)F)ccn2)C1)S(=O)(=O)c1cnn(C)c1] |
| 7 | 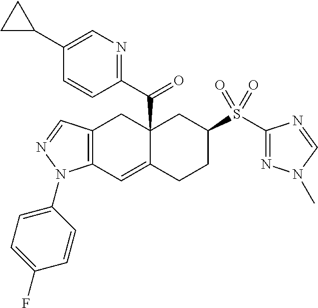 [SMILES: Cn1cnc(S(=O)(=O)[C@H]2CCC3=Cc4c(cnn4-c4ccc(F)cc4)C[C@]3(C(=O)c3ccc(C4CC4)cn3)C2)n1] |
| 8 | 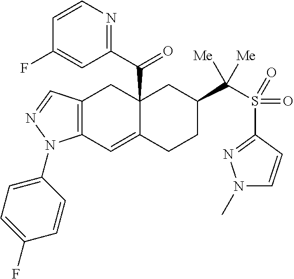 [SMILES: Cn1ccc(S(=O)(=O)C(C)(C)[C@H]2CCC3=Cc4c(cnn4-c4ccc(F)cc4)C[C@]3(C(=O)c3cc(F)ccn3)C2)n1] |
| 9 | 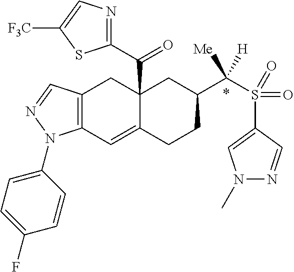 [SMILES: C[C@H]([C@H]1CCC2=Cc3c(cnn3-c3ccc(F)cc3)C[C@]2(C(=O)c2ncc(C(F)(F)F)s2)C1)S(=O)(=O)c1cnn(C)c1] |
| 10 | 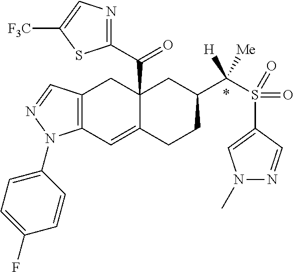 [SMILES: C[C@@H]([C@H]1CCC2=Cc3c(cnn3-c3ccc(F)cc3)C[C@]2(C(=O)c2ncc(C(F)(F)F)s2)C1)S(=O)(=O)c1cnn(C)c1] |
| 31 | 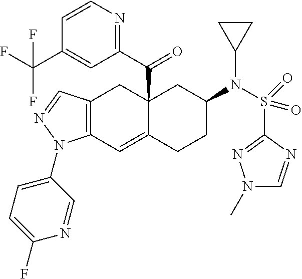 [SMILES: Cn1cnc(S(=O)(=O)N(C2CC2)[C@H]2CCC3=Cc4c(cnn4-c4ccc(F)nc4)C[C@]3(C(=O)c3cc(C(F)(F)F)ccn3)C2)n1] |
| 12 | 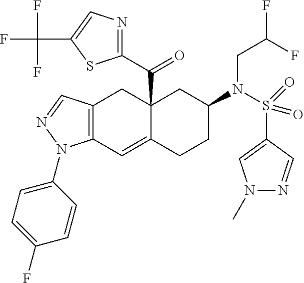 [SMILES: Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)cn1] |
| 13 | 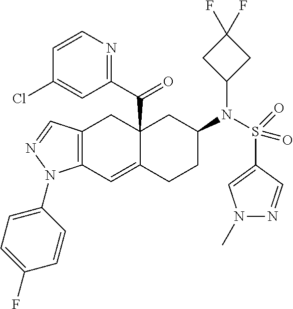 [SMILES: Cn1cc(S(=O)(=O)N(C2CC(F)(F)C2)[C@H]2CCC3=Cc4c(cnn4-c4ccc(F)cc4)C[C@]3(C(=O)c3cc(Cl)ccn3)C2)cn1] |
| 14 | 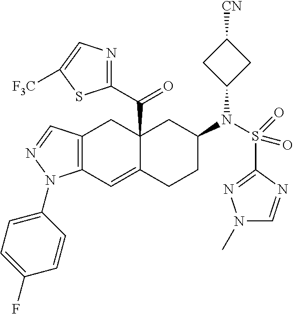 [SMILES: Cn1cnc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@H](C#N)C2)n1] |
| 15 | 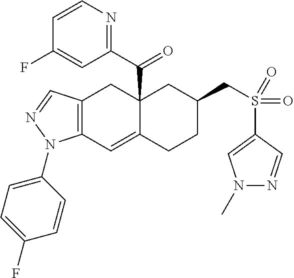 [SMILES: Cn1cc(S(=O)(=O)C[C@H]2CCC3=Cc4c(cnn4-c4ccc(F)cc4)C[C@]3(C(=O)c3cc(F)ccn3)C2)cn1] |
| 16 | 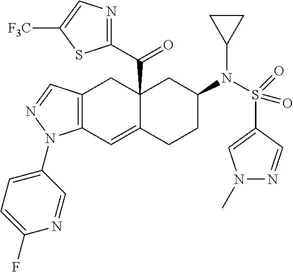 [SMILES: Cn1cc(S(=O)(=O)N(C2CC2)[C@H]2CCC3=Cc4c(cnn4-c4ccc(F)nc4)C[C@]3(C(=O)c3ncc(C(F)(F)F)s3)C2)cn1] |
| 17 | 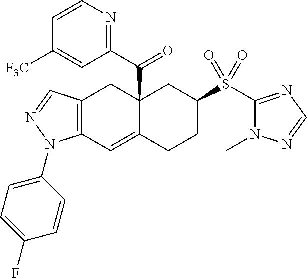 [SMILES: Cn1ncnc1S(=O)(=O)[C@H]1CCC2=Cc3c(cnn3-c3ccc(F)cc3)C[C@]2(C(=O)c2cc(C(F)(F)F)ccn2)C1] |
| 18 | 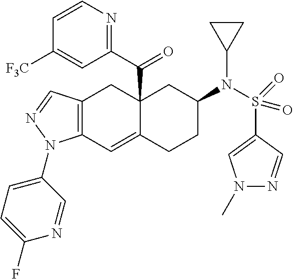 [SMILES: Cn1cc(S(=O)(=O)N(C2CC2)[C@H]2CCC3=Cc4c(cnn4-c4ccc(F)nc4)C[C@]3(C(=O)c3cc(C(F)(F)F)ccn3)C2)cn1] |
| 19 | 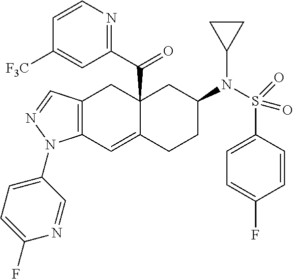 [SMILES: O=C(c1cc(C(F)(F)F)ccn1)[C@]12Cc3cnn(-c4ccc(F)nc4)c3C=C1CC[C@H](N(C1CC1)S(=O)(=O)c1ccc(F)cc1)C2] |
| 20 | 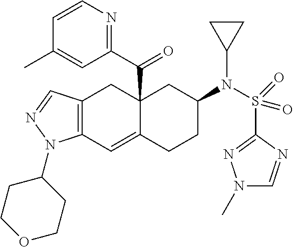 [SMILES: Cc1ccnc(C(=O)[C@]23Cc4cnn(C5CCOCC5)c4C=C2CC[C@H](N(C2CC2)S(=O)(=O)c2ncn(C)n2)C3)c1] |
| 21 | 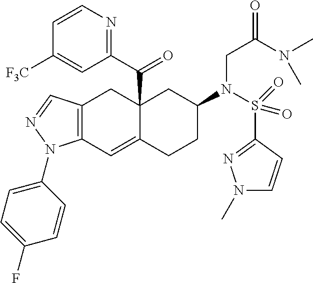 [SMILES: CN(C)C(=O)CN([C@H]1CCC2=Cc3c(cnn3-c3ccc(F)cc3)C[C@]2(C(=O)c2cc(C(F)(F)F)ccn2)C1)S(=O)(=O)c1ccn(C)n1] |
| 22 | 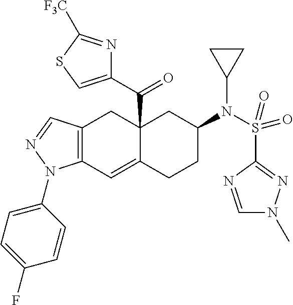 [SMILES: Cn1cnc(S(=O)(=O)N(C2CC2)[C@H]2CCC3=Cc4c(cnn4-c4ccc(F)cc4)C[C@]3(C(=O)c3csc(C(F)(F)F)n3)C2)n1] |
| 23 | 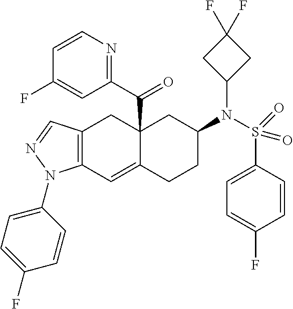 [SMILES: O=C(c1cc(F)ccn1)[C@]12Cc3cnn(-c4ccc(F)cc4)c3C=C1CC[C@H](N(C1CC(F)(F)C1)S(=O)(=O)c1ccc(F)cc1)C2] |
| 24 | 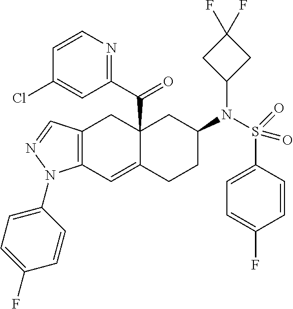 [SMILES: O=C(c1cc(Cl)ccn1)[C@]12Cc3cnn(-c4ccc(F)cc4)c3C=C1CC[C@H](N(C1CC(F)(F)C1)S(=O)(=O)c1ccc(F)cc1)C2] |
| 25 | 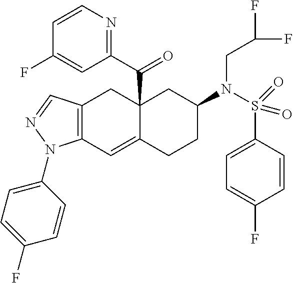 [SMILES: O=C(c1cc(F)ccn1)[C@]12Cc3cnn(-c4ccc(F)cc4)c3C=C1CC[C@H](N(CC(F)F)S(=O)(=O)c1ccc(F)cc1)C2] |
| 26 | 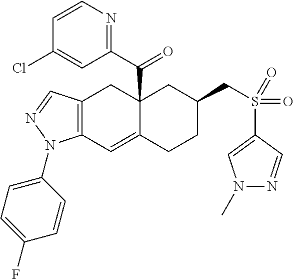 [SMILES: Cn1cc(S(=O)(=O)C[C@H]2CCC3=Cc4c(cnn4-c4ccc(F)cc4)C[C@]3(C(=O)c3cc(Cl)ccn3)C2)cn1] |
| 27 | 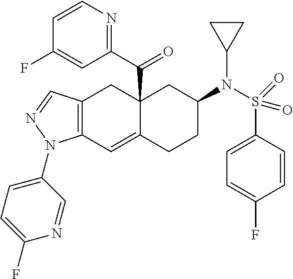 [SMILES: O=C(c1cc(F)ccn1)[C@]12Cc3cnn(-c4ccc(F)nc4)c3C=C1CC[C@H](N(C1CC1)S(=O)(=O)c1ccc(F)cc1)C2] |
| 28 | 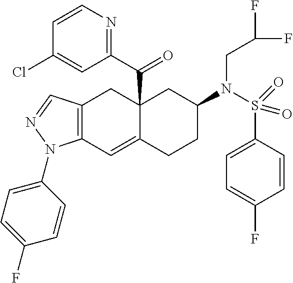 [SMILES: O=C(c1cc(Cl)ccn1)[C@]12Cc3cnn(-c4ccc(F)cc4)c3C=C1CC[C@H](N(CC(F)F)S(=O)(=O)c1ccc(F)cc1)C2] |
| 29 | 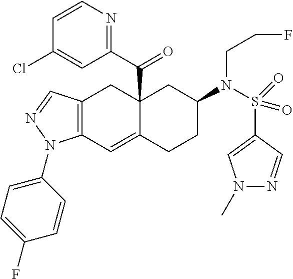 [SMILES: Cn1cc(S(=O)(=O)N(CCF)[C@H]2CCC3=Cc4c(cnn4-c4ccc(F)cc4)C[C@]3(C(=O)c3cc(Cl)ccn3)C2)cn1] |
| 30 | 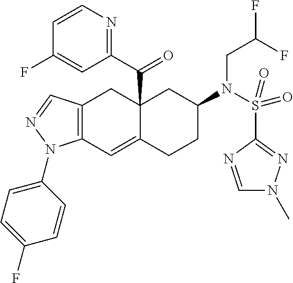 [SMILES: Cn1cnc(S(=O)(=O)N(CC(F)F)[C@H]2CCC3=Cc4c(cnn4-c4ccc(F)cc4)C[C@]3(C(=O)c3cc(F)ccn3)C2)n1] |
| 31 | 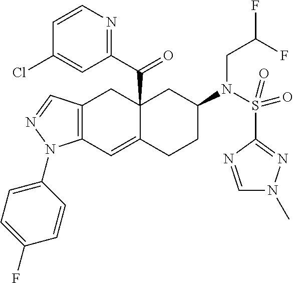 [SMILES: Cn1cnc(S(=O)(=O)N(CC(F)F)[C@H]2CCC3=Cc4c(cnn4-c4ccc(F)cc4)C[C@]3(C(=O)c3cc(Cl)ccn3)C2)n1] |
| 32 | 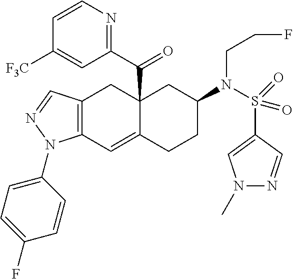 [SMILES: Cn1cc(S(=O)(=O)N(CCF)[C@H]2CCC3=Cc4c(cnn4-c4ccc(F)cc4)C[C@]3(C(=O)c3cc(C(F)(F)F)ccn3)C2)cn1] |
| 33 | 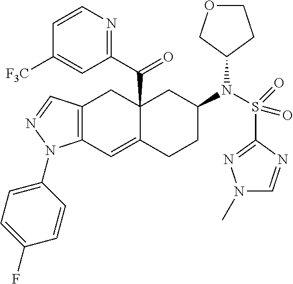 [SMILES: Cn1cnc(S(=O)(=O)N([C@H]2CCOC2)[C@H]2CCC3=Cc4c(cnn4-c4ccc(F)cc4)C[C@]3(C(=O)c3cc(C(F)(F)F)ccn3)C2)n1] |
| 34 | 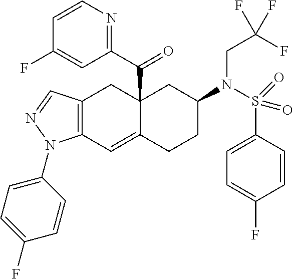 [SMILES: O=C(c1cc(F)ccn1)[C@]12Cc3cnn(-c4ccc(F)cc4)c3C=C1CC[C@H](N(CC(F)(F)F)S(=O)(=O)c1ccc(F)cc1)C2] |
| 35 | 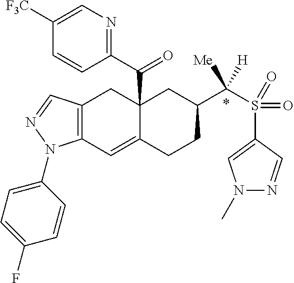 [SMILES: C[C@H]([C@H]1CCC2=Cc3c(cnn3-c3ccc(F)cc3)C[C@]2(C(=O)c2ccc(C(F)(F)F)cn2)C1)S(=O)(=O)c1cnn(C)c1] |
| 36 | 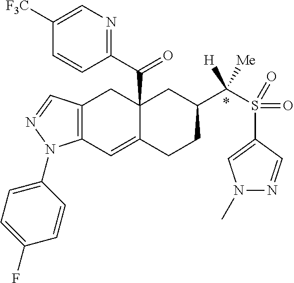 [SMILES: C[C@@H]([C@H]1CCC2=Cc3c(cnn3-c3ccc(F)cc3)C[C@]2(C(=O)c2ccc(C(F)(F)F)cn2)C1)S(=O)(=O)c1cnn(C)c1] |
| 37 | 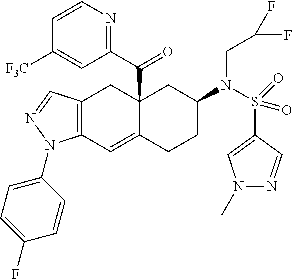 [SMILES: Cn1cc(S(=O)(=O)N(CC(F)F)[C@H]2CCC3=Cc4c(cnn4-c4ccc(F)cc4)C[C@]3(C(=O)c3cc(C(F)(F)F)ccn3)C2)cn1] |
| 38 | 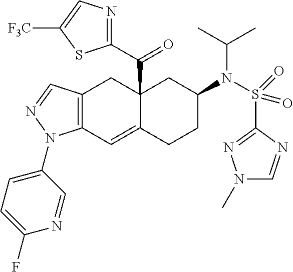 [SMILES: CC(C)N([C@H]1CCC2=Cc3c(cnn3-c3ccc(F)nc3)C[C@]2(C(=O)c2ncc(C(F)(F)F)s2)C1)S(=O)(=O)c1ncn(C)n1] |
| 39 | 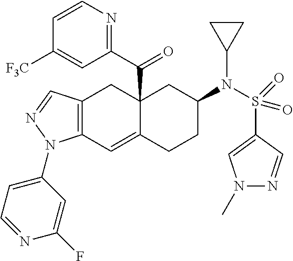 [SMILES: Cn1cc(S(=O)(=O)N(C2CC2)[C@H]2CCC3=Cc4c(cnn4-c4ccnc(F)c4)C[C@]3(C(=O)c3cc(C(F)(F)F)ccn3)C2)cn1] |
| 40 | 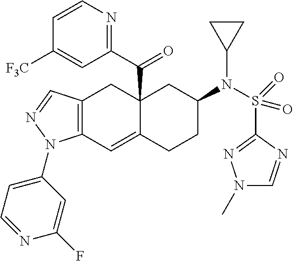 [SMILES: Cn1cnc(S(=O)(=O)N(C2CC2)[C@H]2CCC3=Cc4c(cnn4-c4ccnc(F)c4)C[C@]3(C(=O)c3cc(C(F)(F)F)ccn3)C2)n1] |
| 41 | 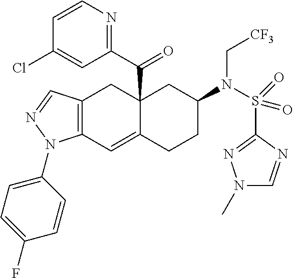 [SMILES: Cn1cnc(S(=O)(=O)N(CC(F)(F)F)[C@H]2CCC3=Cc4c(cnn4-c4ccc(F)cc4)C[C@]3(C(=O)c3cc(Cl)ccn3)C2)n1] |
| 42 | 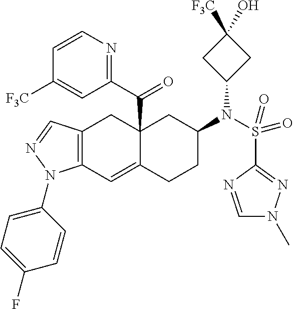 [SMILES: Cn1cnc(S(=O)(=O)N([C@H]2CCC3=Cc4c(cnn4-c4ccc(F)cc4)C[C@]3(C(=O)c3cc(C(F)(F)F)ccn3)C2)[C@H]2C[C@](O)(C(F)(F)F)C2)n1] |
| 43 | 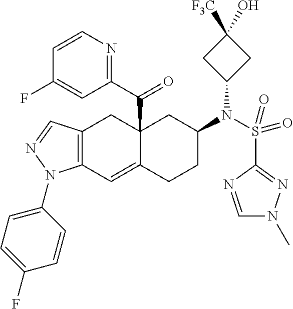 [SMILES: Cn1cnc(S(=O)(=O)N([C@H]2CCC3=Cc4c(cnn4-c4ccc(F)cc4)C[C@]3(C(=O)c3cc(F)ccn3)C2)[C@H]2C[C@](O)(C(F)(F)F)C2)n1] |
| 44 | 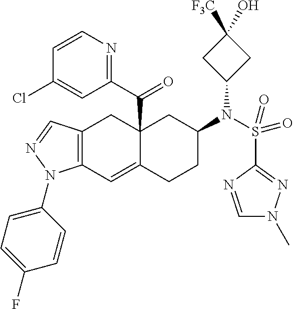 [SMILES: Cn1cnc(S(=O)(=O)N([C@H]2CCC3=Cc4c(cnn4-c4ccc(F)cc4)C[C@]3(C(=O)c3cc(Cl)ccn3)C2)[C@H]2C[C@](O)(C(F)(F)F)C2)n1] |
| 45 | 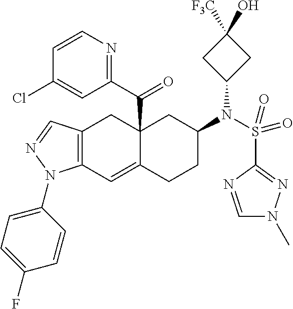 [SMILES: Cn1cnc(S(=O)(=O)N([C@H]2CCC3=Cc4c(cnn4-c4ccc(F)cc4)C[C@]3(C(=O)c3cc(Cl)ccn3)C2)[C@H]2C[C@@](O)(C(F)(F)F)C2)n1] |
| 46 | 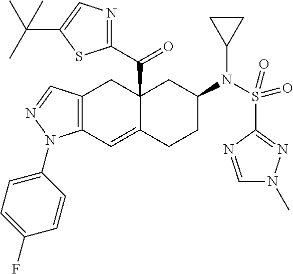 [SMILES: Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)n1] |
| 47 | 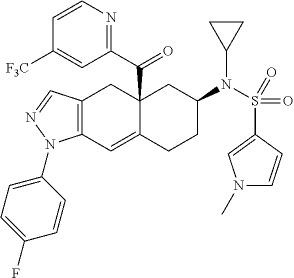 [SMILES: Cn1ccc(S(=O)(=O)N(C2CC2)[C@H]2CCC3=Cc4c(cnn4-c4ccc(F)cc4)C[C@]3(C(=O)c3cc(C(F)(F)F)ccn3)C2)c1] |
| 48 | 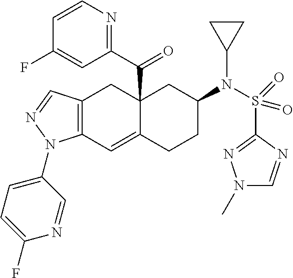 [SMILES: Cn1cnc(S(=O)(=O)N(C2CC2)[C@H]2CCC3=Cc4c(cnn4-c4ccc(F)nc4)C[C@]3(C(=O)c3cc(F)ccn3)C2)n1] |
| 49 | 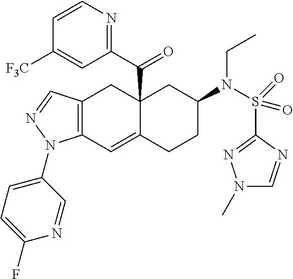 [SMILES: CCN([C@H]1CCC2=Cc3c(cnn3-c3ccc(F)nc3)C[C@]2(C(=O)c2cc(C(F)(F)F)ccn2)C1)S(=O)(=O)c1ncn(C)n1] |
| 50 | 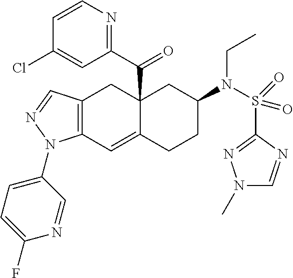 [SMILES: CCN([C@H]1CCC2=Cc3c(cnn3-c3ccc(F)nc3)C[C@]2(C(=O)c2cc(Cl)ccn2)C1)S(=O)(=O)c1ncn(C)n1] |
| 51 | 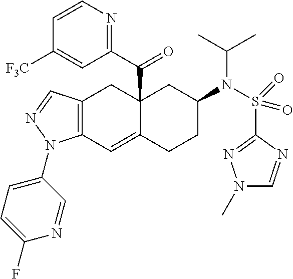 [SMILES: CC(C)N([C@H]1CCC2=Cc3c(cnn3-c3ccc(F)nc3)C[C@]2(C(=O)c2cc(C(F)(F)F)ccn2)C1)S(=O)(=O)c1ncn(C)n1] |
| 52 | 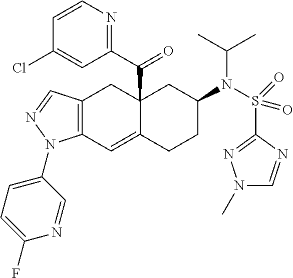 [SMILES: CC(C)N([C@H]1CCC2=Cc3c(cnn3-c3ccc(F)nc3)C[C@]2(C(=O)c2cc(Cl)ccn2)C1)S(=O)(=O)c1ncn(C)n1] |
| 53 | 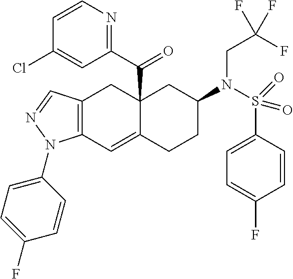 [SMILES: O=C(c1cc(Cl)ccn1)[C@]12Cc3cnn(-c4ccc(F)cc4)c3C=C1CC[C@H](N(CC(F)(F)F)S(=O)(=O)c1ccc(F)cc1)C2] |
| 54 | 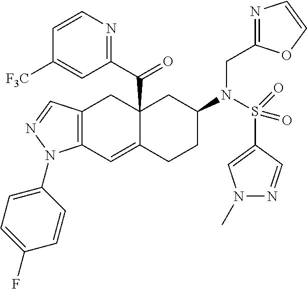 [SMILES: Cn1cc(S(=O)(=O)N(Cc2ncco2)[C@H]2CCC3=Cc4c(cnn4-c4ccc(F)cc4)C[C@]3(C(=O)c3cc(C(F)(F)F)ccn3)C2)cn1] |
| 55 | 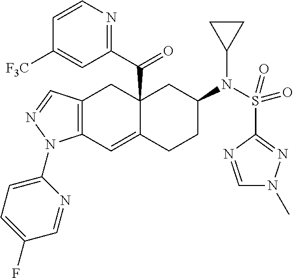 [SMILES: Cn1cnc(S(=O)(=O)N(C2CC2)[C@H]2CCC3=Cc4c(cnn4-c4ccc(F)cn4)C[C@]3(C(=O)c3cc(C(F)(F)F)ccn3)C2)n1] |
| 56 | 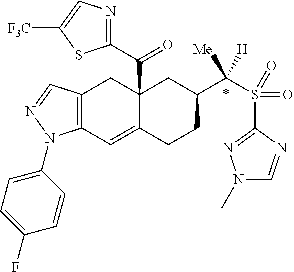 [SMILES: C[C@H]([C@H]1CCC2=Cc3c(cnn3-c3ccc(F)cc3)C[C@]2(C(=O)c2ncc(C(F)(F)F)s2)C1)S(=O)(=O)c1ncn(C)n1] |
| 57 | 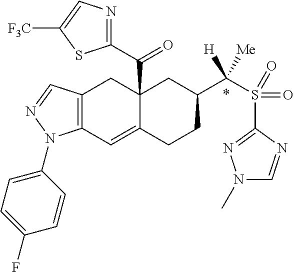 [SMILES: C[C@@H]([C@H]1CCC2=Cc3c(cnn3-c3ccc(F)cc3)C[C@]2(C(=O)c2ncc(C(F)(F)F)s2)C1)S(=O)(=O)c1ncn(C)n1] |
| 58 | 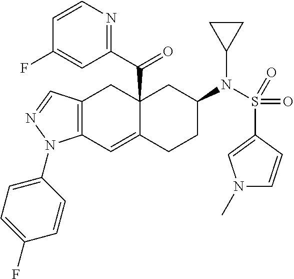 [SMILES: Cn1ccc(S(=O)(=O)N(C2CC2)[C@H]2CCC3=Cc4c(cnn4-c4ccc(F)cc4)C[C@]3(C(=O)c3cc(F)ccn3)C2)c1] |
| 59 | 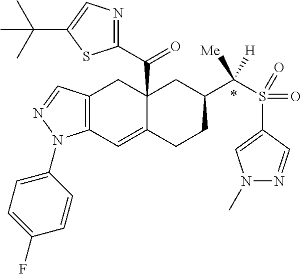 [SMILES: C[C@H]([C@H]1CCC2=Cc3c(cnn3-c3ccc(F)cc3)C[C@]2(C(=O)c2ncc(C(C)(C)C)s2)C1)S(=O)(=O)c1cnn(C)c1] |
| 60 | 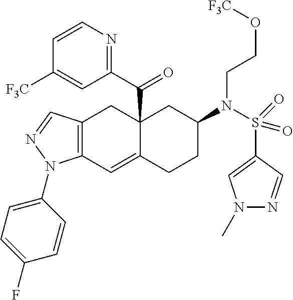 [SMILES: Cn1cc(S(=O)(=O)N(CCOC(F)(F)F)[C@H]2CCC3=Cc4c(cnn4-c4ccc(F)cc4)C[C@]3(C(=O)c3cc(C(F)(F)F)ccn3)C2)cn1] |
| 61 | 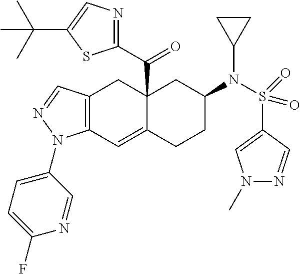 [SMILES: Cn1cc(S(=O)(=O)N(C2CC2)[C@H]2CCC3=Cc4c(cnn4-c4ccc(F)nc4)C[C@]3(C(=O)c3ncc(C(C)(C)C)s3)C2)cn1] |
| 62 | 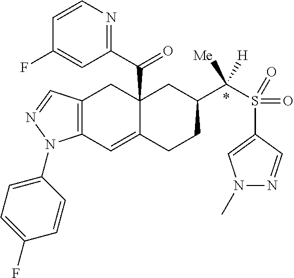 [SMILES: C[C@H]([C@H]1CCC2=Cc3c(cnn3-c3ccc(F)cc3)C[C@]2(C(=O)c2cc(F)ccn2)C1)S(=O)(=O)c1cnn(C)c1] |
| 63 | 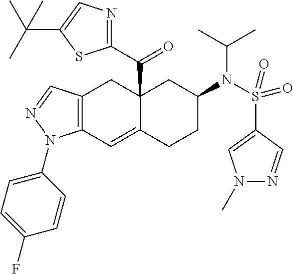 [SMILES: CC(C)N([C@H]1CCC2=Cc3c(cnn3-c3ccc(F)cc3)C[C@]2(C(=O)c2ncc(C(C)(C)C)s2)C1)S(=O)(=O)c1cnn(C)c1] |
| 64 | 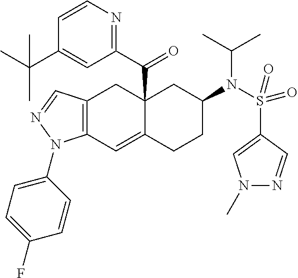 [SMILES: CC(C)N([C@H]1CCC2=Cc3c(cnn3-c3ccc(F)cc3)C[C@]2(C(=O)c2cc(C(C)(C)C)ccn2)C1)S(=O)(=O)c1cnn(C)c1] |
| 65 | 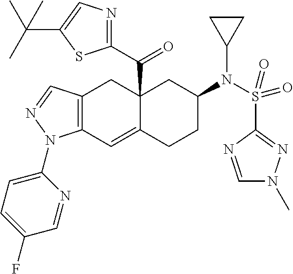 [SMILES: Cn1cnc(S(=O)(=O)N(C2CC2)[C@H]2CCC3=Cc4c(cnn4-c4ccc(F)cn4)C[C@]3(C(=O)c3ncc(C(C)(C)C)s3)C2)n1] |
| 66 | 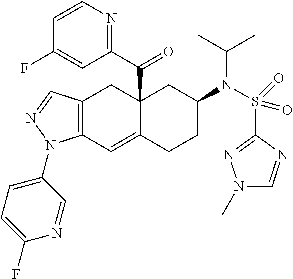 [SMILES: CC(C)N([C@H]1CCC2=Cc3c(cnn3-c3ccc(F)nc3)C[C@]2(C(=O)c2cc(F)ccn2)C1)S(=O)(=O)c1ncn(C)n1] |
| 67 | 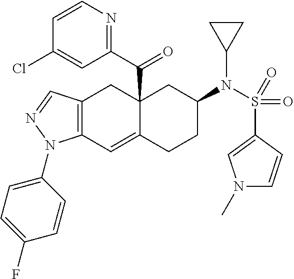 [SMILES: Cn1ccc(S(=O)(=O)N(C2CC2)[C@H]2CCC3=Cc4c(cnn4-c4ccc(F)cc4)C[C@]3(C(=O)c3cc(Cl)ccn3)C2)c1] |
| 68 | 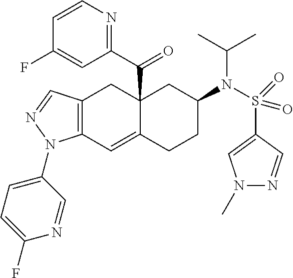 [SMILES: CC(C)N([C@H]1CCC2=Cc3c(cnn3-c3ccc(F)nc3)C[C@]2(C(=O)c2cc(F)ccn2)C1)S(=O)(=O)c1cnn(C)c1] |
| 69 | 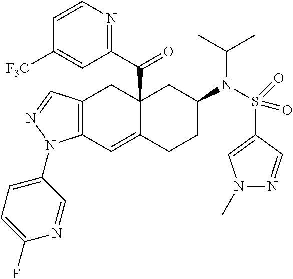 [SMILES: CC(C)N([C@H]1CCC2=Cc3c(cnn3-c3ccc(F)nc3)C[C@]2(C(=O)c2cc(C(F)(F)F)ccn2)C1)S(=O)(=O)c1cnn(C)c1] |
| 70 | 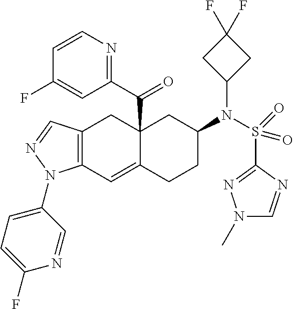 [SMILES: Cn1cnc(S(=O)(=O)N(C2CC(F)(F)C2)[C@H]2CCC3=Cc4c(cnn4-c4ccc(F)nc4)C[C@]3(C(=O)c3cc(F)ccn3)C2)n1] |
| 71 | 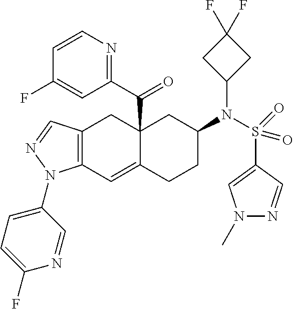 [SMILES: Cn1cc(S(=O)(=O)N(C2CC(F)(F)C2)[C@H]2CCC3=Cc4c(cnn4-c4ccc(F)nc4)C[C@]3(C(=O)c3cc(F)ccn3)C2)cn1] |
| 72 | 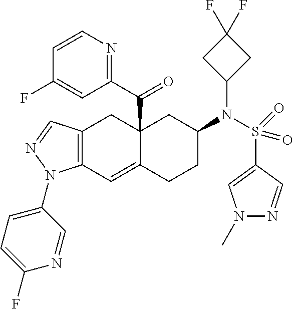 [SMILES: Cn1cc(S(=O)(=O)N(C2CC(F)(F)C2)[C@H]2CCC3=Cc4c(cnn4-c4ccc(F)nc4)C[C@]3(C(=O)c3cc(F)ccn3)C2)cn1] |
| 73 | 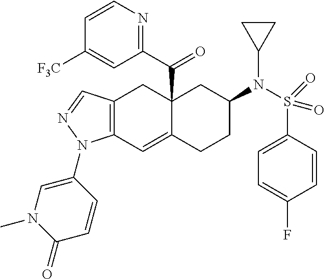 [SMILES: Cn1cc(-n2ncc3c2C=C2CC[C@H](N(C4CC4)S(=O)(=O)c4ccc(F)cc4)C[C@@]2(C(=O)c2cc(C(F)(F)F)ccn2)C3)ccc1=O] |
| 74 | 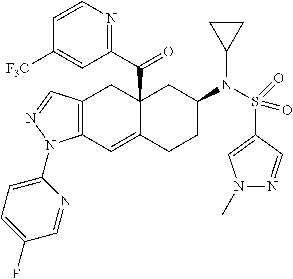 [SMILES: Cn1cc(S(=O)(=O)N(C2CC2)[C@H]2CCC3=Cc4c(cnn4-c4ccc(F)cn4)C[C@]3(C(=O)c3cc(C(F)(F)F)ccn3)C2)cn1] |
| 75 | 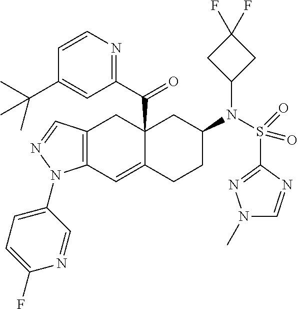 [SMILES: Cn1cnc(S(=O)(=O)N(C2CC(F)(F)C2)[C@H]2CCC3=Cc4c(cnn4-c4ccc(F)nc4)C[C@]3(C(=O)c3cc(C(C)(C)C)ccn3)C2)n1] |
| 76 | 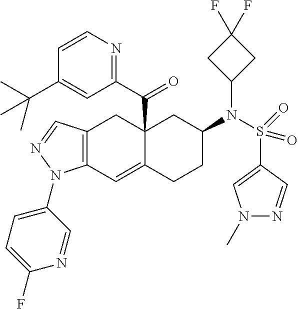 [SMILES: Cn1cc(S(=O)(=O)N(C2CC(F)(F)C2)[C@H]2CCC3=Cc4c(cnn4-c4ccc(F)nc4)C[C@]3(C(=O)c3cc(C(C)(C)C)ccn3)C2)cn1] |
| 77 | 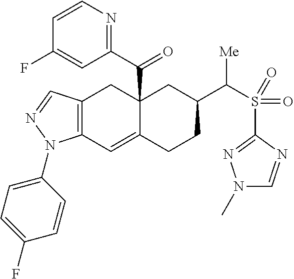 [SMILES: CC([C@H]1CCC2=Cc3c(cnn3-c3ccc(F)cc3)C[C@]2(C(=O)c2cc(F)ccn2)C1)S(=O)(=O)c1ncn(C)n1] |
| 78 | 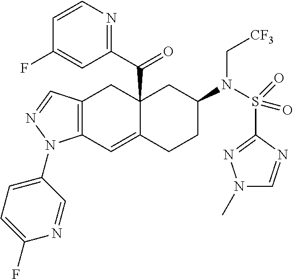 [SMILES: Cn1cnc(S(=O)(=O)N(CC(F)(F)F)[C@H]2CCC3=Cc4c(cnn4-c4ccc(F)nc4)C[C@]3(C(=O)c3cc(F)ccn3)C2)n1] |
| 79 | 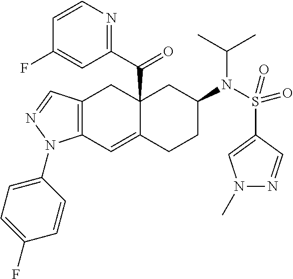 [SMILES: CC(C)N([C@H]1CCC2=Cc3c(cnn3-c3ccc(F)cc3)C[C@]2(C(=O)c2cc(F)ccn2)C1)S(=O)(=O)c1cnn(C)c1] |
| 80 | 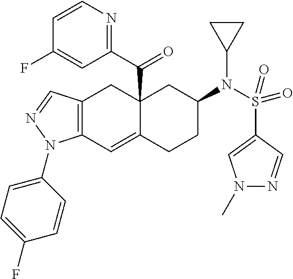 [SMILES: Cn1cc(S(=O)(=O)N(C2CC2)[C@H]2CCC3=Cc4c(cnn4-c4ccc(F)cc4)C[C@]3(C(=O)c3cc(F)ccn3)C2)cn1] |
| 81 | 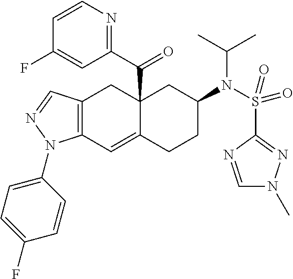 [SMILES: CC(C)N([C@H]1CCC2=Cc3c(cnn3-c3ccc(F)cc3)C[C@]2(C(=O)c2cc(F)ccn2)C1)S(=O)(=O)c1ncn(C)n1] |
| 82 | 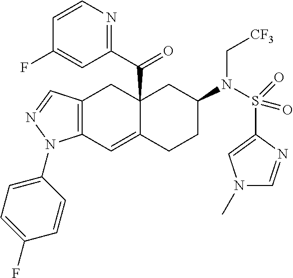 [SMILES: Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)c1] |
| 83 | 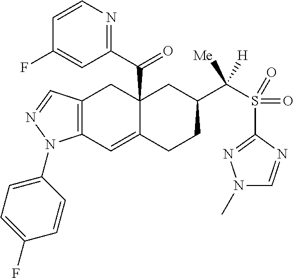 [SMILES: C[C@H]([C@H]1CCC2=Cc3c(cnn3-c3ccc(F)cc3)C[C@]2(C(=O)c2cc(F)ccn2)C1)S(=O)(=O)c1ncn(C)n1] |
| 84 | 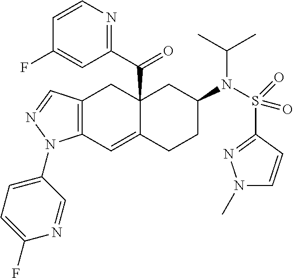 [SMILES: CC(C)N([C@H]1CCC2=Cc3c(cnn3-c3ccc(F)nc3)C[C@]2(C(=O)c2cc(F)ccn2)C1)S(=O)(=O)c1ccn(C)n1] |
| 85 | 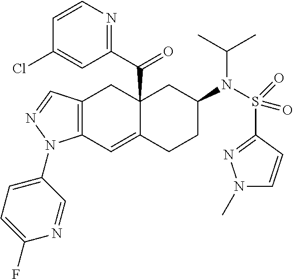 [SMILES: CC(C)N([C@H]1CCC2=Cc3c(cnn3-c3ccc(F)nc3)C[C@]2(C(=O)c2cc(Cl)ccn2)C1)S(=O)(=O)c1ccn(C)n1] |
| 86 | 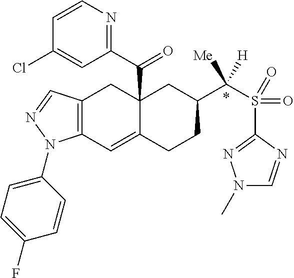 [SMILES: C[C@H]([C@H]1CCC2=Cc3c(cnn3-c3ccc(F)cc3)C[C@]2(C(=O)c2cc(Cl)ccn2)C1)S(=O)(=O)c1ncn(C)n1] |
| 87 | 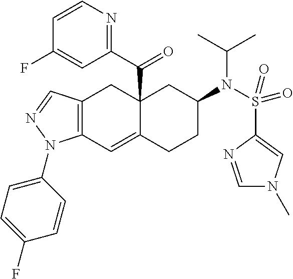 [SMILES: CC(C)N([C@H]1CCC2=Cc3c(cnn3-c3ccc(F)cc3)C[C@]2(C(=O)c2cc(F)ccn2)C1)S(=O)(=O)c1cn(C)cn1] |
| 88 | 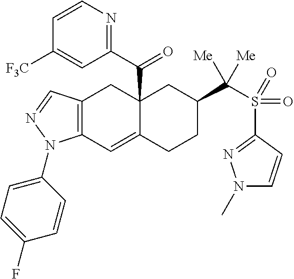 [SMILES: Cn1ccc(S(=O)(=O)C(C)(C)[C@H]2CCC3=Cc4c(cnn4-c4ccc(F)cc4)C[C@]3(C(=O)c3cc(C(F)(F)F)ccn3)C2)n1] |
| 89 | 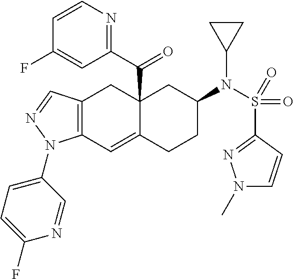 [SMILES: Cn1ccc(S(=O)(=O)N(C2CC2)[C@H]2CCC3=Cc4c(cnn4-c4ccc(F)nc4)C[C@]3(C(=O)c3cc(F)ccn3)C2)n1] |
| 90 | 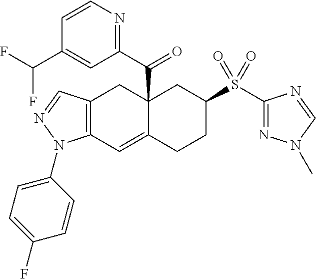 [SMILES: Cn1cnc(S(=O)(=O)[C@H]2CCC3=Cc4c(cnn4-c4ccc(F)cc4)C[C@]3(C(=O)c3cc(C(F)F)ccn3)C2)n1] |
| 91 | 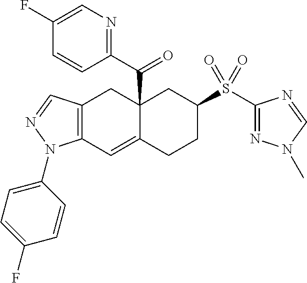 [SMILES: Cn1cnc(S(=O)(=O)[C@H]2CCC3=Cc4c(cnn4-c4ccc(F)cc4)C[C@]3(C(=O)c3ccc(F)cn3)C2)n1] |
| 92 | 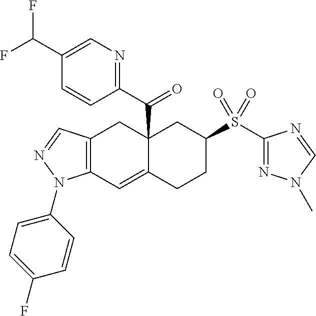 [SMILES: Cn1cnc(S(=O)(=O)[C@H]2CCC3=Cc4c(cnn4-c4ccc(F)cc4)C[C@]3(C(=O)c3ccc(C(F)F)cn3)C2)n1] |
| 93 | 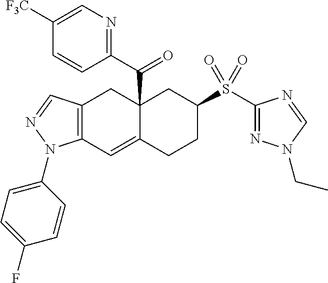 [SMILES: CCn1cnc(S(=O)(=O)[C@H]2CCC3=Cc4c(cnn4-c4ccc(F)cc4)C[C@]3(C(=O)c3ccc(C(F)(F)F)cn3)C2)n1] |
| 94 | 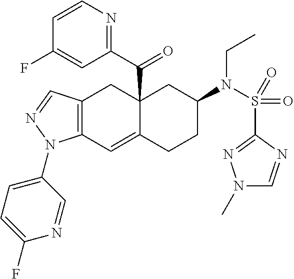 [SMILES: CCN([C@H]1CCC2=Cc3c(cnn3-c3ccc(F)nc3)C[C@]2(C(=O)c2cc(F)ccn2)C1)S(=O)(=O)c1ncn(C)n1] |
| 95 | 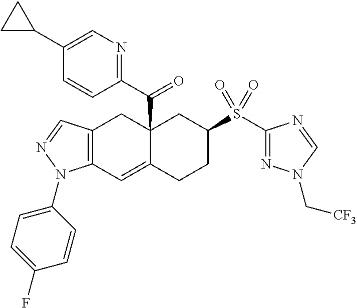 [SMILES: O=C(c1ccc(C2CC2)cn1)[C@]12Cc3cnn(-c4ccc(F)cc4)c3C=C1CC[C@H](S(=O)(=O)c1ncn(CC(F)(F)F)n1)C2] |
| 96 | 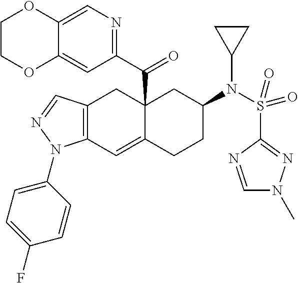 [SMILES: Cn1cnc(S(=O)(=O)N(C2CC2)[C@H]2CCC3=Cc4c(cnn4-c4ccc(F)cc4)C[C@]3(C(=O)c3cc4c(cn3)OCCO4)C2)n1] |
| 97 | 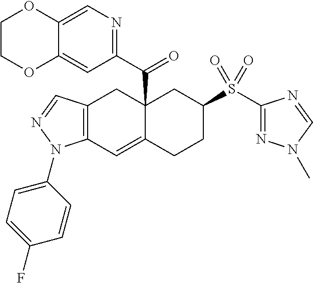 [SMILES: Cn1cnc(S(=O)(=O)[C@H]2CCC3=Cc4c(cnn4-c4ccc(F)cc4)C[C@]3(C(=O)c3cc4c(cn3)OCCO4)C2)n1] |
| 98 | 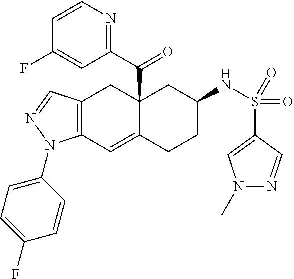 [SMILES: Cn1cc(S(=O)(=O)N[C@H]2CCC3=Cc4c(cnn4-c4ccc(F)cc4)C[C@]3(C(=O)c3cc(F)ccn3)C2)cn1] |
| 99 | 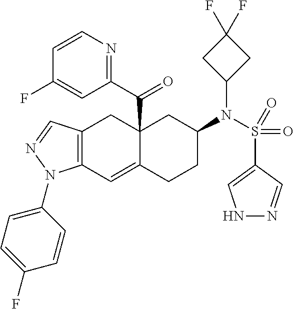 [SMILES: O=C(c1cc(F)ccn1)[C@]12Cc3cnn(-c4ccc(F)cc4)c3C=C1CC[C@H](N(C1CC(F)(F)C1)S(=O)(=O)c1cn[nH]c1)C2] |

*Single enantiomer of undetermined stereochemistry

In other embodiments provided herein is a compound having the structure of Formula (A), or a pharmaceutically acceptable salt, solvate, stereoisomer, or isotopic variant thereof:

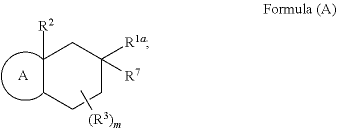

wherein:

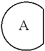

is

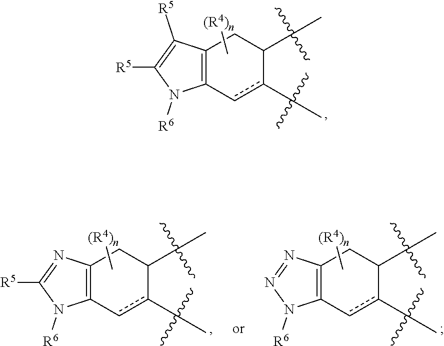

[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
each R³ and each R⁴ is independently halogen or C₁₋₆alkyl;
each R⁵ is independently hydrogen, C₁₋₆alkyl, or C₁₋₆haloalkyl;
R⁶ is C₆₋₁₀aryl or C₂₋₉heteroaryl; wherein C₆₋₁₀aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, C₂₋₉heterocycloalkyl, C₆₋₁₀aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl;
R¹ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12a};
m is 0, 1, 2, 3, or 4; and
n is 0, 1, 2, or 3.

In some embodiments of a compound of Formula (A), R^{1a} is —NR¹⁶S(O)₂R¹⁷.

In some embodiments of a compound of Formula (A), R^{1a} is —NR¹⁶C(O)R¹⁷.

In some embodiments of a compound of Formula (A), R¹⁶ is C₁₋₆alkyl or C₃₋₈cycloalkyl, wherein C₁₋₆alkyl and C₃₋₈cycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵.

In some embodiments of a compound of Formula (A), R¹⁶ is unsubstituted C₁₋₆alkyl.

In some embodiments of a compound of Formula (A), R¹⁶ is C₁₋₆haloalkyl.

In some embodiments of a compound of Formula (A), R¹⁶ is unsubstituted C₃₋₈cycloalkyl.

In some embodiments of a compound of Formula (A), R¹⁶ is unsubstituted cyclopropyl.

In some embodiments of a compound of Formula (A), R^{1a} is —C(R²⁰)₂S(O)₂R¹⁷.

In some embodiments of a compound of Formula (A), R²⁰ is hydrogen, C₁₋₆alkyl, or C₃₋₈cycloalkyl.

In some embodiments of a compound of Formula (A), R¹⁷ is C₆₋₁₀aryl or C₂₋₉heteroaryl, and the C₆₋₁₀aryl and C₂₋₉heteroaryl are optionally substituted with one, two, or three R^{12f}.

In some embodiments of a compound of Formula (A), R¹⁷ is phenyl optionally substituted with one, two, or three R^{12f}.

In some embodiments of a compound of Formula (A), R¹⁷ is phenyl optionally substituted with one, two, or three groups selected from halogen, C₁₋₆alkyl, and C₁₋₆haloalkyl.

In some embodiments of a compound of Formula (A), R¹⁷ is C₂₋₉heteroaryl optionally substituted with one, two, or three R^{12f}.

In some embodiments of a compound of Formula (A), R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}.

In some embodiments of a compound of Formula (A), R¹⁷ is selected from pyrazole, triazole, and pyridine, wherein pyrazole, triazole, and pyridine are optionally substituted with one, two, or three groups selected from halogen, C₁₋₆alkyl, and C₁₋₆haloalkyl.

In some embodiments of a compound of Formula (A), R^{1a} is —S(O)₂NR¹⁸R¹⁹.

In some embodiments of a compound of Formula (A), R¹⁸ and R¹⁹ is each independently hydrogen, C₁₋₆alkyl, C₆₋₁₀aryl, or C₂₋₉heteroaryl; wherein C₁₋₆alkyl, C₆₋₁₀aryl, and C₂₋₉heteroaryl are optionally substituted with one, two, or three R^{12g}.

In some embodiments of a compound of Formula (A), R¹⁸ and R¹⁹ is each independently hydrogen, C₁₋₆alkyl, C₆₋₁₀aryl, or C₂₋₉heteroaryl; wherein C₁₋₆alkyl, C₆₋₁₀aryl, and C₂₋₉heteroaryl are optionally substituted with one or two groups selected from halogen, C₁₋₆alkyl, and C₁₋₆haloalkyl.

In some embodiments of a compound of Formula (A), R¹⁸ is C₁₋₆alkyl, and R¹⁹ is C₆₋₁₀aryl or C₂₋₉heteroaryl; wherein C₆₋₁₀aryl and C₂₋₉heteroaryl are optionally substituted with one or two groups selected from halogen, C₁₋₆alkyl, and C₁₋₆haloalkyl.

In some embodiments of a compound of Formula (A), R^{1a} is —S(O)₂R¹.

In some embodiments of a compound of Formula (A), R¹ is C₆₋₁₀aryl optionally substituted with one, two, or three R^{12a}. In some embodiments of a compound of Formula (A), R¹ is phenyl optionally substituted with one, two, or three R^{12a}. In some embodiments of a compound of Formula (A), R¹ is unsubstituted phenyl. In some embodiments of a compound of Formula (A), R¹ is phenyl substituted with one, two, or three groups selected from halogen, —CN, C₁₋₆alkyl, and C₁₋₆haloalkyl. In some embodiments of a compound of Formula (A), R¹ is phenyl substituted with one, two, or three groups selected from halogen, C₁₋₆alkyl, and C₁₋₆haloalkyl. In some embodiments of a compound of Formula (A), R¹ is phenyl substituted with one, two, or three halogens. In some embodiments of a compound of Formula (A), R¹ is phenyl substituted with two halogens. In some embodiments of a compound of Formula (A), R¹ is phenyl substituted with one halogen. In some embodiments of a compound of Formula (A), R¹ is phenyl substituted with one C₁₋₆alkyl. In some embodiments of a compound of Formula (A), R¹ is phenyl substituted with one —CH₃. In some embodiments of a compound of Formula (A), R¹ is phenyl substituted with one C₁₋₆haloalkyl. In some embodiments of a compound of Formula (A), R¹ is phenyl substituted with one —CF₃. In some embodiments of a compound of Formula (A), R¹ is phenyl substituted with one —CN.

In some embodiments of a compound of Formula (A), R¹ is C₂₋₉heteroaryl optionally substituted with one, two, or three R^{12a}. In some embodiments of a compound of Formula (A), R¹ is selected from pyrazole, thiazole, oxazole, isoxazole, imidazole, triazole, and pyridine, wherein pyrazole, thiazole, oxazole, isoxazole, imidazole, triazole, and pyridine are optionally substituted with one, two, or three R^{12a}. In some embodiments of a compound of Formula (A), R¹ is selected from pyrazole, triazole, and pyridine, wherein pyrazole, triazole, and pyridine are optionally substituted with one, two, or three R^{12a}. In some embodiments of a compound of Formula (A), R¹ is unsubstituted pyrazole, triazole, or pyridine. In some embodiments of a compound of Formula (A), R¹ is pyrazole substituted with one or two groups selected from halogen, C₁₋₆alkyl, and C₁₋₆haloalkyl. In some embodiments of a compound of Formula (A), R¹ is pyrazole substituted with one C₁₋₆alkyl. In some embodiments of a compound of Formula (A), R¹ is pyrazole substituted with one —CH₃. In some embodiments of a compound of Formula (A), R¹ is triazole substituted with one or two groups selected from halogen, C₁₋₆alkyl, and C₁₋₆haloalkyl. In some embodiments of a compound of Formula (A), R¹ is triazole substituted with one C₁₋₆alkyl. In some embodiments of a compound of Formula (A), R¹ is triazole substituted with one —CH₃. In some embodiments of a compound of Formula (A), R¹ is pyridine substituted with one or two groups selected from halogen, C₁₋₆alkyl, and C₁₋₆haloalkyl. In some embodiments of a compound of Formula (A), R¹ is pyridine substituted with one or two halogens. In some embodiments of a compound of Formula (A), R¹ is pyridine substituted with one halogen. In some embodiments of a compound of Formula (A), R¹ is pyridine substituted with one C₁₋₆alkyl. In some embodiments of a compound of Formula (A), R¹ is pyridine substituted with one —CH₃. In some embodiments of a compound of Formula (A), R¹ is pyridine substituted with one C₁₋₆haloalkyl. In some embodiments of a compound of Formula (A), R¹ is pyridine substituted with one —CF₃.

In some embodiments of a compound of Formula (A), R¹ is C₃₋₈cycloalkyl optionally substituted with one, two, or three R^{12a}. In some embodiments of a compound of Formula (A), R¹ is selected from cyclobutyl, cyclopentyl and cyclohexyl, wherein cyclobutyl, cyclopentyl and cyclohexyl, are optionally substituted with one, two, or three R^{12a}. In some embodiments of a compound of Formula (A), R¹ is unsubstituted cyclobutyl. In some embodiments of a compound of Formula (A), R¹ is unsubstituted cyclopentyl. In some embodiments of a compound of Formula (A), R¹ is unsubstituted cyclohexyl.

In some embodiments of a compound of Formula (A), R¹ is C₁₋₆alkyl optionally substituted with one, two, or three R^{12a}. In some embodiments of a compound of Formula (A), R¹ is C₁₋₆alkyl substituted with phenyl, wherein phenyl is optionally substituted with one, two, or three groups selected from halogen, C₁₋₆alkyl, and C₁₋₆haloalkyl. In some embodiments of a compound of Formula (A), R¹ is C₁₋₆alkyl substituted with phenyl, wherein phenyl is unsubstituted. In some embodiments of a compound of Formula (A), R¹ is C₁₋₆alkyl substituted with phenyl, wherein phenyl is substituted with one, two, or three groups selected from halogen, C₁₋₆alkyl, and C₁₋₆haloalkyl.

In some embodiments of a compound of Formula (A), R² is C₁₋₆alkyl, C₂₋₆alkenyl, C₃₋₈cycloalkyl, or —C(O)R¹¹, wherein C₁₋₆alkyl, C₂₋₆alkenyl, and C₃₋₈cycloalkyl are optionally substituted with one, two, or three R^{12b}. In some embodiments of a compound of Formula (A), R² is C₁₋₆alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three R^{12b}. In some embodiments of a compound of Formula (A), R² is C₁₋₆alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three groups selected from halogen, C₁₋₆alkyl, C₁₋₆haloalkyl, —OR¹³, —NR¹³R¹⁴, and C₂₋₉heteroaryl. In some embodiments of a compound of Formula (A), R² is C₁₋₆alkyl optionally substituted with one, two, or three groups selected from halogen, —OR¹³, —NR¹³R¹⁴, and C₂₋₉heteroaryl. In some embodiments of a compound of Formula (A), R² is C₁₋₃alkyl substituted with one, two, or three groups selected from halogen, —OR¹³, —NR¹³R¹⁴, and C₂₋₉heteroaryl. In some embodiments of a compound of Formula (A), R² is C₁₋₃alkyl substituted with one, two, or three groups selected from halogen, —OH, and C₂₋₉heteroaryl. In some embodiments of a compound of Formula (A), R² is C₁₋₃alkyl substituted with one, two, or three groups selected from halogen, —OH, and pyridine. In some embodiments of a compound of Formula (A), R² is C₂₋₆alkenyl optionally substituted with one, two, or three groups selected from halogen, —OR¹³, —NR¹³R¹⁴, and C₂₋₉heteroaryl. In some embodiments of a compound of Formula (A), R² is C₂₋₆alkenyl substituted with one C₂₋₉heteroaryl. In some embodiments of a compound of Formula (A), R² is C₂₋₆alkenyl substituted with one pyridine. In some embodiments of a compound of Formula (A), R² is C₃₋₈cycloalkyl optionally substituted with one, two, or three groups selected from halogen, —OR¹³, —NR¹³R¹⁴, and C₂₋₉heteroaryl. In some embodiments of a compound of Formula (A), R² is C₃₋₈cycloalkyl substituted with one C₂₋₉heteroaryl. In some embodiments of a compound of Formula (A), R² is C₃₋₈cycloalkyl substituted with one pyridine. In some embodiments of a compound of Formula (A), R² is cyclopropyl substituted with one C₂₋₉heteroaryl. In some embodiments of a compound of Formula (A), R² is cyclopropyl substituted with one pyridine.

In some embodiments of a compound of Formula (A), R² is —C(O)R¹¹. In some embodiments of a compound of Formula (A), R² is —C(O)R¹¹ and R¹¹ is C₆₋₁₀aryl or C₂₋₉heteroaryl; wherein C₆₋₁₀aryl and C₂₋₉heteroaryl are optionally substituted with one, two, or three R^{12e}. In some embodiments of a compound of Formula (A), R² is —C(O)R¹¹ and R¹¹ is C₂₋₉heteroaryl optionally substituted with one, two, or three R^{12e}. In some embodiments of a compound of Formula (A), R² is —C(O)R¹¹ and R¹¹ is C₂₋₉heteroaryl optionally substituted with one or two R^{12e}. In some embodiments of a compound of Formula (A), R² is —C(O)R¹¹ and R¹¹ is C₂₋₉heteroaryl optionally substituted with one or two groups selected from halogen, C₁₋₆alkyl, and C₁₋₆haloalkyl. In some embodiments of a compound of Formula (A), R² is —C(O)R¹¹ and R¹¹ is selected from thiazole and pyridine, wherein thiazole and pyridine are optionally substituted with one or two groups selected from halogen, C₁₋₆alkyl, and C₁₋₆haloalkyl. In some embodiments of a compound of Formula (A), R² is —C(O)R¹¹ and R¹¹ is thiazole optionally substituted with one or two groups selected from halogen, C₁₋₆alkyl, and C₁₋₆haloalkyl. In some embodiments of a compound of Formula (A), R² is —C(O)R¹¹ and R¹¹ is unsubstituted thiazole. In some embodiments of a compound of Formula (A), R² is —C(O)R¹¹ and R¹¹ is thiazole substituted with one or two groups selected from halogen, C₁₋₆alkyl, and C₁₋₆haloalkyl. In some embodiments of a compound of Formula (A), R² is —C(O)R¹¹ and R¹¹ is pyridine optionally substituted with one or two groups selected from halogen, C₁₋₆alkyl, and C₁₋₆haloalkyl. In some embodiments of a compound of Formula (A), R² is —C(O)R¹¹ and R¹¹ is unsubstituted pyridine. In some embodiments of a compound of Formula (A), R² is —C(O)R¹¹ and R¹¹ is pyridine substituted with one or two groups selected from halogen, C₁₋₆alkyl, and C₁₋₆haloalkyl. In some embodiments of a compound of Formula (A), R² is —C(O)R¹¹ and R¹¹ is pyridine substituted with one halogen. In some embodiments of a compound of Formula (A), R² is —C(O)R¹¹ and R¹¹ is pyridine substituted with one C₁₋₆alkyl. In some embodiments of a compound of Formula (A), R² is —C(O)R¹¹ and R¹¹ is pyridine substituted with one —CH₃. In some embodiments of a compound of Formula (A), R² is —C(O)R¹¹ and R¹¹ is pyridine substituted with one C₁₋₆haloalkyl. In some embodiments of a compound of Formula (A), R² is —C(O)R¹¹ and R¹¹ is pyridine substituted with one —CF₃.

In some embodiments of a compound of Formula (A), R⁶ is C₆₋₁₀aryl or C₂₋₉heteroaryl; wherein C₆₋₁₀aryl and C₂₋₉heteroaryl are optionally substituted with one, two, or three R^{12c}. In some embodiments of a compound of Formula (A), R⁶ is C₆₋₁₀aryl optionally substituted with one, two, or three R^{12c}. In some embodiments of a compound of Formula (A), R⁶ is phenyl optionally substituted with one, two, or three R^{12c}. In some embodiments of a compound of Formula (A), R⁶ is unsubstituted phenyl. In some embodiments of a compound of Formula (A), R⁶ is phenyl optionally substituted with one, two, or three groups selected from halogen, C₁₋₆alkyl, and C₁₋₆haloalkyl. In some embodiments of a compound of Formula (A), R⁶ is phenyl substituted with one or two groups selected from halogen, C₁₋₆alkyl, and C₁₋₆haloalkyl. In some embodiments of a compound of Formula (A), R⁶ is phenyl substituted with one group selected from halogen, C₁₋₆alkyl, and C₁₋₆haloalkyl. In some embodiments of a compound of Formula (A), R⁶ is phenyl substituted with one halogen. In some embodiments of a compound of Formula (A), R⁶ is phenyl substituted with one F. In some embodiments of a compound of Formula (A), R⁶ is phenyl substituted with one Cl. In some embodiments of a compound of Formula (A), R⁶ is phenyl substituted with one C₁₋₆alkyl. In some embodiments of a compound of Formula (A), R⁶ is phenyl substituted with one —CH₃. In some embodiments of a compound of Formula (A), R⁶ is phenyl substituted with one C₁₋₆haloalkyl. In some embodiments of a compound of Formula (A), R⁶ is phenyl substituted with one —CF₃.

In some embodiments of a compound of Formula (A), R⁵ is hydrogen. In some embodiments of a compound of Formula (A), R⁵ is C₁₋₆alkyl. In some embodiments of a compound of Formula (A), R⁵ is —CH₃. In some embodiments of a compound of Formula (A), R⁵ is C₁₋₆halolkyl. In some embodiments of a compound of Formula (A), R⁵ is —CF₃.

In some embodiments of a compound of Formula (A), R⁷ is hydrogen, halogen, or C₁₋₆alkyl. In some embodiments of a compound of Formula (A), R⁷ is hydrogen or C₁₋₆alkyl. In some embodiments of a compound of Formula (A), R⁷ is hydrogen. In some embodiments of a compound of Formula (A), R⁷ is C₁₋₆alkyl. In some embodiments of a compound of Formula (A), R⁷ is —CH₃.

In some embodiments of a compound of Formula (A), m is 0. In some embodiments of a compound of Formula (A), m is 1. In some embodiments of a compound of Formula (A), m is 2. In some embodiments of a compound of Formula (A), m is 3. In some embodiments of a compound of Formula (A), m is 4. In some embodiments of a compound of Formula (A), n is 0. In some embodiments of a compound of Formula (A), n is 1. In some embodiments of a compound of Formula (A), m is 2. In some embodiments of a compound of Formula (A), n is 3. In some embodiments of a compound of Formula (A), m is 0 and n is 0.

In some embodiments of a compound of Formula (A),

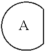

is

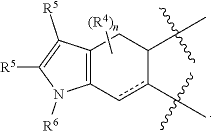

In some embodiments of a compound of Formula (A),

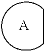

is

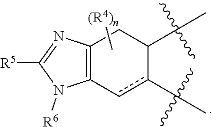

In some embodiments of a compound of Formula (A),

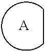

is

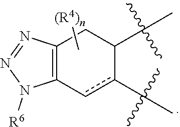

In some embodiments of a compound of Formula (A), [symbol] is a single bond. In some embodiments of a compound of Formula (A), [symbol] is a double bond.

In some embodiments of a compound of Formula (A),

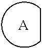

is

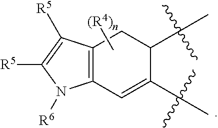

In some embodiments of a compound of Formula (A),

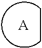

is

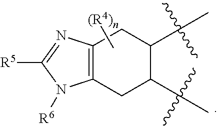

In some embodiments of a compound of Formula (A),

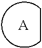

is

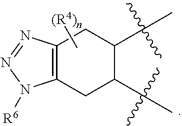

In some embodiments of a compound of Formula (A),

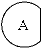

is

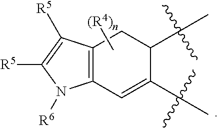

In some embodiments of a compound of Formula (A),

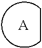

is

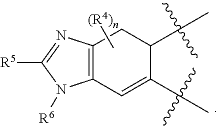

In some embodiments of a compound of Formula (A),

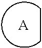

is

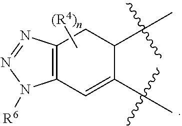

In some embodiments provided herein is a compound having the structure of Formula (B), or a pharmaceutically acceptable salt, solvate, stereoisomer, or isotopic variant thereof:

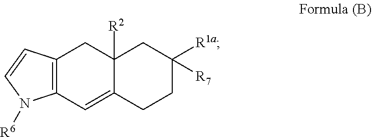

wherein:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R¹ is C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl or C₂₋₉heteroaryl; wherein C₆₋₁₀aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, C₂₋₉heterocycloalkyl, C₆₋₁₀aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a heterocycloalkyl optionally substituted with one, two, or three groups selected from halogen, C₁₋₆alkyl, and C₁₋₆haloalkyl; and
each R¹⁵ is independently C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl; R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl; and
R¹ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12a}.

In some embodiments provided herein is a compound having the structure of Formula (C), or a pharmaceutically acceptable salt, solvate, stereoisomer, or isotopic variant thereof:

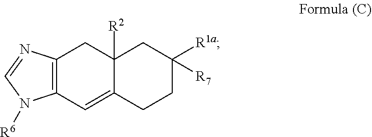

wherein:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl or C₂₋₉heteroaryl; wherein C₆₋₁₀aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, C₂₋₉heterocycloalkyl, C₆₋₁₀aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl; and
R¹ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12a}.

In some embodiments provided herein is a compound having the structure of Formula (D), or a pharmaceutically acceptable salt, solvate, stereoisomer, or isotopic variant thereof:

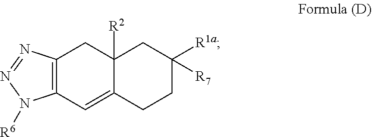

wherein:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl or C₂₋₉heteroaryl; wherein C₆₋₁₀aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, C₂₋₉heterocycloalkyl, C₆₋₁₀aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl; wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl; and
R¹ is alkyl, cycloalkyl, heterocycloalkyl, aryl, or heteroaryl; wherein alkyl, cycloalkyl, heterocycloalkyl, aryl, and heteroaryl are optionally substituted with one, two, or three R^{12a}.

In some embodiments of a compound of Formula (B), (C), or (D), R^{1a} is —NR¹⁶S(O)₂R¹⁷.

In some embodiments of a compound of Formula (B), (C), or (D), R^{1a} is —NR¹⁶C(O)R¹⁷.

In some embodiments of a compound of Formula (B), (C), or (D), R¹⁶ is C₁₋₆alkyl or C₃₋₈cycloalkyl, wherein C₁₋₆alkyl and C₃₋₈cycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵.

In some embodiments of a compound of Formula (B), (C), or (D), R¹⁶ is unsubstituted C₁₋₆alkyl.

In some embodiments of a compound of Formula (B), (C), or (D), R¹⁶ is C₁₋₆haloalkyl.

In some embodiments of a compound of Formula (B), (C), or (D), R¹⁶ is unsubstituted C₃₋₈cycloalkyl.

In some embodiments of a compound of Formula (B), (C), or (D), R¹⁶ is unsubstituted cyclopropyl.

In some embodiments of a compound of Formula (B), (C), or (D), R^{1a} is —C(R²⁰)₂S(O)₂R¹⁷.

In some embodiments of a compound of Formula (B), (C), or (D), R²⁰ is hydrogen, C₁₋₆alkyl, or C₃₋₈cycloalkyl.

In some embodiments of a compound of Formula (B), (C), or (D), R¹⁷ is C₆₋₁₀aryl or C₂₋₉heteroaryl, and the C₆₋₁₀aryl and C₂₋₉heteroaryl are optionally substituted with one, two, or three R^{12f}.

In some embodiments of a compound of Formula (B), (C), or (D), R¹⁷ is phenyl optionally substituted with one, two, or three R^{12f}.

In some embodiments of a compound of Formula (B), (C), or (D), R¹⁷ is phenyl optionally substituted with one, two, or three groups selected from halogen, C₁₋₆alkyl, and C₁₋₆haloalkyl.

In some embodiments of a compound of Formula (B), (C), or (D), R¹⁷ is C₂₋₉heteroaryl optionally substituted with one, two, or three R^{12f}.

In some embodiments of a compound of Formula (B), (C), or (D), R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}.

In some embodiments of a compound of Formula (B), (C), or (D), R¹⁷ is selected from pyrazole, triazole, and pyridine, wherein pyrazole, triazole, and pyridine are optionally substituted with one, two, or three groups selected from halogen, C₁₋₆alkyl, and C₁₋₆haloalkyl.

In some embodiments of a compound of Formula (B), (C), or (D), R^{1a} is —S(O)₂NR¹⁸R¹⁹.

In some embodiments of a compound of Formula (B), (C), or (D), R¹⁸ and R¹⁹ is each independently hydrogen, C₁₋₆alkyl, C₆₋₁₀aryl, or C₂₋₉heteroaryl; wherein C₁₋₆alkyl, C₆₋₁₀aryl, and C₂₋₉heteroaryl are optionally substituted with one, two, or three R^{12g}.

In some embodiments of a compound of Formula (B), (C), or (D), R¹⁸ and R¹⁹ is each independently hydrogen, C₁₋₆alkyl, C₆₋₁₀aryl, or C₂₋₉heteroaryl; wherein C₁₋₆alkyl, C₆₋₁₀aryl, and C₂₋₉heteroaryl are optionally substituted with one or two groups selected from halogen, C₁₋₆alkyl, and C₁₋₆haloalkyl.

In some embodiments of a compound of Formula (B), (C), or (D), R¹⁸ is C₁₋₆alkyl, and R¹⁹ is C₆₋₁₀aryl or C₂₋₉heteroaryl; wherein C₆₋₁₀aryl and C₂₋₉heteroaryl are optionally substituted with one or two groups selected from halogen, C₁₋₆alkyl, and C₁₋₆haloalkyl.

In some embodiments of a compound of Formula (B), (C), or (D), R^{1a} is —S(O)₂R¹.

In some embodiments of a compound of Formula (B), (C), or (D), R¹ is C₆₋₁₀aryl optionally substituted with one, two, or three R^{12a}. In some embodiments of a compound of Formula (B), (C), or (D), R¹ is phenyl optionally substituted with one, two, or three R^{12a}. In some embodiments of a compound of Formula (B), (C), or (D), R¹ is unsubstituted phenyl. In some embodiments of a compound of Formula (B), (C), or (D), R¹ is phenyl substituted with one, two, or three groups selected from halogen, —CN, C₁₋₆alkyl, and C₁₋₆haloalkyl. In some embodiments of a compound of Formula (B), (C), or (D), R¹ is phenyl substituted with one, two, or three groups selected from halogen, C₁₋₆alkyl, and C₁₋₆haloalkyl. In some embodiments of a compound of Formula (B), (C), or (D), R¹ is phenyl substituted with one, two, or three halogens. In some embodiments of a compound of Formula (B), (C), or (D), R¹ is phenyl substituted with two halogens. In some embodiments of a compound of Formula (B), (C), or (D), R¹ is phenyl substituted with one halogen. In some embodiments of a compound of Formula (B), (C), or (D), R¹ is phenyl substituted with one C₁₋₆alkyl. In some embodiments of a compound of Formula (B), (C), or (D), R¹ is phenyl substituted with one —CH₃. In some embodiments of a compound of Formula (B), (C), or (D), R¹ is phenyl substituted with one C₁₋₆haloalkyl. In some embodiments of a compound of Formula (B), (C), or (D), R¹ is phenyl substituted with one —CF₃. In some embodiments of a compound of Formula (B), (C), or (D), R¹ is phenyl substituted with one —CN.

In some embodiments of a compound of Formula (B), (C), or (D), R¹ is C₂₋₉heteroaryl optionally substituted with one, two, or three R^{12a}. In some embodiments of a compound of Formula (B), (C), or (D), R¹ is selected from pyrazole, thiazole, oxazole, isoxazole, imidazole, triazole, and pyridine, wherein pyrazole, thiazole, oxazole, isoxazole, imidazole, triazole, and pyridine are optionally substituted with one, two, or three R^{12a}. In some embodiments of a compound of Formula (B), (C), or (D), R¹ is selected from pyrazole, triazole, and pyridine, wherein pyrazole, triazole, and pyridine are optionally substituted with one, two, or three R^{12a}. In some embodiments of a compound of Formula (B), (C), or (D), R¹ is unsubstituted pyrazole, triazole, or pyridine. In some embodiments of a compound of Formula (B), (C), or (D), R¹ is pyrazole substituted with one or two groups selected from halogen, C₁₋₆alkyl, and C₁₋₆haloalkyl. In some embodiments of a compound of Formula (B), (C), or (D), R¹ is pyrazole substituted with one C₁₋₆alkyl. In some embodiments of a compound of Formula (B), (C), or (D), R¹ is pyrazole substituted with one —CH₃. In some embodiments of a compound of Formula (B), (C), or (D), R¹ is triazole substituted with one or two groups selected from halogen, C₁₋₆alkyl, and C₁₋₆haloalkyl. In some embodiments of a compound of Formula (B), (C), or (D), R¹ is triazole substituted with one C₁₋₆alkyl. In some embodiments of a compound of Formula (B), (C), or (D), R¹ is triazole substituted with one —CH₃. In some embodiments of a compound of Formula (B), (C), or (D), R¹ is pyridine substituted with one or two groups selected from halogen, C₁₋₆alkyl, and C₁₋₆haloalkyl. In some embodiments of a compound of Formula (B), (C), or (D), R¹ is pyridine substituted with one or two halogens. In some embodiments of a compound of Formula (B), (C), or (D), R¹ is pyridine substituted with one halogen. In some embodiments of a compound of Formula (B), (C), or (D), R¹ is pyridine substituted with one C₁₋₆alkyl. In some embodiments of a compound of Formula (B), (C), or (D), R¹ is pyridine substituted with one —CH₃. In some embodiments of a compound of Formula (B), (C), or (D), R¹ is pyridine substituted with one C₁₋₆haloalkyl. In some embodiments of a compound of Formula (B), (C), or (D), R¹ is pyridine substituted with one —CF₃.

In some embodiments of a compound of Formula (B), (C), or (D), R¹ is C₃₋₈cycloalkyl optionally substituted with one, two, or three R^{12a}. In some embodiments of a compound of Formula (B), (C), or (D), R¹ is selected from cyclobutyl, cyclopentyl and cyclohexyl, wherein cyclobutyl, cyclopentyl and cyclohexyl, are optionally substituted with one, two, or three R^{12a}. In some embodiments of a compound of Formula (B), (C), or (D), R¹ is unsubstituted cyclobutyl. In some embodiments of a compound of Formula (B), (C), or (D), R¹ is unsubstituted cyclopentyl. In some embodiments of a compound of Formula (B), (C), or (D), R¹ is unsubstituted cyclohexyl.

In some embodiments of a compound of Formula (B), (C), or (D), R¹ is C₁₋₆alkyl optionally substituted with one, two, or three R^{12a}. In some embodiments of a compound of Formula (B), (C), or (D), R¹ is C₁₋₆alkyl substituted with phenyl, wherein phenyl is optionally substituted with one, two, or three groups selected from halogen, C₁₋₆alkyl, and C₁₋₆haloalkyl. In some embodiments of a compound of Formula (B), (C), or (D), R¹ is C₁₋₆alkyl substituted with phenyl, wherein phenyl is unsubstituted. In some embodiments of a compound of Formula (B), (C), or (D), R¹ is C₁₋₆alkyl substituted with phenyl, wherein phenyl is substituted with one, two, or three groups selected from halogen, C₁₋₆alkyl, and C₁₋₆haloalkyl.

In some embodiments of a compound of Formula (B), (C), or (D), R² is C₁₋₆alkyl, C₂₋₆alkenyl, C₃₋₈cycloalkyl, or —C(O)R¹¹, wherein C₁₋₆alkyl, C₂₋₆alkenyl, and C₃₋₈cycloalkyl are optionally substituted with one, two, or three R^{12b}. In some embodiments of a compound of Formula (B), (C), or (D), R² is C₁₋₆alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three R^{12b}. In some embodiments of a compound of Formula (B), (C), or (D), R² is C₁₋₆alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three groups selected from halogen, C₁₋₆alkyl, C₁₋₆haloalkyl, —OR¹³, —NR¹³R¹⁴, and C₂₋₉heteroaryl. In some embodiments of a compound of Formula (B), (C), or (D), R² is C₁₋₆alkyl optionally substituted with one, two, or three groups selected from halogen, —OR¹³, —NR¹³R¹⁴, and C₂₋₉heteroaryl. In some embodiments of a compound of Formula (B), (C), or (D), R² is C₁₋₃alkyl substituted with one, two, or three groups selected from halogen, —OR¹³, —NR¹³R¹⁴, and C₂₋₉heteroaryl. In some embodiments of a compound of Formula (B), (C), or (D), R² is C₁₋₃alkyl substituted with one, two, or three groups selected from halogen, —OH, and C₂₋₉heteroaryl. In some embodiments of a compound of Formula (B), (C), or (D), R² is C₁₋₃alkyl substituted with one, two, or three groups selected from halogen, —OH, and pyridine. In some embodiments of a compound of Formula (B), (C), or (D), R² is C₂₋₆alkenyl optionally substituted with one, two, or three groups selected from halogen, —OR¹³, —NR¹³R¹⁴, and C₂₋₉heteroaryl. In some embodiments of a compound of Formula (B), (C), or (D), R² is C₂₋₆alkenyl substituted with one C₂₋₉heteroaryl. In some embodiments of a compound of Formula (B), (C), or (D), R² is C₂₋₆alkenyl substituted with one pyridine. In some embodiments of a compound of Formula (B), (C), or (D), R² is C₃₋₈cycloalkyl optionally substituted with one, two, or three groups selected from halogen, —OR¹³, —NR¹³R¹⁴, and C₂₋₉heteroaryl. In some embodiments of a compound of Formula (B), (C), or (D), R² is C₃₋₈cycloalkyl substituted with one C₂₋₉heteroaryl. In some embodiments of a compound of Formula (B), (C), or (D), R² is C₃₋₈cycloalkyl substituted with one pyridine. In some embodiments of a compound of Formula (B), (C), or (D), R² is cyclopropyl substituted with one C₂₋₉heteroaryl. In some embodiments of a compound of Formula (B), (C), or (D), R² is cyclopropyl substituted with one pyridine.

In some embodiments of a compound of Formula (B), (C), or (D), R² is —C(O)R¹¹. In some embodiments of a compound of Formula (B), (C), or (D), R² is —C(O)R¹¹ and R¹¹ is C₆₋₁₀aryl or C₂₋₉heteroaryl; wherein C₆₋₁₀aryl and C₂₋₉heteroaryl are optionally substituted with one, two, or three R^{12e}. In some embodiments of a compound of Formula (B), (C), or (D), R² is —C(O)R¹¹ and R¹¹ is C₂₋₉heteroaryl optionally substituted with one, two, or three R^{12e}. In some embodiments of a compound of Formula (B), (C), or (D), R² is —C(O)R¹¹ and R¹¹ is C₂₋₉heteroaryl optionally substituted with one or two R^{12e}. In some embodiments of a compound of Formula (B), (C), or (D), R² is —C(O)R¹¹ and R¹¹ is C₂₋₉heteroaryl optionally substituted with one or two groups selected from halogen, C₁₋₆alkyl, and C₁₋₆haloalkyl. In some embodiments of a compound of Formula (B), (C), or (D), R² is —C(O)R¹¹ and R¹¹ is selected from thiazole and pyridine, wherein thiazole and pyridine are optionally substituted with one or two groups selected from halogen, C₁₋₆alkyl, and C₁₋₆haloalkyl. In some embodiments of a compound of Formula (B), (C), or (D), R² is —C(O)R¹¹ and R¹¹ is thiazole optionally substituted with one or two groups selected from halogen, C₁₋₆alkyl, and C₁₋₆haloalkyl. In some embodiments of a compound of Formula (B), (C), or (D), R² is —C(O)R¹¹ and R¹¹ is unsubstituted thiazole. In some embodiments of a compound of Formula (B), (C), or (D), R² is —C(O)R¹¹ and R¹¹ is thiazole substituted with one or two groups selected from halogen, C₁₋₆alkyl, and C₁₋₆haloalkyl. In some embodiments of a compound of Formula (B), (C), or (D), R² is —C(O)R¹¹ and R¹¹ is pyridine optionally substituted with one or two groups selected from halogen, C₁₋₆alkyl, and C₁₋₆haloalkyl. In some embodiments of a compound of Formula (B), (C), or (D), R² is —C(O)R¹¹ and R¹¹ is unsubstituted pyridine. In some embodiments of a compound of Formula (B), (C), or (D), R² is —C(O)R¹¹ and R¹¹ is pyridine substituted with one or two groups selected from halogen, C₁₋₆alkyl, and C₁₋₆haloalkyl. In some embodiments of a compound of Formula (B), (C), or (D), R² is —C(O)R¹¹ and R¹¹ is pyridine substituted with one halogen. In some embodiments of a compound of Formula (B), (C), or (D), R² is —C(O)R¹¹ and R¹¹ is pyridine substituted with one C₁₋₆alkyl. In some embodiments of a compound of Formula (B), (C), or (D), R² is —C(O)R¹¹ and R¹¹ is pyridine substituted with one —CH₃. In some embodiments of a compound of Formula (B), (C), or (D), R² is —C(O)R¹¹ and R¹¹ is pyridine substituted with one C₁₋₆haloalkyl. In some embodiments of a compound of Formula (B), (C), or (D), R² is —C(O)R¹¹ and R¹¹ is pyridine substituted with one —CF₃.

In some embodiments of a compound of Formula (B), (C), or (D), R⁶ is C₆₋₁₀aryl or C₂₋₉heteroaryl; wherein C₆₋₁₀aryl and C₂₋₉heteroaryl are optionally substituted with one, two, or three R^{12c}. In some embodiments of a compound of Formula (B), (C), or (D), R⁶ is C₆₋₁₀aryl optionally substituted with one, two, or three R^{12c}. In some embodiments of a compound of Formula (B), (C), or (D), R⁶ is phenyl optionally substituted with one, two, or three R^{12c}. In some embodiments of a compound of Formula (B), (C), or (D), R⁶ is unsubstituted phenyl. In some embodiments of a compound of Formula (B), (C), or (D), R⁶ is phenyl optionally substituted with one, two, or three groups selected from halogen, C₁₋₆alkyl, and C₁₋₆haloalkyl. In some embodiments of a compound of Formula (B), (C), or (D), R⁶ is phenyl substituted with one or two groups selected from halogen, C₁₋₆alkyl, and C₁₋₆haloalkyl. In some embodiments of a compound of Formula (B), (C), or (D), R⁶ is phenyl substituted with one group selected from halogen, C₁₋₆alkyl, and C₁₋₆haloalkyl. In some embodiments of a compound of Formula (B), (C), or (D), R⁶ is phenyl substituted with one halogen. In some embodiments of a compound of Formula (B), (C), or (D), R⁶ is phenyl substituted with one F. In some embodiments of a compound of Formula (B), (C), or (D), R⁶ is phenyl substituted with one Cl. In some embodiments of a compound of Formula (B), (C), or (D), R⁶ is phenyl substituted with one C₁₋₆alkyl. In some embodiments of a compound of Formula (B), (C), or (D), R⁶ is phenyl substituted with one —CH₃. In some embodiments of a compound of Formula (B), (C), or (D), R⁶ is phenyl substituted with one C₁₋₆haloalkyl. In some embodiments of a compound of Formula (B), (C), or (D), R⁶ is phenyl substituted with one —CF₃.

In some embodiments of a compound of Formula (B), (C), or (D), R⁷ is hydrogen, halogen, or C₁₋₆alkyl. In some embodiments of a compound of Formula (B), (C), or (D), R⁷ is hydrogen or C₁₋₆alkyl. In some embodiments of a compound of Formula (B), (C), or (D), R⁷ is hydrogen. In some embodiments of a compound of Formula (B), (C), or (D), R⁷ is C₁₋₆alkyl. In some embodiments of a compound of Formula (B), (C), or (D), R⁷ is —CH₃.

In some embodiments is a compound, or a pharmaceutically acceptable salt, solvate, stereoisomer, or isotopic variant thereof, having a structure selected from:

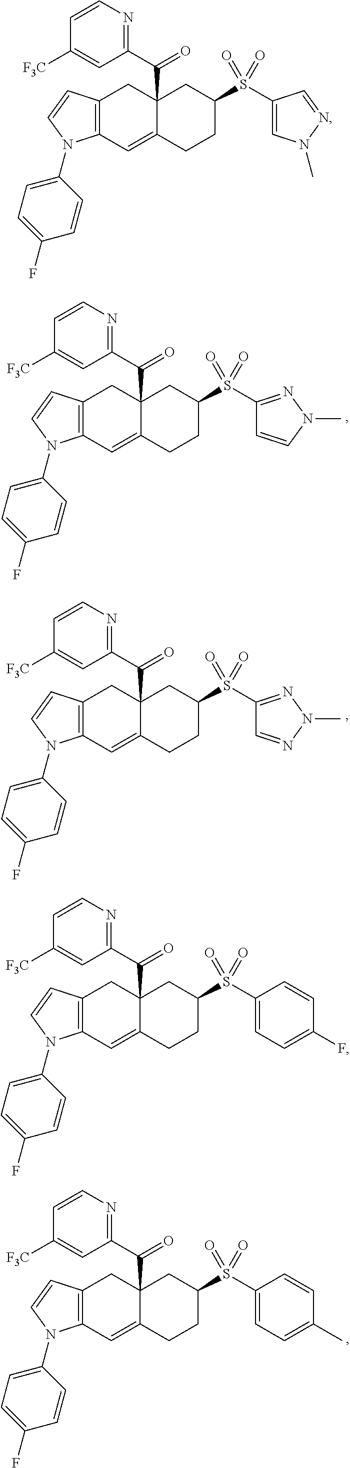 [SMILES: Cn1cc(S(=O)(=O)[C@H]2CCC3=Cc4c(ccn4-c4ccc(F)cc4)C[C@]3(C(=O)c3cc(C(F)(F)F)ccn3)C2)cn1 ; Cn1ccc(S(=O)(=O)[C@H]2CCC3=Cc4c(ccn4-c4ccc(F)cc4)C[C@]3(C(=O)c3cc(C(F)(F)F)ccn3)C2)n1 ; Cn1ncc(S(=O)(=O)[C@H]2CCC3=Cc4c(ccn4-c4ccc(F)cc4)C[C@]3(C(=O)c3cc(C(F)(F)F)ccn3)C2)n1 ; O=C(c1cc(C(F)(F)F)ccn1)[C@]12Cc3ccn(-c4ccc(F)cc4)c3C=C1CC[C@H](S(=O)(=O)c1ccc(F)cc1)C2 ; Cc1ccc(S(=O)(=O)[C@H]2CCC3=Cc4c(ccn4-c4ccc(F)cc4)C[C@]3(C(=O)c3cc(C(F)(F)F)ccn3)C2)cc1]

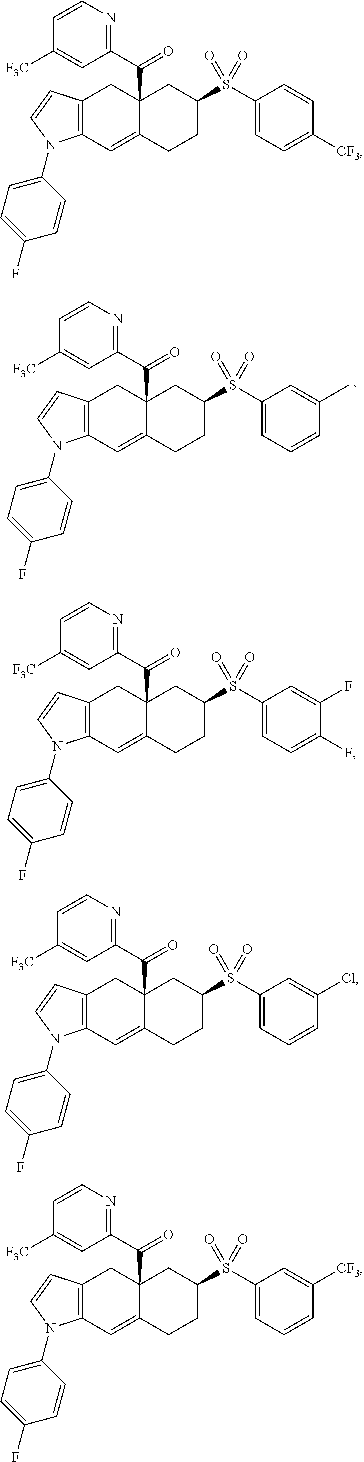 [SMILES: O=C(c1cc(C(F)(F)F)ccn1)[C@]12Cc3ccn(-c4ccc(F)cc4)c3C=C1CC[C@H](S(=O)(=O)c1ccc(C(F)(F)F)cc1)C2 ; Cc1cccc(S(=O)(=O)[C@H]2CCC3=Cc4c(ccn4-c4ccc(F)cc4)C[C@]3(C(=O)c3cc(C(F)(F)F)ccn3)C2)c1 ; O=C(c1cc(C(F)(F)F)ccn1)[C@]12Cc3ccn(-c4ccc(F)cc4)c3C=C1CC[C@H](S(=O)(=O)c1ccc(F)c(F)c1)C2 ; O=C(c1cc(C(F)(F)F)ccn1)[C@]12Cc3ccn(-c4ccc(F)cc4)c3C=C1CC[C@H](S(=O)(=O)c1cccc(Cl)c1)C2 ; O=C(c1cc(C(F)(F)F)ccn1)[C@]12Cc3ccn(-c4ccc(F)cc4)c3C=C1CC[C@H](S(=O)(=O)c1cccc(C(F)(F)F)c1)C2]

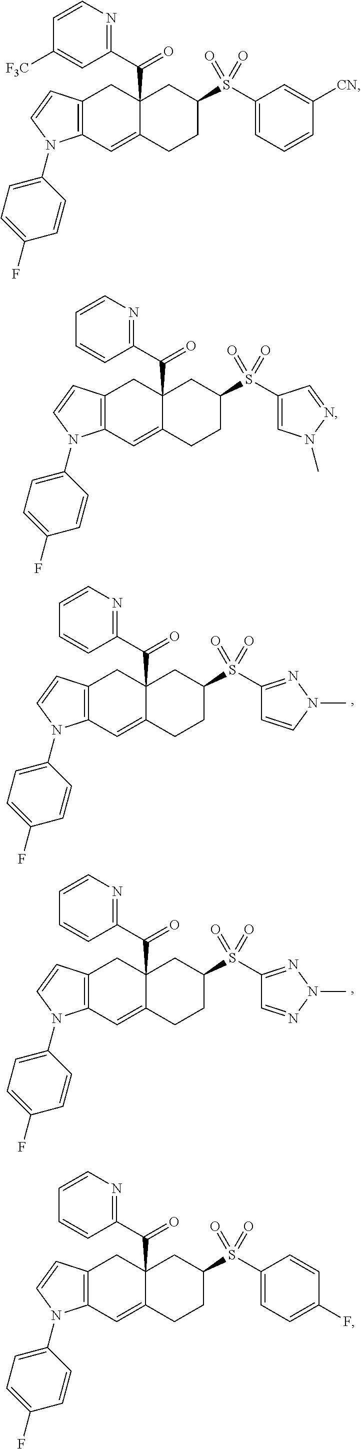

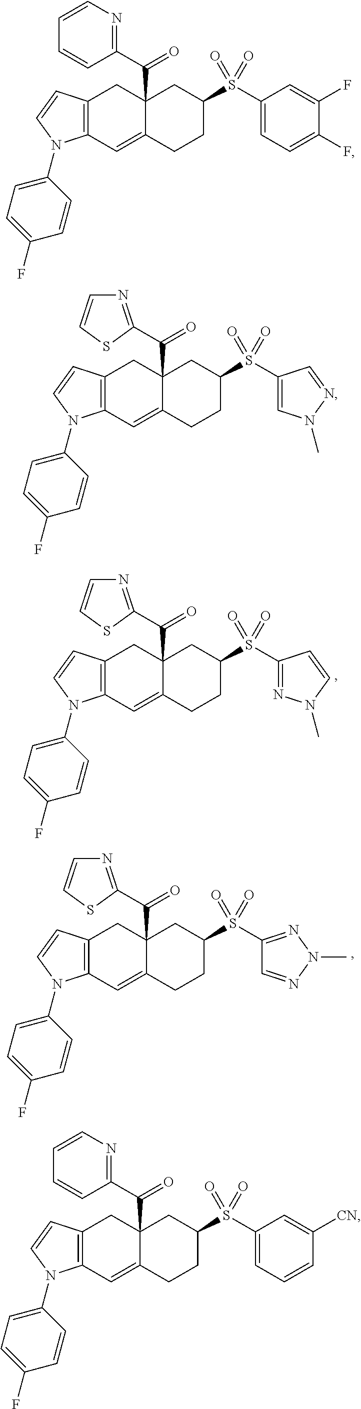

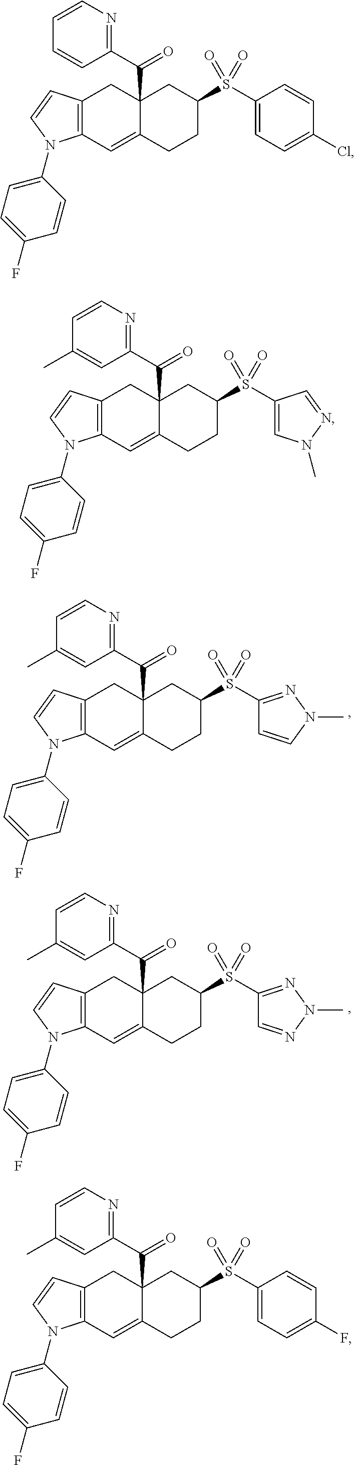 [SMILES: O=C(c1ccccn1)[C@]12Cc3ccn(-c4ccc(F)cc4)c3C=C1CC[C@H](S(=O)(=O)c1ccc(Cl)cc1)C2 ; Cc1ccnc(C(=O)[C@]23Cc4ccn(-c5ccc(F)cc5)c4C=C2CC[C@H](S(=O)(=O)c2cnn(C)c2)C3)c1 ; Cc1ccnc(C(=O)[C@]23Cc4ccn(-c5ccc(F)cc5)c4C=C2CC[C@H](S(=O)(=O)c2ccn(C)n2)C3)c1 ; Cc1ccnc(C(=O)[C@]23Cc4ccn(-c5ccc(F)cc5)c4C=C2CC[C@H](S(=O)(=O)c2cnn(C)n2)C3)c1 ; Cc1ccnc(C(=O)[C@]23Cc4ccn(-c5ccc(F)cc5)c4C=C2CC[C@H](S(=O)(=O)c2ccc(F)cc2)C3)c1]

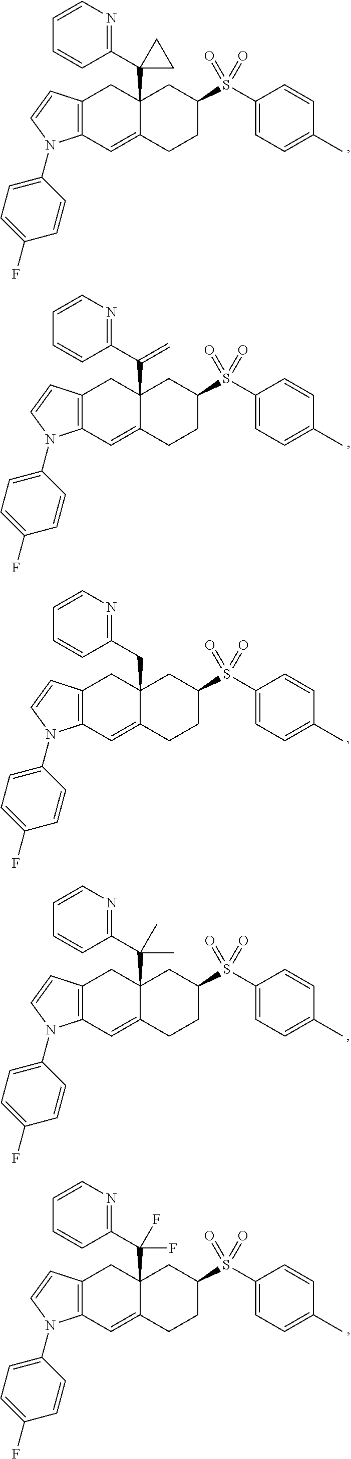 [SMILES: Cc1ccc(S(=O)(=O)[C@H]2CCC3=Cc4c(ccn4-c4ccc(F)cc4)C[C@]3(C3(c4ccccn4)CC3)C2)cc1 ; C=C(c1ccccn1)[C@]12Cc3ccn(-c4ccc(F)cc4)c3C=C1CC[C@H](S(=O)(=O)c1ccc(C)cc1)C2 ; Cc1ccc(S(=O)(=O)[C@H]2CCC3=Cc4c(ccn4-c4ccc(F)cc4)C[C@]3(Cc3ccccn3)C2)cc1 ; Cc1ccc(S(=O)(=O)[C@H]2CCC3=Cc4c(ccn4-c4ccc(F)cc4)C[C@]3(C(C)(C)c3ccccn3)C2)cc1 ; Cc1ccc(S(=O)(=O)[C@H]2CCC3=Cc4c(ccn4-c4ccc(F)cc4)C[C@]3(C(F)(F)c3ccccn3)C2)cc1]

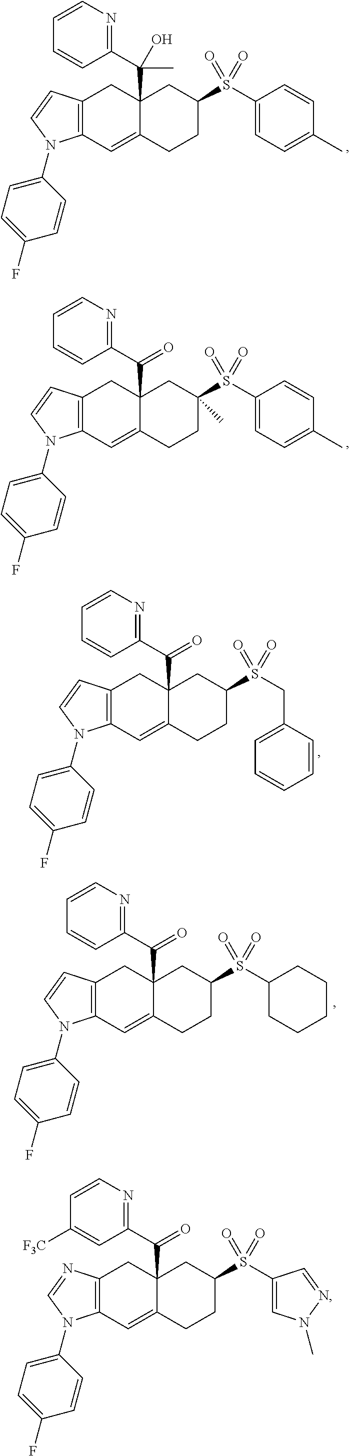

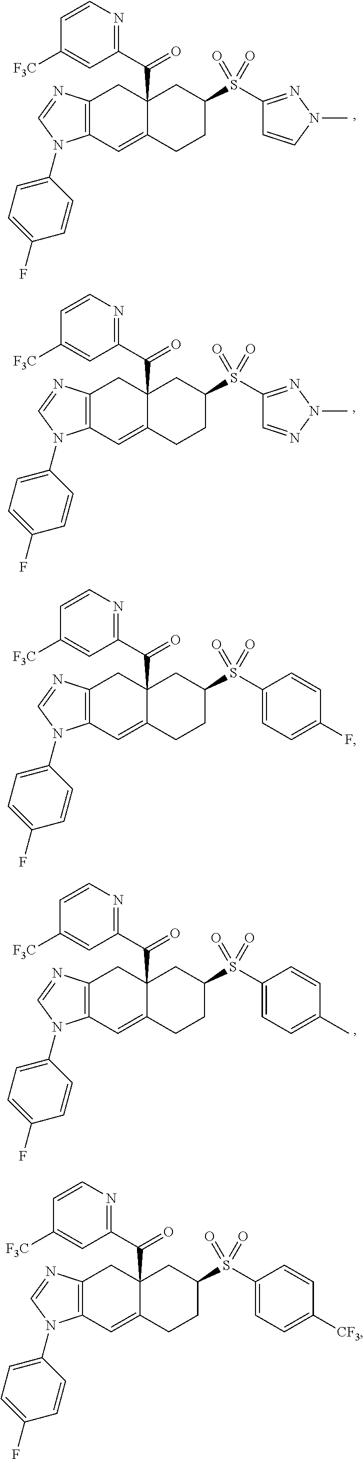 [SMILES: Cn1ccc(S(=O)(=O)[C@H]2CCC3=Cc4c(ncn4-c4ccc(F)cc4)C[C@]3(C(=O)c3cc(C(F)(F)F)ccn3)C2)n1 ; Cn1ncc(S(=O)(=O)[C@H]2CCC3=Cc4c(ncn4-c4ccc(F)cc4)C[C@]3(C(=O)c3cc(C(F)(F)F)ccn3)C2)n1 ; O=C(c1cc(C(F)(F)F)ccn1)[C@]12Cc3ncn(-c4ccc(F)cc4)c3C=C1CC[C@H](S(=O)(=O)c1ccc(F)cc1)C2 ; Cc1ccc(S(=O)(=O)[C@H]2CCC3=Cc4c(ncn4-c4ccc(F)cc4)C[C@]3(C(=O)c3cc(C(F)(F)F)ccn3)C2)cc1 ; O=C(c1cc(C(F)(F)F)ccn1)[C@]12Cc3ncn(-c4ccc(F)cc4)c3C=C1CC[C@H](S(=O)(=O)c1ccc(C(F)(F)F)cc1)C2]

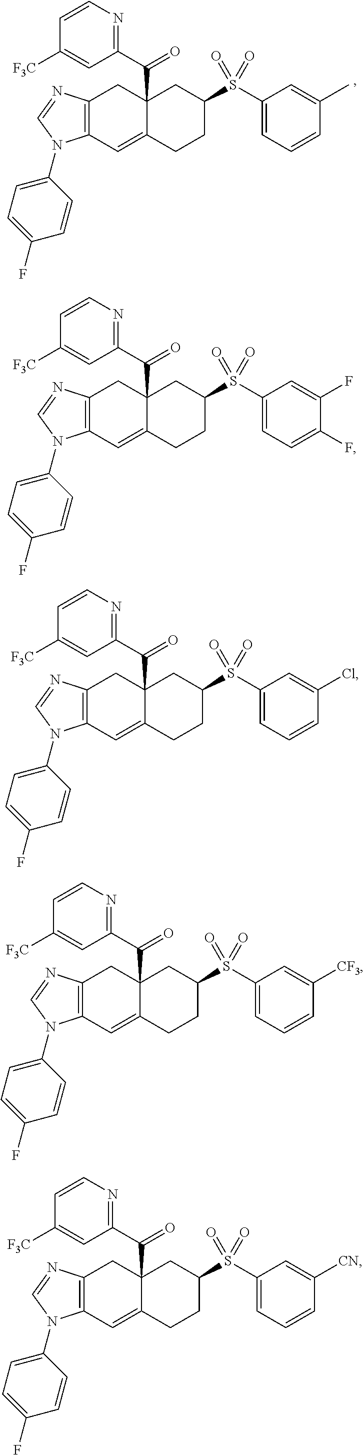 [SMILES: Cc1cccc(S(=O)(=O)[C@H]2CCC3=Cc4c(ncn4-c4ccc(F)cc4)C[C@]3(C(=O)c3cc(C(F)(F)F)ccn3)C2)c1 ; O=C(c1cc(C(F)(F)F)ccn1)[C@]12Cc3ncn(-c4ccc(F)cc4)c3C=C1CC[C@H](S(=O)(=O)c1ccc(F)c(F)c1)C2 ; O=C(c1cc(C(F)(F)F)ccn1)[C@]12Cc3ncn(-c4ccc(F)cc4)c3C=C1CC[C@H](S(=O)(=O)c1cccc(Cl)c1)C2 ; O=C(c1cc(C(F)(F)F)ccn1)[C@]12Cc3ncn(-c4ccc(F)cc4)c3C=C1CC[C@H](S(=O)(=O)c1cccc(C(F)(F)F)c1)C2 ; N#Cc1cccc(S(=O)(=O)[C@H]2CCC3=Cc4c(ncn4-c4ccc(F)cc4)C[C@]3(C(=O)c3cc(C(F)(F)F)ccn3)C2)c1]

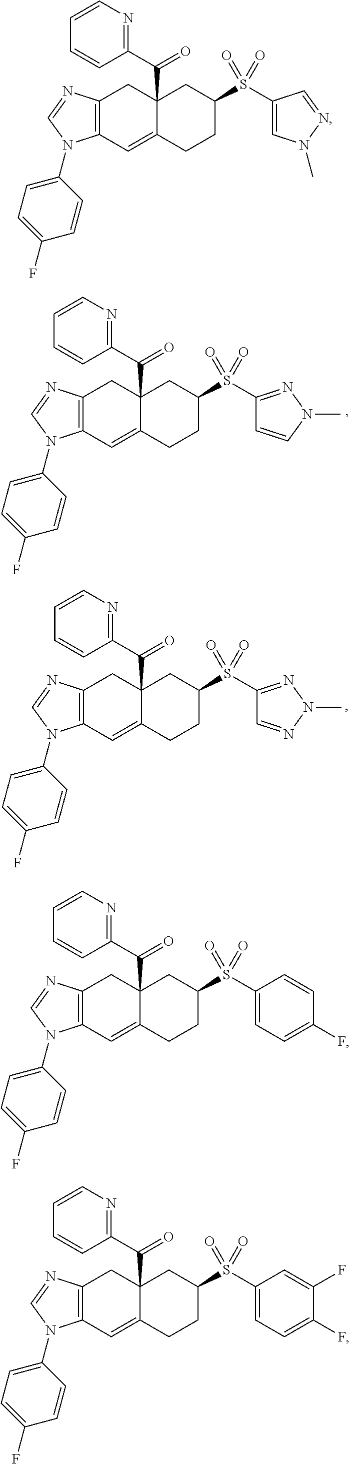

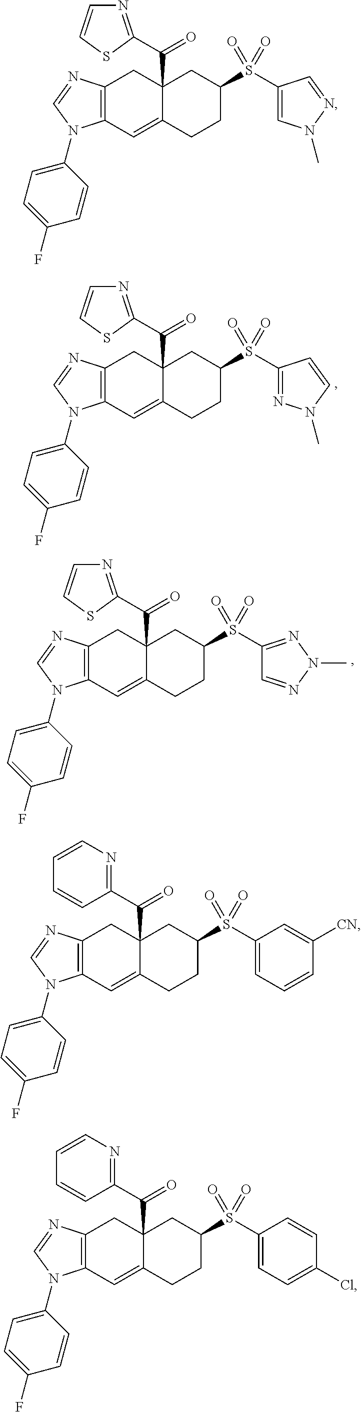

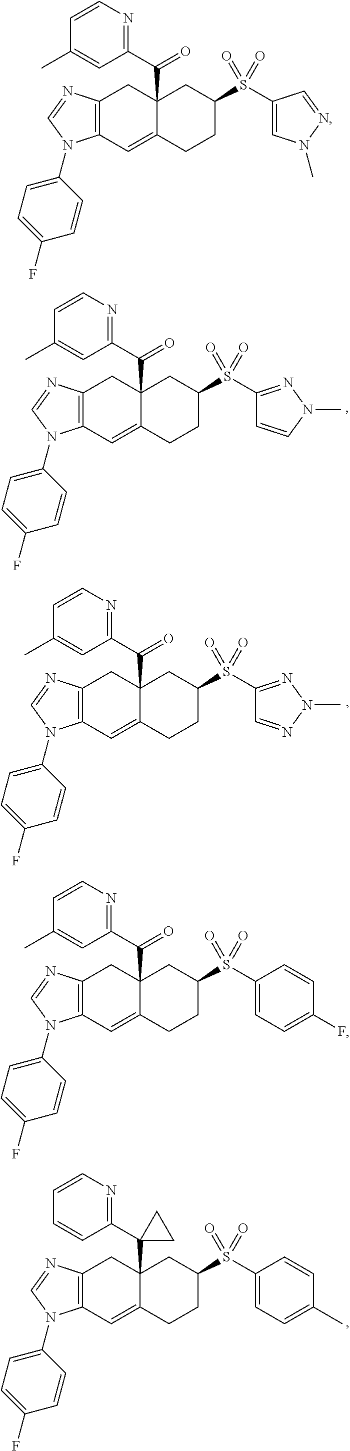

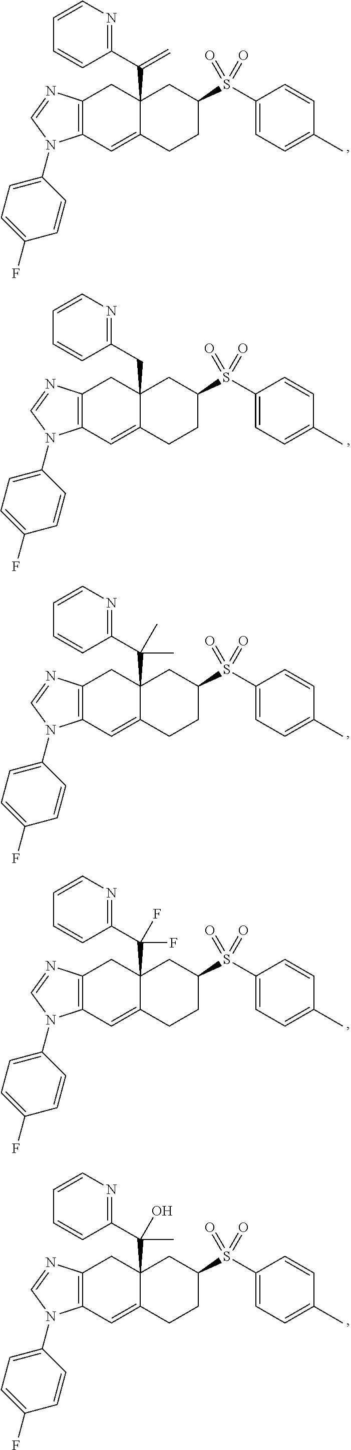 [SMILES: C=C(c1ccccn1)[C@]12Cc3ncn(-c4ccc(F)cc4)c3C=C1CC[C@H](S(=O)(=O)c1ccc(C)cc1)C2 ; Cc1ccc(S(=O)(=O)[C@H]2CCC3=Cc4c(ncn4-c4ccc(F)cc4)C[C@]3(Cc3ccccn3)C2)cc1 ; Cc1ccc(S(=O)(=O)[C@H]2CCC3=Cc4c(ncn4-c4ccc(F)cc4)C[C@]3(C(C)(C)c3ccccn3)C2)cc1 ; Cc1ccc(S(=O)(=O)[C@H]2CCC3=Cc4c(ncn4-c4ccc(F)cc4)C[C@]3(C(F)(F)c3ccccn3)C2)cc1 ; Cc1ccc(S(=O)(=O)[C@H]2CCC3=Cc4c(ncn4-c4ccc(F)cc4)C[C@]3(C(C)(O)c3ccccn3)C2)cc1]

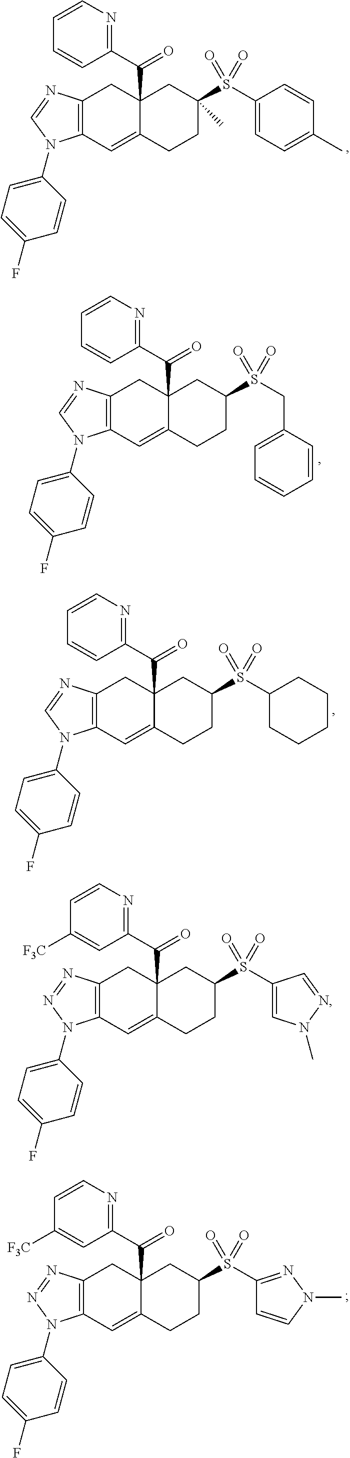

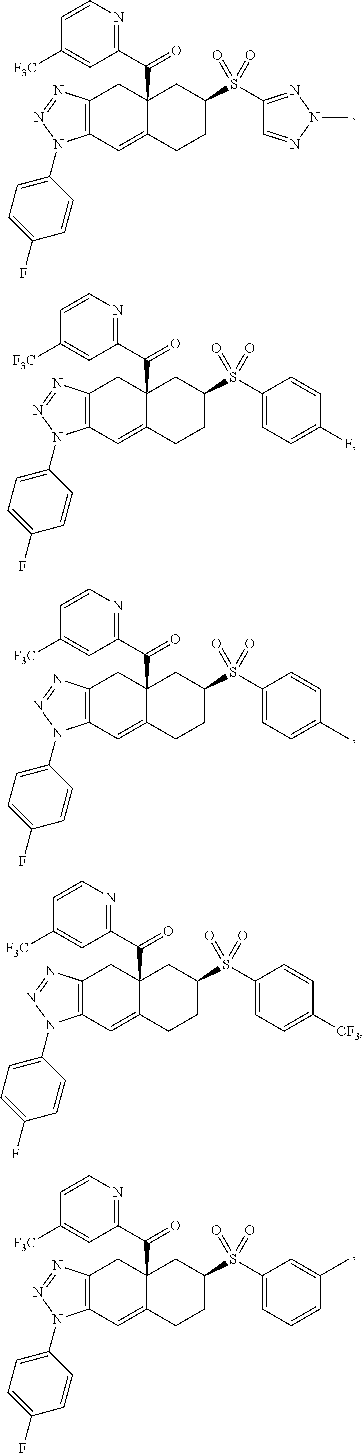 [SMILES: Cn1ncc(S(=O)(=O)[C@H]2CCC3=Cc4c(nnn4-c4ccc(F)cc4)C[C@]3(C(=O)c3cc(C(F)(F)F)ccn3)C2)n1 ; O=C(c1cc(C(F)(F)F)ccn1)[C@]12Cc3nnn(-c4ccc(F)cc4)c3C=C1CC[C@H](S(=O)(=O)c1ccc(F)cc1)C2 ; Cc1ccc(S(=O)(=O)[C@H]2CCC3=Cc4c(nnn4-c4ccc(F)cc4)C[C@]3(C(=O)c3cc(C(F)(F)F)ccn3)C2)cc1 ; O=C(c1cc(C(F)(F)F)ccn1)[C@]12Cc3nnn(-c4ccc(F)cc4)c3C=C1CC[C@H](S(=O)(=O)c1ccc(C(F)(F)F)cc1)C2 ; Cc1cccc(S(=O)(=O)[C@H]2CCC3=Cc4c(nnn4-c4ccc(F)cc4)C[C@]3(C(=O)c3cc(C(F)(F)F)ccn3)C2)c1]

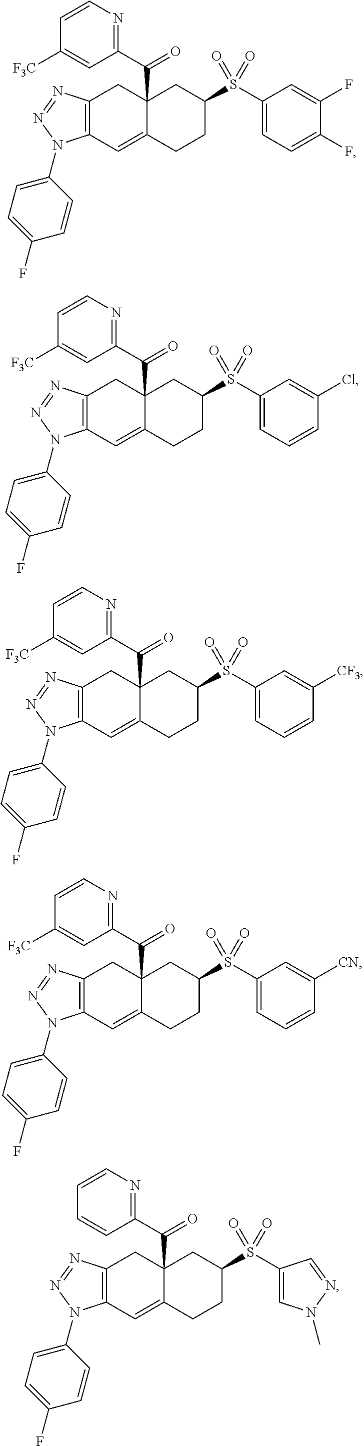 [SMILES: O=C(c1cc(C(F)(F)F)ccn1)[C@]12Cc3nnn(-c4ccc(F)cc4)c3C=C1CC[C@H](S(=O)(=O)c1ccc(F)c(F)c1)C2 ; O=C(c1cc(C(F)(F)F)ccn1)[C@]12Cc3nnn(-c4ccc(F)cc4)c3C=C1CC[C@H](S(=O)(=O)c1cccc(Cl)c1)C2 ; O=C(c1cc(C(F)(F)F)ccn1)[C@]12Cc3nnn(-c4ccc(F)cc4)c3C=C1CC[C@H](S(=O)(=O)c1cccc(C(F)(F)F)c1)C2 ; N#Cc1cccc(S(=O)(=O)[C@H]2CCC3=Cc4c(nnn4-c4ccc(F)cc4)C[C@]3(C(=O)c3cc(C(F)(F)F)ccn3)C2)c1 ; Cn1cc(S(=O)(=O)[C@H]2CCC3=Cc4c(nnn4-c4ccc(F)cc4)C[C@]3(C(=O)c3ccccn3)C2)cn1]

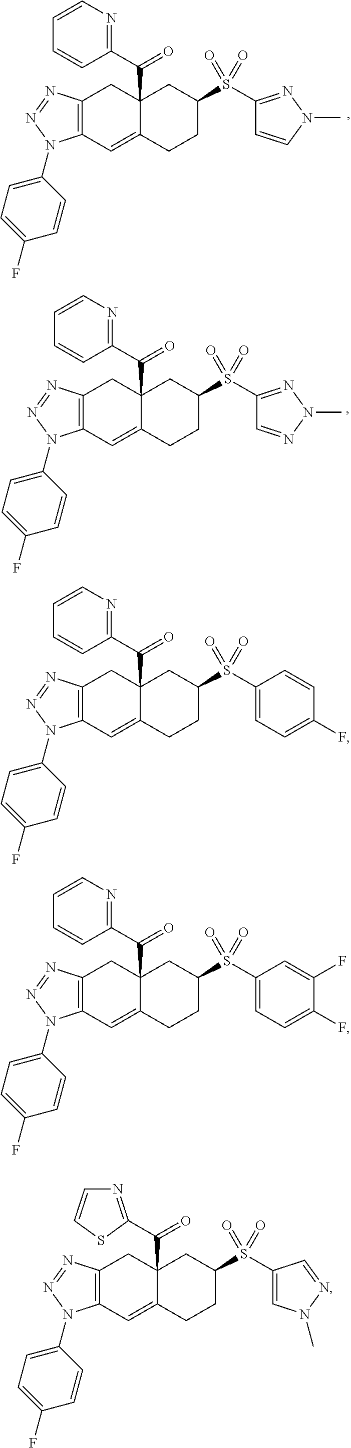 [SMILES: Cn1ccc(S(=O)(=O)[C@H]2CCC3=Cc4c(nnn4-c4ccc(F)cc4)C[C@]3(C(=O)c3ccccn3)C2)n1 ; Cn1ncc(S(=O)(=O)[C@H]2CCC3=Cc4c(nnn4-c4ccc(F)cc4)C[C@]3(C(=O)c3ccccn3)C2)n1 ; O=C(c1ccccn1)[C@]12Cc3nnn(-c4ccc(F)cc4)c3C=C1CC[C@H](S(=O)(=O)c1ccc(F)cc1)C2 ; O=C(c1ccccn1)[C@]12Cc3nnn(-c4ccc(F)cc4)c3C=C1CC[C@H](S(=O)(=O)c1ccc(F)c(F)c1)C2 ; Cn1cc(S(=O)(=O)[C@H]2CCC3=Cc4c(nnn4-c4ccc(F)cc4)C[C@]3(C(=O)c3nccs3)C2)cn1]

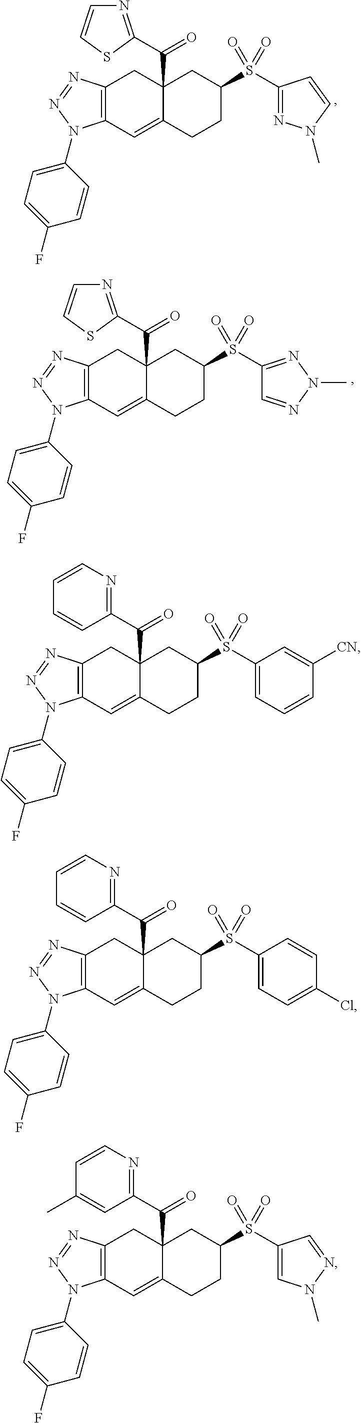

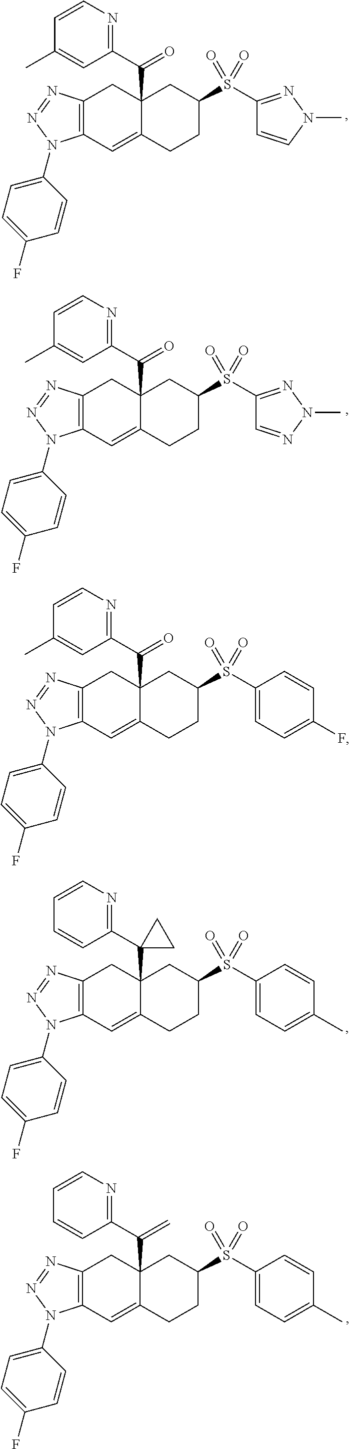 [SMILES: Cc1ccnc(C(=O)[C@]23Cc4nnn(-c5ccc(F)cc5)c4C=C2CC[C@H](S(=O)(=O)c2ccn(C)n2)C3)c1 ; Cc1ccnc(C(=O)[C@]23Cc4nnn(-c5ccc(F)cc5)c4C=C2CC[C@H](S(=O)(=O)c2cnn(C)n2)C3)c1 ; Cc1ccnc(C(=O)[C@]23Cc4nnn(-c5ccc(F)cc5)c4C=C2CC[C@H](S(=O)(=O)c2ccc(F)cc2)C3)c1 ; Cc1ccc(S(=O)(=O)[C@H]2CCC3=Cc4c(nnn4-c4ccc(F)cc4)C[C@]3(C3(c4ccccn4)CC3)C2)cc1 ; C=C(c1ccccn1)[C@]12Cc3nnn(-c4ccc(F)cc4)c3C=C1CC[C@H](S(=O)(=O)c1ccc(C)cc1)C2]

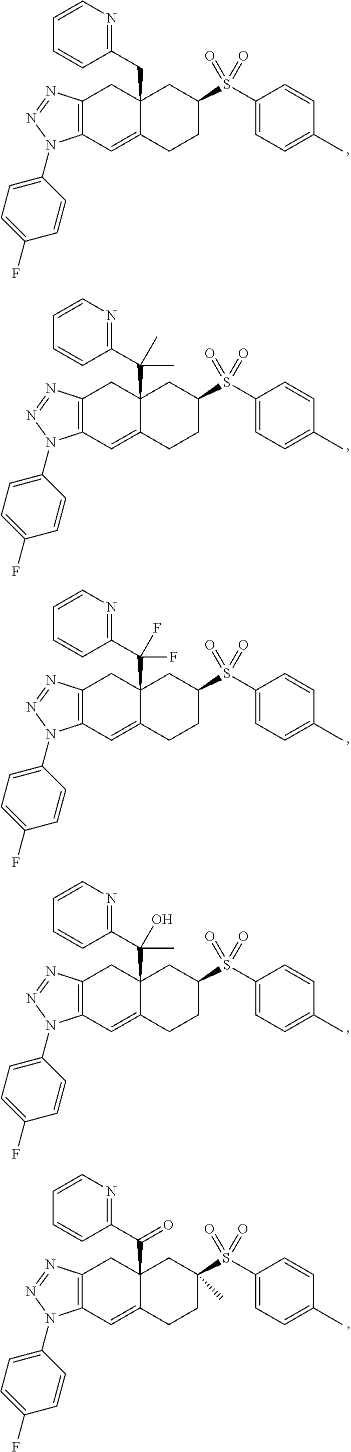 [SMILES: Cc1ccc(S(=O)(=O)[C@H]2CCC3=Cc4c(nnn4-c4ccc(F)cc4)C[C@]3(Cc3ccccn3)C2)cc1 ; Cc1ccc(S(=O)(=O)[C@H]2CCC3=Cc4c(nnn4-c4ccc(F)cc4)C[C@]3(C(C)(C)c3ccccn3)C2)cc1 ; Cc1ccc(S(=O)(=O)[C@H]2CCC3=Cc4c(nnn4-c4ccc(F)cc4)C[C@]3(C(F)(F)c3ccccn3)C2)cc1 ; Cc1ccc(S(=O)(=O)[C@H]2CCC3=Cc4c(nnn4-c4ccc(F)cc4)C[C@]3(C(C)(O)c3ccccn3)C2)cc1 ; Cc1ccc(S(=O)(=O)[C@@]2(C)CCC3=Cc4c(nnn4-c4ccc(F)cc4)C[C@]3(C(=O)c3ccccn3)C2)cc1]

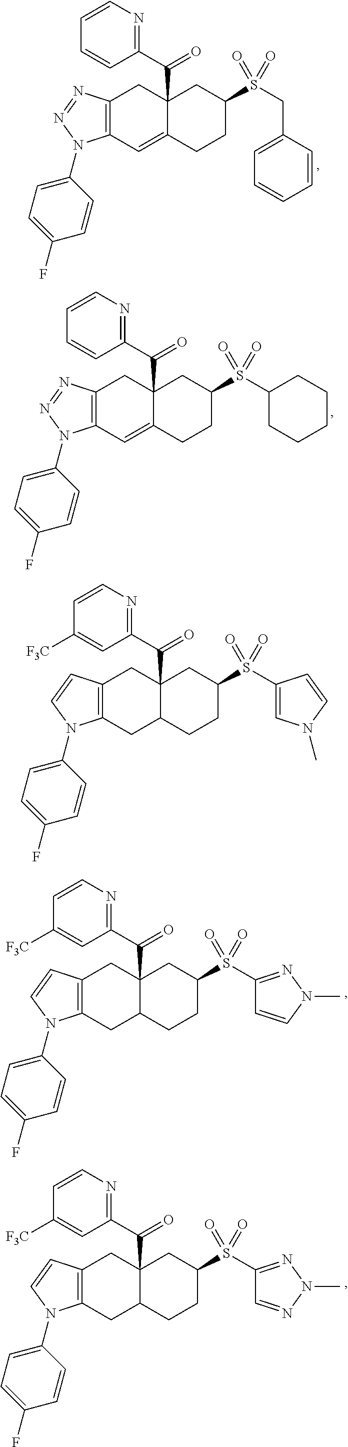

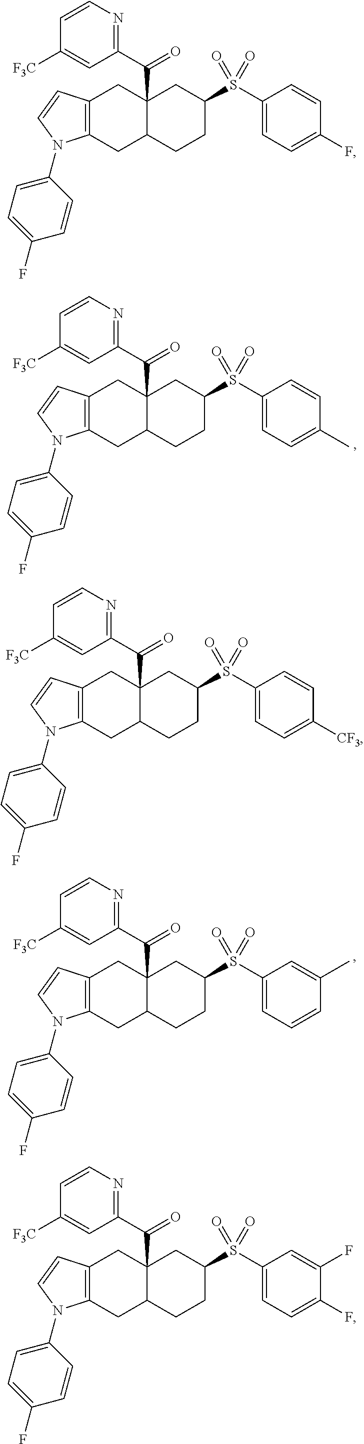 [SMILES: O=C(c1cc(C(F)(F)F)ccn1)[C@]12Cc3ccn(-c4ccc(F)cc4)c3CC1CC[C@H](S(=O)(=O)c1ccc(F)cc1)C2 ; Cc1ccc(S(=O)(=O)[C@H]2CCC3Cc4c(ccn4-c4ccc(F)cc4)C[C@]3(C(=O)c3cc(C(F)(F)F)ccn3)C2)cc1 ; O=C(c1cc(C(F)(F)F)ccn1)[C@]12Cc3ccn(-c4ccc(F)cc4)c3CC1CC[C@H](S(=O)(=O)c1ccc(C(F)(F)F)cc1)C2 ; Cc1cccc(S(=O)(=O)[C@H]2CCC3Cc4c(ccn4-c4ccc(F)cc4)C[C@]3(C(=O)c3cc(C(F)(F)F)ccn3)C2)c1 ; O=C(c1cc(C(F)(F)F)ccn1)[C@]12Cc3ccn(-c4ccc(F)cc4)c3CC1CC[C@H](S(=O)(=O)c1ccc(F)c(F)c1)C2]

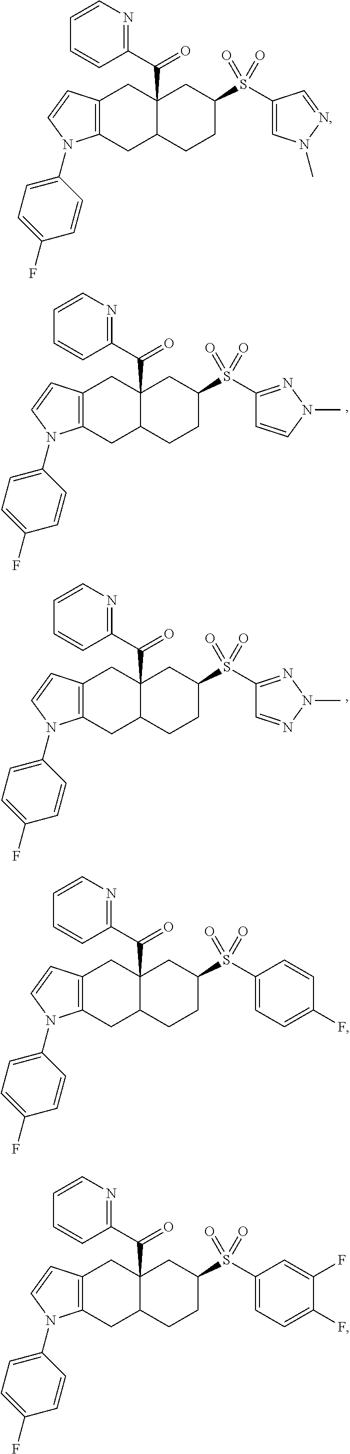

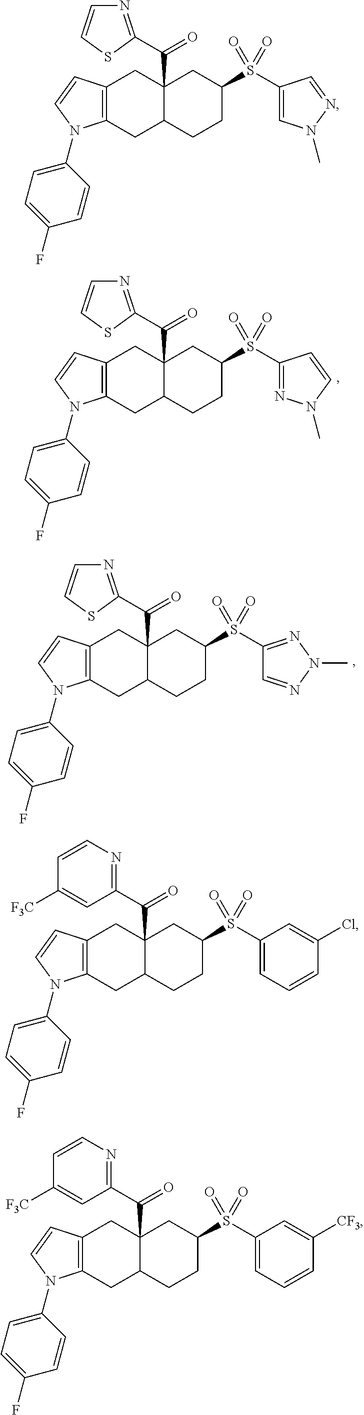

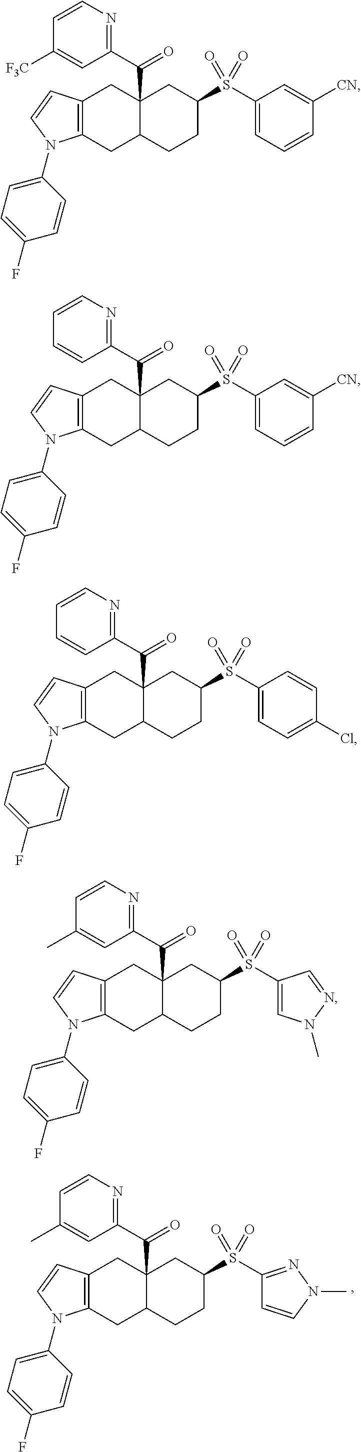

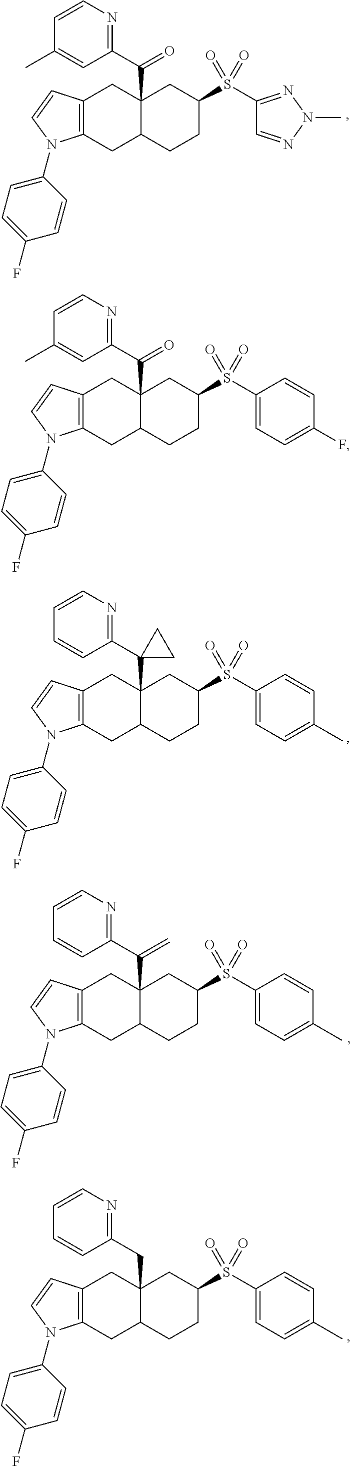 [SMILES: Cc1ccnc(C(=O)[C@]23Cc4ccn(-c5ccc(F)cc5)c4CC2CC[C@H](S(=O)(=O)c2cnn(C)n2)C3)c1 ; Cc1ccnc(C(=O)[C@]23Cc4ccn(-c5ccc(F)cc5)c4CC2CC[C@H](S(=O)(=O)c2ccc(F)cc2)C3)c1 ; Cc1ccc(S(=O)(=O)[C@H]2CCC3Cc4c(ccn4-c4ccc(F)cc4)C[C@]3(C3(c4ccccn4)CC3)C2)cc1 ; C=C(c1ccccn1)[C@]12Cc3ccn(-c4ccc(F)cc4)c3CC1CC[C@H](S(=O)(=O)c1ccc(C)cc1)C2 ; Cc1ccc(S(=O)(=O)[C@H]2CCC3Cc4c(ccn4-c4ccc(F)cc4)C[C@]3(Cc3ccccn3)C2)cc1]

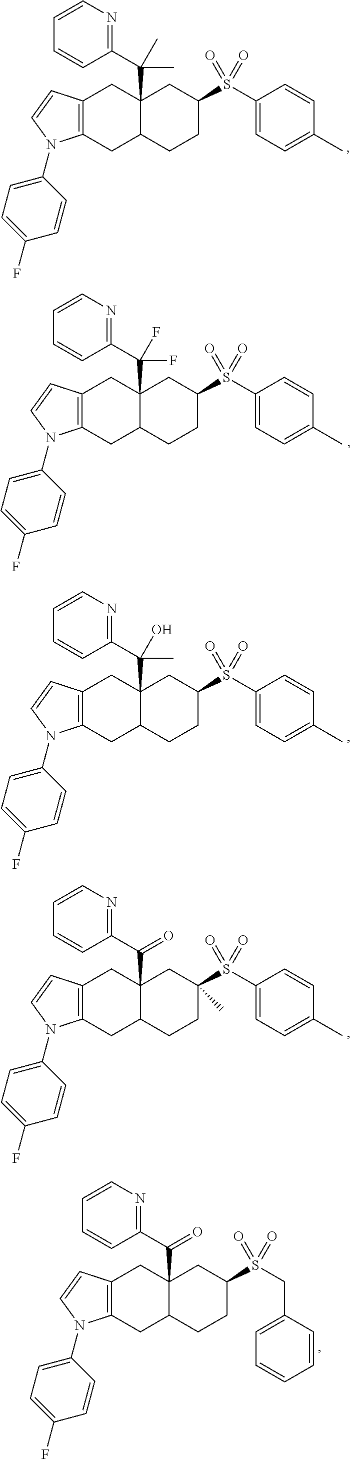 [SMILES: Cc1ccc(S(=O)(=O)[C@H]2CCC3Cc4c(ccn4-c4ccc(F)cc4)C[C@]3(C(C)(C)c3ccccn3)C2)cc1 ; Cc1ccc(S(=O)(=O)[C@H]2CCC3Cc4c(ccn4-c4ccc(F)cc4)C[C@]3(C(F)(F)c3ccccn3)C2)cc1 ; Cc1ccc(S(=O)(=O)[C@H]2CCC3Cc4c(ccn4-c4ccc(F)cc4)C[C@]3(C(C)(O)c3ccccn3)C2)cc1 ; Cc1ccc(S(=O)(=O)[C@@]2(C)CCC3Cc4c(ccn4-c4ccc(F)cc4)C[C@]3(C(=O)c3ccccn3)C2)cc1 ; O=C(c1ccccn1)[C@]12Cc3ccn(-c4ccc(F)cc4)c3CC1CC[C@H](S(=O)(=O)Cc1ccccc1)C2]

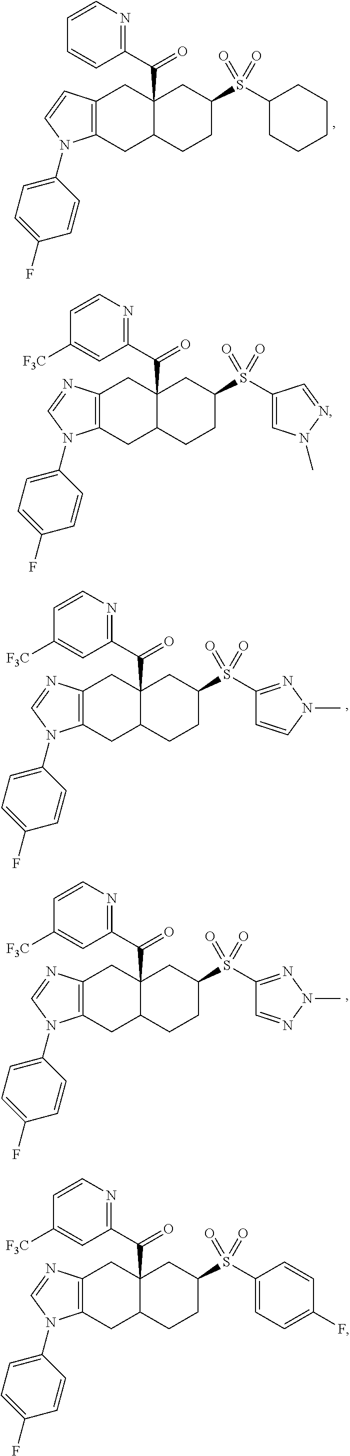

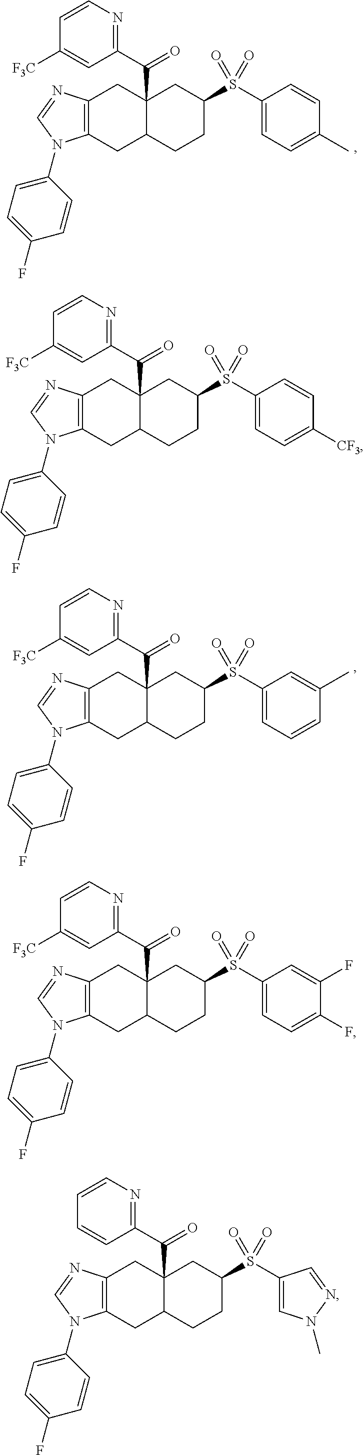 [SMILES: Cc1ccc(S(=O)(=O)[C@H]2CCC3Cc4c(ncn4-c4ccc(F)cc4)C[C@]3(C(=O)c3cc(C(F)(F)F)ccn3)C2)cc1 ; O=C(c1cc(C(F)(F)F)ccn1)[C@]12Cc3ncn(-c4ccc(F)cc4)c3CC1CC[C@H](S(=O)(=O)c1ccc(C(F)(F)F)cc1)C2 ; Cc1cccc(S(=O)(=O)[C@H]2CCC3Cc4c(ncn4-c4ccc(F)cc4)C[C@]3(C(=O)c3cc(C(F)(F)F)ccn3)C2)c1 ; O=C(c1cc(C(F)(F)F)ccn1)[C@]12Cc3ncn(-c4ccc(F)cc4)c3CC1CC[C@H](S(=O)(=O)c1ccc(F)c(F)c1)C2 ; Cn1cc(S(=O)(=O)[C@H]2CCC3Cc4c(ncn4-c4ccc(F)cc4)C[C@]3(C(=O)c3ccccn3)C2)cn1]

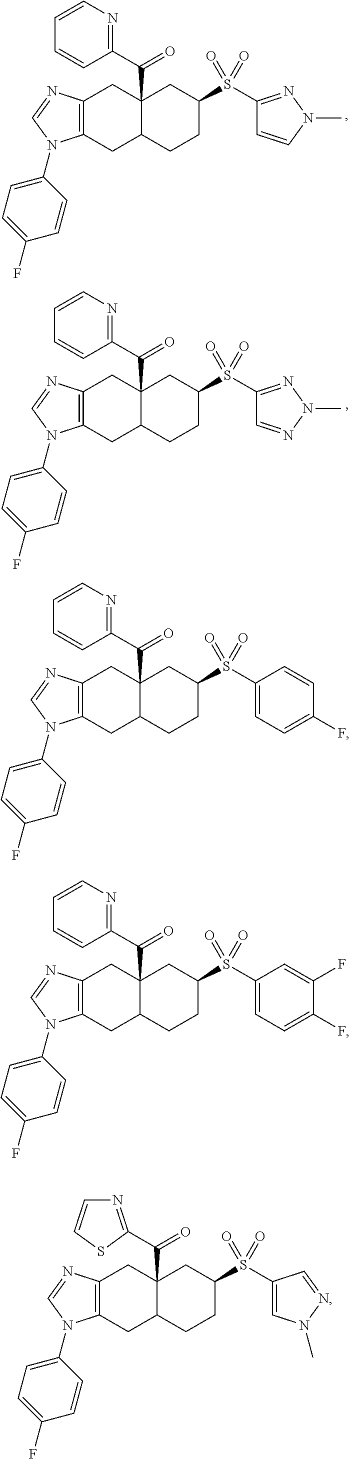 [SMILES: Cn1ccc(S(=O)(=O)[C@H]2CCC3Cc4c(ncn4-c4ccc(F)cc4)C[C@]3(C(=O)c3ccccn3)C2)n1 ; Cn1ncc(S(=O)(=O)[C@H]2CCC3Cc4c(ncn4-c4ccc(F)cc4)C[C@]3(C(=O)c3ccccn3)C2)n1 ; O=C(c1ccccn1)[C@]12Cc3ncn(-c4ccc(F)cc4)c3CC1CC[C@H](S(=O)(=O)c1ccc(F)cc1)C2 ; O=C(c1ccccn1)[C@]12Cc3ncn(-c4ccc(F)cc4)c3CC1CC[C@H](S(=O)(=O)c1ccc(F)c(F)c1)C2 ; Cn1cc(S(=O)(=O)[C@H]2CCC3Cc4c(ncn4-c4ccc(F)cc4)C[C@]3(C(=O)c3nccs3)C2)cn1]

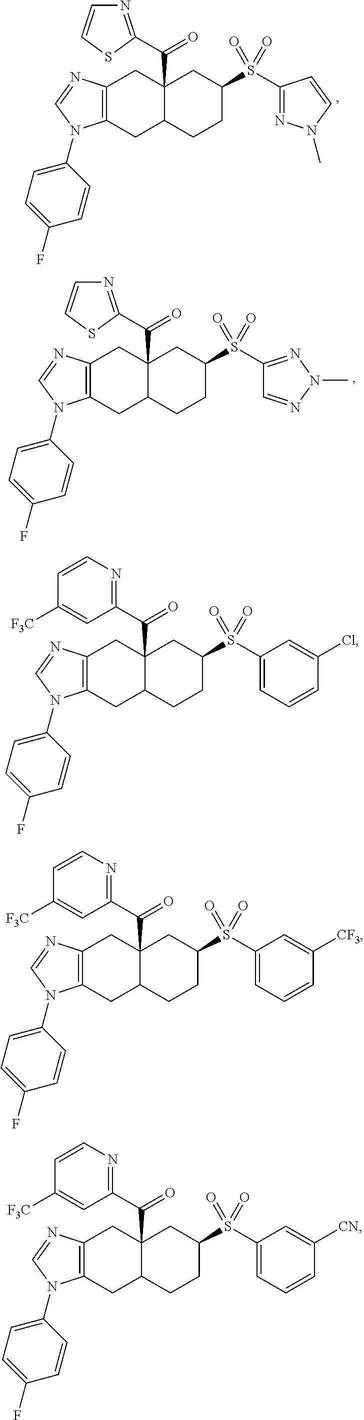

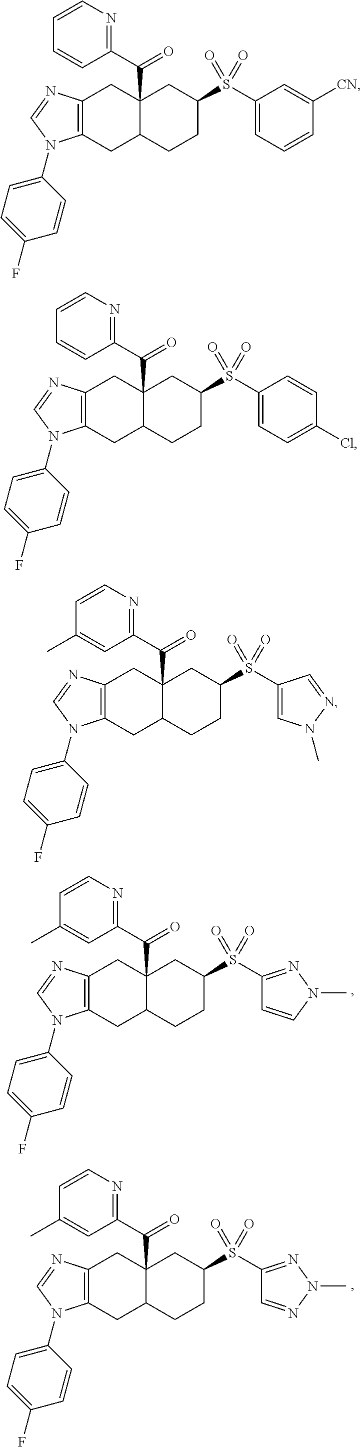

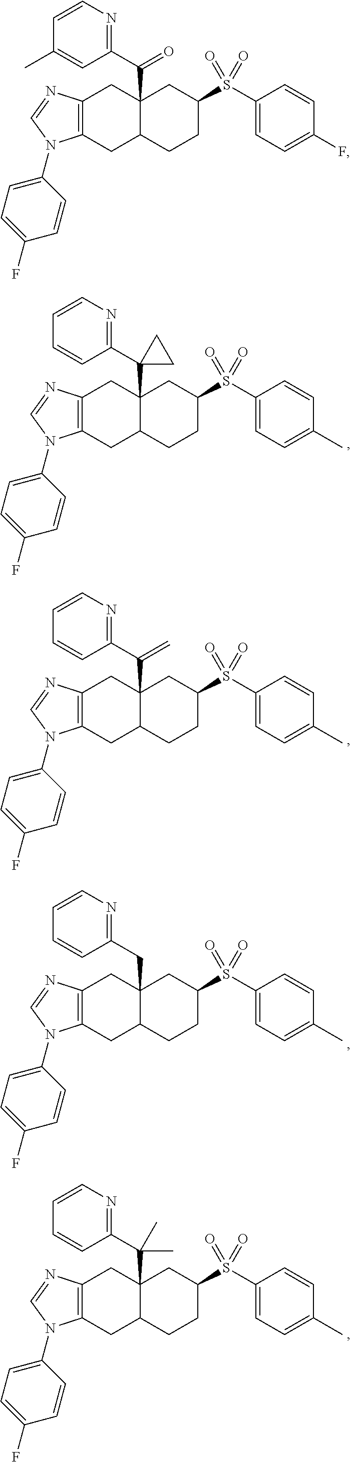 [SMILES: Cc1ccnc(C(=O)[C@]23Cc4ncn(-c5ccc(F)cc5)c4CC2CC[C@H](S(=O)(=O)c2ccc(F)cc2)C3)c1 ; Cc1ccc(S(=O)(=O)[C@H]2CCC3Cc4c(ncn4-c4ccc(F)cc4)C[C@]3(C3(c4ccccn4)CC3)C2)cc1 ; C=C(c1ccccn1)[C@]12Cc3ncn(-c4ccc(F)cc4)c3CC1CC[C@H](S(=O)(=O)c1ccc(C)cc1)C2 ; Cc1ccc(S(=O)(=O)[C@H]2CCC3Cc4c(ncn4-c4ccc(F)cc4)C[C@]3(Cc3ccccn3)C2)cc1 ; Cc1ccc(S(=O)(=O)[C@H]2CCC3Cc4c(ncn4-c4ccc(F)cc4)C[C@]3(C(C)(C)c3ccccn3)C2)cc1]

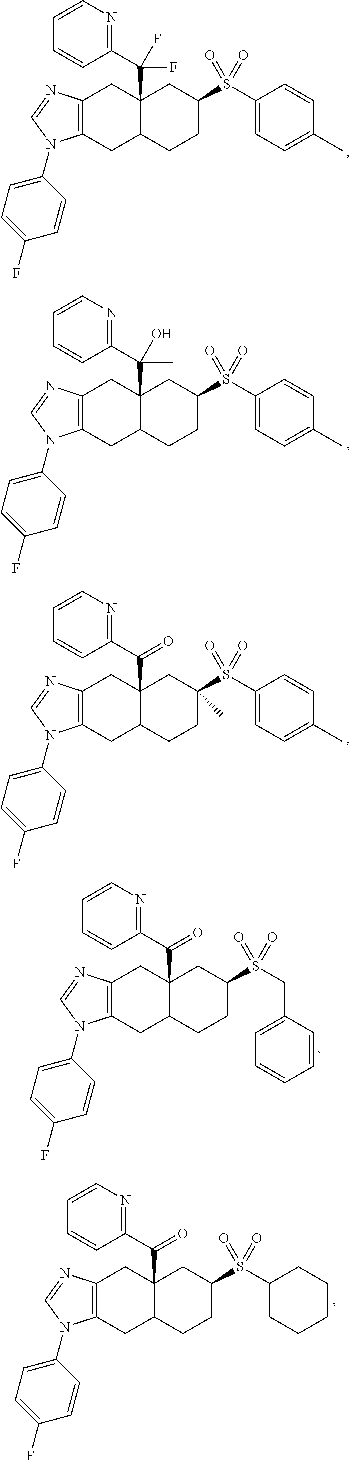 [SMILES: Cc1ccc(S(=O)(=O)[C@H]2CCC3Cc4c(ncn4-c4ccc(F)cc4)C[C@]3(C(F)(F)c3ccccn3)C2)cc1 ; Cc1ccc(S(=O)(=O)[C@H]2CCC3Cc4c(ncn4-c4ccc(F)cc4)C[C@]3(C(C)(O)c3ccccn3)C2)cc1 ; Cc1ccc(S(=O)(=O)[C@@]2(C)CCC3Cc4c(ncn4-c4ccc(F)cc4)C[C@]3(C(=O)c3ccccn3)C2)cc1 ; O=C(c1ccccn1)[C@]12Cc3ncn(-c4ccc(F)cc4)c3CC1CC[C@H](S(=O)(=O)Cc1ccccc1)C2 ; O=C(c1ccccn1)[C@]12Cc3ncn(-c4ccc(F)cc4)c3CC1CC[C@H](S(=O)(=O)C1CCCCC1)C2]

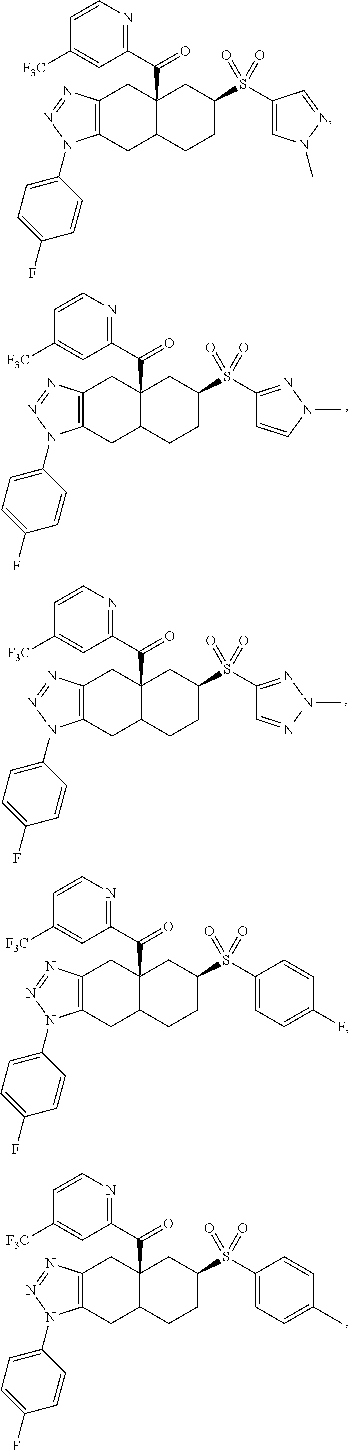

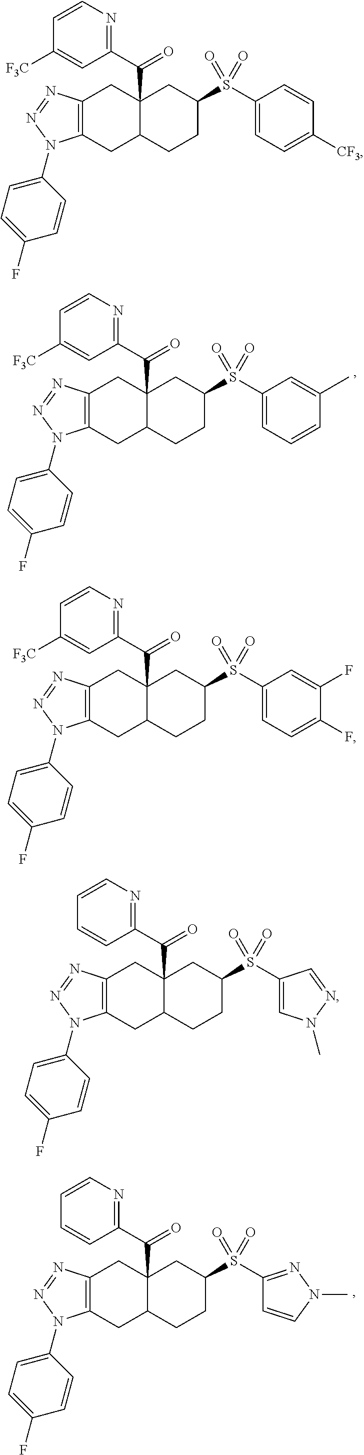 [SMILES: O=C(c1cc(C(F)(F)F)ccn1)[C@]12Cc3nnn(-c4ccc(F)cc4)c3CC1CC[C@H](S(=O)(=O)c1ccc(C(F)(F)F)cc1)C2 ; Cc1cccc(S(=O)(=O)[C@H]2CCC3Cc4c(nnn4-c4ccc(F)cc4)C[C@]3(C(=O)c3cc(C(F)(F)F)ccn3)C2)c1 ; O=C(c1cc(C(F)(F)F)ccn1)[C@]12Cc3nnn(-c4ccc(F)cc4)c3CC1CC[C@H](S(=O)(=O)c1ccc(F)c(F)c1)C2 ; Cn1cc(S(=O)(=O)[C@H]2CCC3Cc4c(nnn4-c4ccc(F)cc4)C[C@]3(C(=O)c3ccccn3)C2)cn1 ; Cn1ccc(S(=O)(=O)[C@H]2CCC3Cc4c(nnn4-c4ccc(F)cc4)C[C@]3(C(=O)c3ccccn3)C2)n1]

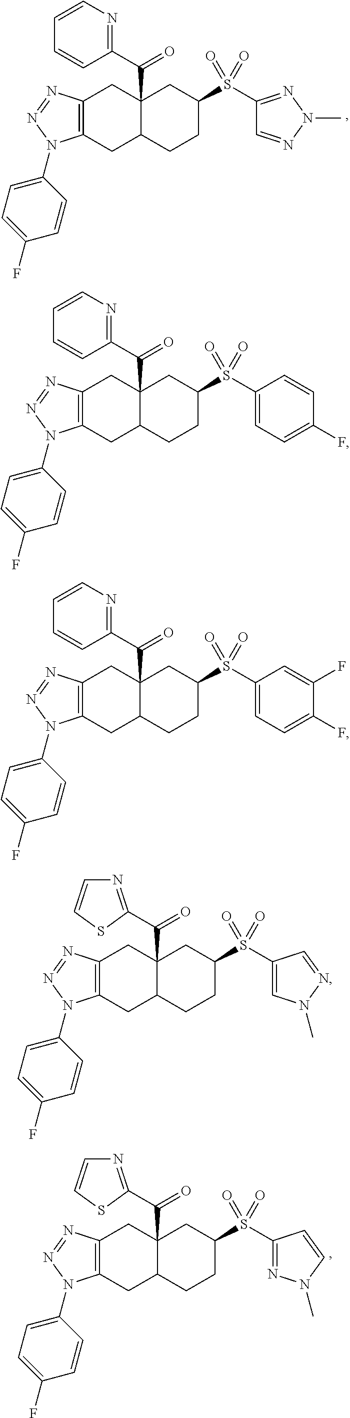 [SMILES: Cn1ncc(S(=O)(=O)[C@H]2CCC3Cc4c(nnn4-c4ccc(F)cc4)C[C@]3(C(=O)c3ccccn3)C2)n1 ; O=C(c1ccccn1)[C@]12Cc3nnn(-c4ccc(F)cc4)c3CC1CC[C@H](S(=O)(=O)c1ccc(F)cc1)C2 ; O=C(c1ccccn1)[C@]12Cc3nnn(-c4ccc(F)cc4)c3CC1CC[C@H](S(=O)(=O)c1ccc(F)c(F)c1)C2 ; Cn1cc(S(=O)(=O)[C@H]2CCC3Cc4c(nnn4-c4ccc(F)cc4)C[C@]3(C(=O)c3nccs3)C2)cn1 ; Cn1ccc(S(=O)(=O)[C@H]2CCC3Cc4c(nnn4-c4ccc(F)cc4)C[C@]3(C(=O)c3nccs3)C2)n1]

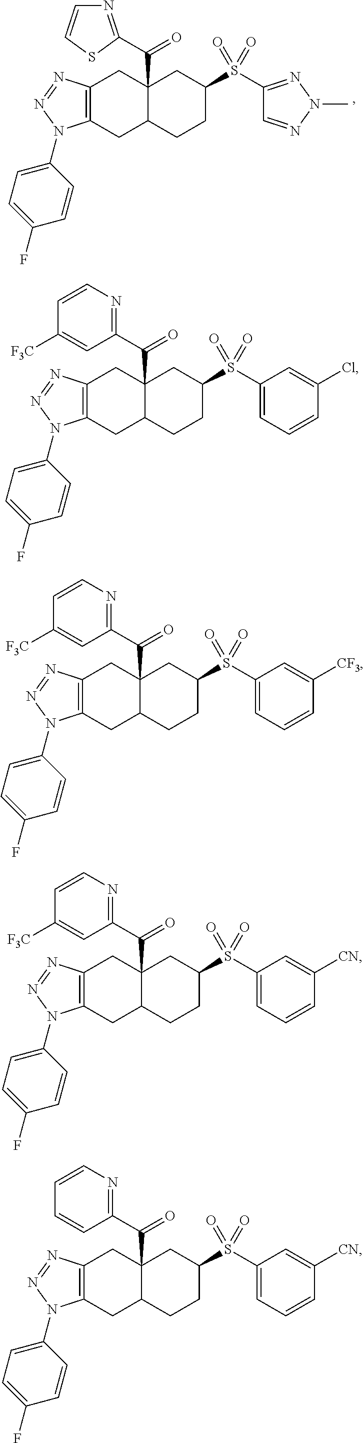 [SMILES: Cn1ncc(S(=O)(=O)[C@H]2CCC3Cc4c(nnn4-c4ccc(F)cc4)C[C@]3(C(=O)c3nccs3)C2)n1 ; O=C(c1cc(C(F)(F)F)ccn1)[C@]12Cc3nnn(-c4ccc(F)cc4)c3CC1CC[C@H](S(=O)(=O)c1cccc(Cl)c1)C2 ; O=C(c1cc(C(F)(F)F)ccn1)[C@]12Cc3nnn(-c4ccc(F)cc4)c3CC1CC[C@H](S(=O)(=O)c1cccc(C(F)(F)F)c1)C2 ; N#Cc1cccc(S(=O)(=O)[C@H]2CCC3Cc4c(nnn4-c4ccc(F)cc4)C[C@]3(C(=O)c3cc(C(F)(F)F)ccn3)C2)c1 ; N#Cc1cccc(S(=O)(=O)[C@H]2CCC3Cc4c(nnn4-c4ccc(F)cc4)C[C@]3(C(=O)c3ccccn3)C2)c1]

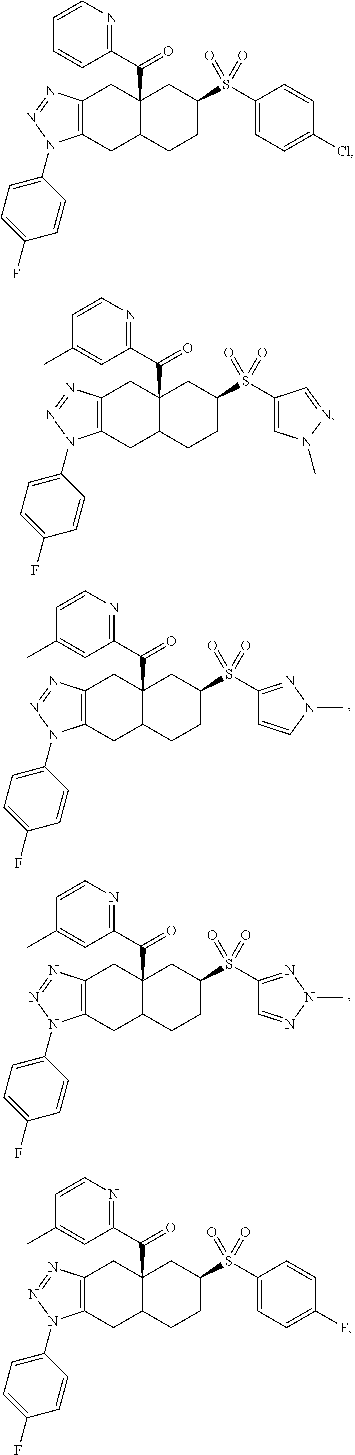

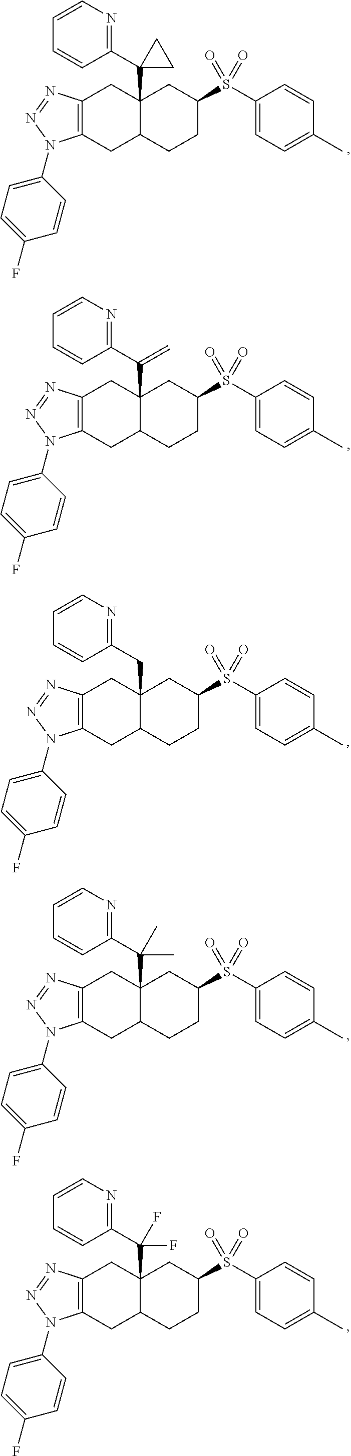 [SMILES: Cc1ccc(S(=O)(=O)[C@H]2CCC3Cc4c(nnn4-c4ccc(F)cc4)C[C@]3(C3(c4ccccn4)CC3)C2)cc1 ; C=C(c1ccccn1)[C@]12Cc3nnn(-c4ccc(F)cc4)c3CC1CC[C@H](S(=O)(=O)c1ccc(C)cc1)C2 ; Cc1ccc(S(=O)(=O)[C@H]2CCC3Cc4c(nnn4-c4ccc(F)cc4)C[C@]3(Cc3ccccn3)C2)cc1 ; Cc1ccc(S(=O)(=O)[C@H]2CCC3Cc4c(nnn4-c4ccc(F)cc4)C[C@]3(C(C)(C)c3ccccn3)C2)cc1 ; Cc1ccc(S(=O)(=O)[C@H]2CCC3Cc4c(nnn4-c4ccc(F)cc4)C[C@]3(C(F)(F)c3ccccn3)C2)cc1]

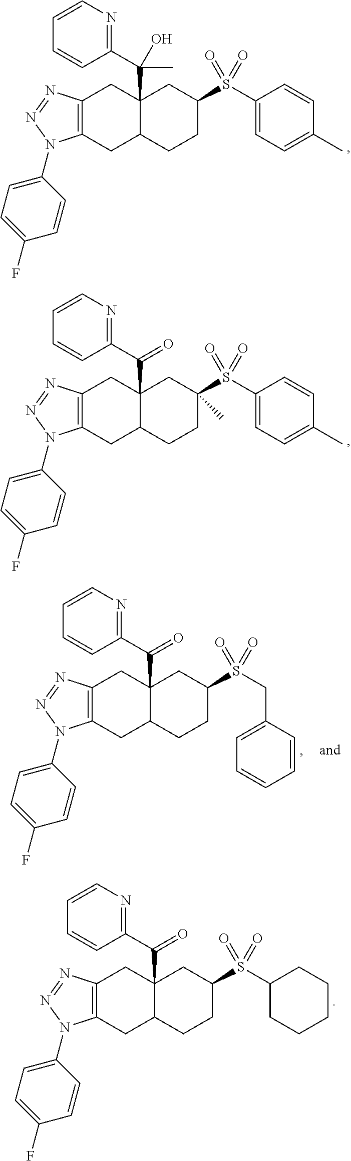 [SMILES: Cc1ccc(S(=O)(=O)[C@H]2CCC3Cc4c(nnn4-c4ccc(F)cc4)C[C@]3(C(C)(O)c3ccccn3)C2)cc1 ; Cc1ccc(S(=O)(=O)[C@@]2(C)CCC3Cc4c(nnn4-c4ccc(F)cc4)C[C@]3(C(=O)c3ccccn3)C2)cc1 ; O=C(c1ccccn1)[C@]12Cc3nnn(-c4ccc(F)cc4)c3CC1CC[C@H](S(=O)(=O)Cc1ccccc1)C2 ; O=C(c1ccccn1)[C@]12Cc3nnn(-c4ccc(F)cc4)c3CC1CC[C@H](S(=O)(=O)C1CCCCC1)C2]

In some embodiments is a compound, or a pharmaceutically acceptable salt or solvate thereof, having a structure selected from:

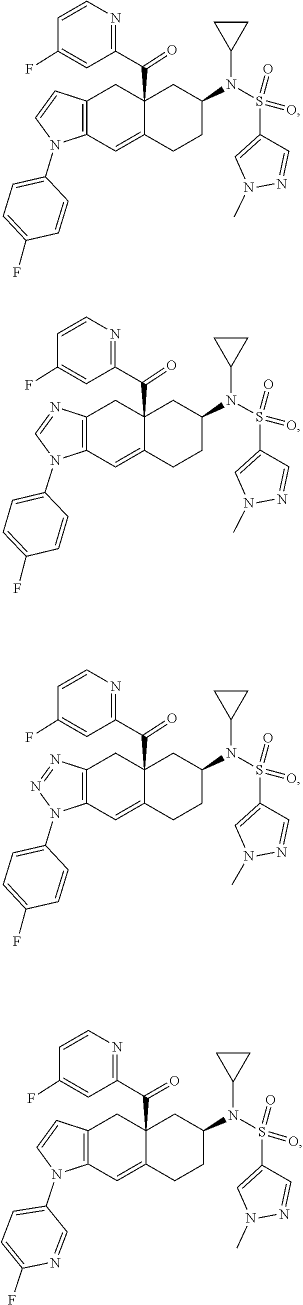 [SMILES: Cn1cc(S(=O)(=O)N(C2CC2)[C@H]2CCC3=Cc4c(ccn4-c4ccc(F)cc4)C[C@]3(C(=O)c3cc(F)ccn3)C2)cn1 ; Cn1cc(S(=O)(=O)N(C2CC2)[C@H]2CCC3=Cc4c(ncn4-c4ccc(F)cc4)C[C@]3(C(=O)c3cc(F)ccn3)C2)cn1 ; Cn1cc(S(=O)(=O)N(C2CC2)[C@H]2CCC3=Cc4c(nnn4-c4ccc(F)cc4)C[C@]3(C(=O)c3cc(F)ccn3)C2)cn1 ; Cn1cc(S(=O)(=O)N(C2CC2)[C@H]2CCC3=Cc4c(ccn4-c4ccc(F)nc4)C[C@]3(C(=O)c3cc(F)ccn3)C2)cn1]

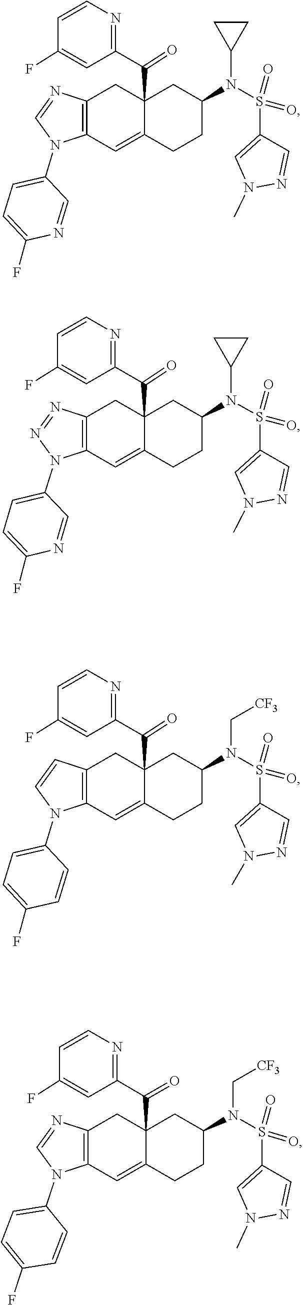 [SMILES: Cn1cc(S(=O)(=O)N(C2CC2)[C@H]2CCC3=Cc4c(ncn4-c4ccc(F)nc4)C[C@]3(C(=O)c3cc(F)ccn3)C2)cn1 ; Cn1cc(S(=O)(=O)N(C2CC2)[C@H]2CCC3=Cc4c(nnn4-c4ccc(F)nc4)C[C@]3(C(=O)c3cc(F)ccn3)C2)cn1 ; Cn1cc(S(=O)(=O)N(CC(F)(F)F)[C@H]2CCC3=Cc4c(ccn4-c4ccc(F)cc4)C[C@]3(C(=O)c3cc(F)ccn3)C2)cn1 ; Cn1cc(S(=O)(=O)N(CC(F)(F)F)[C@H]2CCC3=Cc4c(ncn4-c4ccc(F)cc4)C[C@]3(C(=O)c3cc(F)ccn3)C2)cn1]

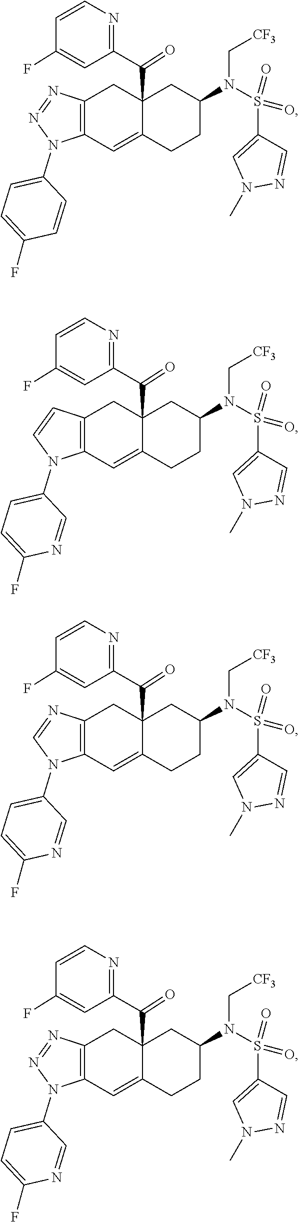 [SMILES: Cn1cc(S(=O)(=O)N(CC(F)(F)F)[C@H]2CCC3=Cc4c(nnn4-c4ccc(F)cc4)C[C@]3(C(=O)c3cc(F)ccn3)C2)cn1 ; Cn1cc(S(=O)(=O)N(CC(F)(F)F)[C@H]2CCC3=Cc4c(ccn4-c4ccc(F)nc4)C[C@]3(C(=O)c3cc(F)ccn3)C2)cn1 ; Cn1cc(S(=O)(=O)N(CC(F)(F)F)[C@H]2CCC3=Cc4c(ncn4-c4ccc(F)nc4)C[C@]3(C(=O)c3cc(F)ccn3)C2)cn1 ; Cn1cc(S(=O)(=O)N(CC(F)(F)F)[C@H]2CCC3=Cc4c(nnn4-c4ccc(F)nc4)C[C@]3(C(=O)c3cc(F)ccn3)C2)cn1]

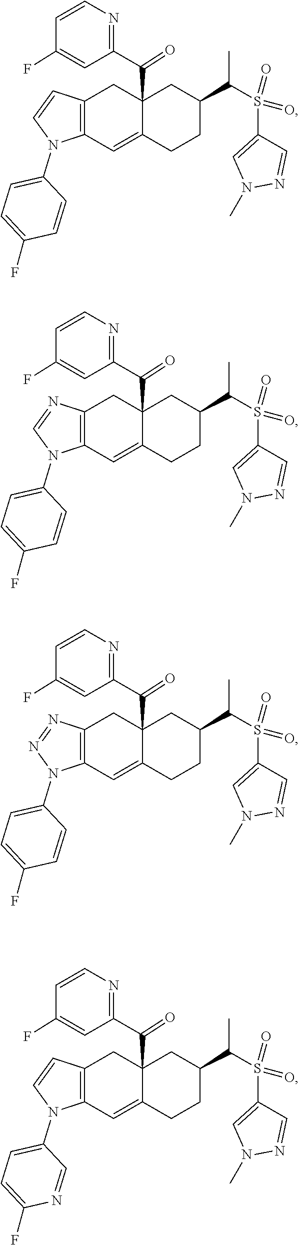 [SMILES: CC([C@H]1CCC2=Cc3c(ccn3-c3ccc(F)cc3)C[C@]2(C(=O)c2cc(F)ccn2)C1)S(=O)(=O)c1cnn(C)c1 ; CC([C@H]1CCC2=Cc3c(ncn3-c3ccc(F)cc3)C[C@]2(C(=O)c2cc(F)ccn2)C1)S(=O)(=O)c1cnn(C)c1 ; CC([C@H]1CCC2=Cc3c(nnn3-c3ccc(F)cc3)C[C@]2(C(=O)c2cc(F)ccn2)C1)S(=O)(=O)c1cnn(C)c1 ; CC([C@H]1CCC2=Cc3c(ccn3-c3ccc(F)nc3)C[C@]2(C(=O)c2cc(F)ccn2)C1)S(=O)(=O)c1cnn(C)c1]

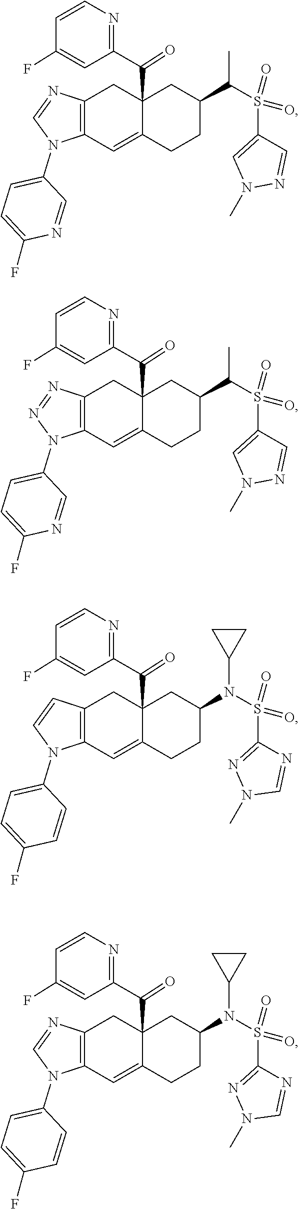 [SMILES: CC([C@H]1CCC2=Cc3c(ncn3-c3ccc(F)nc3)C[C@]2(C(=O)c2cc(F)ccn2)C1)S(=O)(=O)c1cnn(C)c1 ; CC([C@H]1CCC2=Cc3c(nnn3-c3ccc(F)nc3)C[C@]2(C(=O)c2cc(F)ccn2)C1)S(=O)(=O)c1cnn(C)c1 ; Cn1cnc(S(=O)(=O)N(C2CC2)[C@H]2CCC3=Cc4c(ccn4-c4ccc(F)cc4)C[C@]3(C(=O)c3cc(F)ccn3)C2)n1 ; Cn1cnc(S(=O)(=O)N(C2CC2)[C@H]2CCC3=Cc4c(ncn4-c4ccc(F)cc4)C[C@]3(C(=O)c3cc(F)ccn3)C2)n1]

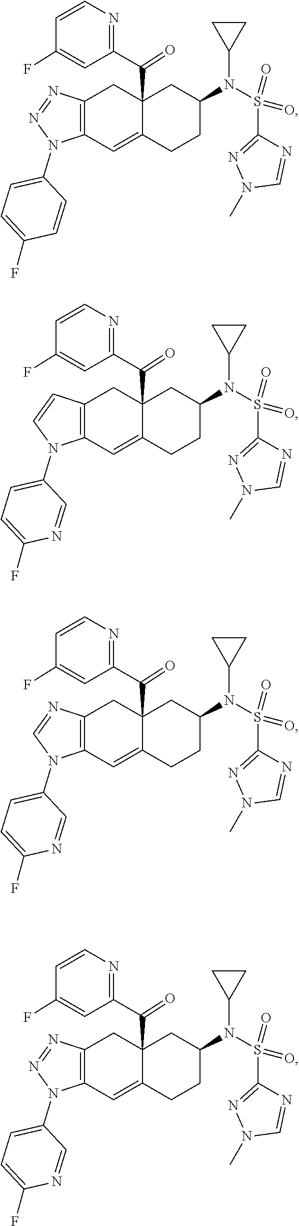 [SMILES: Cn1cnc(S(=O)(=O)N(C2CC2)[C@H]2CCC3=Cc4c(nnn4-c4ccc(F)cc4)C[C@]3(C(=O)c3cc(F)ccn3)C2)n1 ; Cn1cnc(S(=O)(=O)N(C2CC2)[C@H]2CCC3=Cc4c(ccn4-c4ccc(F)nc4)C[C@]3(C(=O)c3cc(F)ccn3)C2)n1 ; Cn1cnc(S(=O)(=O)N(C2CC2)[C@H]2CCC3=Cc4c(ncn4-c4ccc(F)nc4)C[C@]3(C(=O)c3cc(F)ccn3)C2)n1 ; Cn1cnc(S(=O)(=O)N(C2CC2)[C@H]2CCC3=Cc4c(nnn4-c4ccc(F)nc4)C[C@]3(C(=O)c3cc(F)ccn3)C2)n1]

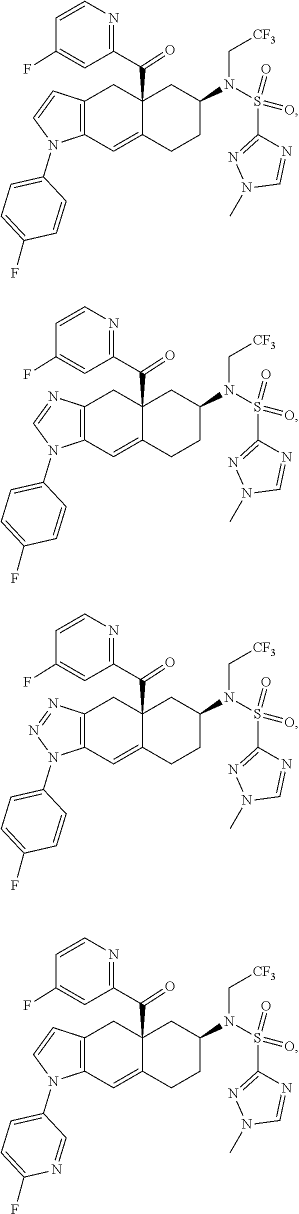 [SMILES: Cn1cnc(S(=O)(=O)N(CC(F)(F)F)[C@H]2CCC3=Cc4c(ccn4-c4ccc(F)cc4)C[C@]3(C(=O)c3cc(F)ccn3)C2)n1 ; Cn1cnc(S(=O)(=O)N(CC(F)(F)F)[C@H]2CCC3=Cc4c(ncn4-c4ccc(F)cc4)C[C@]3(C(=O)c3cc(F)ccn3)C2)n1 ; Cn1cnc(S(=O)(=O)N(CC(F)(F)F)[C@H]2CCC3=Cc4c(nnn4-c4ccc(F)cc4)C[C@]3(C(=O)c3cc(F)ccn3)C2)n1 ; Cn1cnc(S(=O)(=O)N(CC(F)(F)F)[C@H]2CCC3=Cc4c(ccn4-c4ccc(F)nc4)C[C@]3(C(=O)c3cc(F)ccn3)C2)n1]

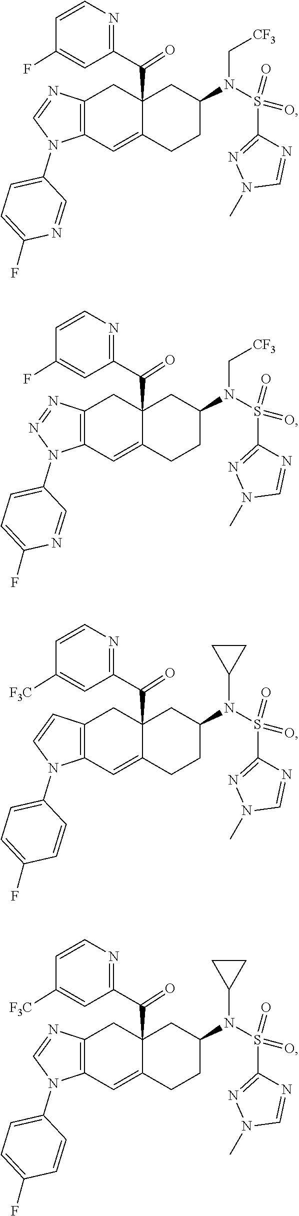 [SMILES: Cn1cnc(S(=O)(=O)N(CC(F)(F)F)[C@H]2CCC3=Cc4c(ncn4-c4ccc(F)nc4)C[C@]3(C(=O)c3cc(F)ccn3)C2)n1 ; Cn1cnc(S(=O)(=O)N(CC(F)(F)F)[C@H]2CCC3=Cc4c(nnn4-c4ccc(F)nc4)C[C@]3(C(=O)c3cc(F)ccn3)C2)n1 ; Cn1cnc(S(=O)(=O)N(C2CC2)[C@H]2CCC3=Cc4c(ccn4-c4ccc(F)cc4)C[C@]3(C(=O)c3cc(C(F)(F)F)ccn3)C2)n1 ; Cn1cnc(S(=O)(=O)N(C2CC2)[C@H]2CCC3=Cc4c(ncn4-c4ccc(F)cc4)C[C@]3(C(=O)c3cc(C(F)(F)F)ccn3)C2)n1]

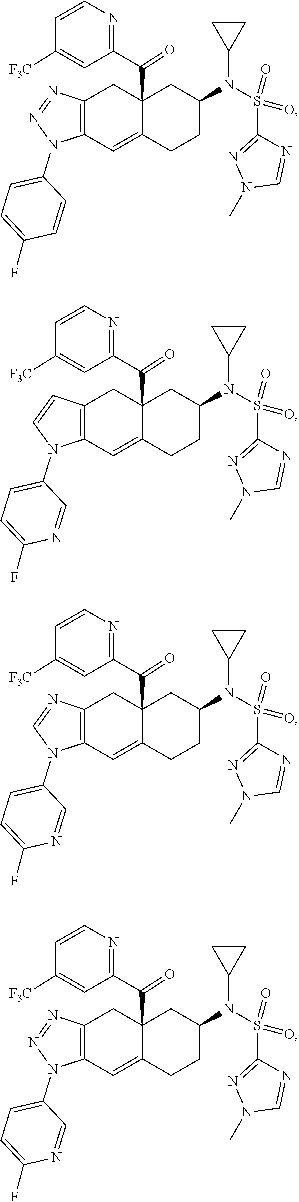 [SMILES: Cn1cnc(S(=O)(=O)N(C2CC2)[C@H]2CCC3=Cc4c(nnn4-c4ccc(F)cc4)C[C@]3(C(=O)c3cc(C(F)(F)F)ccn3)C2)n1 ; Cn1cnc(S(=O)(=O)N(C2CC2)[C@H]2CCC3=Cc4c(ccn4-c4ccc(F)nc4)C[C@]3(C(=O)c3cc(C(F)(F)F)ccn3)C2)n1 ; Cn1cnc(S(=O)(=O)N(C2CC2)[C@H]2CCC3=Cc4c(ncn4-c4ccc(F)nc4)C[C@]3(C(=O)c3cc(C(F)(F)F)ccn3)C2)n1 ; Cn1cnc(S(=O)(=O)N(C2CC2)[C@H]2CCC3=Cc4c(nnn4-c4ccc(F)nc4)C[C@]3(C(=O)c3cc(C(F)(F)F)ccn3)C2)n1]

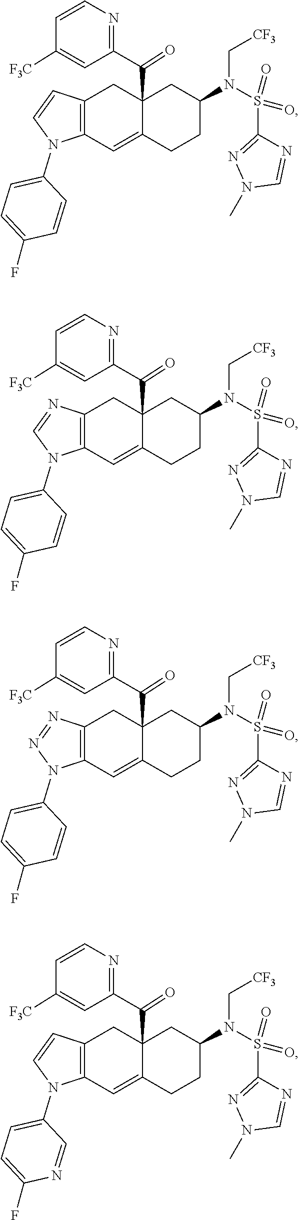 [SMILES: Cn1cnc(S(=O)(=O)N(CC(F)(F)F)[C@H]2CCC3=Cc4c(ccn4-c4ccc(F)cc4)C[C@]3(C(=O)c3cc(C(F)(F)F)ccn3)C2)n1 ; Cn1cnc(S(=O)(=O)N(CC(F)(F)F)[C@H]2CCC3=Cc4c(ncn4-c4ccc(F)cc4)C[C@]3(C(=O)c3cc(C(F)(F)F)ccn3)C2)n1 ; Cn1cnc(S(=O)(=O)N(CC(F)(F)F)[C@H]2CCC3=Cc4c(nnn4-c4ccc(F)cc4)C[C@]3(C(=O)c3cc(C(F)(F)F)ccn3)C2)n1 ; Cn1cnc(S(=O)(=O)N(CC(F)(F)F)[C@H]2CCC3=Cc4c(ccn4-c4ccc(F)nc4)C[C@]3(C(=O)c3cc(C(F)(F)F)ccn3)C2)n1]

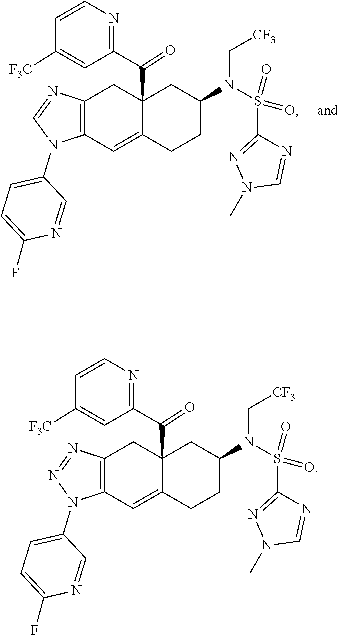 [SMILES: Cn1cnc(S(=O)(=O)N(CC(F)(F)F)[C@H]2CCC3=Cc4c(ncn4-c4ccc(F)nc4)C[C@]3(C(=O)c3cc(C(F)(F)F)ccn3)C2)n1 ; "and" ; Cn1cnc(S(=O)(=O)N(CC(F)(F)F)[C@H]2CCC3=Cc4c(nnn4-c4ccc(F)nc4)C[C@]3(C(=O)c3cc(C(F)(F)F)ccn3)C2)n1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, stereoisomer, or isotopic variant thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propane sulfonate, salicylate, succinate, sulfate, sulfite, succinate, suberate, sebacate, sulfonate, tartrate, thiocyanate, tosylateundeconate, and xylene sulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acres Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g. those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, the compounds described herein are prepared as outlined in Schemes 1-5.

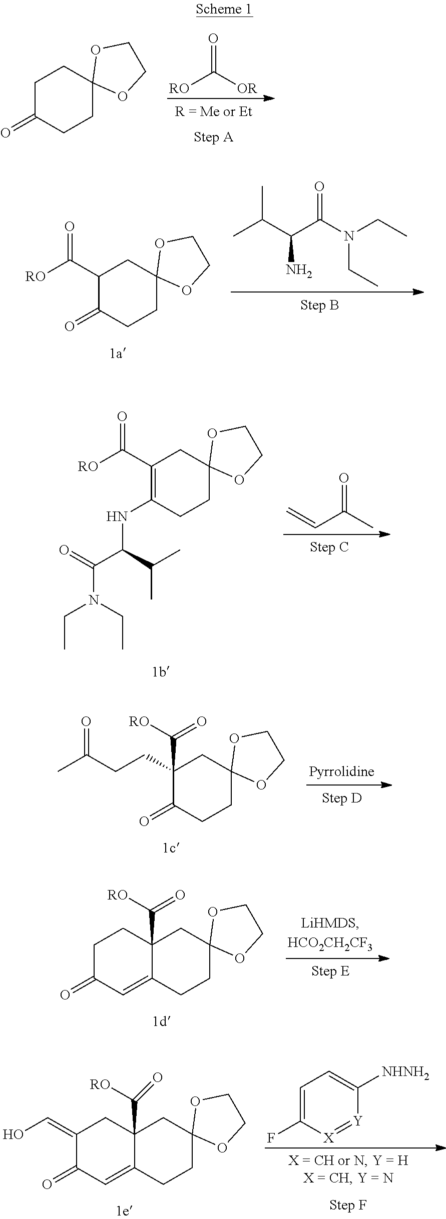

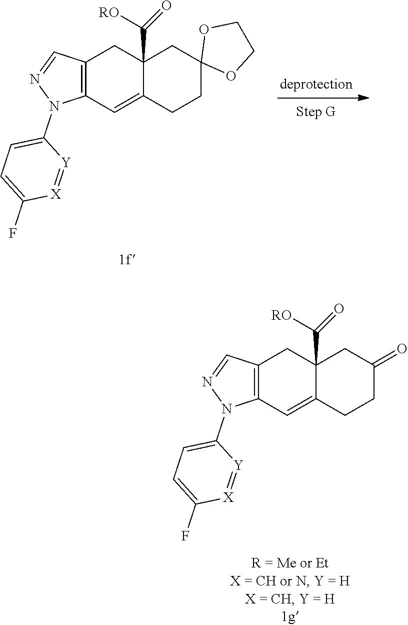

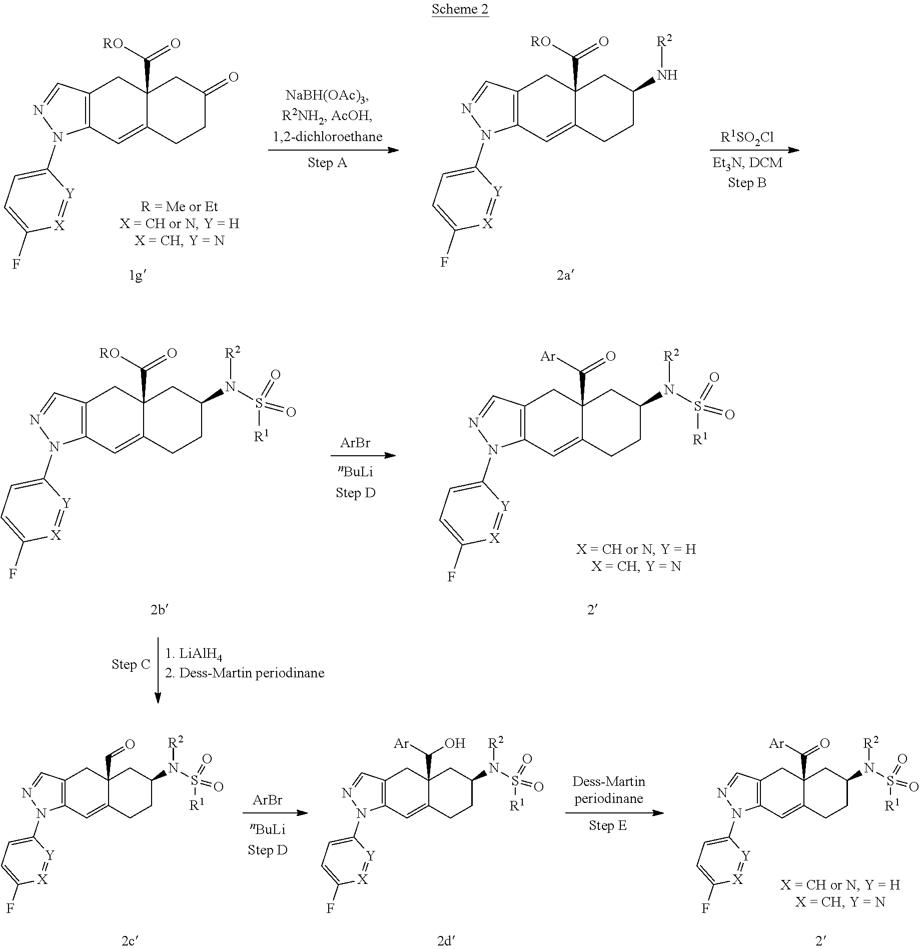

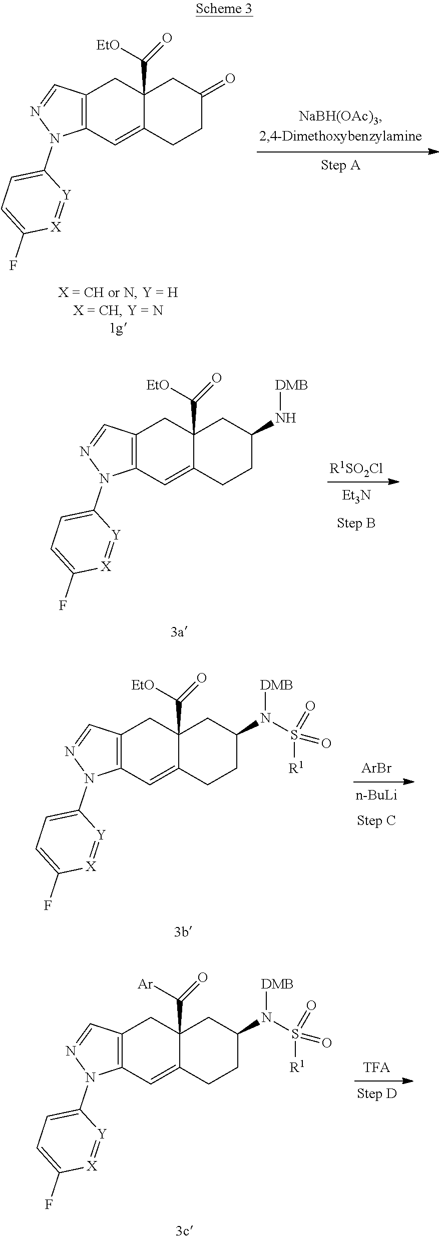

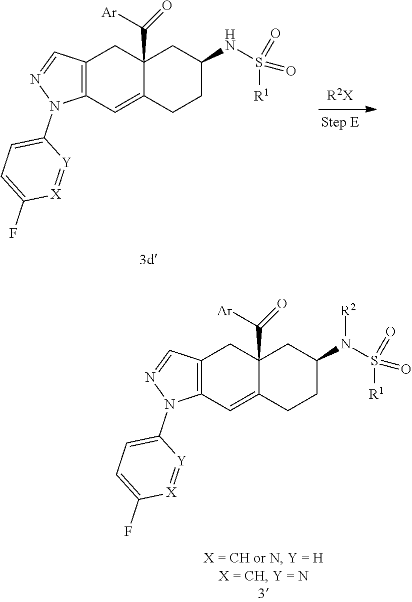

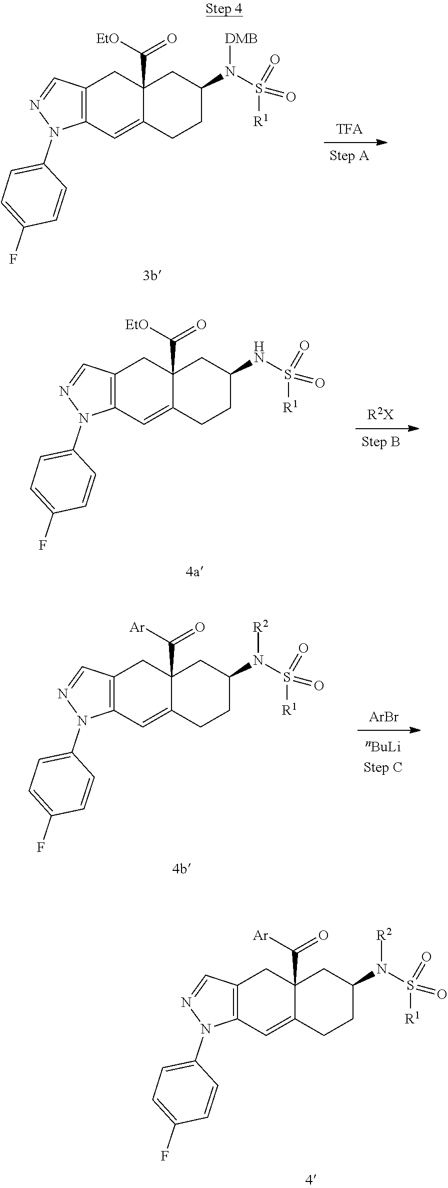

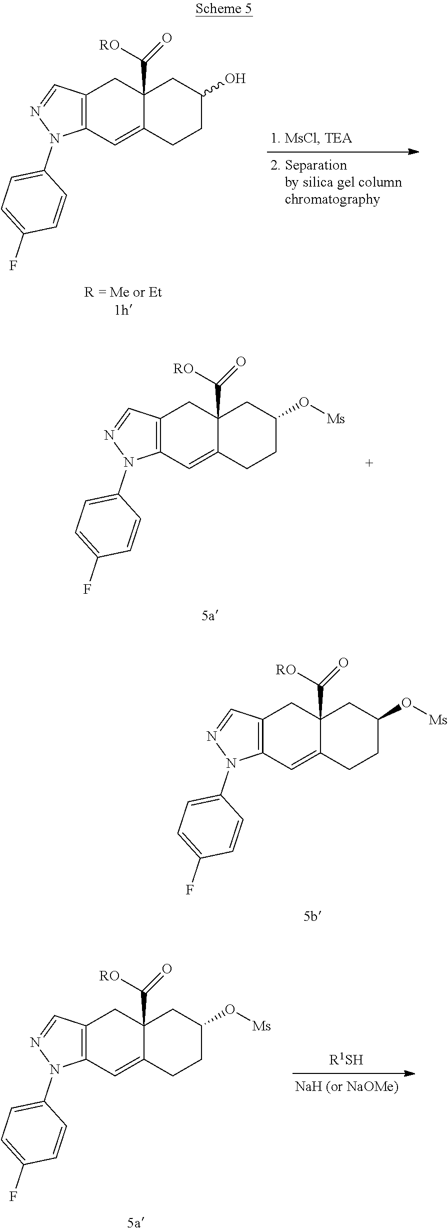

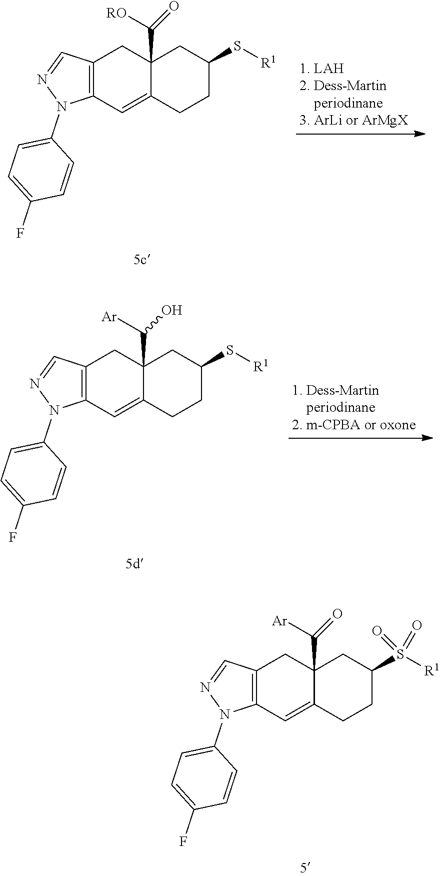

### Pharmaceutical Compositions

In certain embodiments, the compound disclosed herein is administered as a pure chemical. In some embodiments, the compound disclosed herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising at least one compound disclosed herein, or a pharmaceutically acceptable salt, solvate, stereoisomer, or isotopic variant thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable excipient and a compound disclosed herein, or a pharmaceutically acceptable salt, solvate, stereoisomer, or isotopic variant thereof.

In certain embodiments, the compound disclosed herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Use of the Compounds

### Glucocorticoid Receptor Modulators

Mifepristone is a non-selective modulator of several nuclear receptors. Mifepristone has been referred to as a GR antagonist, a progesterone receptor (PR) antagonist, a GR partial agonist, an androgen receptor (AR) antagonist and an AR partial agonist in the scientific literature. The activity observed at multiple hormone receptors leads to various undesirable side effects and in some instances, the promotion of cancer. Thus, AR agonism is an undesirable feature for GR antagonists used in the treatment of cancer (e.g., AR positive or AR dependent cancers including “castration resistant” prostate cancer (CRPC), breast cancer, or ovarian cancer). Antagonists of GR that have minimized binding to other hormone receptors, such as the androgen receptor (AR), are needed to effectively treat the diseases described herein with reduced side effects.

Some embodiments provided herein describe compounds disclosed herein that are modulators of glucocorticoid receptors (GR). In some embodiments, the compounds disclosed herein alter the level and/or activity of GR. In some embodiments, the compounds disclosed herein are GR antagonists. In some instances, glucocorticoid receptor antagonists bind to the receptor and prevent glucocorticoid receptor agonists from binding and eliciting GR mediated events, including regulation of transcription. Thus, in some embodiments, the compounds disclosed herein inhibit GR transcriptional activation activity. In some embodiments, the compounds disclosed herein are selective GR antagonists. In some embodiments, the compounds disclosed herein are not GR agonists. In some embodiments, the compounds disclosed herein are not GR partial agonists. In some embodiments, the compounds disclosed herein lessen cortisol activity in cells and make secondary therapeutic agents more effective.

In some embodiments, the compounds disclosed herein are useful for treating or preventing weight gain (e.g., Olanzapine induced weight gain), uterine fibrosis, alcoholism, alcohol abuse disorders, cocaine dependence, bipolar depression, adrenal hypercortisolism, post-traumatic stress disorder, anxiety disorders, mood disorders, hyperglycemia, and to induce abortion.

In some embodiments, the compounds disclosed herein are not androgen receptor (AR) signaling inhibitors. In these instances, the compounds disclosed herein do not significantly regulate AR levels and/or activity. In some embodiments, the compounds disclosed herein are not AR agonists. In some embodiments, the compounds disclosed herein have minimized binding to the androgen receptor (AR). In some embodiments, the compounds disclosed herein are not partial AR agonists. In some embodiments, the compounds disclosed herein have minimized partial AR agonism compared to mifepristone.

In some embodiments, the compounds disclosed herein are not partial AR agonists or partial GR agonists.

In some embodiments, the compounds disclosed herein do not modulate progesterone receptors. In some embodiments, the compounds described herein are not progesterone receptor (PR) inhibitors. In these instances, the compounds disclosed herein do not significantly regulate PR levels and/or activity. In some embodiments, the compounds disclosed herein are not PR agonists. In some embodiments, compounds disclosed herein are not PR partial agonists. In some embodiments, the compounds disclosed herein are not PR antagonists.

In some embodiments, the compounds disclosed herein are selective inhibitors. In some embodiments, use of the compounds disclosed herein in a patient does not cause or result in vaginal bleeding, cramping, nausea, vomiting, diarrhea, dizziness, back pain, weakness, tiredness, or combinations thereof. In certain embodiments, use of the compounds disclosed herein in a patient does not cause or result in vaginal bleeding. In certain embodiments, use of the compounds disclosed herein in a patient does not cause or result in cramping. In some embodiments, use of compounds disclosed herein in a patient does not cause or result in allergic reactions, low blood pressure, loss of consciousness, shortness of breath, rapid heartbeat, or combinations thereof.

### CYP Inhibition

CYPs are the major enzymes involved in drug metabolism, accounting for about 75% of the total metabolism. Most drugs undergo deactivation by CYPs, either directly or by facilitated excretion from the body. Also, many substances are bioactivated by CYPs to form their active compounds. In some instances, drugs increase or decrease the activity of various CYP isozymes either by inducing the biosynthesis of an isozyme (enzyme induction) or by directly inhibiting the activity of the CYP (enzyme inhibition). This activity is a major source of adverse drug interactions, since changes in CYP enzyme activity may affect the metabolism and clearance of various drugs. For example, if one drug inhibits the CYP-mediated metabolism of another drug, the second drug may accumulate within the body to toxic levels. Hence, in some instances, drug interactions necessitate dosage adjustments or choosing drugs that do not interact with the CYP system. Such drug interactions are especially important to take into account when using drugs of vital importance to the patient, drugs with adverse side-effects and drugs with small therapeutic windows, but any drug may be subject to an altered plasma concentration due to altered drug metabolism.

Cytochrome P₄₅₀2C8 (abbreviated CYP2C8), a member of the cytochrome P450 mixed-function oxidase system, is involved in the metabolism of xenobiotics in the body. CYP2C8 is involved in the metabolism and clearance of various cancer drugs such as, for example, enzalutamide, paclitaxel, and sorafenib. In order to avoid drug-to-drug interaction caused by inhibition of the CYP2C8 isoform, a low level of CYP2C8 inhibition is desired.

Some embodiments provided herein describe GR antagonists that do not have clinically significant drug interactions resulting from inhibition or induction of CYP enzymes. In some embodiments, the GR antagonists do not have clinically significant drug interactions resulting from inhibition or induction of CYP2C8. In some embodiments, the compounds disclosed herein have reduced CYP inhibition. In some embodiments, the compounds disclosed herein have reduced CYP2C8 inhibition. In some embodiments, the compounds disclosed herein have <25% inhibition against CYP2C8 when paclitaxel is used as a substrate. In some embodiments, the compounds disclosed herein have <50% inhibition against CYP2C8 when paclitaxel is used as a substrate. In some embodiments, the compounds disclosed herein have <60% inhibition against CYP2C8 when paclitaxel is used as a substrate. In some embodiments, the compounds disclosed herein have <70% inhibition against CYP2C8 when paclitaxel is used as a substrate. In some embodiments, the compounds disclosed herein have <90% inhibition against CYP2C8 when paclitaxel is used as a substrate. In some embodiments, the compounds disclosed herein do not inhibit CYP2C8.

### Methods of Treatment

### Cancer

One embodiment provides a method of treating cancer in a subject in need thereof, comprising administering to the subject a compound disclosed herein provided herein, or a pharmaceutically acceptable salt thereof. In some embodiments, a compound disclosed herein is used in combination with a second therapeutic agent (e.g., an anti-cancer agent) for treating cancer. In some embodiments, the combination of the compound disclosed herein with the second therapeutic agent (e.g., an anti-cancer agent) provides a more effective initial therapy for treating cancer compared to the second therapeutic agent (e.g., an anti-cancer agent) administered alone. In some embodiments, a compound disclosed herein is used in combination with one or more additional therapeutic agents (e.g., anti-cancer agents) for treating cancer. In some embodiments, the combination of the compound disclosed herein with the one or more additional therapeutic agents (e.g., an anti-cancer agents) provides a more effective initial therapy for treating cancer compared to the one or more therapeutic agents (e.g., an anti-cancer agents) administered alone.

In some embodiments, the cancer is chemoresistant cancer, radio resistant cancer, anti-hormonal therapy resistant cancer, or treatment refractory cancer. In some embodiments, the cancer is relapsed cancer, persistent cancer, or recurrent cancer. Another embodiment provided herein describes a method of reducing incidences of cancer recurrence. Also provided here in some embodiments, is a method for treating a therapy-resistant cancer.

### Prostate Cancer

Prostate cancer is the second most common cause of cancer death in men in the United States, and approximately one in every six American men will be diagnosed with the disease during his lifetime. Treatment aimed at eradicating the tumor is unsuccessful in 30% of men.

One embodiment provides a method of treating prostate cancer in a subject in need thereof, comprising administering to the subject a compound disclosed herein provided herein, or a pharmaceutically acceptable salt thereof. In some embodiments, a compound disclosed herein is used in combination with a second therapeutic agent (e.g., an anti-cancer agent) for treating prostate cancer. In some embodiments, the combination of the compound disclosed herein with the second therapeutic agent (e.g., an anti-cancer agent) provides a more effective initial therapy for treating prostate cancer compared to the second therapeutic agent (e.g., an anti-cancer agent) administered alone. In some embodiments, a compound disclosed herein is used in combination with one or more additional therapeutic agents (e.g., anti-cancer agents) for treating prostate cancer. In some embodiments, the combination of the compound disclosed herein with the one or more additional therapeutic agents (e.g., an anti-cancer agents) provides a more effective initial therapy for treating prostate cancer compared to the one or more therapeutic agents (e.g., an anti-cancer agents) administered alone.

In some embodiments, the prostate cancer is chemoresistant cancer, radio resistant cancer, antiandrogen resistant, or refractory cancer. In some embodiments, the prostate cancer is relapsed cancer, persistent cancer, or recurrent cancer.

In some embodiments, the prostate cancer is acinar adenocarcinoma, atrophic carcinoma, foamy carcinoma, colloid carcinoma, or signet ring carcinoma. In some embodiments, the prostate cancer is ductal adenocarcinoma, transitional cell cancer, urothelial cancer, squamous cell cancer, carcinoid cancer, small cell cancer, sarcoma cancer, or sarcomatoid cancer. In some embodiments, the prostate cancer is metastatic castration-resistant prostate cancer, doubly-resistant prostate cancer, castration-resistant prostate cancer, hormone-resistant prostate cancer, androgen-independent, or androgen-refractory cancer.

In some instances, antiandrogens are useful for the treatment of prostate cancer during its early stages. In some instances, prostate cancer cells depend on androgen receptor (AR) for their proliferation and survival. Some prostate cancer patients are physically castrated or chemically castrated by treatment with agents that block production of testosterone (e.g. GnRH agonists), alone or in combination with antiandrogens, which antagonize effects of any residual testosterone.

In some instances, prostate cancer advances to a hormone-refractory state in which the disease progresses despite continued androgen ablation or antiandrogen therapy. The hormone-refractory state to which most patients eventually progress in the presence of continued androgen ablation or anti-androgen therapy is known as “castration resistant” prostate cancer (CRPC). CRPC is associated with an overexpression of AR. AR is expressed in most prostate cancer cells and overexpression of AR is necessary and sufficient for androgen-independent growth of prostate cancer cells. Failure in hormonal therapy, resulting from development of androgen-independent growth, is an obstacle for successful management of advanced prostate cancer.

While a small minority of CRPC does bypass the requirement for AR signaling, the vast majority of CRPC, though frequently termed “androgen independent prostate cancer” or “hormone refractory prostate cancer,” retains its lineage dependence on AR signaling.

Recently approved therapies that target androgen receptor (AR) signaling such as abiraterone acetate and enzalutamide have been utilized for treating CRPC. Despite these successes, sustained response with these agents is limited by acquired resistance which typically develops within 6-12 months. Doubly resistant prostate cancer is characterized in that tumor cells have become castration resistant and resistant when treated with second generation antiandrogens. Doubly resistant prostate cancer cells are characterized by a lack of effectiveness of second generation antiandrogens in inhibiting tumor growth.

In some embodiments, resistant prostate cancer (e.g., doubly resistant and castration resistant prostate cancers) occurs when cancer cells overexpress androgen receptors (AR). In some instances, increased signaling through the glucocorticoid receptor (GR) compensates for inhibition of androgen receptor signaling in resistant prostate cancer. Double resistant prostate cancer develops when expression of a subset of AR target genes is restored through activity of GR. In some instances, GR activation is responsible for this target gene activation. In some embodiments, GR transcription is activated in patients susceptible to or suffering from resistant prostate cancer (e.g., doubly resistant and castration resistant prostate cancers). In some instances, GR upregulation in cancer cells confers resistance to antiandrogens.

Some embodiments provided herein describe the use of a compound disclosed herein for treating prostate cancer in a subject in need thereof, including doubly resistant prostate cancer and castration resistant prostate cancer. In some embodiments, the subject in need has elevated tumor GR expression. In some embodiments, the compound disclosed herein is also an AR signaling inhibitor or antiandrogen.

In some embodiments, the compound disclosed herein is used in combination with a second therapeutic agent. In some embodiments, the compound disclosed herein is used in combination with one or more additional therapeutic agents. In some embodiments, the second or additional agent is an anti-cancer agent. In certain embodiments, the anti-cancer agent is useful for AR positive or AR negative prostate cancer.

### Breast Cancer

Breast cancer is the second leading cause of cancer among women in the United States. Triple-negative breast cancers are among the most aggressive and difficult to treat of all the breast cancer types. Triple-negative breast cancer is a form of the disease in which the three receptors that fuel most breast cancer growth—estrogen, progesterone and the HER-2—are not present. Because the tumor cells lack these receptors, treatments that target estrogen, progesterone and HER-2 are ineffective. Approximately 40,000 women are diagnosed with triple-negative breast cancer each year. It is estimated that more than half of these women's tumor cells express significant amounts of GR.

In some instances, GR expression is associated with a poor prognosis in estrogen receptor (ER)-negative early stage breast cancer. In some instances, GR activation in triple-negative breast cancer cells initiates an anti-apoptotic gene expression profile that is associated with inhibiting chemotherapy-induced tumor cell death. GR activity in these cancer cells correlates with chemotherapy resistance and increased recurrence of cancer.

Provided herein in some embodiments are methods of treating breast cancer, the method comprising administering to a subject in need thereof a compound disclosed herein provided herein, or a pharmaceutically acceptable salt thereof. In some embodiments, a compound disclosed herein is used in combination with a second therapeutic agent (e.g., a chemotherapeutic agent) for treating breast cancer. In some embodiments, the combination of the compound disclosed herein with the second therapeutic agent (e.g., a chemotherapeutic agent) provides a more effective initial therapy for treating breast cancer compared to the second therapeutic agent (e.g., a chemotherapeutic agent) administered alone. In some embodiments, a compound disclosed herein is used in combination with one or more additional therapeutic agents (e.g., anti-cancer agents) for treating breast cancer. In some embodiments, the combination of the compound disclosed herein with the one or more additional therapeutic agents (e.g., an anti-cancer agents) provides a more effective initial therapy for treating breast cancer compared to the one or more therapeutic agents (e.g., an anti-cancer agents) administered alone.

In some embodiments, the breast cancer is chemoresistant cancer, radio resistant cancer, antihormonal therapy resistant cancer, or refractory cancer. In some embodiments, the breast cancer is relapsed cancer, persistent cancer, or recurrent cancer. Breast cancers may include, but are not limited to, ductal carcinoma, invasive ductal carcinoma, tubular carcinoma of the breast, medullary carcinoma of the breast, mecinous carcinoma of the breast, papillary carcinoma of the breast, cribriform carcinoma of the breast, invasive lobular carcinoma, inflammatory breast cancer, lobular carcinoma in situ, male breast cancer, Paget disease of the nipple, phyllodes tumor of the breast, recurrent and metastatic breast cancer, triple-negative breast cancer, or combinations thereof.

In some embodiments, the breast cancer is recurrent and metastatic breast cancer, triple-negative breast cancer, or combinations thereof. In some embodiments, the breast cancer is chemoresistant triple-negative breast cancer or estrogen receptor (ER) negative breast cancer. In some embodiments, the breast cancer is chemoresistant triple-negative breast cancer. In some embodiments, the breast cancer is estrogen receptor (ER) negative breast cancer. In some embodiments, the breast cancer is GR+ triple-negative breast cancer. In some embodiments, the breast cancer is GR+ estrogen receptor (ER) negative breast cancer.

Some embodiments provided herein describe the use of a compound disclosed herein for treating breast cancer in a patient, including triple negative breast cancer or ER negative breast cancer. In some embodiments, the compound described herein inhibits the anti-apoptotic signaling pathways of GR and increase the cytotoxic efficiency of secondary chemotherapeutic agents. In some embodiments, the compounds described herein enhance the efficacy of chemotherapy in breast cancer patients, such as triple negative breast cancer patients. In some embodiments, the breast cancer patient has elevated tumor GR expression.

Some embodiments provided herein describe methods of treating estrogen positive breast cancer. In some instances, estrogen positive breast cancer patients become resistant to estrogen receptor modulators. In some embodiments, the compound disclosed herein enhances the efficacy of estrogen receptor modulators in estrogen positive breast cancer patients. In some embodiments, the breast cancer patient has elevated tumor GR expression. In some embodiments, a GR inhibitor described herein is used in combination with an estrogen receptor modulator. In some embodiments, the estrogen receptor modulator is tamoxifen, raloxifene, toremifene, tibolone, fulvestrant, lasofoxifene, clomifene, ormeloxifene, or ospemifene. In some embodiments, the estrogen receptor modulator is tamoxifen, raloxifene, toremifene, tibolone, or fulvestrant. In some embodiments, the estrogen receptor modulator is tamoxifen, raloxifene, or toremifene. In certain embodiments, the estrogen receptor modulator is tamoxifen.

### Ovarian Cancer

Ovarian cancer is the leading cause of death from gynecologic malignancies. Some ovarian cancers (e.g., high grade serous ovarian cancer) are initially sensitive to platinum-based therapy, but relapse rates remain high.

One embodiment provides a method of treating ovarian cancer in a patient in need thereof, comprising administering to the patient a compound disclosed herein provided herein, or a pharmaceutically acceptable salt thereof. In some embodiments, the patient has elevated tumor GR expression. In some embodiments, a compound disclosed herein is used in combination with a second therapeutic agent (e.g., a chemotherapeutic agent) for treating ovarian cancer. In some embodiments, the combination of the compound disclosed herein with the second therapeutic agent (e.g., a chemotherapeutic agent) provides a more effective initial therapy for treating ovarian cancer compared to the second therapeutic agent (e.g., a chemotherapeutic agent) administered alone. In some embodiments, a compound disclosed herein is used in combination with one or more additional therapeutic agents (e.g., anti-cancer agents) for treating ovarian cancer. In some embodiments, the combination of the compound disclosed herein with the one or more additional therapeutic agents (e.g., an anti-cancer agents) provides a more effective initial therapy for treating ovarian cancer compared to the one or more therapeutic agents (e.g., an anti-cancer agents) administered alone.

In some instances, GR activation increases resistance to chemotherapy in ovarian cancer (e.g., high-grade serous ovarian cancer). In some instances, GR activation significantly inhibits chemotherapy induced apoptosis in ovarian cancer cells. Provided herein in some embodiments are methods of treating ovarian cancer in a subject, the method comprising treating the subject with a compound disclosed herein to improve sensitivity to chemotherapy. In some embodiments, the ovarian cancer has become resistant to chemotherapy. In some embodiments, the ovarian cancer cells are resistant to cisplatin, carboplatin, paclitaxel, docetaxel, nab-paclitaxel, cabazitaxel, gemcitabine, pemetrexed, alone or in combination. In some embodiments, the ovarian cancer cells are resistant to cisplatin, paclitaxel, carboplatin, gemcitabine, alone or in combination. In some embodiments, the compound disclosed herein reverses the cell survival effect.

Ovarian cancers may include, but are not limited to, epithelial ovarian cancers, such as serous epithelial ovarian cancer, endometrioid epithelial ovarian cancer, clear cell epithelial ovarian cancer, mucinous epithelial ovarian cancer, undifferentiated or unclassifiable epithelial ovarian cancer, refractory ovarian cancer, sex cord-stromal tumors, Sertoli and Sertoli-Leydig cell tumors, germ cell tumors, such as dysgerminoma and nondysgerminomatous tumors, Brenner tumors, primary peritoneal carcinoma, fallopian tube cancer, or combinations thereof.

### Non-Small Cell Lung Cancer

One embodiment provides a method of treating non-small cell lung cancer (NSCLC) in a patient in need thereof, comprising administering to the patient a compound disclosed herein provided herein, or a pharmaceutically acceptable salt thereof. In some embodiments, the patient has elevated tumor GR expression. In some embodiments, a compound disclosed herein is used in combination with a second therapeutic agent (e.g., a chemotherapeutic agent) for treating NSCLC. In some embodiments, the combination of the compound disclosed herein with the second therapeutic agent (e.g., a chemotherapeutic agent) provides a more effective initial therapy for treating NSCLC compared to the second therapeutic agent (e.g., a chemotherapeutic agent) administered alone. In some embodiments, a compound disclosed herein is used in combination with one or more additional therapeutic agents (e.g., anti-cancer agents) for treating NSCLC. In some embodiments, the combination of the compound disclosed herein with the one or more additional therapeutic agents (e.g., an anti-cancer agents) provides a more effective initial therapy for treating NSCLC compared to the one or more therapeutic agents (e.g., an anti-cancer agents) administered alone.

### Hypercortisolism/Cushing's Disease

One embodiment provides a method of treating hypercortisolism or Cushing's disease in a patient in need thereof, comprising administering to the patient a compound disclosed herein provided herein, or a pharmaceutically acceptable salt thereof.

Types of Cushing's disease include, but are not limited to, recurrent Cushing's disease, refractory Cushing's disease, persistent Cushing's disease, endogenous Cushing's disease, spontaneous hypercortisolism, Adrenocorticotropic hormone dependent, Adrenocorticotropic hormone independent, or combinations thereof.

Causes of hypercortisolism may include, but are not limited to, prolonged exposure to cortisol, a tumor that produces excessive cortisol, a tumor that results in the excess production of cortisol, or combinations thereof.

### Combination Treatment

In some embodiments, a compound disclosed herein is used in combination with at least a second therapeutic agent, such as a chemotherapeutic agent or immunotherapy. In some embodiments, the compound disclosed herein is used in combination with one or more additional therapeutic agents. In some embodiments, the second or additional therapeutic agent is cisplatin, carboplatin, cyclophosphamide, capecitabine, gemcitabine, paclitaxel, nab-paclitaxel, altretamine, docetaxel, epirubicin, melphalan, methotrexate, mitoxantrone, ixabepilone, ifosfamide, irinotecan, eribulin, etoposide, doxorubicin, liposomal doxorubicin, camptothecin, pemetrexed, topotecan, vinorelbine, vinblastine, daunorubicin, fluorouracil, mitomycin, thiotepa, vincristine, everolimus, veliparib, glembatumumab vedotin, pertuzumab, trastuzumab, or any combinations or any salts thereof. In some embodiments, cisplatin, carboplatin, paclitaxel, docetaxel, nab-paclitaxel, cabazitaxel, gemcitabine, pemetrexed, or any combinations or any salts thereof. In some embodiments, the second or additional therapeutic agent is gemcitabine. In some embodiments, the second or additional therapeutic agent is carboplatin. In some embodiments, the second or additional therapeutic agent is cisplatin. In some embodiments, the second or additional agent is paclitaxel. In some embodiments, the compound disclosed herein is used in combination with gemcitabine and carboplatin. In some embodiments, the compound disclosed herein is used in combination with carboplatin and cisplatin. In some embodiments, the second or additional therapeutic agent is an anti-PD-L1 agent. In certain embodiments, the anti-PD-L1 agent is atezolizumab (MPDL3280A) or avelumab. In some embodiments, the second or additional therapeutic agent is an anti-PD 1 agent. In certain embodiments, the anti-PD 1 agent is nivolumab or pembrolizumab. In some embodiments, the second or additional therapeutic agent is an anti-CTLA-4 agent. In some embodiments, the second or additional therapeutic agent is a CAR-T cells therapy. In some embodiments, the second or additional therapeutic agent is a cancer vaccine. In some embodiments, the second or additional therapeutic agent is an IDO-1 inhibitor.

In some embodiments, the second or additional agent is an AR signaling inhibitor or antiandrogen. In certain embodiments, the AR signaling inhibitor is an AR antagonist. In some embodiments, the second or additional therapeutic agent is selected from finasteride, dutasteride, alfatradiol, cyproterone acetate, spironolactone, danazol, gestrinone, ketoconazole, abiraterone acetate, enzalutamide, apalutamide, danazol, gestrinone, danazol, simvastatin, aminoglutethimide, atorvastatin, simvastatin, progesterone, cyproterone acetate, medroxyprogesterone acetate, megestrol acetate, chlormadinone acetate, spironolactone, drospirenone, estradiol, ethinyl estradiol, diethylstilbestrol, conjugated equine estrogens, buserelin, deslorelin, gonadorelin, goserelin, histrelin, leuprorelin, nafarelin, triptorelin, abarelix, cetrorelix, degarelix, ganirelix, or any combinations or any salts thereof. In some embodiments, the second or additional therapeutic agent is selected from flutamide, nilutamide, bicalutamide, enzalutamide, apalutamide, cyproterone acetate, megestrol acetate, chlormadinone acetate, spironolactone, canrenone, drospirenone, ketoconazole, topilutamide, cimetidine, or any combinations or any salts thereof. In some embodiments, the AR signaling inhibitor is 3,3′-diindolylmethane (DIM), abiraterone acetate, apalutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof. In some embodiments, the AR signaling inhibitor is flutamide, nilutamide, bicalutamide, or megestrol. In other embodiments, the androgen receptor signaling inhibitor is enzalutamide and apalutamide. In some embodiments, the AR signaling inhibitor is apalutamide. In other embodiments, the AR signaling inhibitor is enzalutamide.

In some embodiments, the anti-cancer agent is mitoxantrone, estramustine, etoposide, vinblastine, carboplatin, vinorelbine, paclitaxel, daunomycin, darubicin, epirubicin, docetaxel, nab-paclitaxel, cabazitaxel, pemetrexed, or doxorubicin. In some embodiments, the anti-cancer agent is paclitaxel, daunomycin, darubicin, epirubicin, docetaxel, cabazitaxel, or doxorubicin. In certain embodiments, the anti-cancer agent is docetaxel.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1. N-((4aS,6S)-4a-(5-(tert-Butyl)thiazole-2-carbonyl)-1-(4-fluorophenyl)-4,4a,5,6,7,8-hexahydro-1H-benzo[f]indazol-6-yl)-N-cyclopropyl-1-methyl-1H-imidazole-4-sulfonamide (1)

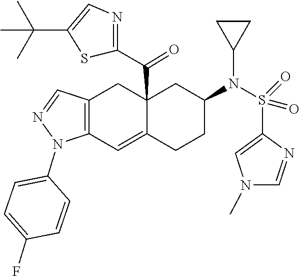 [SMILES: Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)c1]

### Step A: Ethyl 8-oxo-1,4-dioxaspiro[4.5]decane-7-carboxylate (1a)

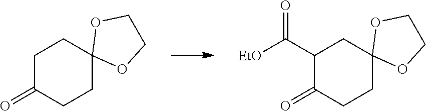 [SMILES: O=C1CCC2(CC1)OCCO2 ; CCOC(=O)C1CC2(CCC1=O)OCCO2]

A solution of diethyl carbonate (568 g, 4.81 mmol) and 1,4-dioxaspiro[4.5]decan-8-one (75.0 g, 0.481 mol) in anhydrous THF (300 mL) was added to a suspension of sodium hydride (60% in mineral oil, 48 g, 1.2 mol) in anhydrous THF (500 mL). After the mixture was refluxed for 3 h, it was cooled down to 0° C., neutralized with AcOH (pH 7), and diluted with water. The solution was extracted with EtOAc and the combined organic layers were washed with saturated NaHCO₃ solution, and brine, dried over Na₂SO₄, filtered and concentrated. The residue was purified by column chromatography to afford the title compound (1a) (76.2 g, 70%) as a colorless oil. m/z (ESI, +ve ion)=250.9 [M+Na]⁺.

### Step B: Ethyl (S)-8-((1-(diethylamino)-3-methyl-1-oxobutan-2-yl)amino)-1,4-dioxaspiro[4.5]dec-7-ene-7-carboxylate (1b)

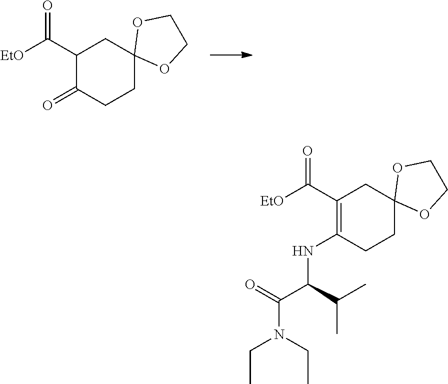 [SMILES: CCOC(=O)C1CC2(CCC1=O)OCCO2 ; CCOC(=O)C1=C(N[C@H](C(=O)N(CC)CC)C(C)C)CCC2(C1)OCCO2]

(S)-2-Amino-N,N-diethyl-3-methylbutanamide (33.0 g, 0.145 mol), molecular sieves (6.0 g, 4 Å), and concentrated hydrochloric acid (2 mL) were successively added to a solution of ethyl 8-oxo-1,4-dioxaspiro[4.5]decane-7-carboxylate (1a) (50 g, 0.29 mol) in toluene (400 mL). After the reaction mixture was stirred for 16 h at 50° C., it was filtrated, the residue was washed with DCM and the filtrate was evaporated under vacuum. The crude product was purified by column chromatography on neutral aluminum oxide to provide the title compound (1b) (28.7 g, 51%).

### Step C: Ethyl (S)-8-oxo-7-(3-oxobutyl)-1,4-dioxaspiro[4.5]decane-7-carboxylate (1c)

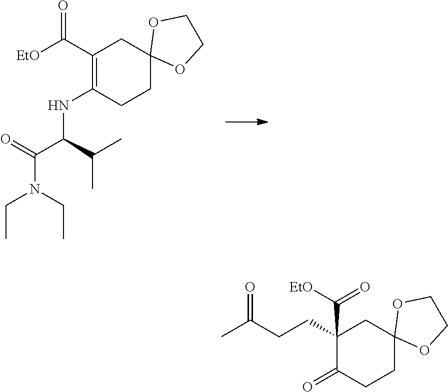 [SMILES: CCOC(=O)C1=C(N[C@H](C(=O)N(CC)CC)C(C)C)CCC2(C1)OCCO2 ; CCOC(=O)[C@@]1(CCC(C)=O)CC2(CCC1=O)OCCO2]

A mixture of ethyl (S)-8-((1-(diethylamino)-3-methyl-1-oxobutan-2-yl)amino)-1,4-dioxaspiro[4.5]dec-7-ene-7-carboxylate (Ib) (25.0 g, 65.4 mmol), Cu(OAc)₂.H₂O (1.19 g, 6.54 mmol) in acetone (250 mL) was stirred for 30 min at rt, methyl vinyl ketone (13.9 g, 0.196 mmol) was added and the mixture was stirred at rt for 3 days. All volatile materials were removed under vacuum and the residue was diluted with 10% aqueous acetic acid. The resulting solution was stirred at rt overnight and extracted with DCM. The combined organic layers were washed with sat. aq NaHCO₃ and brine, dried, and concentrated under reduced pressure. The residue was purified by column chromatography to provide the title compound (1c) (15.4 g, 67%).

### Step D: Ethyl (S)-6-oxo-4,6,7,8-tetrahydro-1H-spiro[naphthalene-2,2′-[1,3]dioxolane]-8a(3H)-carboxylate (1d)

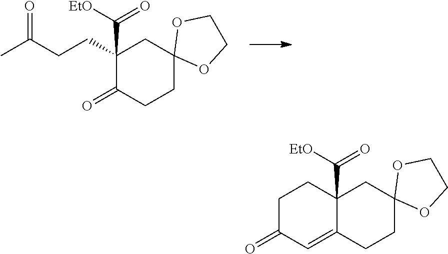 [SMILES: CCOC(=O)[C@@]1(CCC(C)=O)CC2(CCC1=O)OCCO2 ; CCOC(=O)[C@]12CCC(=O)C=C1CCC1(C2)OCCO1]

Pyrrolidine (0.734 g, 10.3 mmol) and AcOH (0.62 g, 10.3 mmol) were added to a solution of ethyl (S)-8-oxo-7-(3-oxobutyl)-1,4-dioxaspiro[4.5]decane-7-carboxylate (1c) (15.4 g, 51.7 mmol) in toluene (160 mL). After being stirred at 100° C. for 2 h, the reaction mixture was cooled down to rt and washed with sat. aq NaHCO₃, and brine, dried, and concentrated under reduced pressure. The residue was purified by column chromatography to give the title compound (1d) (12.2 g, 84%) as a yellow oil.

### Step E: Ethyl (S,Z)-7-(hydroxymethylene)-6-oxo-4,6,7,8-tetrahydro-1H-spiro[naphthalene-2,2′-[1,3]dioxolane]-8a(3H)-carboxylate (1e)

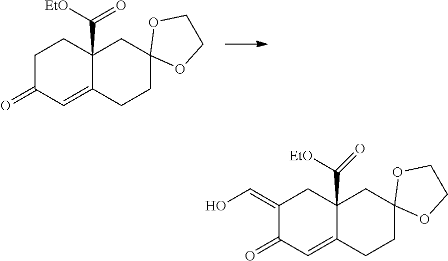 [SMILES: CCOC(=O)[C@]12CCC(=O)C=C1CCC1(C2)OCCO1 ; CCOC(=O)[C@]12C/C(=C/O)C(=O)C=C1CCC1(C2)OCCO1]

A solution of ethyl (S)-6-oxo-4,6,7,8-tetrahydro-1H-spiro[naphthalene-2,2′-[1,3]dioxolane]-8a(3H)-carboxylate (1d) (11.9 g, 42.5 mmol) in ether (80 mL) was added to lithium hexamethyldisilazide (255 mL, 255 mmol) in diethyl ether (200 mL) at −78° C. After 20 min, 2,2,2-trifluoroethyformate (54.4 g, 0.425 mol) was added. The reaction was stirred at −78° C. for 2 h and then allowed to slowly warm to rt. The reaction was quenched with sat. NH₄Cl and extracted with DCM. The organic phase was separated, washed with brine, dried and concentrated to give the title compound (1e) (13.0 g), which was used for the next step without further purification, m/z (ESI, +ve ion)=306.9 [M−H]⁺.

### Step F: Ethyl (S)-1-(4-fluorophenyl)-1,4,7,8-tetrahydrospiro[benzo[f]indazole-6,2′-[1,3]dioxolane]-4a(5H)-carboxylate (1f)

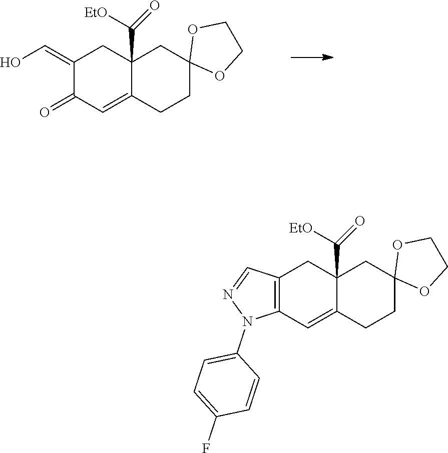 [SMILES: CCOC(=O)[C@]12C/C(=C/O)C(=O)C=C1CCC1(C2)OCCO1 ; CCOC(=O)[C@]12Cc3cnn(-c4ccc(F)cc4)c3C=C1CCC1(C2)OCCO1]

To a suspension of ethyl (S,Z)-7-(hydroxymethylene)-6-oxo-4,6,7,8-tetrahydro-1H-spiro[naphthalene-2,2′-[1,3]dioxolane]-8a(3H)-carboxylate (1e) (13.0 g, 42.2 mmol) in acetic acid (90 mL) were added sodium acetate (3.81 g, 46.4 mmol) and 4-fluorophenylhydrazine (7.20 g, 44.3 mmol). The reaction mixture was stirred at rt for 3 h and was diluted with water, extracted with EtOAc. The combined organic layers were washed with brine, dried, and concentrated under reduced pressure. The resulting oil was purified by column chromatography on silica gel to provide the title compound (If) (13 g, 91%) as a yellow solid, m/z (ESI, +ve ion)=398.7 [M+H]⁺.

### Step G: Ethyl (S)-1-(4-fluorophenyl)-6-oxo-1,4,5,6,7,8-hexahydro-4aH-benzo[f]indazole-4a-carboxylate (1g)

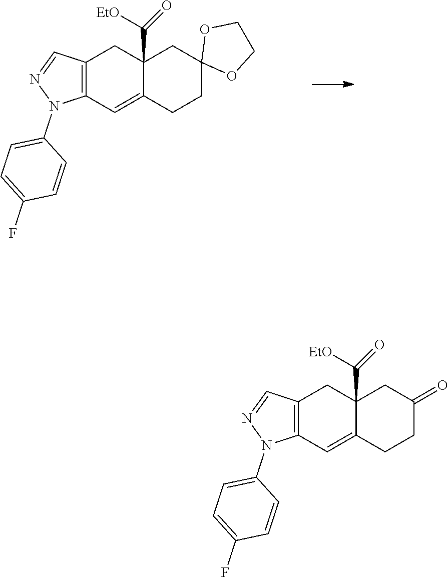 [SMILES: CCOC(=O)[C@]12Cc3cnn(-c4ccc(F)cc4)c3C=C1CCC1(C2)OCCO1 ; CCOC(=O)[C@@]12CC(=O)CCC1=Cc1c(cnn1-c1ccc(F)cc1)C2]

To a solution of ethyl (S)-1-(4-fluorophenyl)-1,4,7,8-tetrahydrospiro[benzo[f]indazole-6,2′-[1,3]dioxolane]-4a(5H)-carboxylate (If) (13.0 g, 32.7 mmol) in acetone (140 mL) was added 4 N aqueous HCl (140 mL). The reaction mixture was stirred at rt overnight and was diluted with EtOAc, basified with sat. aq. NaHCO₃, and extracted EtOAc. The combined organic layers were washed with brine, dried, and concentrated under reduced pressure to give the title compound (1g) which was used in the next step without further purification, m/z (ESI, +ve ion)=354.9 [M+H]⁺.

### Step H: Ethyl (4aS,6S)-6-(cyclopropylamino)-1-(4-fluorophenyl)-1,4,5,6,7,8-hexahydro-4aH-benzo[f]indazole-4a-carboxylate (1h)

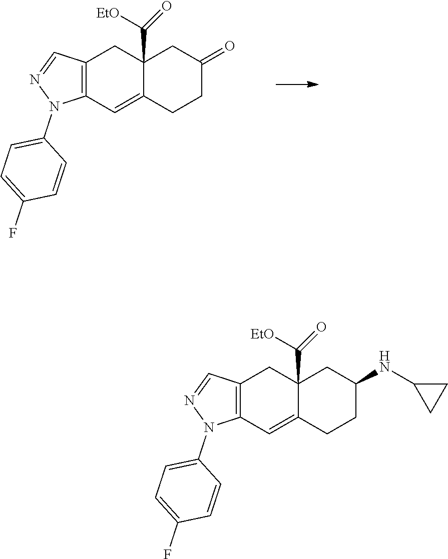 [SMILES: CCOC(=O)[C@@]12CC(=O)CCC1=Cc1c(cnn1-c1ccc(F)cc1)C2 ; CCOC(=O)[C@]12Cc3cnn(-c4ccc(F)cc4)c3C=C1CC[C@H](NC1CC1)C2]

To a solution of 1g (531 mg, 1.50 mmol) and cyclopropylamine (256 mg, 4.50 mmol) in DCE (10 mL) was added acetic acid (0.26 mL, 4.5 mmol). After the reaction was stirred for 2 min and cooled to 0° C., sodium triacetoxyborohydride (952 mg, 4.50 mmol) was added. After 5 min, the solution was allowed to warm to rt and the flask was sonicated for 2 min. After the reaction was stirred at rt for another 18 min, it was quenched (sat. aq. NaHCO₃) and extracted (EtOAc). The combined organic layers were washed (brine), dried (Na₂SO₄) and concentrated under reduced pressure. The crude product was purified by column chromatography (0%-75% EtOAc/hexanes, a gradient) to afford the title compound (1h) as a white solid (487 mg, 82%). m/z (ESI, +ve ion)=396.2 [M+H]⁺.

### Step I: Ethyl (4aS,6S)-6-((N-cyclopropyl-1-methyl-1H-imidazole)-4-sulfonamido)-1-(4-fluorophenyl)-1,4,5,6,7,8-hexahydro-4aH-benzo[f]indazole-4a-carboxylate (1i)

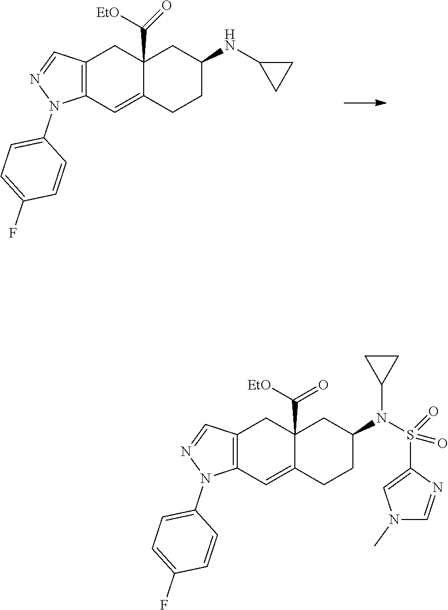 [SMILES: CCOC(=O)[C@]12Cc3cnn(-c4ccc(F)cc4)c3C=C1CC[C@H](NC1CC1)C2 ; CCOC(=O)[C@]12Cc3cnn(-c4ccc(F)cc4)c3C=C1CC[C@H](N(C1CC1)S(=O)(=O)c1cn(C)cn1)C2]

A flask was charged with 1h (215 mg, 0.54 mmol, azeotroped with toluene), 1-methyl-1H-imidazole-4-sulfonyl chloride (65 mg, 0.34 mmol) was added, followed by DCM (3.1 mL) and triethylamine (0.3 mL, 2.2 mmol) successively. The reaction was stirred under argon at rt overnight and then concentrated to dryness. The residue was directly purified by column chromatography (0%-100% EtOAc/hexanes, a gradient elution) to afford the title compound (1i) as a white solid (253 mg, 86%). m/z (ESI, +ve ion)=540.2 [M+H]⁺.

### Step J. N-((4aS,6S)-4a-(5-(tert-Butyl)thiazole-2-carbonyl)-1-(4-fluorophenyl)-4,4a,5,6,7,8-hexahydro-1H-benzo[f]indazol-6-yl)-N-cyclopropyl-1-methyl-1H-imidazole-4-sulfonamide (1)

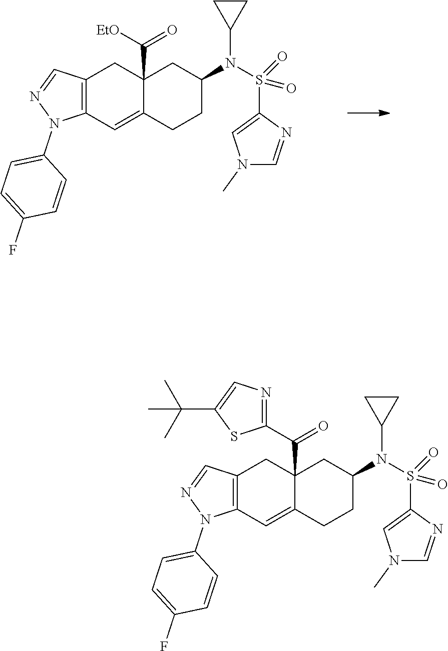 [SMILES: CCOC(=O)[C@]12Cc3cnn(-c4ccc(F)cc4)c3C=C1CC[C@H](N(C1CC1)S(=O)(=O)c1cn(C)cn1)C2 ; Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)c1]

A flask with 1i (30 mg, 0.06 mmol) was azeotroped with toluene and put on high vacuum. A separately dried flask under an argon balloon was charged with anhydrous ether (1 mL), cooled down to −78° C., n-butyllithium (0.14 mL, 0.22 mmol) was added, followed by the dropwise addition of 2-bromo-5-(tert-butyl)-1,3-thiazole (31.4 uL, 0.25 mmol). The solution remained a brownish red and was stirred for 30 min at −78° C. The flask with the ester was flushed with argon and dissolved in anhydrous THF (0.5 mL). The resulting solution was added dropwise to the flask with the lithiated species at −78° C. The mixture was continuously stirred for 25 min and then quenched with water and saturated NH₄Cl. The solution was allowed to warm to rt and extracted with EtOAc. The organic layer was separated, washed with brine, dried and concentrated. The residue was purified by column chromatography (0%-50% EtOAc/hexanes, a gradient elution) to afford the title compound (1) as a light yellow solid (31 mg, 88%). ¹H NMR (400 MHz, CHLOROFORM-d) δ ppm 7.69 (1H, s), 7.51 (1H, d, J=1.5 Hz), 7.44 (3H, m), 7.29 (1H, br s), 7.14 (2H, m), 6.46 (1H, J=1.8 Hz), 4.37 (1H, m), 4.04 (1H, J=16.5 Hz), 3.76 (3H, s), 3.13 (1H, d, J=16.5 Hz), 2.6 (2H, m), 2.45 (2H, m), 2.3 (1H, m), 2.1 (1H, m), 1.88 (1H, m), 1.41 (9H, s), 0.90-1.02 (2H, m), 0.70 (2H, m). m/z (ESI, +ve ion)=635.2 [M+H]⁺.

### Example 2. N-Cyclopropyl-N-((4aS,6S)-1-(4-fluorophenyl)-4a-(4-fluoropicolinoyl)-4,4a,5,6,7,8-hexahydro-1H-benzo[f]indazol-6-yl)-1-methyl-1H-imidazole-4-sulfonamide (2)

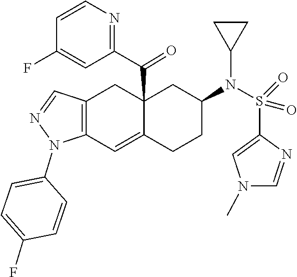 [SMILES: Cn1cnc(S(=O)(=O)N(C2CC2)[C@H]2CCC3=Cc4c(cnn4-c4ccc(F)cc4)C[C@]3(C(=O)c3cc(F)ccn3)C2)c1]

### Step A: N-Cyclopropyl-N-((4aS,6S)-1-(4-fluorophenyl)-4a-(hydroxymethyl)-4,4a,5,6,7,8-hexahydro-1H-benzo[f]indazol-6-yl)-1-methyl-1H-imidazole-4-sulfonamide (2a)

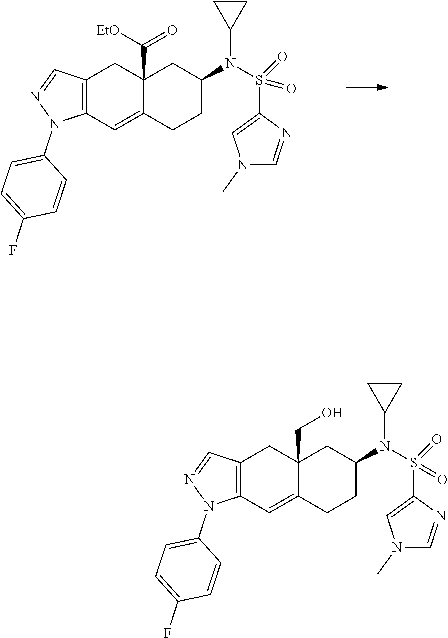 [SMILES: CCOC(=O)[C@]12Cc3cnn(-c4ccc(F)cc4)c3C=C1CC[C@H](N(C1CC1)S(=O)(=O)c1cn(C)cn1)C2 ; Cn1cnc(S(=O)(=O)N(C2CC2)[C@H]2CCC3=Cc4c(cnn4-c4ccc(F)cc4)C[C@]3(CO)C2)c1]

A 20 mL vial was charged with 1i (253 mg, 0.43 mmol) and flushed with argon before adding diethyl ether (5 mL). The solution was cooled to 0° C. and then lithium aluminum hydride (1.0 M in THF, 0.55 mL, 0.55 mmol) was added dropwise to give a cloudy solution. The reaction solution was allowed to warm to rt and stirred under argon for 20 min. The reaction solution was quenched with water, and the precipitate was filtered. The filtrate was extracted with ethyl acetate, and the organic layers were washed (brine), dried (Na₂SO₄) and concentrated under reduced pressure to give the title compound (2a) (193 mg, 86% yield) as a white solid, m/z (ESI, +ve ion)=498.2 [M+H]⁺

### Step B: N-Cyclopropyl-N-((4aS,6S)-1-(4-fluorophenyl)-4a-formyl-4,4a,5,6,7,8-hexahydro-1H-benzo[f]indazol-6-yl)-1-methyl-1H-imidazole-4-sulfonamide (2b)

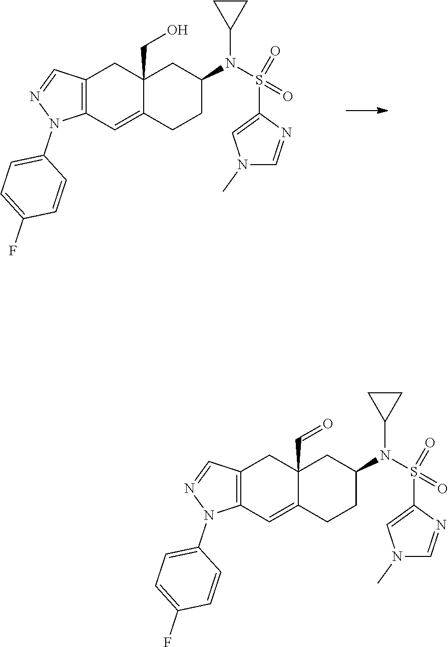 [SMILES: Cn1cnc(S(=O)(=O)N(C2CC2)[C@H]2CCC3=Cc4c(cnn4-c4ccc(F)cc4)C[C@]3(CO)C2)c1 ; Cn1cnc(S(=O)(=O)N(C2CC2)[C@H]2CCC3=Cc4c(cnn4-c4ccc(F)cc4)C[C@]3(C=O)C2)c1]

A round bottom flask charged with 2a (193 mg, 0.35 mmol) was added DCM (2.5 mL) under argon and stirred. Dess-Martin periodinane (195 mg, 0.46 mmol) was added and the reaction solution was allowed to stir for 50 min at rt under argon. The reaction was quenched with saturated NaHCO₃ (aq.) and 10% NaS₂O₃ (aq) and stirred for 15 min. The crude was extracted with ethyl acetate, washed with brine, dried with Na₂SO₄, and concentrated under reduced pressure. The residue was purified by column chromatography (0%-80% EtOAc/hexanes, a gradient elution) to provide the title compound (2b) (159 mg, 91% yield) as a white solid, m/z (ESI, +ve ion)=496.1 [M+H]⁺.

### Step C: N-Cyclopropyl-N-((4aS,6S)-1-(4-fluorophenyl)-4a-((R)-(4-fluoropyridin-2-yl)(hydroxy)methyl)-4,4a,5,6,7,8-hexahydro-1H-benzo[f]indazol-6-yl)-1-methyl-1H-imidazole-4-sulfonamide (2c)

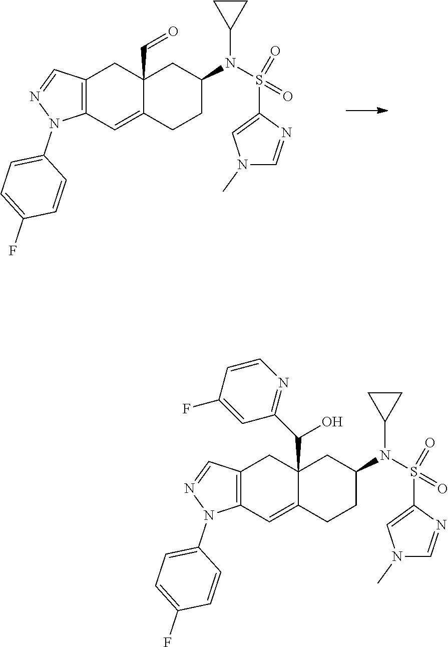 [SMILES: Cn1cnc(S(=O)(=O)N(C2CC2)[C@H]2CCC3=Cc4c(cnn4-c4ccc(F)cc4)C[C@]3(C=O)C2)c1 ; Cn1cnc(S(=O)(=O)N(C2CC2)[C@H]2CCC3=Cc4c(cnn4-c4ccc(F)cc4)C[C@]3(C(O)c3cc(F)ccn3)C2)c1]

A dry, round bottom flask was added ether (1.5 mL) and cooled to −78° C. under Ar. A solution of n-BuLi in hexanes (0.16 M in hexane, 0.20 mL, 0.32 mmol) was added to the flask followed by the addition of 2-bromopyridine (0.06 mL, 0.74 mmol) dropwise. The solution went from yellow to dark maroon upon addition of 2-bromo-4-fluoropyridine. The reaction solution was stirred at −78° C. for 40 min. In a separate, dry flask, 2b (94 mg, 0.18 mmol) (previously azeotroped with toluene) was dissolved with THE (1 mL) under Ar to give a cloudy solution. This solution was added dropwise to the lithiated species and allowed to stir at −78° C. under Ar for 10 min. The reaction was quenched with 2 N HCl in ether (0.34 mL) and allowed to warm to rt with stirring. The solution was added water and extracted with ethyl acetate, washed with brine, dried with Na₂SO₄, and concentrated under reduced pressure to give a viscous oil. The crude product was purified by chromatography (20%-100% EtOAc/hexanes, a gradient elution) followed by reverse preparative HPLC (10-40% acetonitrile in water with 0.1% formic acid) to provide the title compound (2c) as a viscous oil (12 mg, 11% yield), m/z (ESI, +ve ion)=593.2 [M+H]⁺.

### Step D: N-Cyclopropyl-N-((4aS,6S)-1-(4-fluorophenyl)-4a-(4-fluoropicolinoyl)-4,4a,5,6,7,8-hexahydro-1H-benzo[f]indazol-6-yl)-1-methyl-1H-imidazole-4-sulfonamide (2)

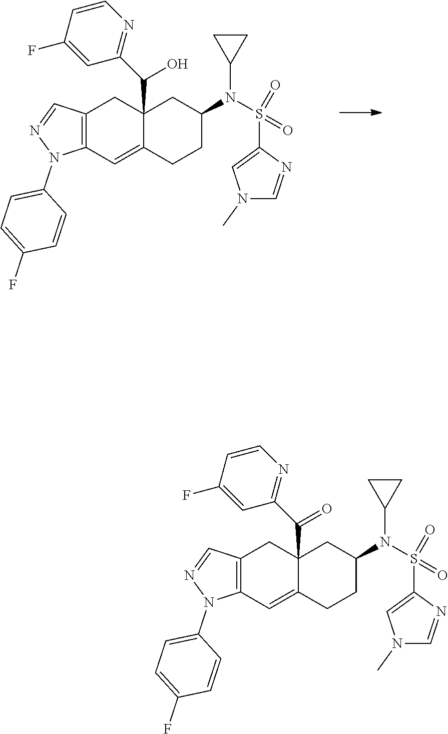 [SMILES: Cn1cnc(S(=O)(=O)N(C2CC2)[C@H]2CCC3=Cc4c(cnn4-c4ccc(F)cc4)C[C@]3(C(O)c3cc(F)ccn3)C2)c1 ; Cn1cnc(S(=O)(=O)N(C2CC2)[C@H]2CCC3=Cc4c(cnn4-c4ccc(F)cc4)C[C@]3(C(=O)c3cc(F)ccn3)C2)c1]

A round bottom flask charged with 2c (11.6 mg, 0.02 mmol) was added DCM (0.5 mL) under Ar. Dess-Martin periodinane (9.0 mg, 0.021 mmol) was added and the reaction solution was allowed to stir for 40 min at rt under argon. The reaction was quenched with saturated NaHCO₃ (aq.) and 10% NaS₂O₃ (aq) and stirred for 15 min. The crude was extracted with ethyl acetate, washed with brine, dried with Na₂SO₄, and concentrated under reduced pressure to give a viscous oil. The crude product was purified by chromatography (2%-90% EtOAc/hexanes, a gradient elution) to provide the title compound (2) as an off-white solid (10 mg, 87% yield). ¹H NMR (400 MHz, CHLOROFORM-d) δ ppm 8.62 (1H, dd, J=7.9, 5.6 Hz), 7.40-7.52 (5H, m), 7.24 (1H, s), 7.11-7.18 (3H, m), 6.43 (1H, d, J=1.8 Hz), 4.47 (1H, br m), 3.91 (1H, d, J=16.5 Hz), 3.76 (3H, s), 3.14 (1H, d, J=16.7 Hz), 2.66 (2H, m), 2.49 (2H, m), 2.3 (1H, tt, J=7, 3.6), 2.13 (1H, m), 1.89 (1H, m), 1.04 (1H, m), 0.9 (1H, m), 0.71 (2H, m). m/z (ESI, +ve ion)=591.2 [M+H]⁺.

### Example 3. N-Cyclopropyl-N-((4aS,6S)-4a-(4-fluoropicolinoyl)-1-(6-fluoropyridin-3-yl)-4,4a,5,6,7,8-hexahydro-1H-benzo[f]indazol-6-yl)-1-methyl-1H-pyrazole-4-sulfonamide (3)

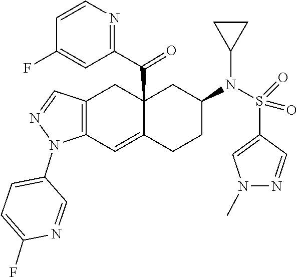 [SMILES: Cn1cc(S(=O)(=O)N(C2CC2)[C@H]2CCC3=Cc4c(cnn4-c4ccc(F)nc4)C[C@]3(C(=O)c3cc(F)ccn3)C2)cn1]

The title compound was prepared from 1e by procedures similar to those described in Example 1, Steps F-J, substituting 2-fluoro-5-hydrazinylpyridine for 4-fluorophenylhydrazine in Step F, substituting 1-methyl-1H-pyrazole-4-sulfonyl chloride for 1-methyl-1H-imidazole-4-sulfonyl chloride in Step I, and substituting 2-bromo-4-fluoropyridine for 2-bromo-5-(tert-butyl)-1,3-thiazole in Step J. ¹H NMR (400 MHz, CHLOROFORM-d) δ ppm 8.65 (dd, J=8.19, 5.55 Hz, 1H), 8.35 (dd, J=2.70, 0.80 Hz, 1H), 7.97 (ddd, 0.7=8.70, 6.80, 2.92 Hz, 1H), 7.77-7.81 (m, 2H), 7.55 (dd, J=9.43, 2.27 Hz, 1H), 7.33 (s, 1H), 7.20 (ddd, 7=8.08, 5.52, 2.63 Hz, 1H), 7.07 (dd, J=8.62, 3.36 Hz, 1H), 6.48 (d, J=1.61 Hz, 1H), 4.32-4.38 (m, 1H), 4.0 (d, J=16 Hz, 1H), 3.96 (s, 3H), 3.16 (d, J=16.52 Hz, 1H), 2.47-2.72 (m, 4H), 2.10-2.22 (m, 1H), 2.00-2.10 (m, 1H), 1.69-1.80 (m, 1H), 1.02-1.18 (m, 1H), 0.93 (m, 1H), 0.70-0.86 (m, 2H). m/z (ESI, +ve ion)=592.3 [M+H]⁺.

### Example 4. N-((4aS,6S)-1-(4-Fluorophenyl)-4a-(4-fluoropicolinoyl)-4,4a,5,6,7,8-hexahydro-1H-benzo[f]indazol-6-yl)-1-methyl-N-(2,2,2-trifluoroethyl)-1H-1,2,4-triazole-3-sulfonamide (4)

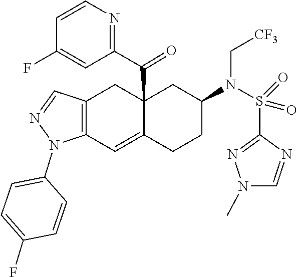 [SMILES: Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)n1]

### Step A: Ethyl (4aS,6S)-6-((2,4-dimethoxybenzyl)amino)-1-(4-fluorophenyl)-1,4,5,6,7,8-hexahydro-4aH-benzo[f]indazole-4a-carboxylate (4a)

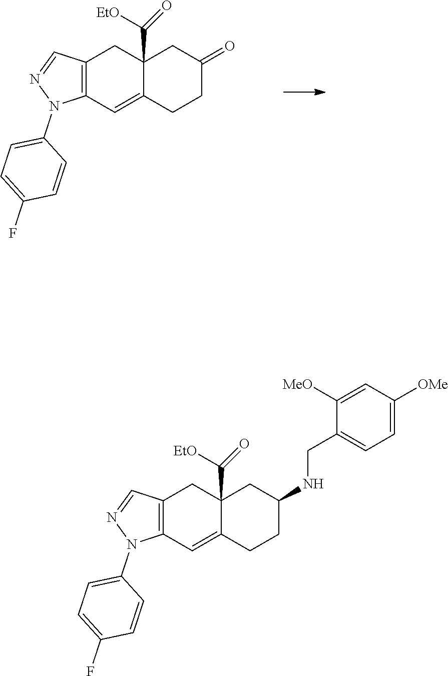 [SMILES: CCOC(=O)[C@@]12CC(=O)CCC1=Cc1c(cnn1-c1ccc(F)cc1)C2 ; CCOC(=O)[C@]12Cc3cnn(-c4ccc(F)cc4)c3C=C1CC[C@H](NCc1ccc(OC)cc1OC)C2]

To a solution of ethyl (4aS)-1-(4-fluorophenyl)-6-oxo-4,5,7,8-tetrahydrobenzo[f]indazole-4a-carboxylate (1g) (3.16 g, 8.92 mmol) and (2,4-dimethoxyphenyl)methanamine (2.68 mL, 17.8 mmol) in DCE (40 mL) was added acetic acid (1.54 mL, 26.8 mmol). After the reaction was stirred at rt for 5 min and cooled down in an ice-bath, sodium triacetoxyborohydride (5.10g, 24.1 mmol) was added in portions. 5 Minute later, the reaction solution was allowed to warm to rt and continued stirring for 30 min. The solution was quenched (sat. aq. NaHCO₃) and extracted (EtOAc). The organic layers were washed (brine), dried (Na₂SO₄) and concentrated under reduced pressure. The crude product was purified by silica gel chromatography (0%-75% EtOAc/hexanes, a gradient elution) to provide the title compound (4a) (4.15 g, 92% yield) as an off-white solid, m/z (ESI, +ve ion)=506.3 [M+H]⁺.

### Step B: Ethyl (4aS,6S)-6-((N-(2,4-dimethoxybenzyl)-1-methyl-1H-1,2,4-triazole)-3-sulfonamido)-1-(4-fluorophenyl)-1,4,5,6,7,8-hexahydro-4aH-benzo[f]indazole-4a-carboxylate (4b)

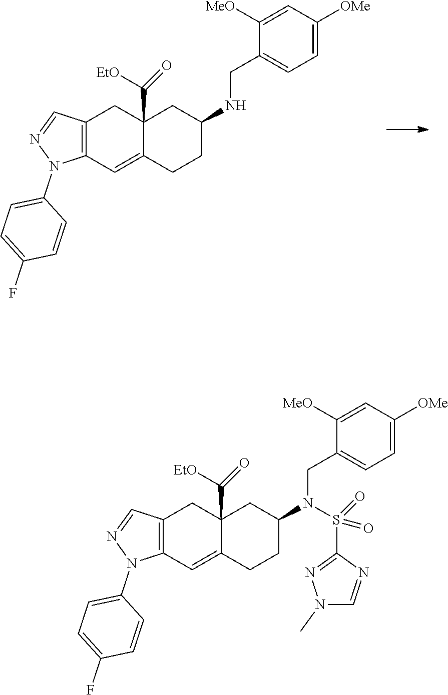 [SMILES: CCOC(=O)[C@]12Cc3cnn(-c4ccc(F)cc4)c3C=C1CC[C@H](NCc1ccc(OC)cc1OC)C2 ; CCOC(=O)[C@]12Cc3cnn(-c4ccc(F)cc4)c3C=C1CC[C@H](N(Cc1ccc(OC)cc1OC)S(=O)(=O)c1ncn(C)n1)C2]

A flask was charged with 4a (1.91 g, 3.78 mmol) and DCM (19 mL). The flask was cooled down in an ice bath and 1-methyl-1,2,4-triazole-3-sulfonyl chloride (0.82g, 4.5 mmol) was added, followed by addition of pyridine (0.91 mL, 11.3 mmol). After the reaction mixture was stirred at rt for 15 h, it was cooled back down in an ice bath and Et₃N (0.9 ml) was added slowly. The mixture was stirred at rt for 1 h and then concentrated. The residue was purified by column chromatography (45%-55% EtOAc/hexanes, a gradient elution) to provide the title compound (4b) (2.2 g, 89%) as a white solid, m/z (ESI, +ve ion)=651.2 [M+H]⁺.

### Step C: N-(2,4-Dimethoxybenzyl)-N-((4aS,6S)-1-(4-fluorophenyl)-4a-(4-fluoropicolinoyl)-4,4a,5,6,7,8-hexahydro-1H-benzo[f]indazol-6-yl)-1-methyl-1H-1,2,4-triazole-3-sulfonamide (4c)

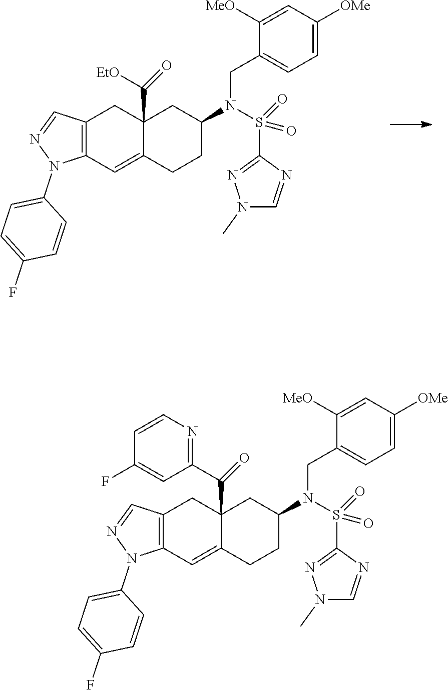 [SMILES: CCOC(=O)[C@]12Cc3cnn(-c4ccc(F)cc4)c3C=C1CC[C@H](N(Cc1ccc(OC)cc1OC)S(=O)(=O)c1ncn(C)n1)C2 ; COc1ccc(CN([C@H]2CCC3=Cc4c(cnn4-c4ccc(F)cc4)C[C@]3(C(=O)c3cc(F)ccn3)C2)S(=O)(=O)c2ncn(C)n2)c(OC)c1]

To a 100 mL dried flask charged with anhydrous ether (48 mL) was added n-butyllithium (6.53 mL, 10.5 mmol), followed by dropwise addition of 2-bromo-4-fluoropyridine (1.08 mL, 10.5 mmol) at −78° C. The reaction was stirred for another 15 min and a solution of 4b (1.7 g, 2.61 mmol) in ether (14 mL) and THE (14 mL) was added dropwise. After the reaction was stirred at −78° C. for 25 min, it was quenched with a small amount of water and then sat. NH₄Cl, extracted (EtOAc), washed (sat. aq. NaCl) and dried (Na₂SO₄). The combined organic layers were concentrated under reduced pressure and the residue was dissolved in acetone (6 mL) and 1 N HCl (6 mL). The solution was stirred for 1 h at 40° C., diluted (EtOAc), quenched (sat. NaHCO₃), extracted (EtOAc), washed (sat. aq. NaCl), and dried (Na₂SO₄). The combined organic layers were concentrated under reduced pressure. The crude product was purified by column chromatography (15%-80% EtOAc/hexanes, a gradient elution) to provide the title compound (4c) (1.29 g, 70% yield) as a yellow solid, m/z (ESI, +ve ion)=702.2 [M+H]⁺.

### Step D: N-((4aS,6S)-1-(4-Fluorophenyl)-4a-(4-fluoropicolinoyl)-4,4a,5,6,7,8-hexahydro-1H-benzo[f]indazol-6-yl)-1-methyl-1H-1,2,4-triazole-3-sulfonamide (4d)

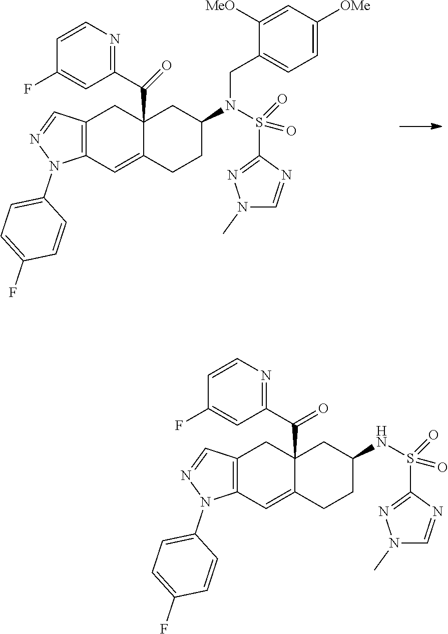 [SMILES: COc1ccc(CN([C@H]2CCC3=Cc4c(cnn4-c4ccc(F)cc4)C[C@]3(C(=O)c3cc(F)ccn3)C2)S(=O)(=O)c2ncn(C)n2)c(OC)c1 ; Cn1cnc(S(=O)(=O)N[C@H]2CCC3=Cc4c(cnn4-c4ccc(F)cc4)C[C@]3(C(=O)c3cc(F)ccn3)C2)n1]

To a pressure vial charged with 4c (1.29 g, 1.84 mmol) was added TFA (8.36 mL) and DCM (16 mL). The reaction was stirred at rt for 1 h and then concentrated under reduced pressure. Then it was diluted with EtOAc and filtered through a pad of Celite. The solution was concentrated and the residue was purified by column chromatography (0%-60% EtOAc/hexanes followed by 1.0-6.5% MeOH/DCM, a gradient elution) to provide the title compound (4d) (925 mg, 91% yield) as a white solid, m/z (ESI, +ve ion)=552.2 [M+H]⁺.

### Step E: N-((4aS,6S)-1-(4-Fluorophenyl)-4a-(4-fluoropicolinoyl)-4,4a,5,6,7,8-hexahydro-1H-benzo[f]indazol-6-yl)-1-methyl-N-(2,2,2-trifluoroethyl)-1H-1,2,4-triazole-3-sulfonamide (4)

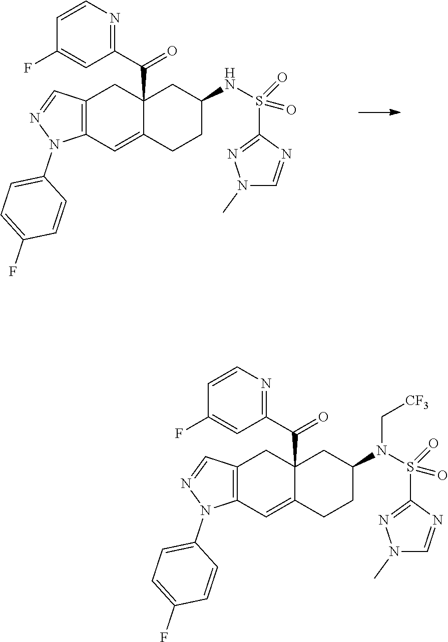 [SMILES: Cn1cnc(S(=O)(=O)N[C@H]2CCC3=Cc4c(cnn4-c4ccc(F)cc4)C[C@]3(C(=O)c3cc(F)ccn3)C2)n1 ; Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)n1]

A flask was charged with 4d (925 mg, 1.68 mmol) and sodium hydride (201 mg, 5.03 mmol). After the flask was put under high vacuum for 20 min and flushed with argon, DMF (16 mL) was added to form a homogeneous solution. The reaction was cooled down in an ice-bath and 2,2,2-trifluoroethyl triflate (1.21 mL, 8.39 mmol) was slowly added. 5 Minute later, the reaction was allowed to warm up to rt and continued stirring for 3 h. The reaction was cooled back down in an ice-bath, quenched with 10% citric acid and extracted with EtOAc. The organic layer was washed (brine), dried, and concentrated. The residue was first purified by silica gel column chromatography (SiO₂, 10%-75% EtOAc/hexanes, a gradient elution) and further purified by reverse HPLC (50%-70% water/acetonitrile with 0.1% formic acid) to provide the title compound (4). ¹H NMR (400 MHz, CHLOROFORM-d) δ ppm 8.63 (dd, J=8.11, 5.48 Hz, 1H), 8.16 (s, 1H), 7.55 (dd, J=9.35, 2.34 Hz, 1H), 7.39-7.46 (m, 2H), 7.26 (S, 1H), 7.15-7.21 (m, 3H), 6.45 (d, J=1.61 Hz, 1H), 4.32 (m, 1H), 4.03 (s, 3H), 4.01-4.04 (m, 2H), 3.91 (d, J=16.66 Hz, 1H), 3.07 (d, J=16.52 Hz, 1H), 2.70 (dd, J=13.45, 3.07 Hz, 1H), 2.55-2.66 (m, 1H), 2.37-2.50 (m, 2H) 1.86-2.00 (m, 2H). m/z (ESI, +ve ion)=634.2 [M+H]⁺.

### Example 5. N-(2-Fluoroethyl)-N-((4aS,6S)-1-(4-fluorophenyl)-4a-(4-fluoropicolinoyl)-4,4a,5,6,7,8-hexahydro-1H-benzo[f]indazol-6-yl)-1-methyl-1H-pyrazole-4-sulfonamide (5)

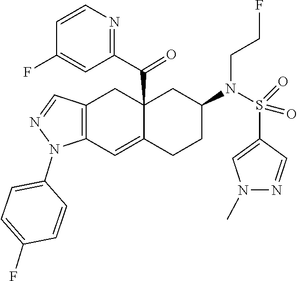 [SMILES: Cn1cc(S(=O)(=O)N(CCF)[C@H]2CCC3=Cc4c(cnn4-c4ccc(F)cc4)C[C@]3(C(=O)c3cc(F)ccn3)C2)cn1]

### Step A: Ethyl (4aS,6S)-1-(4-fluorophenyl)-6-((1-methyl-1H-pyrazole)-4-sulfonamido)-1,4,5,6,7,8-hexahydro-4aH-benzo[f]indazole-4a-carboxylate (5a)

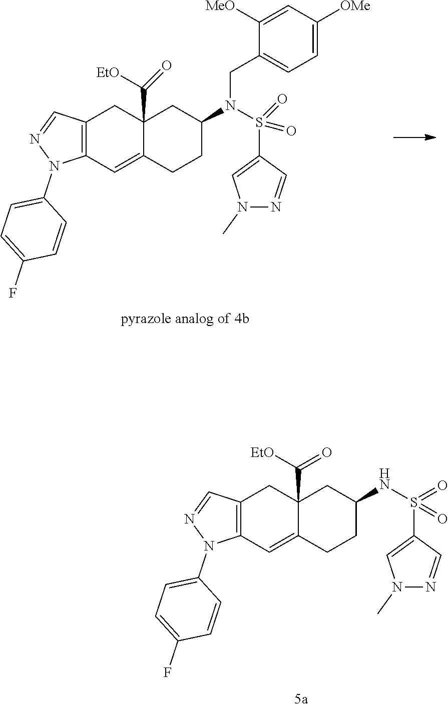 [SMILES: CCOC(=O)[C@]12Cc3cnn(-c4ccc(F)cc4)c3C=C1CC[C@H](N(Cc1ccc(OC)cc1OC)S(=O)(=O)c1cnn(C)c1)C2 ; CCOC(=O)[C@]12Cc3cnn(-c4ccc(F)cc4)c3C=C1CC[C@H](NS(=O)(=O)c1cnn(C)c1)C2]

Ethyl (4aS,6S)-6-((N-(2,4-dimethoxybenzyl)-1-methyl-1H-pyrazole)-4-sulfonamido)-1-(4-fluorophenyl)-1,4,5,6,7,8-hexahydro-4aH-benzo[f]indazole-4a-carboxylate (pyrazole analog of 4b) (460 mg, 0.71 mmol) was dissolved in a mixture of trifluoroacetic acid (3.22 mL, 41.8 mmol) and DCM (7 mL). The reaction was stirred for 1 h and additional TFA (0.1 mL) was added. After the reaction was stirred for another 1.5 h, it was poured into an ice-cold NaHCO₃ solution and extracted (EtOAc). The organic phase was filtered through a pad of Celite, washed (brine), dried (Na₂SO₄) and concentrated. The crude product was purified by column chromatography (0%-7% MeOH/DCM, a gradient elution) to provide the title compound (5a) (355 mg, 100% yield) as a white solid, m/z (ESI, +ve ion)=500.1 [M+H]⁺.

### Step B: Ethyl (4aS,6S)-6-((N-(2-fluoroethyl)-1-methyl-1H-pyrazole)-4-sulfonamido)-1-(4-fluorophenyl)-1,4,5,6,7,8-hexahydro-4aH-benzo[f]indazole-4a-carboxylate (5b)

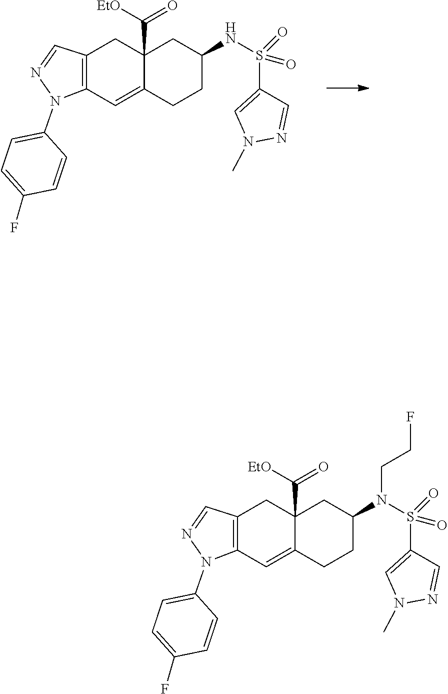

To a flask charged with 5a (245 mg, 0.49 mmol) were added DMF (5 mL) and cesium carbonate (251 mg, 1.1 mmol). The reaction mixture was stirred at rt for 10 min and 1-fluoro-2-iodoethane (0.25 mL, 2.4 mmol) was added. After the reaction was stirred at rt overnight, it was quenched (water) and extracted (EtOAc). The organic phase was washed (brine), dried (Na₂SO₄) and concentrated. The residue was purified by column chromatography (20%-30% EtOAc/hexanes, a gradient elution) to provide the title compound (5b) (264 mg, 98% yield). ¹H NMR (400 MHz, CHLOROFORM-d) δ ppm 7.77 (1H, s), 7.72 (1H, s), 7.37-7.53 (3H, m), 7.13-7.26 (2H, m), 6.36 (1H, d, J=1.61 Hz), 4.64-4.82 (1H, m), 4.58 (1H, td, J=5.33, 1.17 Hz), 4.00-4.16 (2H, m), 3.96 (3H, s), 3.32-3.58 (2H, m), 3.19 (1H, d, J=15.93 Hz), 2.60-2.86 (2H, m), 2.32-2.56 (1H, m), 2.24 (1H, t, J=13.01 Hz), 1.76-1.93 (2H, m), 1.65-1.76 (1H, m), 1.17 (3H, t, 0.7=7.16 Hz), m/z (ESI, +ve ion)=546.2 (M+H)⁺.

### Step C: N-(2-Fluoroethyl)-N-((4aS,6S)-1-(4-fluorophenyl)-4a-(4-fluoropicolinoyl)-4,4a,5,6,7,8-hexahydro-1H-benzo[f]indazol-6-yl)-1-methyl-1H-pyrazole-4-sulfonamide (5)

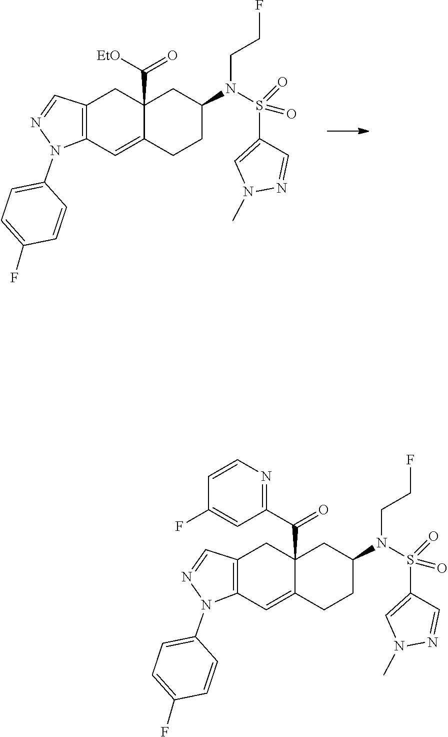

5b (59 mg, 0.11 mmol) was azeotroped with toluene in a 25 mL flask and the flask was put under high vacuum. To a separate dried flask under an argon balloon was added anhydrous ether (1.4 mL) and n-butyllithium (1.6 M, 0.62 mL, 0.98 mmol), followed dropwise addition of 2-bromo-4-fluoropyridine (164 mg, 0.93 mmol) in ether (1.4 mL) at −78° C. The solution was stirred at −78° C. for 30 min. The flask with 5b (59 mg, 0.11 mmol) was flushed with argon and THF (1.4 mL) was added. This solution was added dropwise to the lithiated species in the first flask at −78° C. After the reaction was stirred for 1 h, it was quenched with water/sat NH₄Cl, extracted (EtOAc), washed (brine), dried (MgSO₄), and concentrated. The residue was purified by column chromatography (20%-30% EtOAc/hexanes, a gradient elution) to afford the title compound (5) (45 mg, 70% yield) as a yellow solid. ¹H NMR (400 MHz, CHLOROFORM-d) δ ppm 8.64 (1H, dd, J=8.19, 5.55 Hz), 7.71-7.86 (2H, m), 7.72-7.80 (1H, m), 7.54 (1H, dd, J=9.28, 2.41 Hz), 7.28-7.47 (2H, m), 7.11-7.26 (3H, m), 6.39-6.54 (1H, m), 4.62-4.78 (1H, m), 4.56 (1H, br d, 0.7=4.68 Hz), 4.21 (1H, br d, J=9.50 Hz), 3.98-4.03 (3H, m), 3.93-3.97 (1H, m), 3.35-3.59 (2H, m), 3.05 (1H, d, J=16.52 Hz), 2.50-2.73 (2H, m), 2.39-2.50 (1H, m), 2.27-2.33 (1H, t, J=13.15 Hz), 1.80-1.84 (2H, m). m/z (ESI, +ve ion)=597.2 (M+H)⁺.

### Example 6. N-((4aS,6S)-1-(5-Fluoropyridin-2-yl)-4a-(4-(trifluoromethyl)picolinoyl)-4,4a,5,6,7,8-hexahydro-1H-benzo[f]indazol-6-yl)-N-isopropyl-1-methyl-1H-pyrazole-4-sulfonamide (6)

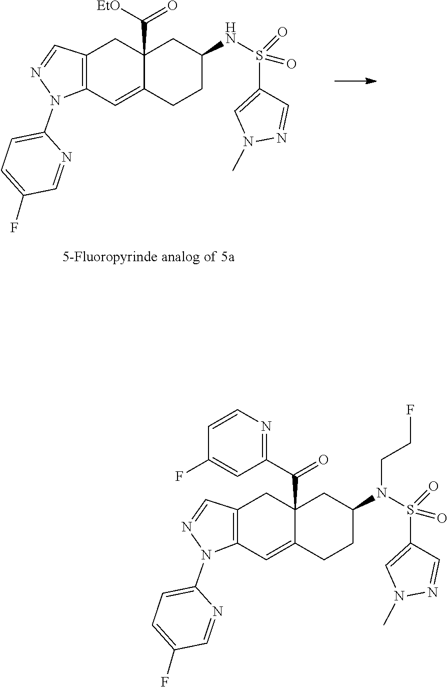

The title compound was prepared from 5-fluoropyridine analog of 5a by procedures similar to those described in Example 5, Steps B and C. ¹H NMR (400 MHz, CHLOROFORM-d) δ ppm 1.24 (d, J=6.72 Hz, 3H) 1.27 (d, J=6.72 Hz, 3H) 1.80-1.93 (m, 1H) 2.29-2.42 (m, 1H) 2.50-2.60 (m, 1H) 2.62-2.74 (m, 2H) 2.94 (t, J=13.23 Hz, 1H) 3.11 (d, J=16.52 Hz, 1H) 3.75-3.91 (m, 2H) 3.93-4.00 (m, 4H) 7.25 (s, 1H) 7.48-7.56 (m, 2H) 7.65-7.71 (m, 2H) 7.75 (s, 1H) 7.84 (dd, J=9.06, 3.95 Hz, 1H) 8.02-8.07 (m, 1H) 8.29 (d, J=2.92 Hz, 1H) 8.89 (d, J=5.12 Hz, 1H). m/z (ESI, +ve ion) 644.2 (M+H)⁺.

### Example 7. (5-Cyclopropylpyridin-2-yl)((4aS,6S)-1-(4-fluorophenyl)-6-((1-methyl-1H-1,2,4-triazol-3-yl)sulfonyl)-1,4,5,6,7,8-hexahydro-4aH-benzo[f]indazol-4a-yl)methanone (7)

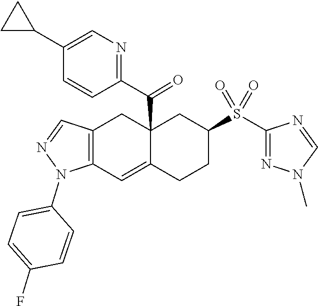 [SMILES: Cn1cnc(S(=O)(=O)[C@H]2CCC3=Cc4c(cnn4-c4ccc(F)cc4)C[C@]3(C(=O)c3ccc(C4CC4)cn3)C2)n1]

### Step A: Ethyl (4aS)-1-(4-fluorophenyl)-6-hydroxy-1,4,5,6,7,8-hexahydro-4aH-benzo[f]indazole-4a-carboxylate (7a)

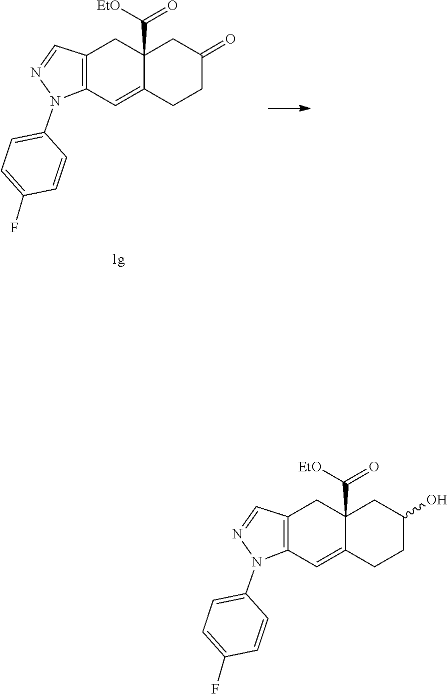

To a solution of ethyl (S)-1-(4-fluorophenyl)-6-oxo-1,4,5,6,7,8-hexahydro-4aH-benzo[f]indazole-4a-carboxylate (1g) (11.6 g, 32.8 mmol) in MeOH (450 mL) was added NaBH₄ (2.24 g, 59 mmol) at 0° C. The mixture was stirred at 0° C. for 2 h and then quenched with water and extracted with EtOAc. The combined organic layers were dried and concentrated under reduced pressure. The residue was purified by chromatography to give the title compound (7a) (9.40 g, 74%) as a mixture of two diastereomers. m/z (ESI, +ve ion)=356.7 [M+H]⁺.

### Step B: Ethyl (4aS,6R)-1-(4-fluorophenyl)-6-((methylsulfonyl)oxy)-1,4,5,6,7,8-hexahydro-4aH-benzo[f]indazole-4a-carboxylate (7b)

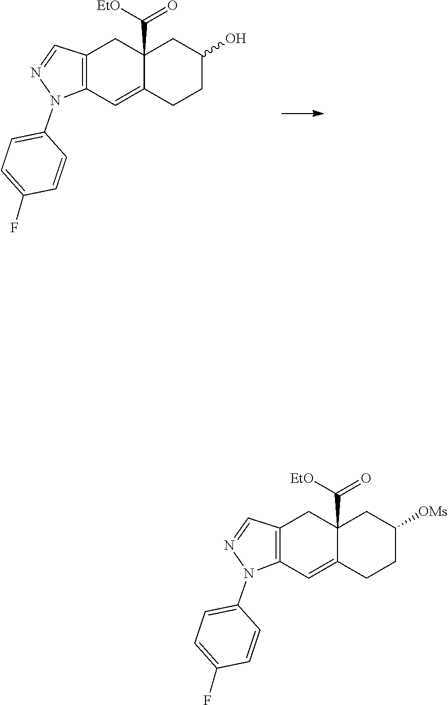

To a solution of 7a (6.00 g, 16.8 mmol, azeotroped with toluene) in DCM (150 mL) was added triethylamine (7.04 mL, 50.6 mmol). After the reaction mixture was cooled to 0° C., methanesulfonyl chloride (1.69 mL, 21.9 mmol) was added dropwise. The reaction was allowed to warm to rt and stirred at the same temperature for 1 h. The reaction was quenched with water and extracted with DCM. The organics were washed with brine, dried over anhydrous sodium sulfate and concentrated under reduced pressure to afford an orange foaming solid, which was purified by silica gel chromatography (30%-100% EtOAc/hexanes, a gradient elution) to afford the title compound (7b) (3.90 g, 53%, the faster eluting isomer) and the other diastereomeric isomer (2.56 g, 35%, the slower eluting isomer) as white solids separately.

### Step C: 3-((4-Methoxybenzyl)thio)-1H-1,2,4-triazole (7c)

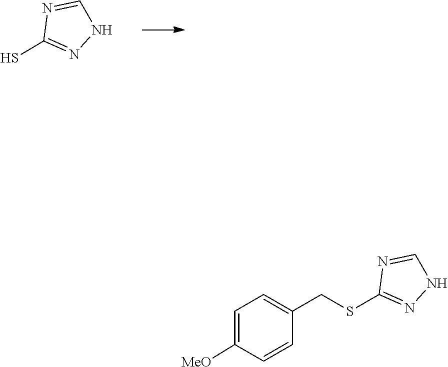 [SMILES: Sc1nc[nH]n1 ; COc1ccc(CSc2nc[nH]n2)cc1]

To a stirred suspension of 177-1,2,4-triazole-3-thiol (15.7 g, 155 mmol) in EtOH (200 mL) at 0° C. was added 4-methoxybenzyl chloride (22 mL, 163 mmol). The reaction was allowed to warm to rt and stirred at the same temperature for 3 h. The reaction mixture was concentrated under reduced pressure to give a white solid. Water (100 mL) and brine (100 mL) were added and the solution was extracted with EtOAc. The combined organic layers were washed (brine), dried (Na₂SO₄), and concentrated under reduced pressure to afford the title compound (7c) (34.5 g, 100% yield) as a white solid, m/z (ESI, +ve ion)=222.1 [M+H]⁺.

### Step D: 3-((4-Methoxybenzyl)thio)-1-methyl-1H-1,2,4-triazole (7d)

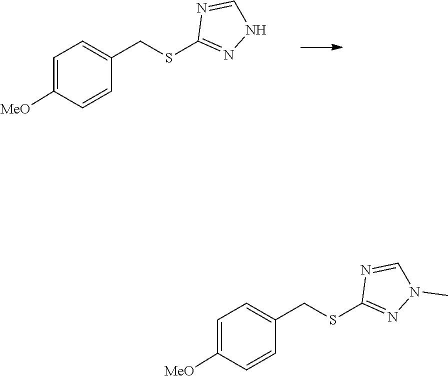 [SMILES: COc1ccc(CSc2nc[nH]n2)cc1 ; COc1ccc(CSc2ncn(C)n2)cc1]

To a stirred solution of 7c (14.8 g, 67 mmol) in DMF (200 mL) was added potassium carbonate (23.2 g, 168 mmol) at rt. After the reaction mixture was cooled to 0° C., iodomethane (12.5 mL, 201 mmol) was added dropwise. The reaction was allowed to warm to rt and stirred at the same temperature for 3 h. Additional potassium carbonate (6.5 g, 47 mmol) and iodomethane (2.9 mL, 47 mmol) were added at rt and the resulting mixture was stirred for two more hours. The mixture was quenched (water) and extracted (EtOAc). The organic layers were washed (water and brine), dried (Na₂SO₄), and concentrated under reduced pressure. Purification of the residue by silica gel column chromatography (30% to 100% EtOAc/hexanes, a gradient elution) afforded the title compound (7d) (5.4 g, 34%, the slower eluting isomer) and the other regioisomer 5-((4-methoxybenzyl)thio)-1-methyl-1H-1,2,4-triazole (5.6 g, 36%, the faster eluting isomer) as off-white solids, m/z (ESI, +ve ion)=236.1 [M+H]⁺.

### Step E: 1-Methyl-1,2-dihydro-3H-1,2,4-triazole-3-thione (7e)

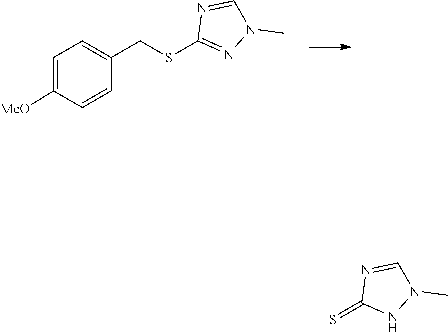

A solution of 7d (4.94 g, 21 mmol) in TFA (64 mL) was heated in a pressure tube at 100° C. for 16 h. After cooling to rt, TFA was removed under reduced pressure. The resulting residue was azeotroped with toluene and dried under high vacuum for 2 h to give title compound (7e) as a dark green solid. This material was used for the next step without further purification, m/z (ESI, +ve ion)=116.2 [M+H]⁺.

### Step F: Ethyl (4aS,6S)-1-(4-fluorophenyl)-6-((1-methyl-1H-1,2,4-triazol-3-yl)thio)-1,4,5,6,7,8-hexahydro-4aH-benzo[f]indazole-4a-carboxylate (7f)

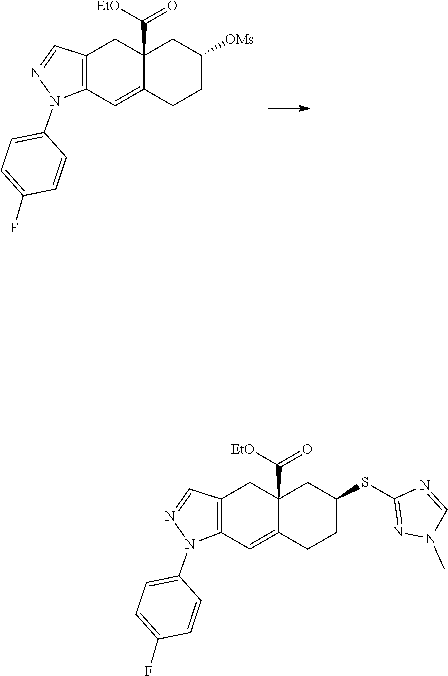

A mixture of ethyl (4aS,6R)-1-(4-fluorophenyl)-6-((methylsulfonyl)oxy)-1,4,5,6,7,8-hexahydro-4aH-benzo[f]indazole-4a-carboxylate (7b) (3.04 g, 7.0 mmol), 1-methyl-1,2-dihydro-3H-1,2,4-triazole-3-thione (7e) (21 mmol) and potassium carbonate (3.87g, 28 mmol) in DMF (24 mL) was heated at 80° C. for 3 h under Ar. After cooling to rt, the reaction mixture was poured into saturated aq. NH₄Cl solution and extracted. The combined organic layer was washed (water and brine), dried (Na₂SO₄), and concentrated under reduced pressure. Purification of the residue by column chromatography (20% to 50% acetone/hexanes, a gradient elution) provided title compound (7f) (2.3 g, 72%) as a light yellow solid, m/z (ESI, +ve ion)=454.1 [M+H]⁺.

### Step G: ((4aS,6S)-1-(4-Fluorophenyl)-6-((1-methyl-1H-1,2,4-triazol-3-yl)thio)-1,4,5,6,7,8-hexahydro-4aH-benzo[f]indazol-4a-yl)methanol (7g)

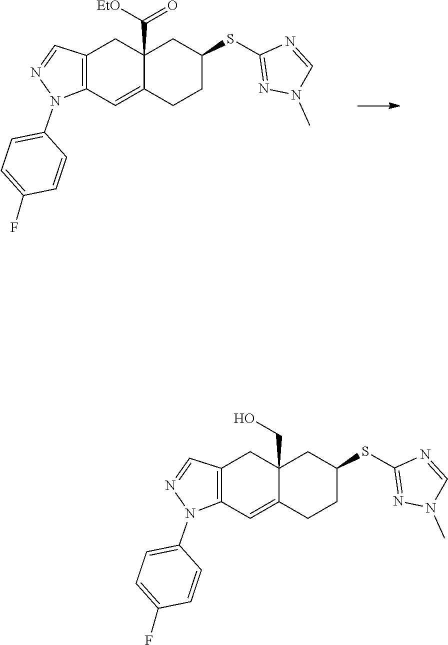

To a stirred solution 7f (2.3 g, 5.07 mmol) in diethyl ether/THF (3/1, 100 mL) and was added lithium aluminum hydride (1.0 M in THF, 6.6 mL, 6.6 mmol) at 0° C. Gas evolution was observed and the reaction mixture became yellow cloudy suspension. The mixture was stirred at 0° C. for 10 min, and then EtOAc (75 mL) was slowly added. The mixture was allowed to warm to rt and stirred for 20 min. Water was added and the resulting suspension was filtered through a small pad of Celite. The organic phase was washed (water and brine), dried (Na₂SO₄), and concentrated under reduced pressure to give the title compound (7g) (2.1 g, 100%) as a yellow foamy solid, m/z (ESI, +ve ion)=412.1 [M+H]⁺.

### Step H: (4aS,6S)-1-(4-Fluorophenyl)-6-((1-methyl-1H-1,2,4-triazol-3-yl)thio)-1,4,5,6,7,8-hexahydro-4aH-benzo[f]indazole-4a-carbaldehyde (7h)

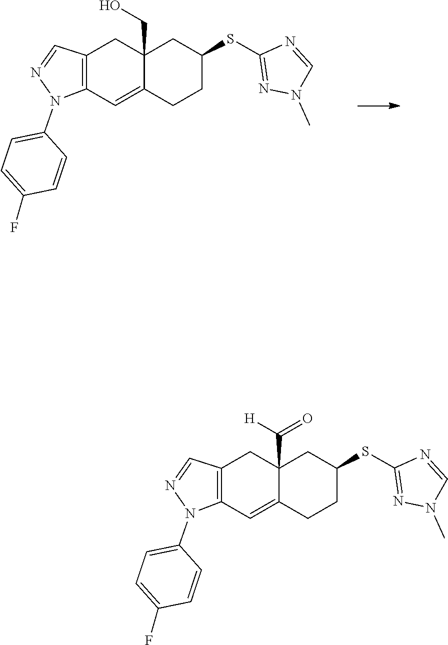

To a stirred solution of 7g (2.10 g, 5.10 mmol) in DCM (75 mL) was added Dess-Martin periodinane (2.27 g, 5.36 mmol) at rt. After the reaction mixture was stirred for 30 min, additional Dess-Martin periodinane (665 mg, 1.57 mmol) was added. After another 20 min, additional Dess-Martin periodinane (333 mg, 0.79 mmol) was added. The reaction was quenched with saturated aq. NaHCO₃ solution and 10% aq. Na₂S₂O₃ solution. The mixture was stirred at rt for 15 min and extracted (DCM). The combined organic layer was washed (brine), dried (Na₂SO₄), and concentrated under reduced pressure. Purification of the residue by column chromatography (20% to 50% acetone/hexanes, a gradient elution) provided the title compound (7h) (1.4 g, 67%) as a yellow solid, m/z (ESI, +ve ion)=410.2 [M+H]⁺.

### Step I: (5-Cyclopropylpyridin-2-yl)((4aS,6S)-1-(4-fluorophenyl)-6-((1-methyl-1H-1,2,4-triazol-3-yl)thio)-1,4,5,6,7,8-hexahydro-4aH-benzo[f]indazol-4a-yl)methanol (7i)

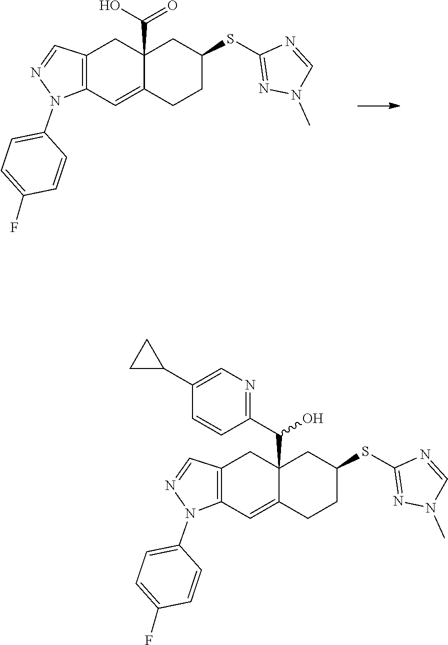

n-Butyllithium solution (1.6 M in hexane, 0.61 mL, 0.98 mmol) was added to a flask with diethyl ether (2.2 mL) at −78° C., followed by the dropwise addition of 2-bromo-5-cyclopropylpyridine (213 mg, 1.07 mmol), and the resulting mixture was stirred at −78° C. for 45 min. To the prepared aryllithium solution was added a solution of (7h) (100 mg, 0.244 mmol) in THF (1.1 mL) dropwise and the mixture was stirred at −78° C. for 10 min. The reaction was quenched by the addition of water. The dry ice bath was removed and then saturated aq. NH₄Cl solution was added. The resulting mixture was warmed to rt and the solution was extracted (EtOAc). The combined organic layer was washed (brine), dried (Na₂SO₄), and concentrated under reduced pressure. Purification of the residue by column chromatography (30% to 60% acetone/hexanes, a gradient elution) provided the title compound (7i) (99 mg, 77%) as a yellow solid, m/z (ESI, +ve ion) 529.2=[M+H]⁺.

### Step J: (5-Cyclopropylpyridin-2-yl)((4aS,6S)-1-(4-fluorophenyl)-6-((1-methyl-1H-1,2,4-triazol-3-yl)sulfonyl)-1,4,5,6,7,8-hexahydro-4aH-benzo[f]indazol-4a-yl)methanol (7j)

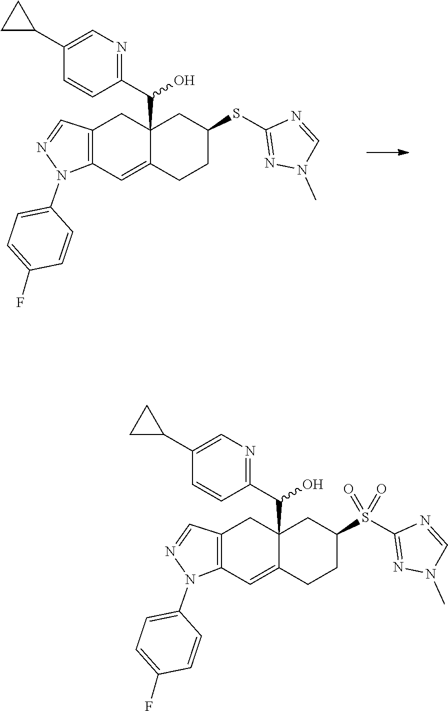

To a stirred solution of 7i (99 mg, 0.187 mmol) in 1,4-dioxane/water (4/1, 1.5 mL) was added oxone (171 mg, 1.12 mmol), then it was heated at 50° C. for 1 h. Then the reaction mixture was cooled to 0° C. and quenched by dropwise addition of sat. aq. NaHCO₃ (1.0 mL) and 10% aq. Na₂S₂O₃ solution (2.0 mL). The mixture was extracted (EtOAc) and the combined organic layer was washed (brine), dried (Na₂SO₄), and concentrated under reduced pressure. Purification of the residue by column chromatography (20% to 80% acetone/hexanes, a gradient elution) provided the title compound (7j) (82 mg, 78%) as a white solid, m/z (ESI, +ve ion)=561.2 [M+H]⁺.

### Step K: (5-Cyclopropylpyridin-2-yl)((4aS,6S)-1-(4-fluorophenyl)-6-((1-methyl-1H-1,2,4-triazol-3-yl)sulfonyl)-1,4,5,6,7,8-hexahydro-4aH-benzo[f]indazol-4a-yl)methanone (7)

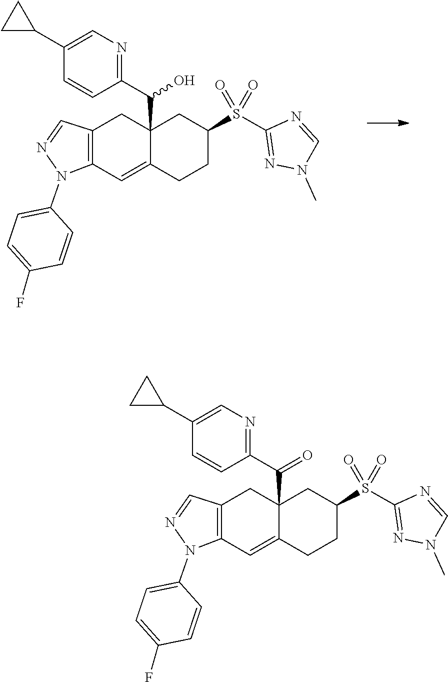

To a stirred solution of 7j (62.4 mg, 0.111 mmol) in DCM (1.6 mL) was added Dess-Martin periodinane (66.1 mg, 0.156 mmol) at rt and the reaction mixture was stirred for 20 min. The reaction was quenched with saturated aq. NaHCO₃ solution and 10% aq. Na₂S₂O₃ solution. The mixture was stirred at rt for 15 min and extracted (DCM). The combined organic layer was washed (brine), dried (Na₂SO₄), and concentrated under reduced pressure. The residue was purified by column chromatography (60% to 100% EtOAc/hexanes, a gradient elution) and further purified by reverse HPLC (40% to 80% MeCN/water with 0.1% formic acid) to provide the title compound (7) (33 mg, 53%) as a yellow solid. ¹H NMR (400 MHz, Chloroform-d) δ 8.40 (1H, d, J=2.4 Hz), 8.22 (1H, d, J=0.4 Hz), 7.73 (1H, d, J=8.1 Hz), 7.44-7.40 (2H, m), 7.34 (1H, dd, J=8.2, 2.2 Hz), 7.27 (1H, s), 7.16-7.12 (2H, m), 6.47 (1H, d, J=1.6 Hz), 4.16 (1H, d, J=16.4 Hz), 4.07 (3H, s), 3.89-3.80 (1H, m), 3.16 (1H, dd, J=14.0, 2.0 Hz), 3.00 (1H, d, J=16.4 Hz), 2.68-2.59 (1H, m), 2.51-2.44 (2H, m), 2.32-2.24 (1H, m), 2.00-1.87 (2H, m), 1.17-1.12 (2H, m), 0.90-0.81 (2H, m); m/z (ESI, +ve ion)=559.2 [M+H]⁺.

### Example 8. ((4aR,6S)-1-(4-Fluorophenyl)-6-(2-((1-methyl-1H-pyrazol-3-yl)sulfonyl)propan-2-yl)-1,4,5,6,7,8-hexahydro-4aH-benzo[f]indazol-4a-yl)(4-fluoropyridin-2-yl)methanone (8)

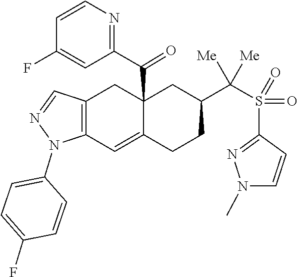 [SMILES: Cn1ccc(S(=O)(=O)C(C)(C)[C@H]2CCC3=Cc4c(cnn4-c4ccc(F)cc4)C[C@]3(C(=O)c3cc(F)ccn3)C2)n1]

### Step A: (S)-(1-(4-Fluorophenyl)-1,4,7,8-tetrahydrospiro[benzo[f]indazole-6,2′-[1,3]dioxolan]-4a(5H)-yl)methanol (8a)

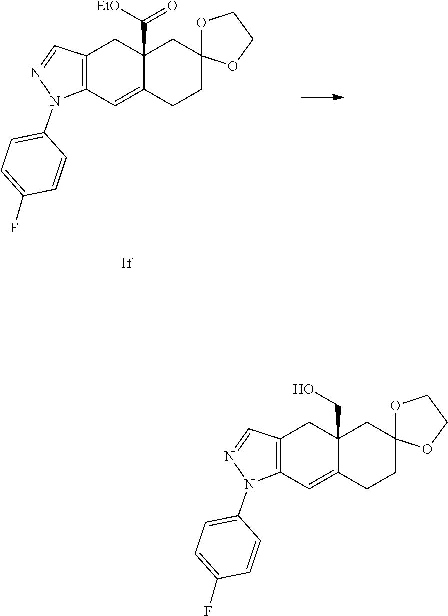

To a stirred solution of ethyl (S)-1-(4-fluorophenyl)-1,4,7,8-tetrahydrospiro[benzo[f]indazole-6,2′-[1,3]dioxolane]-4a(5H)-carboxylate (If) (14.4 g, 36.1 mmol) in diethyl ether (300 mL) was added lithium aluminum hydride (1.0 M in THF, 47.0 mL, 47.0 mmol) at 0° C. The mixture was stirred at 0° C. for 20 min and EtOAc (20 mL) was added. After the mixture was allowed to warm to rt and stirred for 20 min, it was quenched (water) and the resulting suspension was filtered through a small pad of Celite. The organic phase was washed (water, brine), dried (Na₂SO₄), and concentrated under reduced pressure. Purification of the residue by column chromatography afforded (S)-(1-(4-fluorophenyl)-1,4,7,8-tetrahydrospiro[benzo[f]indazole-6,2′-[1,3]dioxolan]-4a(5H)-yl)methanol (8a) (12.9 g, 100%) as a yellow foamy solid, m/z (ESI, +ve ion)=357.2 [M+H]⁺.

### Step B: (S)-1-(4-Fluorophenyl)-4a-(hydroxymethyl)-1,4,4a,5,7,8-hexahydro-6H-benzo[f]indazol-6-one (8b)

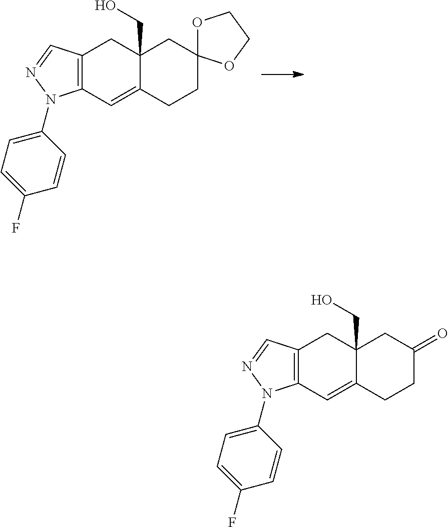 [SMILES: OC[C@]12Cc3cnn(-c4ccc(F)cc4)c3C=C1CCC1(C2)OCCO1 ; O=C1CCC2=Cc3c(cnn3-c3ccc(F)cc3)C[C@]2(CO)C1]

To a stirred solution of (S)-(1-(4-fluorophenyl)-1,4,7,8-tetrahydrospiro[benzo[f]indazole-6,2′-[1.3]dioxolan]-4a(5H)-yl)methanol (8a) (12.9 g, 36.1 mmol) in acetone (200 mL) was added 4 N aqueous HCl (108 mL, 433 mmol). The reaction mixture was stirred at rt overnight and neutralized by 2 N NaOH (55 mL) and sat. aq. NaHCO₃ (500 mL). Acetone was removed under reduced pressure and the remaining mixture was extracted (EtOAc). The organic layer was washed (brine), dried (Na₂SO₄), and concentrated under reduced pressure. Purification of the residue by chromatography (50% to 100% EtOAc/hexane, a gradient elution) provided (S)-1-(4-fluorophenyl)-4a-(hydroxymethyl)-1,4,4a,5,7,8-hexahydro-6H-benzo[f]indazol-6-one (8b) (11.1 g, 98%) as a yellow solid, m/z (ESI, +ve ion)=313.1 [M+H]⁺.

### Step C: (S)-4a-(((tert-Butyldimethylsilyl)oxy)methyl)-1-(4-fluorophenyl)-1,4,4a,5,7,8-hexahydro-6H-benzo[f]indazol-6-one (8c)

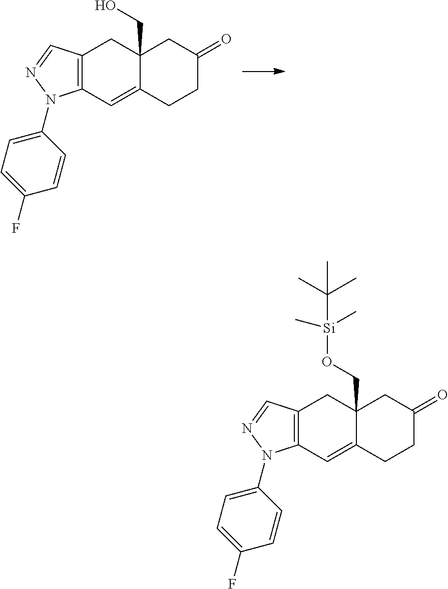 [SMILES: O=C1CCC2=Cc3c(cnn3-c3ccc(F)cc3)C[C@]2(CO)C1 ; CC(C)(C)[Si](C)(C)OC[C@@]12CC(=O)CCC1=Cc1c(cnn1-c1ccc(F)cc1)C2]

To a solution of (S)-1-(4-fluorophenyl)-4a-(hydroxymethyl)-1,4,4a,5,7,8-hexahydro-6H-benzo[f]indazol-6-one (8b) (4.00 g, 12.8 mmol) in DMF (55 mL) were added tert-butyldimethylsilyl chloride (6.76 g, 44.8 mmol) and imidazole (4.10 g, 60.2 mmol) successively at 0° C. After the solution was allowed to warm to rt and stirred overnight, it was quenched (water) and extracted (EtOAc). The organic layer was washed (brine), dried (Na₂SO₄), and concentrated under reduced pressure. Purification of the residue by column chromatography (10% to 40% EtOAc/hexane, a gradient elution) provided (S)-4a-(((tert-butyldimethylsilyl)oxy)methyl)-1-(4-fluorophenyl)-1,4,4a,5,7,8-hexahydro-6H-benzo[f]indazol-6-one (8c) (4.82 g, 88%) as an orange gum. m/z (ESI, +ve ion)=427.2 [M+H]⁺.

### Step D: (S)-4a-(((tert-Butyldimethylsilyl)oxy)methyl)-1-(4-fluorophenyl)-6-(methoxymethylene)-4,4a,5,6,7,8-hexahydro-1H-benzo[f]indazole (8d)

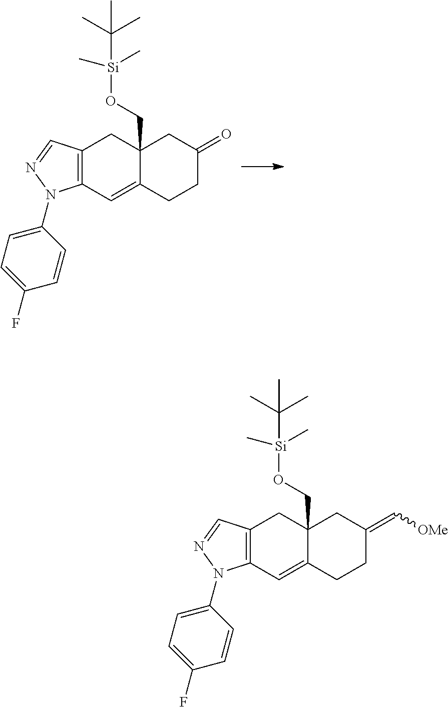 [SMILES: CC(C)(C)[Si](C)(C)OC[C@@]12CC(=O)CCC1=Cc1c(cnn1-c1ccc(F)cc1)C2 ; COC=C1CCC2=Cc3c(cnn3-c3ccc(F)cc3)C[C@]2(CO[Si](C)(C)C(C)(C)C)C1]

To a stirred solution of (S)-4a-(((tert-butyldimethylsilyl)oxy)methyl)-1-(4-fluorophenyl)-1,4,4a,5,7,8-hexahydro-6H-benzo[f]indazol-6-one (8c) (3.40 g, 8.00 mmol) and dimethyl diazomethylphosphonate (3.36 g, 22.4 mmol) in MeOH (14 mL) at 0° C. was added a solution of potassium tert-butoxide (2.5 g, 22.4 mml) in MeOH (12 mL) dropwise over the period of 10 min. After the mixture was allowed to warm to rt and stirred at rt for 30 min, it was poured into sat. aq. NaHCO₃ (80 mL). MeOH was removed under reduced pressure and the remaining was extracted (EtOAc). The organic layer was washed (brine), dried (Na₂SO₄), and concentrated under reduced pressure. Purification of the residue by column chromatography (0% to 15% EtOAc/hexane, a gradient elution) provided (S)-4a-(((tert-butyldimethylsilyl)oxy)methyl)-1-(4-fluorophenyl)-6-(methoxymethylene)-4,4a,5,6,7,8-hexahydro-1H-benzo[f]indazole (8d) (3.08 g, 85%) as a colorless gum. m/z (ESI, +ve ion)=455.1 [M+H]⁺.

### Step E: (4aR)-4a-(((tert-Butyldimethylsilyl)oxy)methyl)-1-(4-fluorophenyl)-4,4a,5,6,7,8-hexahydro-1H-benzo[f]indazole-6-carbaldehyde (8e)

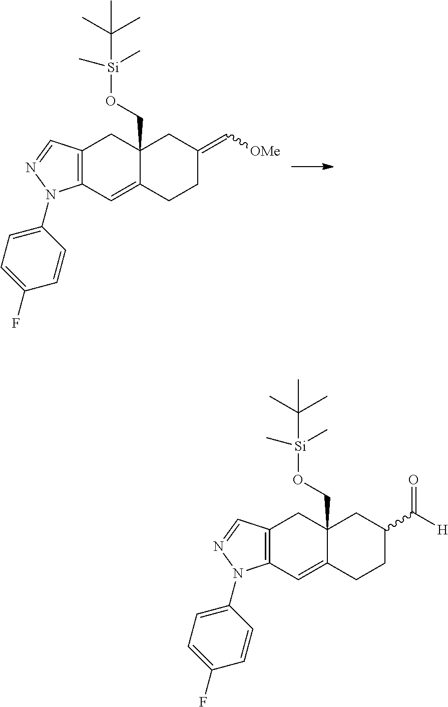 [SMILES: COC=C1CCC2=Cc3c(cnn3-c3ccc(F)cc3)C[C@]2(CO[Si](C)(C)C(C)(C)C)C1 ; CC(C)(C)[Si](C)(C)OC[C@]12Cc3cnn(-c4ccc(F)cc4)c3C=C1CCC(C=O)C2]

To a stirred solution of (S)-4a-(((tert-butyldimethylsilyl)oxy)methyl)-1-(4-fluorophenyl)-6-(methoxymethylene)-4,4a,5,6,7,8-hexahydro-1H-benzo[f]indazole (8d) (2.10g, 4.62 mmol) in wet DCM (220 mL) was added trichloroacetic acid (7.17 g, 43.9 mmol), followed by addition of water (0.45 mL) at rt. After the reaction was stirred at rt for 4 h, it was quenched (sat. aq. NaHCO₃) and extracted (DCM). The organic layer was washed (brine), dried (Na₂SO₄), and concentrated under reduced pressure. Purification of the residue by column chromatography (5% to 20% EtOAc/hexane, a gradient elution) provided (4aR)-4a-(((tert-butyldimethylsilyl)oxy)methyl)-1-(4-fluorophenyl)-4,4a,5,6,7,8-hexahydro-1H-benzo[f]indazole-6-carbaldehyde (8e) as a diastereomeric mixture (cis:trans=2.2:1.0, 1.64 g, 81%). m/z (ESI, +ve ion)=441.1 [M+H]⁺.

Step F: ((4aR,6S)-4a-(((tert-Butyldimethylsilyl)oxy)methyl)-1-(4-fluorophenyl)-4,4a,5,6,7,8-hexahydro-1H-benzo[f]indazol-6-yl)methanol (8f-1) and ((4aR,6R)-4a-(((tert-Butyldimethylsilyl)oxy)methyl)-1-(4-fluorophenyl)-4,4a,5,6,7,8-hexahydro-1H-benzo[f]indazol-6-yl)methanol (8f-2)

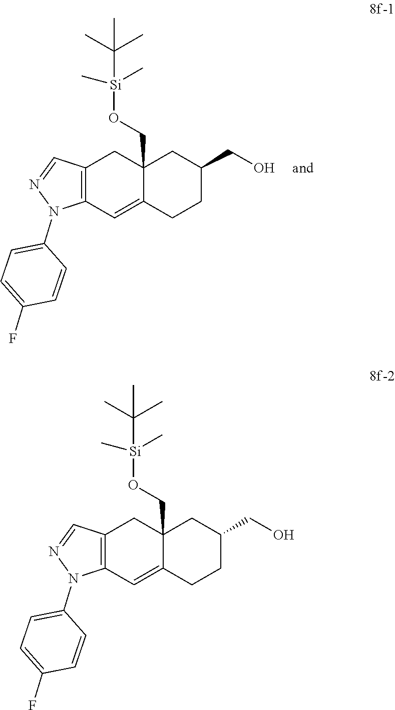 [SMILES: CC(C)(C)[Si](C)(C)OC[C@]12Cc3cnn(-c4ccc(F)cc4)c3C=C1CC[C@H](CO)C2 ; CC(C)(C)[Si](C)(C)OC[C@]12Cc3cnn(-c4ccc(F)cc4)c3C=C1CC[C@@H](CO)C2]

To a stirred solution of (4aR)-4a-(((tert-butyldimethylsilyl)oxy)methyl)-1-(4-fluorophenyl)-4,4a,5,6,7,8-hexahydro-1H-benzo[f]indazole-6-carbaldehyde (8e) (347 mg, 0.788 mmol) in MeOH (8 mL) was added NaBH₄ (44.7 mg, 1.18 mmol) at 0° C. The reaction mixture was allowed to warm to rt and stirred at rt for 15 min. After acetone (0.58 mL) was added, the resulting mixture was stirred at rt for another 30 min. The mixture was poured into water and the solution was extracted (EtOAc). The organic layer was washed (brine), dried (Na₂SO₄), and concentrated under reduced pressure. Purification of the residue by column chromatography (15% to 30% EtOAc/hexane, a gradient elution) provided ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methanol (8f-1) (second eluting isomer, 227 mg, 65%) as a white foamy solid and ((4aR,6R)-4a-(((tert-butyldimethylsilyl)oxy)methyl)-1-(4-fluorophenyl)-4,4a,5,6,7,8-hexahydro-1H-benzo[f]indazol-6-yl)methanol (8f-2) (first eluting isomer, 116 mg, 33%) as a white foamy solid, m/z (ESI, +ve ion)=443.1 [M+H]⁺. The C6 stereochemistry of 8f-1 and 8f-2 is randomly assigned.

### Step G: (4aR,6S)-4a-(((tert-Butyldimethylsilyl)oxy)methyl)-1-(4-fluorophenyl)-4,4a,5,6,7,8-hexahydro-1H-benzo[f]indazole-6-carbaldehyde (8g)

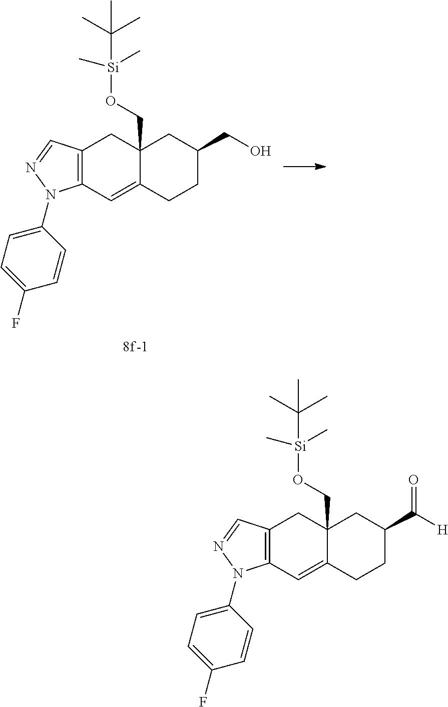 [SMILES: CC(C)(C)[Si](C)(C)OC[C@]12Cc3cnn(-c4ccc(F)cc4)c3C=C1CC[C@H](CO)C2 ; CC(C)(C)[Si](C)(C)OC[C@]12Cc3cnn(-c4ccc(F)cc4)c3C=C1CC[C@H](C=O)C2]

To a stirred solution of ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methanol (8f-1) (267 mg, 0.60 mmol) in DCM (9.5 mL) was added Dess-Martin periodinane (269 mg, 0.63 mmol) at rt. After the reaction mixture was stirred for 30 min, it was quenched (sat. aq. NaHCO₃ and 10% aq. Na₂S₂O₃). The solution was stirred at rt for another 15 min and extracted (DCM). The organic layer was washed (brine), dried (Na₂SO₄), and concentrated under reduced pressure. Purification of the residue by column chromatography (5% to 30% EtOAc/hexanes, a gradient elution) provided (4aR,6S)-4a-(((tert-butyldimethylsilyl)oxy)methyl)-1-(4-fluorophenyl)-4,4a,5,6,7,8-hexahydro-1H-benzo[f]indazole-6-carbaldehyde (8g) (235 mg, 88%) as a colorless gum. m/z (ESI, +ve ion)=441.3 [M+H]⁺.

### Step H: 1-((4aR,6S)-4a-(((tert-Butyldimethylsilyl)oxy)methyl)-1-(4-fluorophenyl)-4,4a,5,6,7,8-hexahydro-1H-benzo[f]indazol-6-yl)ethan-1-ol (8h)

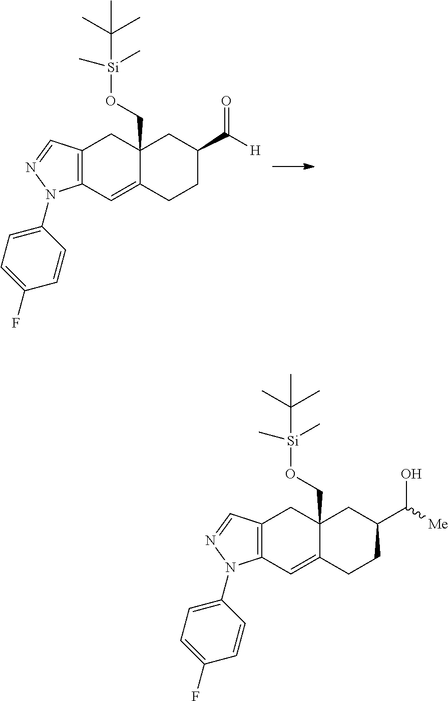 [SMILES: CC(C)(C)[Si](C)(C)OC[C@]12Cc3cnn(-c4ccc(F)cc4)c3C=C1CC[C@H](C=O)C2 ; CC(O)[C@H]1CCC2=Cc3c(cnn3-c3ccc(F)cc3)C[C@]2(CO[Si](C)(C)C(C)(C)C)C1]

To a stirred solution of (4aR,6S)-4a-(((tert-butyldimethylsilyl)oxy)methyl)-1-(4-fluorophenyl)-4,4a,5,6,7,8-hexahydro-1H-benzo[f]indazole-6-carbaldehyde (8g) (191 mg, 0.434 mmol) in THF (4 mL) at 0° C. was added methylmagnesium bromide solution (3.0 M in diethyl ether, 0.51 mL, 1.52 mmol) dropwise at 0° C. After the mixture was allowed to warm to rt and stirred for 1 h, it was quenched (sat. NH₄Cl aq.) and extracted (EtOAc). The organic layer was washed (brine), dried (Na₂SO₄), and concentrated under reduced pressure. Purification of the residue by column chromatography (5% to 20% acetone/hexanes, a gradient elution) provided 1-((4aR,6S)-4a-(((tert-butyldimethylsilyl)oxy)methyl)-1-(4-fluorophenyl)-4,4a,5,6,7,8-hexahydro-1H-benzo[f]indazol-6-yl)ethan-1-ol (8h) (127 mg, 64%) as a white foamy solid, m/z (ESI, +ve ion)=457.3 [M+H]⁺.

### Step I: 3-((4-Methoxybenzyl)thio)-1-methyl-1H-pyrazole (8i)

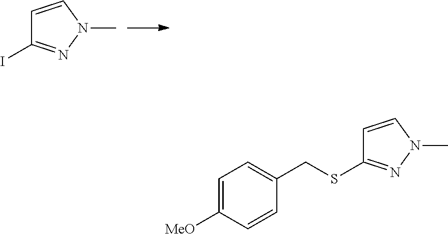

A round bottom flask was charged with 3-iodo-1-methyl-1H-pyrazole (5.08 g, 24.4 mmol), 4-methoxybenzylmercaptan (4.40 mL, 31.8 mmol), xantphos (707 mg, 1.20 mmol) and 1,4-dioxane (130 mL). N-ethyldiisopropylamine (8.50 mL, 48.8 mmol) and tris(dibenzylideneacetone)dipalladium(0) (559 mg, 0.60 mmol) were successively added under argon. The flask was purged with argon and the mixture was heated at 90° C. for 5 h. After the reaction mixture was cooled down to rt, it was filtered over a pad of Celite and rinsed with EtOAc. The filtrate was concentrated under reduced pressure and the orange residue was purified by column chromatography (20% to 50% EtOAc/hexanes, a gradient elution) to provide the title compound (8i) (5.30 g, 93%) as an orange solid, m/z (ESI, +ve ion)=235.1 [M+H]⁺.

### Step J: 1,2-Bis(1-methyl-1H-pyrazol-3-yl)disulfane (8j)

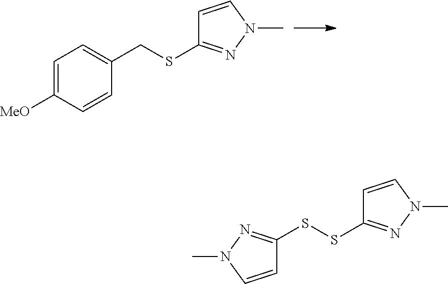

A pressure tube was charged with a solution of 3-((4-methoxybenzyl)thio)-1-methyl-1H-pyrazole (8i) (5.30 g, 22.6 mmol) in TFA (70 mL) and the solution was heated at 100° C. for 20 h. After the solution was cooled down to rt, TFA was removed under reduced pressure and the residue was azeotroped with toluene. This deep greenish residue was dissolved in DCM (200 mL) and iodobenzene diacetate (7.29 g, 22.6 mmol) was added in one portion. After the mixture was stirred at rt for 10 min, the reaction was quenched (sat. aq. NaHCO₃, 10% aq. NaS₂O₃). The resulting solution was stirred at rt for 20 min and extracted (DCM). The combined organic layers were washed (brine), dried (Na₂SO₄), and concentrated under reduced pressure. The residue was purified by column chromatography (2% to 5% MeOH/DCM followed by 50% to 100% EtOAc/hexanes, a gradient elution) to provide the title compound (8j) (1.80 g, 70%) as an orange solid, m/z (ESI, +ve ion)=227.1 [M+H]⁺.

### Step K: (4aR,6S)-4a-(((tert-Butyldimethylsilyl)oxy)methyl)-1-(4-fluorophenyl)-6-(1-((1-methyl-1H-pyrazol-3-yl)thio)ethyl)-4,4a,5,6,7,8-hexahydro-1H-benzo[f]indazole (8k)

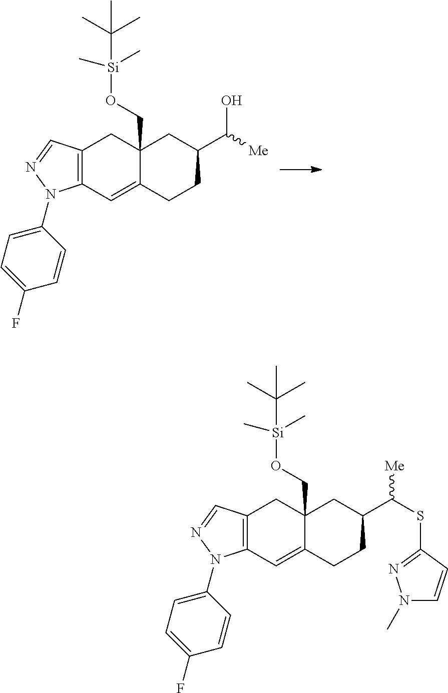

To a mixture of 1-((4aR,6S)-4a-(((tert-butyldimethylsilyl)oxy)methyl)-1-(4-fluorophenyl)-4,4a,5,6,7,8-hexahydro-1H-benzo[f]indazol-6-yl)ethan-1-ol (8h) (850 mg, 1.86 mmol) and 1,2-bis(1-methyl-1H-pyrazol-3-yl)disulfane (8j) (969 mg, 4.28 mmol) in toluene (9.6 mL) was added a solution of n-Bu₃P (1.07 mL, 4.28 mmol) in toluene (4.8 mL) dropwise over the period of 5 min under argon. The mixture was heated at 100° C. overnight. After the mixture was cooled down to rt, toluene was removed under reduced pressure. Purification of the residue by column chromatography (10% to 50% EtOAc/hexanes, a gradient elution) provided the title compound (8k) (1.03 g, quant.) as a colorless oil. m/z (ESI, +ve ion)=553.2 [M+H]⁺.

### Step L: (4aR,6S)-4a-(((tert-Butyldimethylsilyl)oxy)methyl)-1-(4-fluorophenyl)-6-(1-((1-methyl-1H-pyrazol-3-yl)sulfonyl)ethyl)-4,4a,5,6,7,8-hexahydro-1H-benzo[f]indazole (8l)

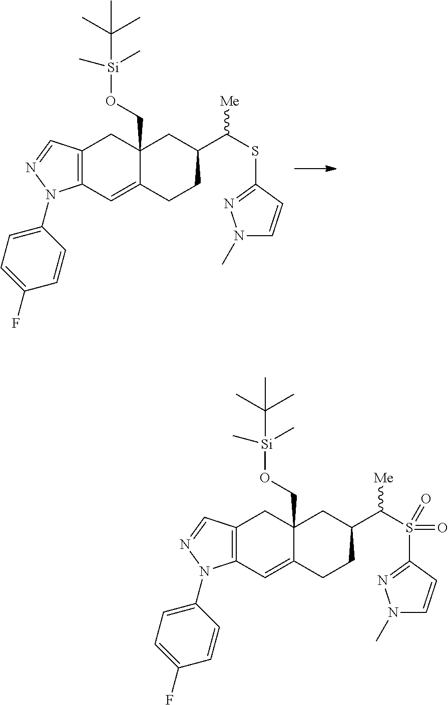

To a stirred solution of (8k) (1.03 g, 1.86 mmol) in MeOH (4.1 mL), water (4.1 mL), and THF (8.2 mL) was added oxone (1.99 g, 13.0 mmol) in one portion at rt. The mixture was heated at 45° C. for 1 h. Additional oxone (424 mg, 2.79 mmol) was added and the mixture was heated at 53° C. for 2 h. Then another portion of oxone (707 mg, 4.65 mmol) was added and the mixture was heated at 60° C. for 30 min. After the mixture was cooled down to 0° C., it was quenched (10% aq. Na₂S₂O₃, sat. aq. NaHCO₃) and extracted (EtOAc). The organic layers were washed (brine), dried (Na₂SO₄), and concentrated under reduced pressure. To a solution of the residue in DMF (10 mL) were added tert-butyldimethylsilyl chloride (841 mg, 5.58 mmol) and imidazole (507 mg, 7.44 mmol) successively at 0° C. After the solution was allowed to warm to rt and stirred for 3 h, it was quenched (water) and extracted (EtOAc). The organic layer was washed (water and brine), dried (Na₂SO₄), and concentrated under reduced pressure. Purification of the residue by column chromatography (80% to 20% EtOAc/hexanes, a gradient elution) provided (81) (757 mg, 70%) as a white foamy solid, m/z (ESI, +ve ion)=585.3 [M+H]⁺.

### Step M: ((4aR,6S)-1-(4-Fluorophenyl)-6-(2-((1-methyl-1H-pyrazol-3-yl)sulfonyl)propan-2-yl)-1,4,5,6,7,8-hexahydro-4aH-benzo[f]indazol-4a-yl)methanol (8m)

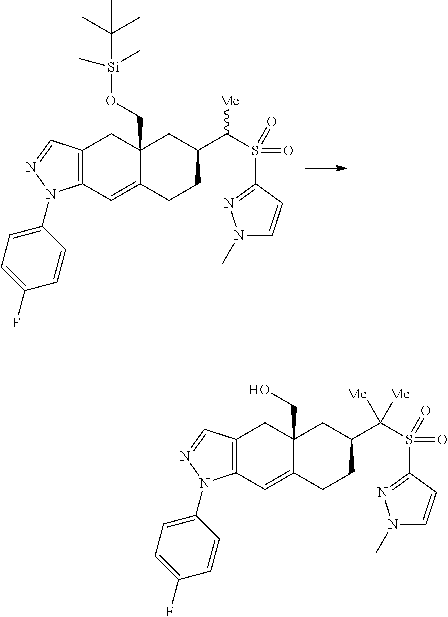 [SMILES: CC([C@H]1CCC2=Cc3c(cnn3-c3ccc(F)cc3)C[C@]2(CO[Si](C)(C)C(C)(C)C)C1)S(=O)(=O)c1ccn(C)n1 ; Cn1ccc(S(=O)(=O)C(C)(C)[C@H]2CCC3=Cc4c(cnn4-c4ccc(F)cc4)C[C@]3(CO)C2)n1]

To a stirred solution of (8l) (117 mg, 0.20 mmol) in THF (1.5 mL) at −78° C. was added n-butyllithium solution (1.6 M in hexane, 0.175 mL, 0.28 mmol) dropwise. After the reaction was stirred at −78° C. for 30 min, a solution of iodomethane (39.7 mg, 0.28 mmol) in THF (0.5 mL) was added dropwise. After the reaction was stirred at −78° C. for 20 min, the reaction was quenched with water. The dry ice bath was removed and sat. aq. NH₄Cl solution was added. After the solution was allowed to warm to rt, it was extracted (EtOAc). The organic layer was washed (brine), dried (Na₂SO₄), and concentrated under reduced pressure. To a stirred solution of the residue in MeOH (9 mL) was added 3N aq. HCl solution (1.67 mL, 5.0 mmol) dropwise. The reaction was stirred at rt for 2 h, quenched (sat. aq. NaHCO₃) and extracted (EtOAc). The organic layers were washed (brine), dried (Na₂SO₄), and concentrated under reduced pressure. The residue was purified by column chromatography (1% to 5% MeOH/DCM, a gradient elution). The crude product was purified by reverse HPLC (15% to 50% MeCN/water with 0.1% formic acid) to provide the title compound (8m) (68 mg, 70%) as a white solid, m/z (ESI, +ve ion)=485.2 [M+H]⁺.

### Step N: ((4aR,6S)-1-(4-Fluorophenyl)-6-(2-((1-methyl-1H-pyrazol-3-yl)sulfonyl)propan-2-yl)-1,4,5,6,7,8-hexahydro-4aH-benzo[f]indazol-4a-yl)(4-fluoropyridin-2-yl)methanone (8)

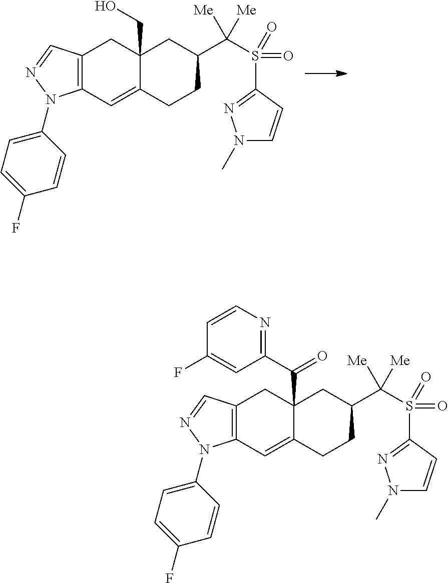 [SMILES: Cn1ccc(S(=O)(=O)C(C)(C)[C@H]2CCC3=Cc4c(cnn4-c4ccc(F)cc4)C[C@]3(CO)C2)n1 ; Cn1ccc(S(=O)(=O)C(C)(C)[C@H]2CCC3=Cc4c(cnn4-c4ccc(F)cc4)C[C@]3(C(=O)c3cc(F)ccn3)C2)n1]

The title compound was prepared from 8m by procedures similar to those described in Example 2, Steps B, C, and D. ¹H NMR (400 MHz, Chloroform-d) δ 8.66 (1H, dd, J=8.4, 5.6 Hz), 7.50 (1H, dd, J=7.5, 2.6 Hz), 7.48 (1H, d, J=2.4 Hz), 7.46-7.42 (2H, m), 7.17-7.13 (3H, m), 6.78 (1H, d, J=2.4 Hz), 6.44 (1H, s), 4.04 (1H, d, J=16.4 Hz), 3.99 (3H, s), 3.24 (1H, d, J=16.8 Hz), 3.19 (1H, d, J=14.0 Hz), 2.58-2.42 (3H, m), 1.96 (1H, t, J=13.2 Hz), 1.85-1.68 (2H, m), 1.38 (3H, s), 1.31 (3H, s). m/z (ESI, +ve ion)=578.2 [M+H]⁺.

### Example 9. ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone (9)

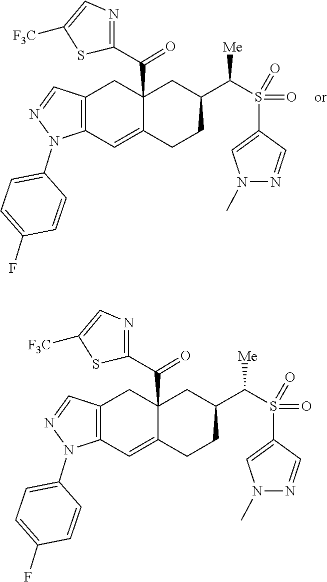

### Step A: ((4aR,6S)-1-(4-Fluorophenyl)-6-(2-((1-methyl-1H-pyrazol-3-yl)sulfonyl)propan-2-yl)-1,4,5,6,7,8-hexahydro-4aH-benzo[f]indazol-4a-yl)(4-fluoropyridin-2-yl)methanone (9a)

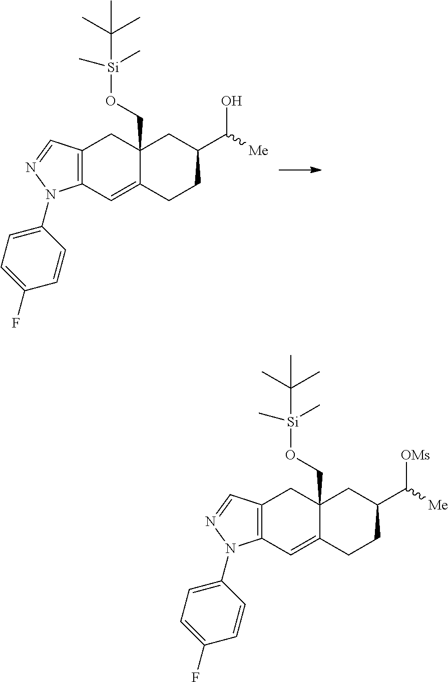

To a stirred solution of 1-((4aR,6S)-4a-(((tert-butyldimethylsilyl)oxy)methyl)-1-(4-fluorophenyl)-4,4a,5,6,7,8-hexahydro-1H-benzo[f]indazol-6-yl)ethan-1-ol (8h) (127 mg, 0.278 mmol) in DCM (4 mL) was added triethylamine (0.23 mL, 1.67 mmol). After the reaction mixture was cooled to 0° C., methanesulfonyl chloride (54 μL. 0.7 mmol) was added dropwise. The reaction was allowed to warm to rt and stirred at the same temperature for 30 min. The reaction was quenched with water and extracted with DCM. The organics were washed with brine, dried over anhydrous sodium sulfate and concentrated under reduced pressure to afford an orange foaming solid, which was purified by chromatography (10% to 40% EtOAc/hexanes, a gradient elution) provided the title compound (9a) (127 mg, 85%) as a white foamy solid, m/z (ESI, +ve ion) 535.3 [M+H]⁺.

### Step B: (4aR,6S)-4a-(((tert-Butyldimethylsilyl)oxy)methyl)-1-(4-fluorophenyl)-6-(1-((1-methyl-1H-pyrazol-4-yl)thio)ethyl)-4,4a,5,6,7,8-hexahydro-1H-benzo[f]indazole (9b)

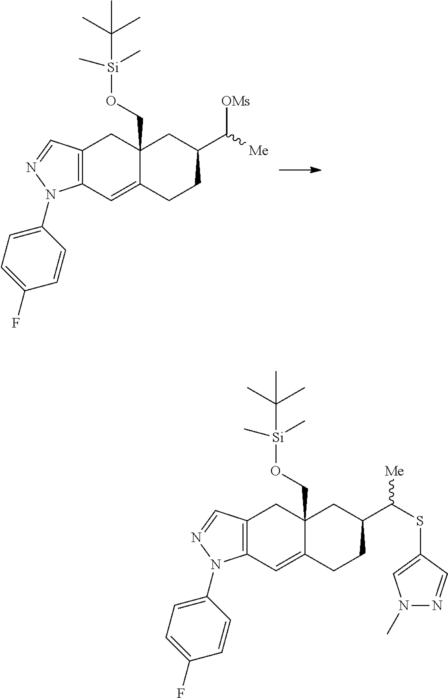

To a stirred suspension of sodium hydride (60% in mineral oil, 35.2 mg, 0.88 mmol) in DMF (1.5 mL) was added 1-methylpyrazole-4-thiol (111 mg, 0.97 mmol) at rt under Ar. The mixture was stirred at rt until gas evolution was ceased (about 5 min). To the prepared thiolate solution was added a solution of 9a (157 mg, 0.29 mmol) in DMF (1.5 mL) and the resulting mixture was heated at 50° C. for 40 min. After cooling to rt, the reaction mixture was poured into saturated aq. NH₄Cl solution and extracted (3×EtOAc). The combined organic layer was washed (water and brine), dried (Na₂SO₄), and concentrated under reduced pressure. Purification of the residue by column chromatography (15% to 80% EtOAc/hexanes, a gradient elution) provided (9b) (127 mg, 78%) as a colorless gum. m/z (ESI, +ve ion)=553.3 [M+H]⁺.

### Step C: ((4aR,6S)-1-(4-Fluorophenyl)-6-(1-((1-methyl-1H-pyrazol-4-yl)thio)ethyl)-1,4,5,6,7,8-hexahydro-4aH-benzo[f]indazol-4a-yl)methanol (9c)

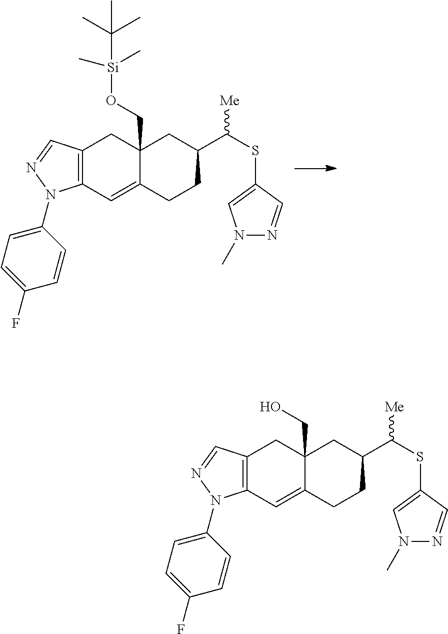

To a stirred solution of 9b (127 mg, 0.23 mmol) in MeOH (15 mL) was added 3 N aq. HCl solution (2.3 mL, 6.9 mmol) dropwise. The reaction was stirred at rt for 3 h, quenched (sat. aq. NaHCO₃) and extracted (EtOAc). The combined organic layers were washed (brine), dried (Na₂SO₄), and concentrated under reduced pressure. ((4aR,6S)-1-(4-fluorophenyl)-6-(1-((1-methyl-1H-pyrazol-4-yl)thio)ethyl)-1,4,5,6,7,8-hexahydro-4aH-benzo[f]indazol-4a-yl)methanol (9c) (101 mg, 100%) was obtained as an off-white foamy solid, m/z (ESI, +ve ion)=439.3 [M+H]⁺.

### Step D: ((4aR,6S)-1-(4-fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (9d-1) and ((4aR,6S)-1-(4-fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (9d-2)

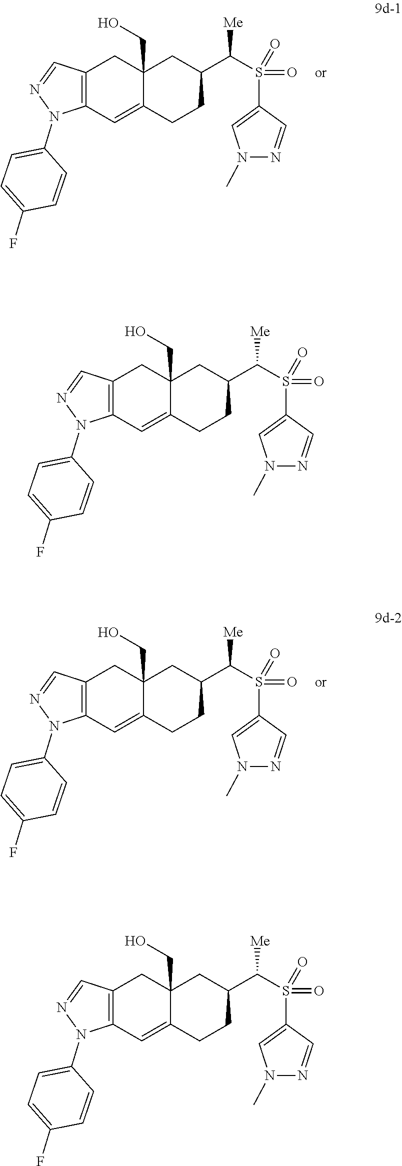

To a stirred solution of ((4aR,6S)-1-(4-fluorophenyl)-6-(1-((1-methyl-1H-pyrazol-4-yl)thio)ethyl)-1,4,5,6,7,8-hexahydro-4aH-benzo[f]indazol-4a-yl)methanol (9c) (101 mg, 0.23 mmol) in MeOH (0.7 mL)/water (0.7 mL)/THF (1.4 mL) was added oxone (186 mg, 1.22 mmol) in one portion at rt. After the mixture was heated at 40° C. for 1 h and cooled down to 0° C., it was quenched (10% aq. Na₂S₂O₃, sat. aq. NaHCO₃) and extracted (EtOAc). The organic layer was washed (brine), dried (Na₂SO₄), and concentrated under reduced pressure. Purification of the residue by column chromatography (0% to 15% MeOH/EtOAc, a gradient elution) provided the title compound (9d-1) (first eluting isomer, 40.4 mg, 35%) and (9d-2) (second eluting isomer, 53.2 mg, 46%). m/z (ESI, +ve ion)=471.3 [M+H]⁺.

### Step E: ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone (9)

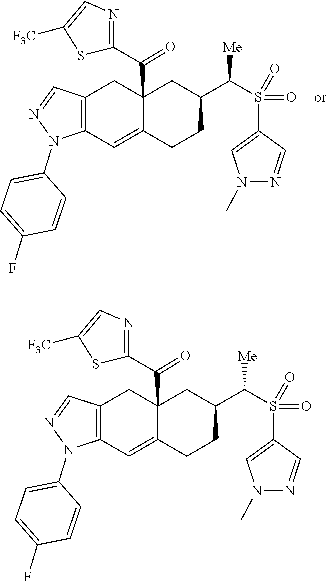

The title compound was prepared from 9d-1 by procedures similar to those described in Example 2, Steps B, C, and D. ¹H NMR (400 MHz, Chloroform-d) δ 8.30 (1H, q, J=1.2 Hz), 7.84 (1H, s), 7.82 (1H, d, J=0.4 Hz), 7.47-7.44 (2H, m), 7.33 (1H, s), 7.19-7.14 (2H, m), 6.52 (1H, s), 4.08 (1H, d, J=16.8 Hz), 3.99 (3H, s), 3.22 (1H, d, J=16.4 Hz), 2.99 (1H, qd, J=6.8, 2.4 Hz), 2.91-2.81 (1H, m), 2.79 (1H, d, J=13.6 Hz), 2.52-2.49 (2H, m), 1.90 (1H, t, J=13.2 Hz), 1.78-1.57 (2H, m), 1.30 (3H, d, J=7.2 Hz), m/z (ESI, +ve ion)=620.0 [M+H]⁺.

### Example 10: ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(5-(trifluoromethyl)thiazol-2-yl)methanone (10)

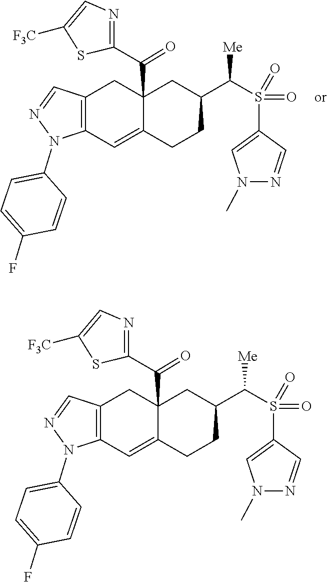

The title compound was prepared from 9d-2 by procedures similar to those described in Example 2, Steps B, C, and D. Tl NMR (400 MHz, Chloroform-d) δ 8.26 (1H, q, J=1.2 Hz), 7.82 (1H, m), 7.79 (1H, d, J=0.4 Hz), 7.48-7.44 (2H, m), 7.32 (1H, s), 7.19-7.15 (2H, m), 6.54 (1H, d, J=1.2 Hz), 4.01 (1H, d, J=16.8 Hz), 3.97 (3H, s), 3.17 (1H, d, J=16.4 Hz), 3.09 (1H, qd, J=6.8, 4.0 Hz), 2.86-2.77 (1H, m), 2.53-2.38 (3H, m), 2.12 (1H, t, J=13.2 Hz), 1.91-1.73 (2H, m), 1.29 (3H, d, J=6.8 Hz), m/z (ESI, +ve ion)=620.0 [M+H]⁺.

### Example 11: N-cyclopropyl-N-((4aS,6S)-1-(6-fluoropyridin-3-yl)-4a-(4-(trifluoromethyl)picolinoyl)-4,4a,5,6,7,8-hexahydro-1H-benzo[f]indazol-6-yl)-1-methyl-1H-1,2,4-triazole-3-sulfonamide

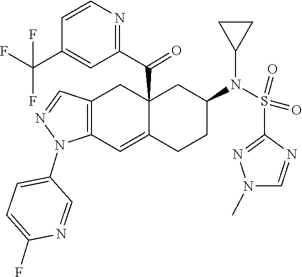 [SMILES: Cn1cnc(S(=O)(=O)N(C2CC2)[C@H]2CCC3=Cc4c(cnn4-c4ccc(F)nc4)C[C@]3(C(=O)c3cc(C(F)(F)F)ccn3)C2)n1]

The title compound was prepared by procedures similar to those described in Example 3. ¹H NMR (400 MHz, CHLOROFORM-d) δ ppm 8.87 (d, J=5.12 Hz, 1H), 8.32 (dd, J=2.56, 1.10 Hz, 1H), 8.17 (s, 1H), 8.03-8.06 (m, 1H), 7.92-8.00 (m, 1H), 7.31 (s, 1H), 7.69 (dd, J=5.26, 1.46 Hz, 1H), 7.07 (dd, J=8.70, 3.29 Hz, 1H), 6.46 (d, J=1.61 Hz, 1H), 4.47-4.51 (m, 1H), 4.04 (s, 3H), 3.94 (d, J=16.66 Hz, 1H), 3.23 (d, J=16.81 Hz, 1H), 2.38-2.78 (m, 5H), 2.13-2.22 (m, 1H), 1.88-1.94 (m, 1H), 1.11-1.14 (m, 1H), 0.87-0.96 (m, 1H), 0.66-0.85 (m, 2H).

Example 12-99 were synthesized as described in examples 1-11.

| Ex. | [M + H]⁺ |
|---|---|
| 12 | 671.0 |
| 13 | 657.2 |
| 14 | 687.0 |
| 15 | 550.2 |
| 16 | 648.1 |
| 17 | 587.2 |
| 18 | 642.2 |
| 19 | 656.2 |
| 20 | 632.2 |
| 21 | 686.2 |
| 22 | 648.0 |
| 23 | 655.2 |
| 24 | 671.1 |
| 25 | 629.2 |
| 26 | 566.0 |
| 27 | 606.2 |
| 28 | 645.0 |
| 29 | 613.1 |
| 30 | 616.1 |
| 31 | 632.2 |
| 32 | 647.2 |
| 33 | 672.2 |
| 34 | 647.2 |
| 35 | 614.2 |
| 36 | 614.2 |
| 37 | 665.2 |
| 38 | 651.1 |
| 39 | 642.2 |
| 40 | 643.2 |
| 41 | 650.1 |
| 42 | 740.2 |
| 43 | 690.2 |
| 44 | 706.1 |
| 45 | 706.1 |
| 46 | 636.2 |
| 47 | 640.3 |
| 48 | 593.2 |
| 49 | 631.1 |
| 50 | 597.2 |
| 51 | 645.3 |
| 52 | 611.2 |
| 53 | 663.0 |
| 54 | 682.1 |
| 55 | 643.2 |
| 56 | 621.1 |
| 57 | 621.1 |
| 58 | 590.2 |
| 59 | 608.2 |
| 60 | 713.2 |
| 61 | 636.2 |
| 62 | 564.3 |
| 63 | 637.3 |
| 64 | 631.3 |
| 65 | 637.3 |
| 66 | 595.3 |
| 67 | 606.1 |
| 68 | 594.2 |
| 69 | 644.2 |
| 70 | 643.2 |
| 71 | 642.2 |
| 72 | 638.3 |
| 73 | 668.2 |
| 74 | 642.2 |
| 75 | 681.2 |
| 76 | 680.3 |
| 77 | 565.2 |
| 78 | 635.2 |
| 79 | 593.3 |
| 80 | 591.2 |
| 81 | 594.2 |
| 82 | 633.2 |
| 83 | 565.2 |
| 84 | 594.2 |
| 85 | 610.2 |
| 86 | 581.2 |
| 87 | 593.2 |
| 88 | 628.2 |
| 89 | 592.2 |
| 90 | 569.0 |
| 91 | 537.2 |
| 92 | 569.2 |
| 93 | 601.2 |
| 94 | 581.2 |
| 95 | 627.1 |
| 96 | 632.2 |
| 97 | 577.2 |
| 98 | 551.2 |
| 99 | 627.1 |

### II. Biological Evaluation

### Example A: In Vitro GR Luciferase Reporter Assay

Cell Line: CHO-K1-GR-MMTV-Luc reporter cells

Culture Media: DMEM (with phenol red)+10% FBS

Assay Media: DMEM (without phenol red)+10% CSS

Culture CHO-K1-GR-MMTV-Luc reporter cells in T175 flasks in Culture Media at conditions less than 90% confluence.

Using Bravo (Agilent) liquid handler:

200× DMSO 1:3 serial dilutions of control and test compounds in 96-well non-sterile V bottom plate in DMSO, 12 points for each compound were prepared.

Prepared 5× compound serial dilutions: Add 97.5 μL/well of Assay Media into 96-well non-sterile V bottom plate then added 2.5 ul of 200× concentration of compounds and mix well.

Seeded cells for Antagonist Assay: 5200 CHO-K1-GR-MMTV-Luc reporter cells were seeded in Greiner #781080 flat clear bottom 384-well white TC plate in 20 ul of Assay Media containing 12.5 nM Dexamethasone (final concentration=10 nM).

Added compounds: 5 μl of 5× compounds were added to appropriate wells and followed with a quick spin (1000 rpm, 10 sec) to bring media and cells to the bottom of plate. The plates were covered with SealMate film to avoid evaporation and placed in 37° C. incubator for approximately 18-24 hours.

Read plates: Equilibrate appropriate amount of Promega OneGlo luciferase reagent to room temperature. Remove the plates from incubator and add 25 uL of OneGlo reagent/well by Bravo and read the plates on Tecan Spark plate reader within 15 minutes.

The ability of the compounds disclosed herein to inhibit GR activity was quantified and the respective IC₅₀ value was determined. Table 1 provides the cellular IC₅₀ values of compounds disclosed herein.

TABLE 1

| Ex. | GR IC₅₀ (nM) |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | B |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | B |
| 43 | B |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | C |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | B |
| 99 | A |

A = IC₅₀ is less than or equal to 100 nM;
B = IC₅₀ is greater than 100 nM and less than or equal to 1 μM;
C = IC₅₀ is greater than 1 μM

The examples and embodiments described herein are for illustrative purposes only and in some embodiments, various modifications or changes are to be included within the purview of disclosure and scope of the appended claims.

What is claimed is:

1. A compound having the structure of Formula (I), or a pharmaceutically acceptable salt, solvate, stereoisomer, or isotopic variant thereof: 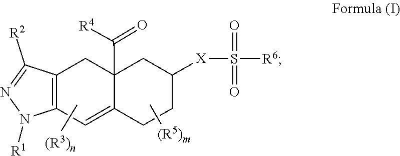
wherein:
R¹ is cycloalkyl, heterocycloalkyl, or heteroaryl; wherein the cycloalkyl, heterocycloalkyl, and heteroaryl are independently optionally substituted with one, two, or three R^{1a};
each R^{1a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{1a} on the same carbon form an oxo;
R² is hydrogen, halogen, C₁-C₆ alkyl, or C₁-C₆ haloalkyl;
each R³ is independently halogen, C₁-C₆ alkyl, or C₁-C₆ haloalkyl;
R⁴ is cycloalkyl, heterocycloalkyl, aryl, or heteroaryl; wherein the cycloalkyl, heterocycloalkyl, aryl, and heteroaryl are independently optionally substituted with one, two, or three R^{4a};
each R^{4a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{4a} on the same carbon form an oxo;
or two R^{4a} are taken together to form a cycloalkyl or a heterocycloalkyl;
each R⁵ is independently halogen, C₁-C₆ alkyl, or C₁-C₆ haloalkyl;
R⁶ is aryl, heteroaryl, cycloalkyl, or heterocycloalkyl; wherein the aryl, heteroaryl, cycloalkyl, and heterocycloalkyl are independently optionally substituted with one, two, or three R^{6a};
each R^{6a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{6a} on the same carbon form an oxo;
X is a bond, —C(R⁷)₂—, or —NR⁸—;
each R⁷ is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{7a};
each R^{7a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{7a} on the same carbon form an oxo;
R⁸ is hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three R^{8a};
each R^{8a} is independently halogen, —CN, —OR^{a}, —NR^{c}R^{d}, —C(═O)R^{b}, —C(═O)OR^{a}, —C(═O)NR^{c}R^{d}, C₁-C₆ alkyl, C₁-C₆ haloalkyl, C₁-C₆ hydroxyalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl;
or two R^{8a} on the same carbon form an oxo;
each R^{a} is independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{b} is independently C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three halogen, —OH, —NH₂, or C₁-C₆ alkyl;
each R^{c} and R^{d} are independently hydrogen, C₁-C₆ alkyl, C₁-C₆ haloalkyl, C₁-C₆ hydroxyalkyl, C₁-C₆ aminoalkyl, cycloalkyl, heterocycloalkyl, aryl, or heteroaryl; wherein the alkyl, cycloalkyl, heterocycloalkyl, aryl, and heteroaryl are independently optionally substituted with one, two, or three, halogen, —OH, —NH₂, or C₁-C₆ alkyl;
or R^{c} and R^{d} are taken together with the nitrogen atom to which they are attached to form a heterocycloalkyl optionally substituted with one, two, or three halogen, —OH, —NH₂, or C₁-C₆ alkyl;
m is 0-4; and
n is 0-3;
provided that the compound is not

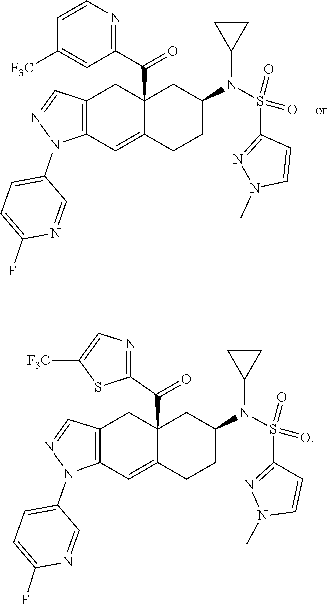

2. The compound of claim 1, or a pharmaceutically acceptable salt, solvate, stereoisomer, or isotopic variant thereof, wherein:
R² is hydrogen.

3. The compound of claim 1 or 2, or a pharmaceutically acceptable salt, solvate, stereoisomer, or isotopic variant thereof, wherein:
each R³ is independently halogen or C₁-C₆ alkyl.

4. The compound of any one of claims 1-3, or a pharmaceutically acceptable salt, solvate, stereoisomer, or isotopic variant thereof, wherein:
n is 0.

5. The compound of any one of claims 1-4, or a pharmaceutically acceptable salt, solvate, stereoisomer, or isotopic variant thereof, wherein:
each R⁵ is independently halogen or C₁-C₆ alkyl.

6. The compound of any one of claims 1-5, or a pharmaceutically acceptable salt, solvate, stereoisomer, or isotopic variant thereof, wherein:
m is 0.

7. The compound of any one of claims 1-6, or a pharmaceutically acceptable salt, solvate, stereoisomer, or isotopic variant thereof, wherein:
X is a bond.

8. The compound of any one of claims 1-6, or a pharmaceutically acceptable salt, solvate, stereoisomer, or isotopic variant thereof, wherein:
X is —C(R⁷)₂—.

9. The compound of any one of claims 1-6 or 8, or a pharmaceutically acceptable salt, solvate, stereoisomer, or isotopic variant thereof, wherein:
each R⁷ is independently hydrogen, C₁-C₆ alkyl, C₁-C₆ haloalkyl, cycloalkyl, or heterocycloalkyl, wherein the alkyl, cycloalkyl, and heterocycloalkyl are independently optionally substituted with one, two, or three R^{7a}.

10. The compound of any one of claims 1-6 or 8 or 9, or a pharmaceutically acceptable salt, solvate, stereoisomer, or isotopic variant thereof, wherein:
each R⁷ is independently hydrogen, C₁-C₆ alkyl, or C₁-C₆ haloalkyl.

11. The compound of any one of claims 1-6 or 8-10, or a pharmaceutically acceptable salt, solvate, stereoisomer, or isotopic variant thereof, wherein:
each R⁷ is hydrogen.

12. The compound of any one of claims 1-6, or a pharmaceutically acceptable salt, solvate, stereoisomer, or isotopic variant thereof, wherein:
X is —NR⁸—.

13. The compound of any one of claims 1-6 or 12, or a pharmaceutically acceptable salt, solvate, stereoisomer, or isotopic variant thereof, wherein:
R⁸ is hydrogen, C₁-C₆ alkyl, C₁-C₆ haloalkyl, cycloalkyl, or heterocycloalkyl, wherein the alkyl, cycloalkyl, and heterocycloalkyl are independently optionally substituted with one, two, or three R^{8a}.

14. The compound of any one of claims 1-6 or 12 or 13, or a pharmaceutically acceptable salt, solvate, stereoisomer, or isotopic variant thereof, wherein:
R⁸ is C₁-C₆ alkyl, C₁-C₆ haloalkyl, or cycloalkyl, wherein the alkyl, and cycloalkyl are independently optionally substituted with one, two, or three R^{8a}.

15. The compound of any one of claims 1-6 or 12-14, or a pharmaceutically acceptable salt, solvate, stereoisomer, or isotopic variant thereof, wherein:
R⁸ is C₁-C₆ haloalkyl.

16. The compound of any one of claims 1-6 or 12-14, or a pharmaceutically acceptable salt, solvate, stereoisomer, or isotopic variant thereof, wherein:
R⁸ is cycloalkyl.

17. The compound of any one of claims 1-6 or 12-14, or a pharmaceutically acceptable salt, solvate, stereoisomer, or isotopic variant thereof, wherein:
R⁸ is C₁-C₆ alkyl.

18. The compound of any one of claims 1-17, or a pharmaceutically acceptable salt, solvate, stereoisomer, or isotopic variant thereof, wherein:
R⁶ is aryl or heteroaryl; wherein the aryl and heteroaryl are independently optionally substituted with one, two, or three R^{6a}.

19. The compound of any one of claims 1-18, or a pharmaceutically acceptable salt, solvate, stereoisomer, or isotopic variant thereof, wherein:
R⁶ is heteroaryl optionally substituted with one, two, or three R^{6a}.

20. The compound of any one of claims 1-19, or a pharmaceutically acceptable salt, solvate, stereoisomer, or isotopic variant thereof, wherein:
R⁶ is a 5-membered heteroaryl optionally substituted with one, two, or three R^{6a}.

21. The compound of any one of claims 1-18, or a pharmaceutically acceptable salt, solvate, stereoisomer, or isotopic variant thereof, wherein:
R⁶ is aryl optionally substituted with one, two, or three R^{6a}.

22. The compound of any one of claims 1-21, or a pharmaceutically acceptable salt, solvate, stereoisomer, or isotopic variant thereof, wherein:
each R^{6a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆haloalkyl.

23. The compound of any one of claims 1-22, or a pharmaceutically acceptable salt, solvate, stereoisomer, or isotopic variant thereof, wherein:
each R^{6a} is independently halogen, C₁-C₆ alkyl, or C₁-C₆ haloalkyl.

24. The compound of any one of claims 1-23, or a pharmaceutically acceptable salt, solvate, stereoisomer, or isotopic variant thereof, wherein:
R⁴ is heteroaryl optionally substituted with one, two, or three R^{4a}.

25. The compound of any one of claims 1-24, or a pharmaceutically acceptable salt, solvate, stereoisomer, or isotopic variant thereof, wherein:
each R^{4a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁-C₆ alkyl, or C₁-C₆ haloalkyl.

26. The compound of any one of claims 1-25, or a pharmaceutically acceptable salt, solvate, stereoisomer, or isotopic variant thereof, wherein:
each R^{4a} is independently halogen, C₁-C₆ alkyl, or C₁-C₆ haloalkyl.

27. The compound of any one of claims 1-24, or a pharmaceutically acceptable salt, solvate, stereoisomer, or isotopic variant thereof, wherein:
two R^{4a} are taken together to form a heterocycloalkyl.

28. The compound of any one of claims 1-27, or a pharmaceutically acceptable salt, solvate, stereoisomer, or isotopic variant thereof, wherein:
R¹ is heterocycloalkyl or heteroaryl; wherein the heterocycloalkyl and heteroaryl are independently optionally substituted with one, two, or three R^{1a}.

29. The compound of any one of claims 1-28, or a pharmaceutically acceptable salt, solvate, stereoisomer, or isotopic variant thereof, wherein:
R¹ is heteroaryl optionally substituted with one, two, or three R^{1a}.

30. The compound of any one of claims 1-28, or a pharmaceutically acceptable salt, solvate, stereoisomer, or isotopic variant thereof, wherein:
R¹ is heterocycloalkyl optionally substituted with one, two, or three R^{1a}.

31. The compound of any one of claims 1-30, or a pharmaceutically acceptable salt, solvate, stereoisomer, or isotopic variant thereof, wherein:
each R^{1a} is independently halogen, —CN, —OR^{a}, —NR^{c}R^{d}, C₁-C₆ alkyl, C₁-C₆ haloalkyl, or C₁-C₆ hydroxyalkyl; wherein the alkyl are independently optionally substituted with one, two, or three halogen, —CN, —OR^{a}, —NR^{c}R^{d}, —C(═O)R^{b}, —C(═O)OR^{a}, —C(═O)NR^{c}R^{d}, C₁₋₆ alkyl, or C₁-C₆ haloalkyl.

32. The compound of any one of claims 1-31, or a pharmaceutically acceptable salt, solvate, stereoisomer, or isotopic variant thereof, wherein:
each R^{1a} is independently halogen, C₁-C₆ alkyl, or C₁-C₆ haloalkyl.

33. The compound of any one of claims 1-32, or a pharmaceutically acceptable salt, solvate, stereoisomer, or isotopic variant thereof, wherein:
each R^{1a} is independently halogen.

34. The compound of claim 1, or a pharmaceutically acceptable salt, solvate, stereoisomer, or isotopic variant thereof, wherein the compound is:

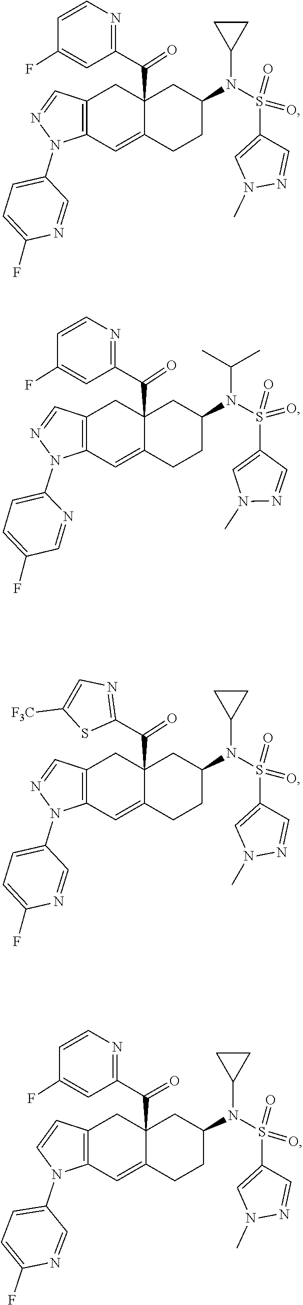 [SMILES: Cn1cc(S(=O)(=O)N(C2CC2)[C@H]2CCC3=Cc4c(cnn4-c4ccc(F)nc4)C[C@]3(C(=O)c3cc(F)ccn3)C2)cn1 ; CC(C)N([C@H]1CCC2=Cc3c(cnn3-c3ccc(F)cn3)C[C@]2(C(=O)c2cc(F)ccn2)C1)S(=O)(=O)c1cnn(C)c1 ; Cn1cc(S(=O)(=O)N(C2CC2)[C@H]2CCC3=Cc4c(cnn4-c4ccc(F)nc4)C[C@]3(C(=O)c3ncc(C(F)(F)F)s3)C2)cn1 ; Cn1cc(S(=O)(=O)N(C2CC2)[C@H]2CCC3=Cc4c(ccn4-c4ccc(F)nc4)C[C@]3(C(=O)c3cc(F)ccn3)C2)cn1]

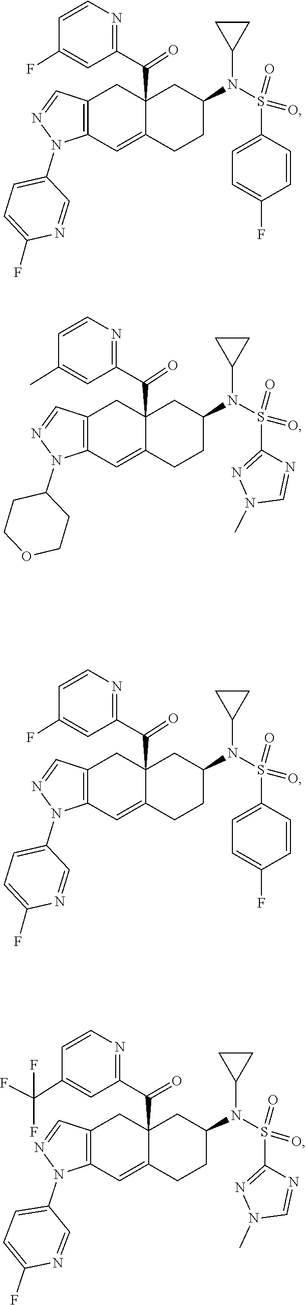 [SMILES: O=C(c1cc(F)ccn1)[C@]12Cc3cnn(-c4ccc(F)nc4)c3C=C1CC[C@H](N(C1CC1)S(=O)(=O)c1ccc(F)cc1)C2 ; Cc1ccnc(C(=O)[C@]23Cc4cnn(C5CCOCC5)c4C=C2CC[C@H](N(C2CC2)S(=O)(=O)c2ncn(C)n2)C3)c1 ; O=C(c1cc(F)ccn1)[C@]12Cc3cnn(-c4ccc(F)nc4)c3C=C1CC[C@H](N(C1CC1)S(=O)(=O)c1ccc(F)cc1)C2 ; Cn1cnc(S(=O)(=O)N(C2CC2)[C@H]2CCC3=Cc4c(cnn4-c4ccc(F)nc4)C[C@]3(C(=O)c3cc(C(F)(F)F)ccn3)C2)n1]

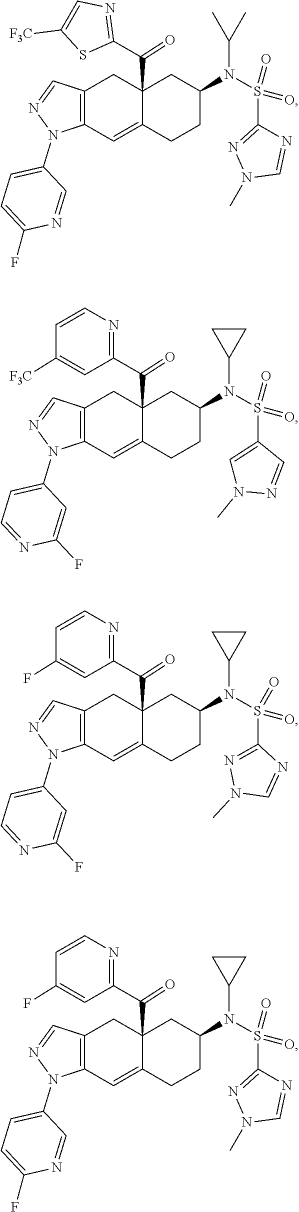 [SMILES: CC(C)N([C@H]1CCC2=Cc3c(cnn3-c3ccc(F)nc3)C[C@]2(C(=O)c2ncc(C(F)(F)F)s2)C1)S(=O)(=O)c1ncn(C)n1 ; Cn1cc(S(=O)(=O)N(C2CC2)[C@H]2CCC3=Cc4c(cnn4-c4ccnc(F)c4)C[C@]3(C(=O)c3cc(C(F)(F)F)ccn3)C2)cn1 ; Cn1cnc(S(=O)(=O)N(C2CC2)[C@H]2CCC3=Cc4c(cnn4-c4ccnc(F)c4)C[C@]3(C(=O)c3cc(F)ccn3)C2)n1 ; Cn1cnc(S(=O)(=O)N(C2CC2)[C@H]2CCC3=Cc4c(cnn4-c4ccc(F)nc4)C[C@]3(C(=O)c3cc(F)ccn3)C2)n1]

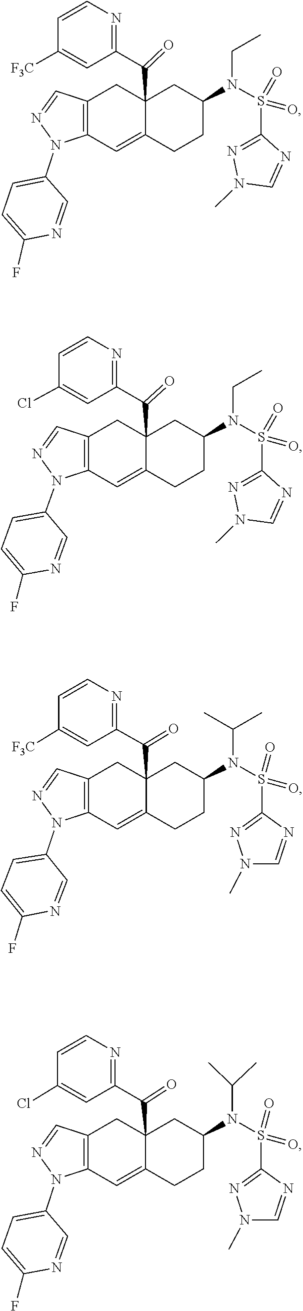 [SMILES: CCN([C@H]1CCC2=Cc3c(cnn3-c3ccc(F)nc3)C[C@]2(C(=O)c2cc(C(F)(F)F)ccn2)C1)S(=O)(=O)c1ncn(C)n1 ; CCN([C@H]1CCC2=Cc3c(cnn3-c3ccc(F)nc3)C[C@]2(C(=O)c2cc(Cl)ccn2)C1)S(=O)(=O)c1ncn(C)n1 ; CC(C)N([C@H]1CCC2=Cc3c(cnn3-c3ccc(F)nc3)C[C@]2(C(=O)c2cc(C(F)(F)F)ccn2)C1)S(=O)(=O)c1ncn(C)n1 ; CC(C)N([C@H]1CCC2=Cc3c(cnn3-c3ccc(F)nc3)C[C@]2(C(=O)c2cc(Cl)ccn2)C1)S(=O)(=O)c1ncn(C)n1]

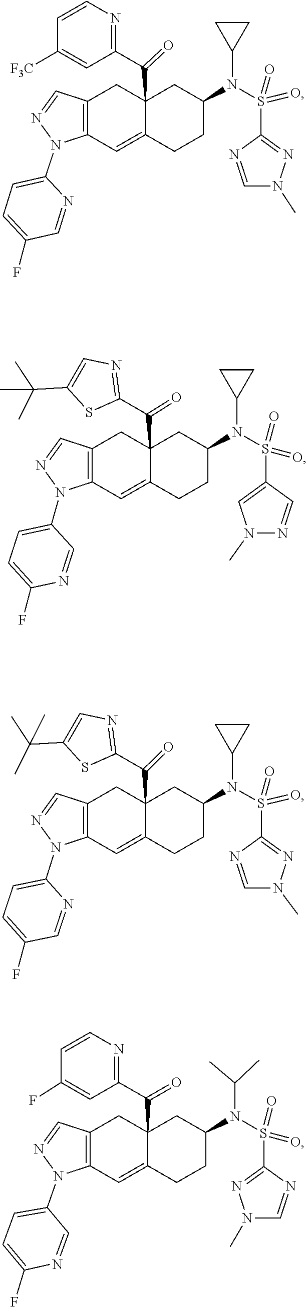 [SMILES: Cn1cnc(S(=O)(=O)N(C2CC2)[C@H]2CCC3=Cc4c(cnn4-c4ccc(F)cn4)C[C@]3(C(=O)c3cc(C(F)(F)F)ccn3)C2)n1 ; Cn1cc(S(=O)(=O)N(C2CC2)[C@H]2CCC3=Cc4c(cnn4-c4ccc(F)nc4)C[C@]3(C(=O)c3ncc(C(C)(C)C)s3)C2)cn1 ; Cn1cnc(S(=O)(=O)N(C2CC2)[C@H]2CCC3=Cc4c(cnn4-c4ccc(F)cn4)C[C@]3(C(=O)c3ncc(C(C)(C)C)s3)C2)n1 ; CC(C)N([C@H]1CCC2=Cc3c(cnn3-c3ccc(F)nc3)C[C@]2(C(=O)c2cc(F)ccn2)C1)S(=O)(=O)c1ncn(C)n1]

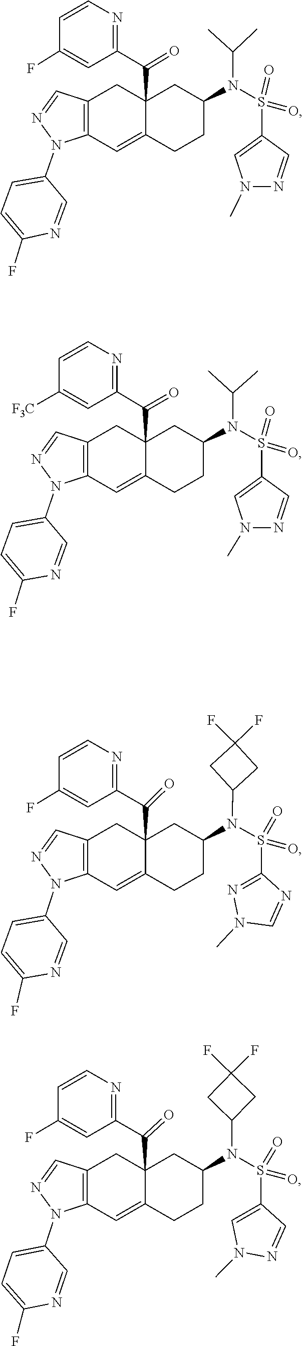 [SMILES: CC(C)N([C@H]1CCC2=Cc3c(cnn3-c3ccc(F)nc3)C[C@]2(C(=O)c2cc(F)ccn2)C1)S(=O)(=O)c1cnn(C)c1 ; CC(C)N([C@H]1CCC2=Cc3c(cnn3-c3ccc(F)nc3)C[C@]2(C(=O)c2cc(C(F)(F)F)ccn2)C1)S(=O)(=O)c1cnn(C)c1 ; Cn1cnc(S(=O)(=O)N(C2CC(F)(F)C2)[C@H]2CCC3=Cc4c(cnn4-c4ccc(F)nc4)C[C@]3(C(=O)c3cc(F)ccn3)C2)n1 ; Cn1cc(S(=O)(=O)N(C2CC(F)(F)C2)[C@H]2CCC3=Cc4c(cnn4-c4ccc(F)nc4)C[C@]3(C(=O)c3cc(F)ccn3)C2)cn1]

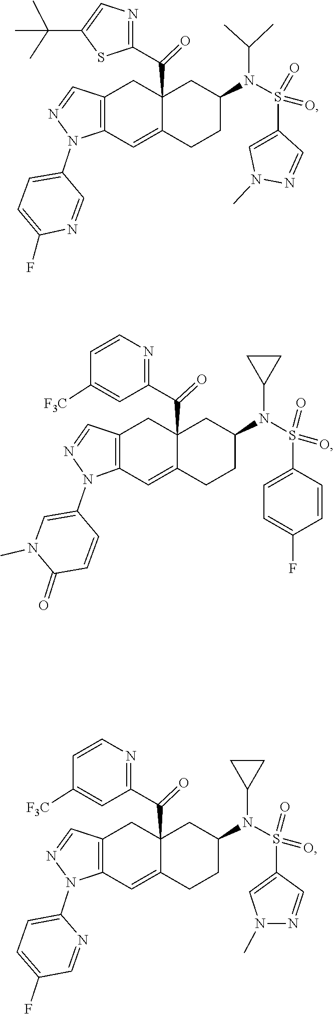

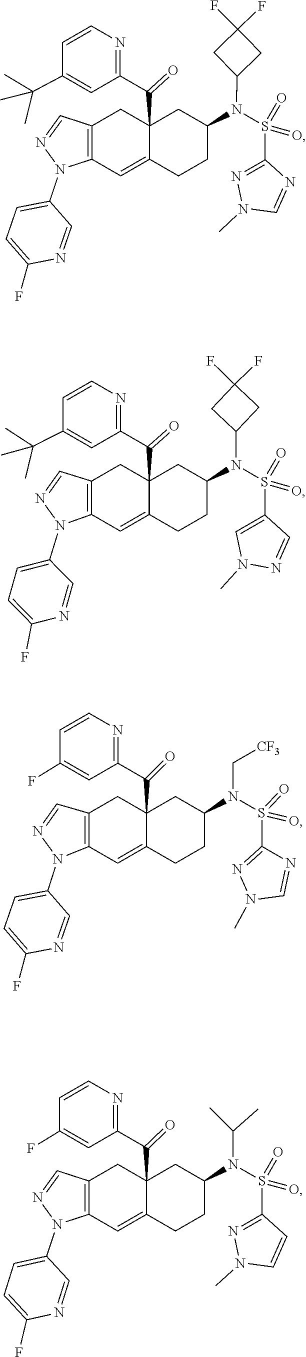

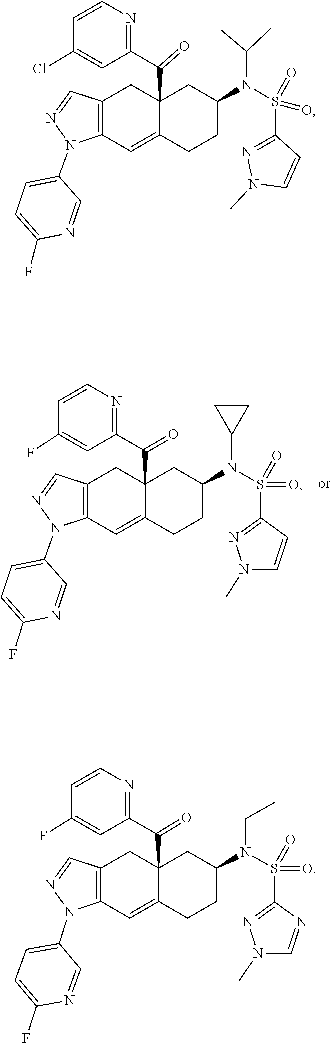 [SMILES: CC(C)N([C@H]1CCC2=Cc3c(cnn3-c3ccc(F)nc3)C[C@]2(C(=O)c2cc(Cl)ccn2)C1)S(=O)(=O)c1ccn(C)n1 ; Cn1ccc(S(=O)(=O)N(C2CC2)[C@H]2CCC3=Cc4c(cnn4-c4ccc(F)nc4)C[C@]3(C(=O)c3cc(F)ccn3)C2)n1 ; "or" ; CCN([C@H]1CCC2=Cc3c(cnn3-c3ccc(F)nc3)C[C@]2(C(=O)c2cc(F)ccn2)C1)S(=O)(=O)c1ncn(C)n1]

35. A compound, or a pharmaceutically acceptable salt, solvate, stereoisomer, or isotopic variant thereof, wherein the compound is:

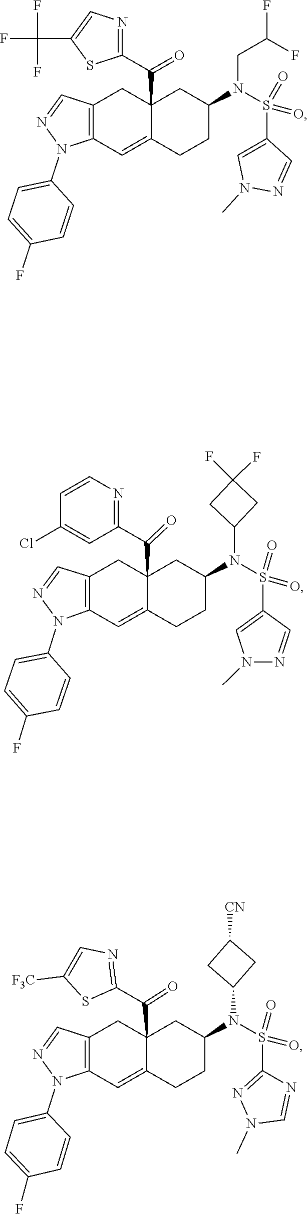

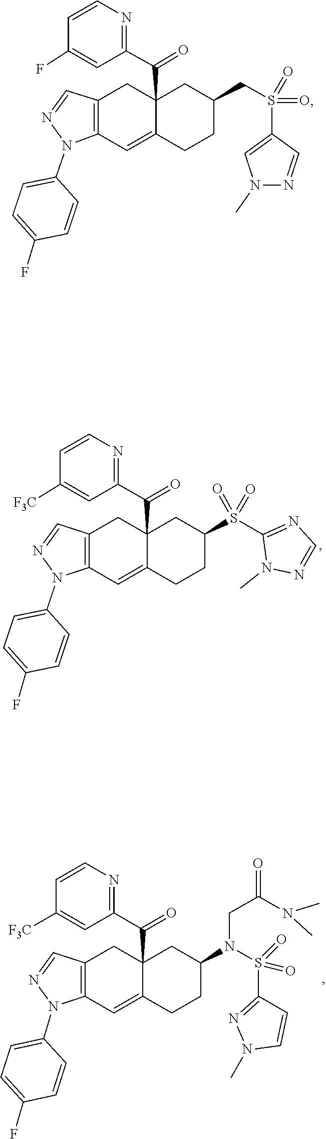

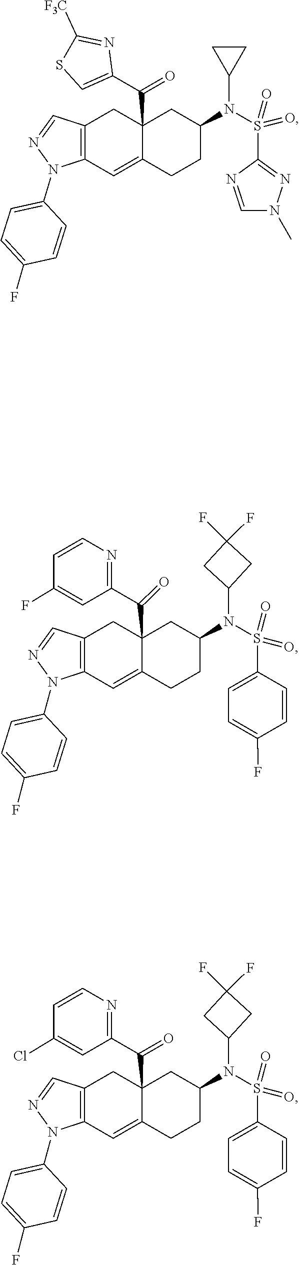 [SMILES: Cn1cnc(S(=O)(=O)N(C2CC2)[C@H]2CCC3=Cc4c(cnn4-c4ccc(F)cc4)C[C@]3(C(=O)c3csc(C(F)(F)F)n3)C2)n1 ; O=C(c1cc(F)ccn1)[C@]12Cc3cnn(-c4ccc(F)cc4)c3C=C1CC[C@H](N(C1CC(F)(F)C1)S(=O)(=O)c1ccc(F)cc1)C2 ; O=C(c1cc(Cl)ccn1)[C@]12Cc3cnn(-c4ccc(F)cc4)c3C=C1CC[C@H](N(C1CC(F)(F)C1)S(=O)(=O)c1ccc(F)cc1)C2]

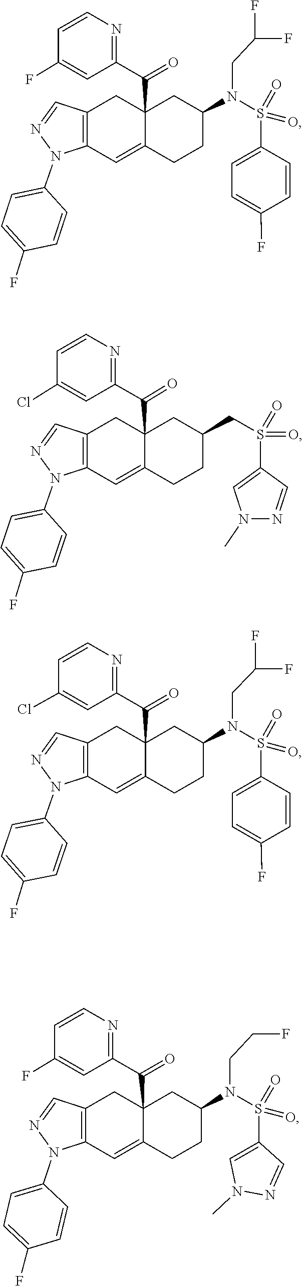 [SMILES: O=C(c1cc(F)ccn1)[C@]12Cc3cnn(-c4ccc(F)cc4)c3C=C1CC[C@H](N(CC(F)F)S(=O)(=O)c1ccc(F)cc1)C2 ; Cn1cc(S(=O)(=O)C[C@H]2CCC3=Cc4c(cnn4-c4ccc(F)cc4)C[C@]3(C(=O)c3cc(Cl)ccn3)C2)cn1 ; O=C(c1cc(Cl)ccn1)[C@]12Cc3cnn(-c4ccc(F)cc4)c3C=C1CC[C@H](N(CC(F)F)S(=O)(=O)c1ccc(F)cc1)C2 ; Cn1cc(S(=O)(=O)N(CCF)[C@H]2CCC3=Cc4c(cnn4-c4ccc(F)cc4)C[C@]3(C(=O)c3cc(F)ccn3)C2)cn1]

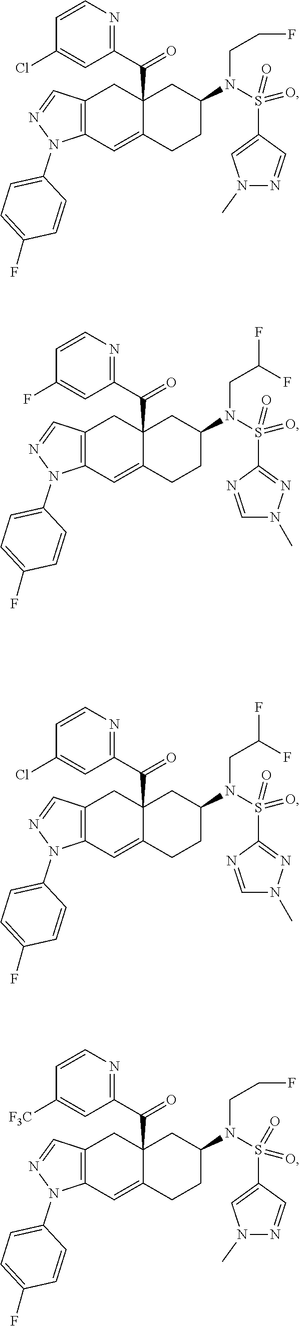 [SMILES: Cn1cc(S(=O)(=O)N(CCF)[C@H]2CCC3=Cc4c(cnn4-c4ccc(F)cc4)C[C@]3(C(=O)c3cc(Cl)ccn3)C2)cn1 ; Cn1cnc(S(=O)(=O)N(CC(F)F)[C@H]2CCC3=Cc4c(cnn4-c4ccc(F)cc4)C[C@]3(C(=O)c3cc(F)ccn3)C2)n1 ; Cn1cnc(S(=O)(=O)N(CC(F)F)[C@H]2CCC3=Cc4c(cnn4-c4ccc(F)cc4)C[C@]3(C(=O)c3cc(Cl)ccn3)C2)n1 ; Cn1cc(S(=O)(=O)N(CCF)[C@H]2CCC3=Cc4c(cnn4-c4ccc(F)cc4)C[C@]3(C(=O)c3cc(C(F)(F)F)ccn3)C2)cn1]

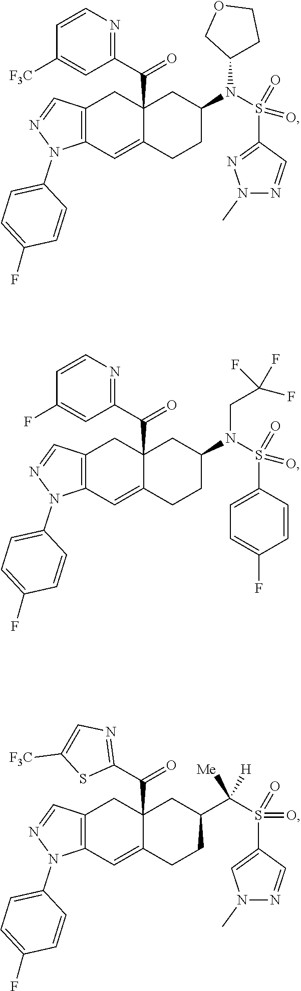 [SMILES: Cn1ncc(S(=O)(=O)N([C@H]2CCOC2)[C@H]2CCC3=Cc4c(cnn4-c4ccc(F)cc4)C[C@]3(C(=O)c3cc(C(F)(F)F)ccn3)C2)n1 ; O=C(c1cc(F)ccn1)[C@]12Cc3cnn(-c4ccc(F)cc4)c3C=C1CC[C@H](N(CC(F)(F)F)S(=O)(=O)c1ccc(F)cc1)C2 ; C[C@H]([C@H]1CCC2=Cc3c(cnn3-c3ccc(F)cc3)C[C@]2(C(=O)c2ncc(C(F)(F)F)s2)C1)S(=O)(=O)c1cnn(C)c1]

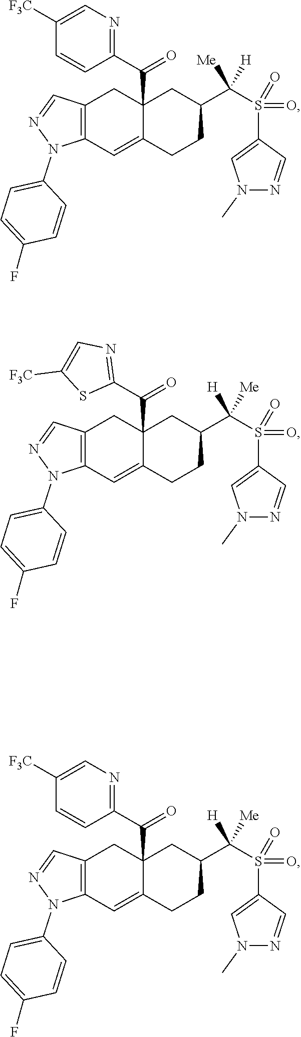 [SMILES: C[C@H]([C@H]1CCC2=Cc3c(cnn3-c3ccc(F)cc3)C[C@]2(C(=O)c2ccc(C(F)(F)F)cn2)C1)S(=O)(=O)c1cnn(C)c1 ; C[C@@H]([C@H]1CCC2=Cc3c(cnn3-c3ccc(F)cc3)C[C@]2(C(=O)c2ncc(C(F)(F)F)s2)C1)S(=O)(=O)c1cnn(C)c1 ; C[C@@H]([C@H]1CCC2=Cc3c(cnn3-c3ccc(F)cc3)C[C@]2(C(=O)c2ccc(C(F)(F)F)cn2)C1)S(=O)(=O)c1cnn(C)c1]

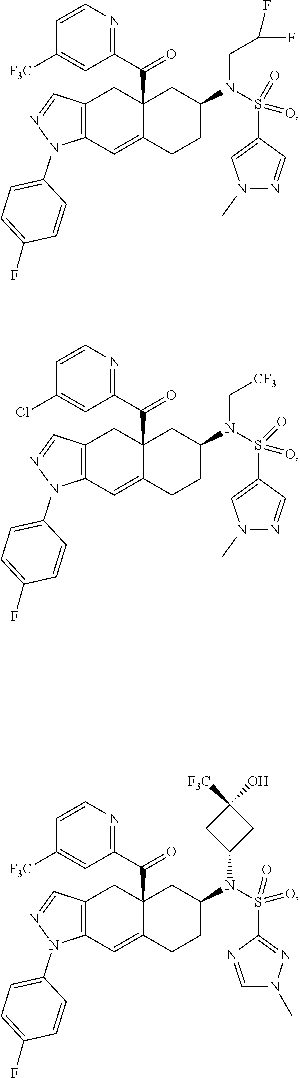 [SMILES: Cn1cc(S(=O)(=O)N(CC(F)F)[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)N(CC(F)(F)F)[C@H]2CCC3=Cc4c(cnn4-c4ccc(F)cc4)C[C@]3(C(=O)c3cc(Cl)ccn3)C2)cn1 ; Cn1cnc(S(=O)(=O)N([C@H]2CCC3=Cc4c(cnn4-c4ccc(F)cc4)C[C@]3(C(=O)c3cc(C(F)(F)F)ccn3)C2)[C@H]2C[C@](O)(C(F)(F)F)C2)n1]

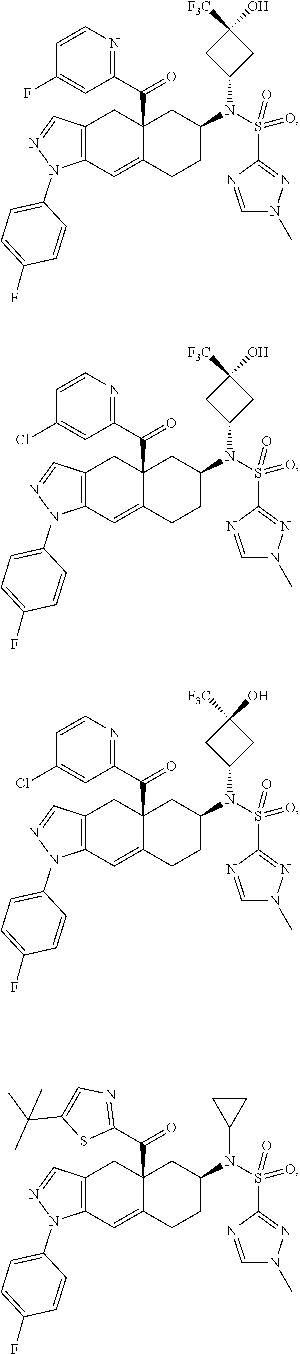 [SMILES: Cn1cnc(S(=O)(=O)N([C@H]2CCC3=Cc4c(cnn4-c4ccc(F)cc4)C[C@]3(C(=O)c3cc(F)ccn3)C2)[C@H]2C[C@](O)(C(F)(F)F)C2)n1 ; Cn1cnc(S(=O)(=O)N([C@H]2CCC3=Cc4c(cnn4-c4ccc(F)cc4)C[C@]3(C(=O)c3cc(Cl)ccn3)C2)[C@H]2C[C@](O)(C(F)(F)F)C2)n1 ; Cn1cnc(S(=O)(=O)N([C@H]2CCC3=Cc4c(cnn4-c4ccc(F)cc4)C[C@]3(C(=O)c3cc(Cl)ccn3)C2)[C@H]2C[C@@](O)(C(F)(F)F)C2)n1 ; Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)n1]

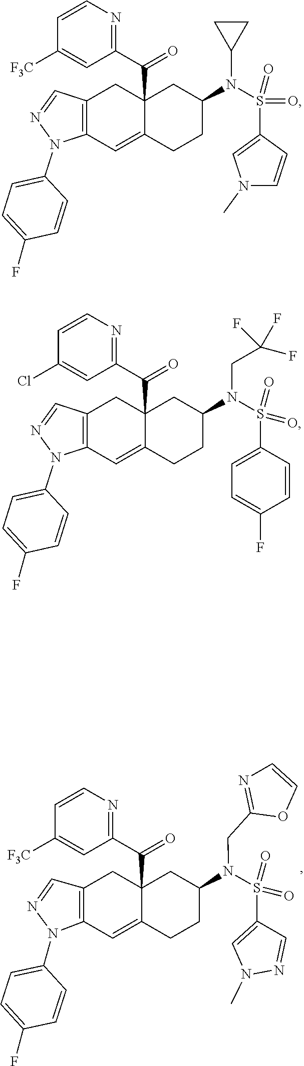

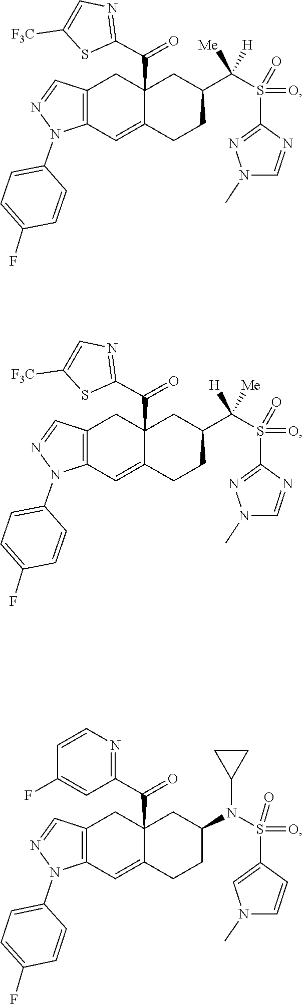 [SMILES: C[C@H]([C@H]1CCC2=Cc3c(cnn3-c3ccc(F)cc3)C[C@]2(C(=O)c2ncc(C(F)(F)F)s2)C1)S(=O)(=O)c1ncn(C)n1 ; C[C@@H]([C@H]1CCC2=Cc3c(cnn3-c3ccc(F)cc3)C[C@]2(C(=O)c2ncc(C(F)(F)F)s2)C1)S(=O)(=O)c1ncn(C)n1 ; Cn1ccc(S(=O)(=O)N(C2CC2)[C@H]2CCC3=Cc4c(cnn4-c4ccc(F)cc4)C[C@]3(C(=O)c3cc(F)ccn3)C2)c1]

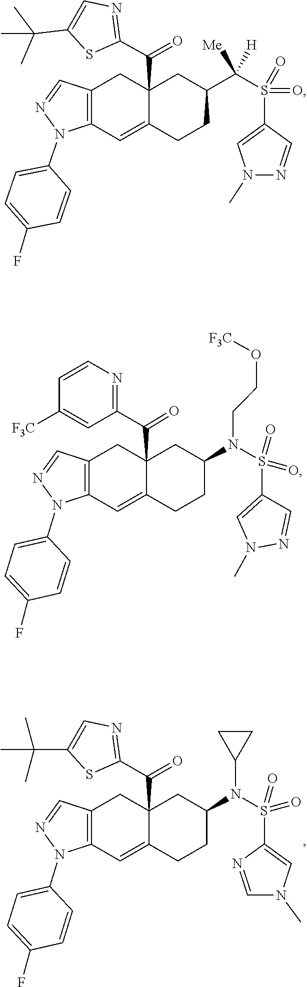 [SMILES: C[C@H]([C@H]1CCC2=Cc3c(cnn3-c3ccc(F)cc3)C[C@]2(C(=O)c2ncc(C(C)(C)C)s2)C1)S(=O)(=O)c1cnn(C)c1 ; Cn1cc(S(=O)(=O)N(CCOC(F)(F)F)[C@H]2CCC3=Cc4c(cnn4-c4ccc(F)cc4)C[C@]3(C(=O)c3cc(C(F)(F)F)ccn3)C2)cn1 ; Cn1cnc(S(=O)(=O)N(C2CC2)[C@H]2CCC3=Cc4c(cnn4-c4ccc(F)cc4)C[C@]3(C(=O)c3ncc(C(C)(C)C)s3)C2)c1]

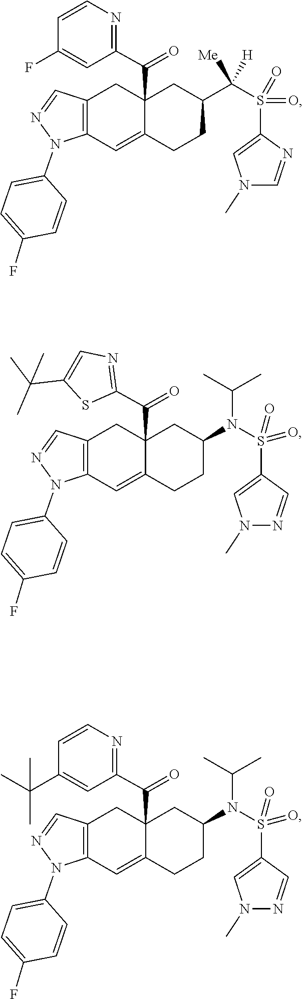 [SMILES: C[C@H]([C@H]1CCC2=Cc3c(cnn3-c3ccc(F)cc3)C[C@]2(C(=O)c2cc(F)ccn2)C1)S(=O)(=O)c1cn(C)cn1 ; CC(C)N([C@H]1CCC2=Cc3c(cnn3-c3ccc(F)cc3)C[C@]2(C(=O)c2ncc(C(C)(C)C)s2)C1)S(=O)(=O)c1cnn(C)c1 ; CC(C)N([C@H]1CCC2=Cc3c(cnn3-c3ccc(F)cc3)C[C@]2(C(=O)c2cc(C(C)(C)C)ccn2)C1)S(=O)(=O)c1cnn(C)c1]

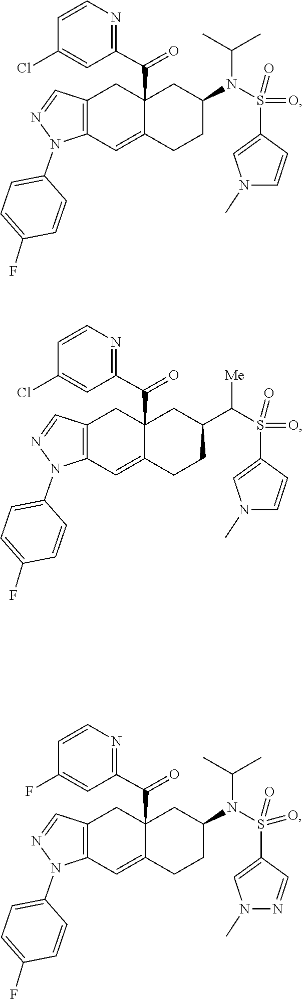 [SMILES: CC(C)N([C@H]1CCC2=Cc3c(cnn3-c3ccc(F)cc3)C[C@]2(C(=O)c2cc(Cl)ccn2)C1)S(=O)(=O)c1ccn(C)c1 ; CC([C@H]1CCC2=Cc3c(cnn3-c3ccc(F)cc3)C[C@]2(C(=O)c2cc(Cl)ccn2)C1)S(=O)(=O)c1ccn(C)c1 ; CC(C)N([C@H]1CCC2=Cc3c(cnn3-c3ccc(F)cc3)C[C@]2(C(=O)c2cc(F)ccn2)C1)S(=O)(=O)c1cnn(C)c1]

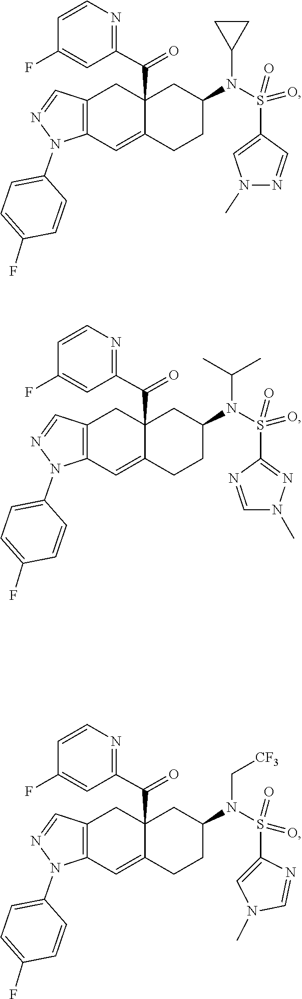 [SMILES: Cn1cc(S(=O)(=O)N(C2CC2)[C@H]2CCC3=Cc4c(cnn4-c4ccc(F)cc4)C[C@]3(C(=O)c3cc(F)ccn3)C2)cn1 ; CC(C)N([C@H]1CCC2=Cc3c(cnn3-c3ccc(F)cc3)C[C@]2(C(=O)c2cc(F)ccn2)C1)S(=O)(=O)c1ncn(C)n1 ; Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)c1]

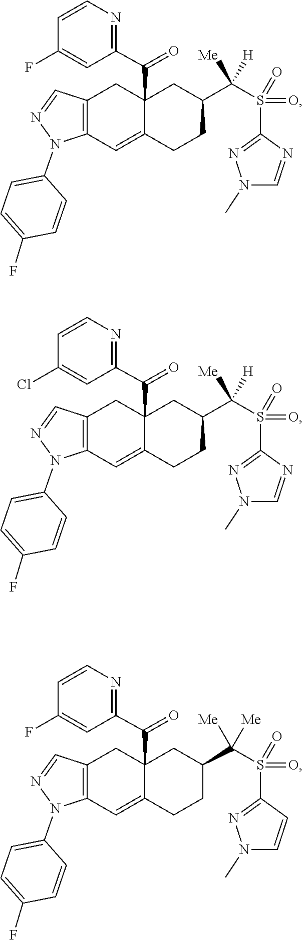 [SMILES: C[C@H]([C@H]1CCC2=Cc3c(cnn3-c3ccc(F)cc3)C[C@]2(C(=O)c2cc(F)ccn2)C1)S(=O)(=O)c1ncn(C)n1 ; C[C@H]([C@H]1CCC2=Cc3c(cnn3-c3ccc(F)cc3)C[C@]2(C(=O)c2cc(Cl)ccn2)C1)S(=O)(=O)c1ncn(C)n1 ; Cn1ccc(S(=O)(=O)C(C)(C)[C@H]2CCC3=Cc4c(cnn4-c4ccc(F)cc4)C[C@]3(C(=O)c3cc(F)ccn3)C2)n1]

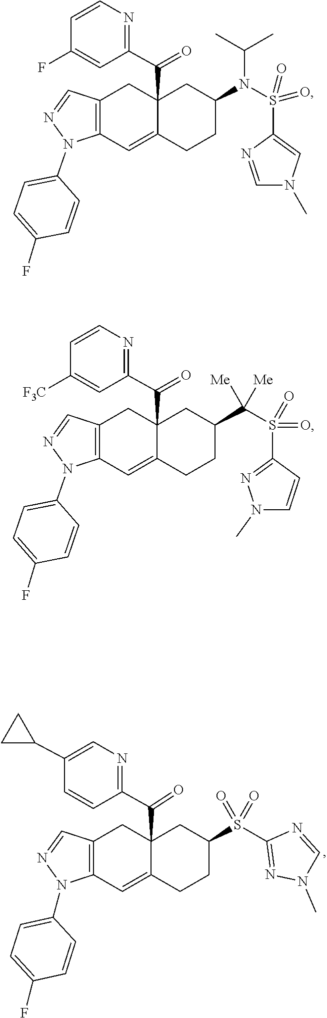 [SMILES: CC(C)N([C@H]1CCC2=Cc3c(cnn3-c3ccc(F)cc3)C[C@]2(C(=O)c2cc(F)ccn2)C1)S(=O)(=O)c1cn(C)cn1 ; Cn1ccc(S(=O)(=O)C(C)(C)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)[C@H]2CCC3=Cc4c(cnn4-c4ccc(F)cc4)C[C@]3(C(=O)c3ccc(C4CC4)cn3)C2)n1]

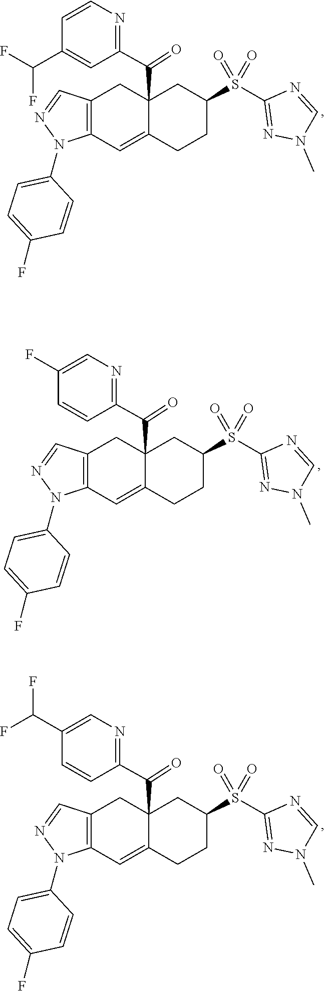 [SMILES: Cn1cnc(S(=O)(=O)[C@H]2CCC3=Cc4c(cnn4-c4ccc(F)cc4)C[C@]3(C(=O)c3cc(C(F)F)ccn3)C2)n1 ; Cn1cnc(S(=O)(=O)[C@H]2CCC3=Cc4c(cnn4-c4ccc(F)cc4)C[C@]3(C(=O)c3ccc(F)cn3)C2)n1 ; Cn1cnc(S(=O)(=O)[C@H]2CCC3=Cc4c(cnn4-c4ccc(F)cc4)C[C@]3(C(=O)c3ccc(C(F)F)cn3)C2)n1]

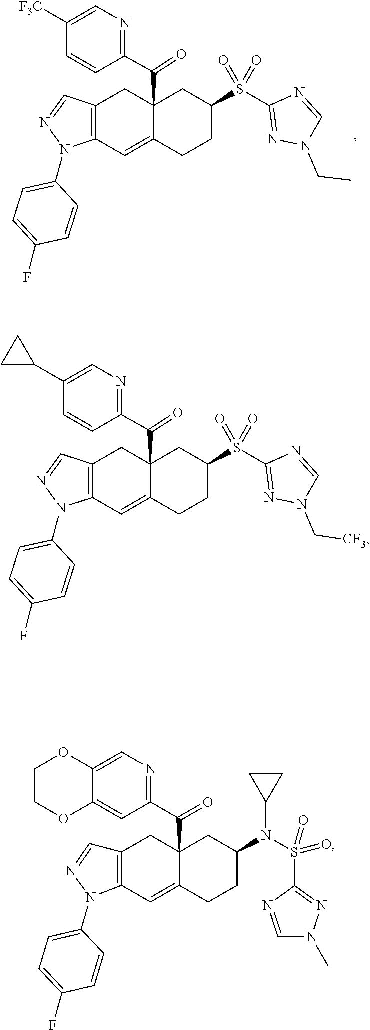

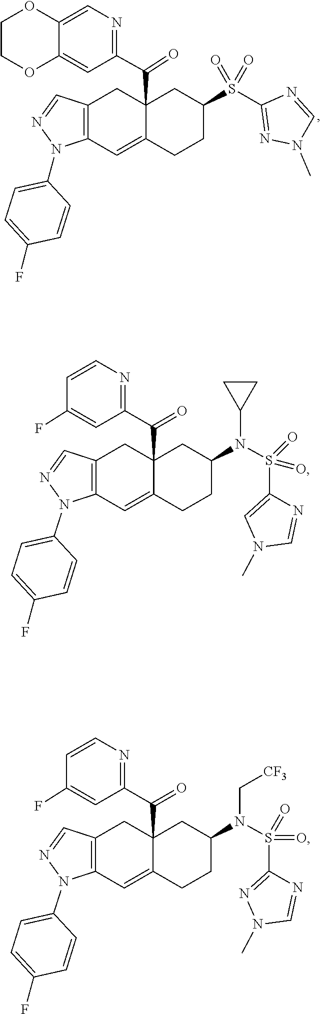 [SMILES: Cn1cnc(S(=O)(=O)[C@H]2CCC3=Cc4c(cnn4-c4ccc(F)cc4)C[C@]3(C(=O)c3cc4c(cn3)OCCO4)C2)n1 ; Cn1cnc(S(=O)(=O)N(C2CC2)[C@H]2CCC3=Cc4c(cnn4-c4ccc(F)cc4)C[C@]3(C(=O)c3cc(F)ccn3)C2)c1 ; Cn1cnc(S(=O)(=O)N(CC(F)(F)F)[C@H]2CCC3=Cc4c(cnn4-c4ccc(F)cc4)C[C@]3(C(=O)c3cc(F)ccn3)C2)n1]

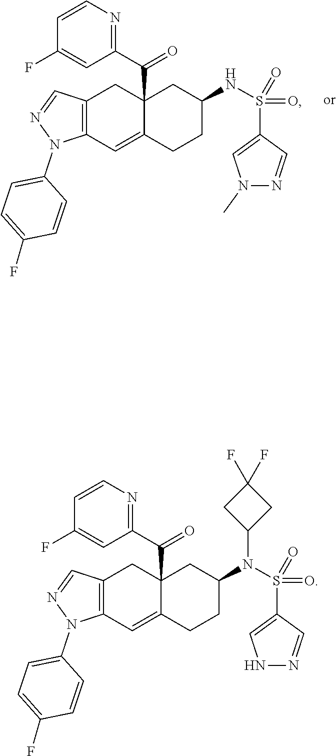 [SMILES: Cn1cc(S(=O)(=O)N[C@H]2CCC3=Cc4c(cnn4-c4ccc(F)cc4)C[C@]3(C(=O)c3cc(F)ccn3)C2)cn1 ; "or" ; O=C(c1cc(F)ccn1)[C@]12Cc3cnn(-c4ccc(F)cc4)c3C=C1CC[C@H](N(C1CC(F)(F)C1)S(=O)(=O)c1cn[nH]c1)C2]

36. A pharmaceutical composition comprising a compound of any one of claims 1-35, or a pharmaceutically acceptable salt, solvate, stereoisomer, or isotopic variant thereof, and at least one pharmaceutically acceptable excipient.

37. A method for treating or preventing cancer in a subject, the method comprising administering a therapeutically effective amount of a compound of any one of claims 1-35, or a pharmaceutically acceptable salt, solvate, stereoisomer, or isotopic variant thereof, to the subject in need thereof.

38. A method of reducing incidences of cancer recurrence, the method comprising administering to a subject in cancer remission a therapeutically effective amount of a compound of any one of claims 1-35, or a pharmaceutically acceptable salt, solvate, stereoisomer, or isotopic variant thereof.

39. A method for treating a therapy-resistant cancer in a subject, the method comprising administering a therapeutically effective amount of a compound of any one of claims 1-35, or a pharmaceutically acceptable salt, solvate, stereoisomer, or isotopic variant thereof, to the subject in need thereof.

40. The method of any one of claims 37-39, wherein the cancer is triple negative breast cancer, ovarian cancer, castration resistant prostate cancer, or doubly resistant prostate cancer.

41. The method of any one of claims 37-39, wherein the cancer is non-small cell lung cancer, clear renal cell carcinoma, hepatocellular carcinoma, melanoma, or bladder cancer.

42. The method of any one of claims 37-41, further comprising administering one or more additional therapeutic agents to the subject.

43. The method of claim 42, wherein the one or more additional therapeutic agents are androgen receptor signaling inhibitors.

44. The method of claim 43, wherein the androgen receptor signaling inhibitor is 3,3′-diindolylmethane (DIM), abiraterone acetate, apalutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof.

45. The method of claim 42, wherein the one or more additional therapeutic agents are chemotherapeutic agents.

46. The method of claim 45, wherein the chemotherapeutic agents are cisplatin, carboplatin, paclitaxel, docetaxel, nab-paclitaxel, gemcitabine, doxorubicin, camptothecin, topotecan, pemetrexed, or a combination thereof.

47. The method of claim 42, wherein the one or more additional therapeutic agents are anti-PD-L1 agents or anti-PD 1 agents, anti-CTLA-4 agents, CAR-T cells therapy, cancer vaccines, or IDO-1 inhibitors.

48. A method for treating a hypercortisolism disease or disorder in a subject, the method comprising administering a therapeutically effective amount a compound of any one of claims 1-35, or a pharmaceutically acceptable salt, solvate, stereoisomer, or isotopic variant thereof, to the subject in need thereof.

49. The method of claim 48, wherein the hypercortisolism disease or disorder is Cushing's syndrome.
